SEQUENCE LISTING (370 sequences)

SEQ ID NO: 1 - DNA, 36519 bp, synthetic construct
  source 1..36519: mol_type = other DNA
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagatgaggcacctgagagacctgcccgatgagaaaatcatcatcgcttccgggaacgagattctggaactggtggtaaatgccatgatgggcgacgaccctccggagccccccaccccatttgagacaccttcgctgcacgatttgtatgatctggaggtggatgtgcccgaggacgatcccaatgaggaggcggtaaatgatttttttagcgatgccgcgctgctagctgccgaggaggcttcgagctctagctcagacagcgactcttcactgcatacccctagacccggcagaggtgagaaaaagatccccgagcttaaaggggaagagatggacttgcgctgctatgaggaatgcttgcccccgagcgatgatgaggacgagcaggcgatccagaacgcagcgagccagggagtgcaagccgccagcgagagctttgcgctggactgcccgcctctgcccggacacggctgtaagtcttgtgaatttcatcgcatgaatactggagataaagctgtgttgtgtgcactttgctatatgagagcttacaaccattgtgtttacagtaagtgtgattaagttgaactttagagggaggcagagagcagggtgactgggcgatgactggtttatttatgtatatatgttctttatataggtcccgtctctgacgcagatgatgagacccccactacaaagtccacttcgtcacccccagaaattggcacatctccacctgagaatattgttagaccagttcctgttagagccactgggaggagagcagctgtggaatgtttggatgacttgctacagggtggggttgaacctttggacttgtgtacccggaaacgccccaggcactaagtgccacacatgtgtgtttacttgaggtgatgtcagtatttatagggtgtggagtgcaataaaaaatgtgttgactttaagtgcgtggtttatgactcaggggtggggactgtgagtatataagcaggtgcagacctgtgtggttagctcagagcggcatggagatttggacggtcttggaagactttcacaagactagacagctgctagagaacgcctcgaacggagtctcttacctgtggagattctgcttcggtggcgacctagctaggctagtctacagggccaaacaggattatagtgaacaatttgaggttattttgagagagtgttctggtctttttgacgctcttaacttgggccatcagtctcactttaaccagaggatttcgagagcccttgattttactactcctggcagaaccactgcagcagtagccttttttgcttttattcttgacaaatggagtcaagaaacccatttcagcagggattaccagctggatttcttagcagtagctttgtggagaacatggaagtgccagcgcctgaatgcaatctccggctacttgccggtacagccgctagacactctgaggatcctgaatctccaggagagtcccagggcacgccaacgtcgccagcagcagcagcaggaggaggatcaagaagagaacccgagagccggcctggaccctccggcggaggaggaggagtagctgacctgtttcctgaactgcgccgggtgctgactaggtcttcgagtggtcgggagagggggattaagcgggagaggcatgatgagactaatcacagaactgaactgactgtgggtctgatgagtcgcaagcgcccagaaacagtgtggtggcatgaggtgcagtcgactggcacagatgaggtgtcggtgatgcatgagaggttttctctagaacaagtcaagacttgttggttagagcctgaggatgattgggaggtagccatcaggaattatgccaagctggctctgaggccagacaagaagtacaagattactaagctgataaatatcagaaatgcctgctacatctcagggaatggggctgaagtggagatctgtctccaggaaagggtggctttcagatgctgcatgatgaatatgtacccgggagtggtgggcatggatggggttacctttatgaacatgaggttcaggggagatgggtataatggcacggtctttatggccaataccaagctgacagtccatggctgctccttctttgggtttaataacacctgcatcgaggcctggggtcaggtcggtgtgaggggctgcagtttttcagccaactggatgggggtcgtgggcaggaccaagagtatgctgtccgtgaagaaatgcttgtttgagaggtgccacctgggggtgatgagcgagggcgaagccagaatccgccactgcgcctctaccgagacgggctgctttgtgctgtgcaagggcaatgctaagatcaagcataatatgatctgtggagcctcggacgagcgcggctaccagatgctgacctgcgccggcgggaacagccatatgctggccaccgtacatgtggcttcccatgctcgcaagccctggcccgagttcgagcacaatgtcatgaccaggtgcaatatgcatctggggtcccgccgaggcatgttcatgccctaccagtgcaacctgaattatgtgaaggtgctgctggagcccgatgccatgtccagagtgagcctgacgggggtgtttgacatgaatgtggaggtgtggaagattctgagatatgatgaatccaagaccaggtgccgagcctgcgagtgcggagggaagcatgccaggttccagcccgtgtgtgtggatgtgacggaggacctgcgacccgatcatttggtgttgccctgcaccgggacggagttcggttccagcggggaagaatctgactagagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacagacccttctgactctgtactgcatctgcaaccaccccggcctgcatgaaagtctttgttgtctgctgtgtactgagtataataaaagctgagatcagcgactactccggacttccgtgtgttcctgaatccatcaaccagtctttgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgtcgctccccgctactaacaaccaaactaacctccaccaacgccaccgtcgcgacctttctgaatctaatactaccacccacaccggaggtgagctccgaggtcaaccaacctctgggatttactacggcccctgggaggtggttgggttaatagcgctaggcctagttgcgggtgggcttttggttctctgctacctatacctcccttgctgttcgtacttagtggtgctgtgttgctggtttaagaaatggggaagatcaccctagtgagctgcggtgcgctggtggcggtgttgctttcgattgtgggactgggcggtgcggctgtagtgaaggagaaggccgatccctgcttgcatttcaatcccaacaaatgccagctgagttttcagcccgatggcaatcggtgcgcggtactgatcaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcgcacatgtgcgacacggtcatgtggatgagcaagcagtacgatatgtggccccccacgaaggagaacatcgtggtcttctccatcgcttacagcctgtgcacggcgctaatcaccgctatcgtgtgcctgagcattcacatgctcatcgctattcgccccagaaataatgccgaaaaagaaaaacagccataacgttttttttcacacctttttcagaccatggcctctgttaaatttttgcttttatttgccagtctcattgccgtcattcatggaatgagtaatgagaaaattactatttacactggcactaatcacacattgaaaggtccagaaaaagccacagaagtttcatggtattgttattttaatgaatcagatgtatctactgaactctgtggaaacaataacaaaaaaaatgagagcattactctcatcaagtttcaatgtggatctgacttaaccctaattaacatcactagagactatgtaggtatgtattatggaactacagcaggcatttcggacatggaattttatcaagtttctgtgtctgaacccaccacgcctagaatgaccacaaccacaaaaactacacctgttaccactatgcagctcactaccaataacatttttgccatgcgtcaaatggtcaacaatagcactcaacccaccccacccagtgaggaaattcccaaatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttaattttttctatcattacctctgctctatgcaattctgacaatgaggacgttactgtcgttgtcggatcaaattatacactgaaaggtccagcgaagggtatgctttcgtggtattgctattttggatctgacactacagaaactgaattatgcaatcttaagaatggcaaaattcaaaattctaaaattaacaattatatatgcaatggtactgatctgatactcctcaatatcacgaaatcatatgctggcagttacacctgccctggagatgatgctgacagtatgattttttacaaagtaactgttgttgatcccactactccacctccacccaccacaactactcacaccacacacacagatcaaaccgcagcagaggaggcagcaaagttagccttgcaggtccaagacagttcatttgttggcattacccctacacctgatcagcggtgtccggggctgctagtcagcggcattgtcggtgtgctttcgggattagcagtcataatcatctgcatgttcatttttgcttgctgctatagaaggctttaccgacaaaaatcagacccactgctgaacctctatgtttaattttttccagagtcatgaaggcagttagcgctctagttttttgttctttgattggcattgttttttgcaatcctattcctaaagttagctttattaaagatgtgaatgttactgaggggggcaatgtgacactggtaggtgtagagggtgctgaaaacaccacctggacaaaataccacctcaatgggtggaaagatatttgcaattggagtgtattagtttatacatgtgagggagttaatcttaccattgtcaatgccacctcagctcaaaatggtagaattcaaggacaaagtgtcagtgtatctaatgggtattttacccaacatacttttatctatgacgttaaagtcataccactgcctacgcctagcccacctagcactaccacacagacaacccacactacacagacaaccacatacagtacattaaatcagcctaccaccactacagcagcagaggttgccagctcgtctggggtccgagtggcatttttgatgtgggccccatctagcagtcccactgctagtaccaatgagcagactactgaatttttgtccactgtcgagagccacaccacagctacctccagtgccttctctagcaccgccaatctctcctcgctttcctctacaccaatcagtcccgctactactcctagccccgctcctcttcccactcccctgaagcaaacagacggcggcatgcaatggcagatcaccctgctcattgtgatcgggttggtcatcctggccgtgttgctctactacatcttctgccgccgcattcccaacgcgcaccgcaagccggtctacaagcccatcattgtcgggcagccggagccgcttcaggtggaagggggtctaaggaatcttctcttctcttttacagtatggtgattgaactatgattcctagacaattcttgatcactattcttatctgcctcctccaagtctgtgccaccctcgctctggtggccaacgccagtccagactgtattgggcccttcgcctcctacgtgctctttgccttcaccacctgcatctgctgctgtagcatagtctgcctgcttatcaccttcttccagttcattgactggatctttgtgcgcatcgcctacctgcgccaccacccccagtaccgcgaccagcgagtggcgcggctgctcaggctcctctgataagcatgcgggctctgctacttctcgcgcttctgctgttagtgctcccccgtcccgtcgacccccggtcccccacccagtcccccgaggaggtccgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcaacctcaggcacacgcactaccaccactacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggatgcggtggccatccaccagtgcaagagaggcatcttctgcctggtgaaacaggccaagatctcctacgaggtcactccaaacgaccatcgcctctcctacgagctcctgcagcagcgccagaagttcacctgcctggtcggagtcaaccccatcgtcatcacccagcagtctggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

SEQ ID NO: 2 - DNA, 31588 bp, synthetic construct
  source 1..31588: mol_type = other DNA
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatggccgggatgttccaggcactgtccgaaggctgcacaccctatgatattaaccagatgctgaatgtcctgggagaccaccaggtctctggcctggagcagctggagagcatcatcaacttcgagaagctgaccgagtggacaagctccaatgtgatgcctatcctgtccccactgaccaagggcatcctgggcttcgtgtttaccctgacagtgccttctgagcggggcctgtcttgcatcagcgaggcagacgcaaccacaccagagtccgccaatctgggcgaggagatcctgtctcagctgtacctgtggccccgggtgacatatcactccccttcttacgcctatcaccagttcgagcggagagccaagtacaagagacacttcccaggctttggccagtctctgctgttcggctaccccgtgtacgtgttcggcgattgcgtgcagggcgactgggatgccatccggtttagatactgcgcaccacctggatatgcactgctgaggtgtaacgacaccaattattccgccctgctggcagtgggcgccctggagggccctcgcaatcaggattggctgggcgtgccaaggcagctggtgacacgcatgcaggccatccagaacgcaggcctgtgcaccctggtggcaatgctggaggagacaatcttctggctgcaggcctttctgatggccctgaccgacagcggccccaagacaaacatcatcgtggattcccagtacgtgatgggcatctccaagccttctttccaggagtttgtggactgggagaacgtgagcccagagctgaattccaccgatcagccattctggcaggcaggaatcctggcaaggaacctggtgcctatggtggccacagtgcagggccagaatctgaagtaccagggccagagcctggtcatcagcgcctccatcatcgtgtttaacctgctggagctggagggcgactatcgggacgatggcaacgtgtgggtgcacaccccactgagccccagaacactgaacgcctgggtgaaggccgtggaggagaagaagggcatcccagtgcacctggagctggcctccatgaccaatatggagctgatgtctagcatcgtgcaccagcaggtgaggacatacggacccgtgttcatgtgcctgggaggcctgctgaccatggtggcaggagccgtgtggctgacagtgcgggtgctggagctgttcagagccgcccagctggccaacgatgtggtgctgcagatcatggagctgtgcggagcagcctttcgccaggtgtgccacaccacagtgccatggcccaatgcctccctgacccccaagtggaacaatgagacaacacagcctcagatcgccaactgtagcgtgtacgacttcttcgtgtggctgcactactatagcgtgagggataccctgtggccccgcgtgacataccacatgaataagtacgcctatcacatgctggagaggcgcgccaagtataagagaggccctggcccaggcgcaaagtttgtggcagcatggaccctgaaggccgccgccggccccggccccggccagtatatcaaggctaacagtaagttcattggaatcacagagctgggacccggacctggataatgagtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgagg

SEQ ID NO: 3 - RNA, 11447 nt, Venezuelan equine encephalitis virus
  source 1..11447: mol_type = unassigned RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaaggagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactccatacatctcgcccgtgtcacattgtggatgggcacggttatttcctgcttgccaggtgcccggcaggggactccatcaccatggaatttaagaaagattccgtcacacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgttgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataacctttggtttcagatcagtgtcactgaaactgcaccctaagaatcccacatatctaaccacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaattaactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctcagatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 4 - RNA, 9577 nt, synthetic construct
  source 1..9577: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaagccaccatggcaggcatgtttcaggcgctgagcgaaggctgcaccccgtatgatattaaccagatgctgaacgtgctgggcgatcatcaggtctcaggccttgagcagcttgagagtataatcaactttgaaaaactgactgaatggaccagttctaatgttatgcctatcctgtctcctctgacaaagggcatcctgggcttcgtgtttaccctgaccgtgccttctgagagaggacttagctgcattagcgaagcggatgcgaccaccccggaaagcgcgaacctgggcgaagaaattctgagccagctgtatctttggccaagggtgacctaccattcccctagttatgcttaccaccaatttgaaagacgagccaaatataaaagacacttccccggctttggccagagcctgctgtttggctaccctgtgtacgtgttcggcgattgcgtgcagggcgattgggatgcgattcgctttcgctattgcgcgccgccgggctatgcgctgctgcgctgcaacgataccaactatagcgctctgctggctgtgggggccctagaaggacccaggaatcaggactggcttggtgtcccaagacaacttgtaactcggatgcaggctattcagaatgccggcctgtgtaccctggtggccatgctggaagagacaatcttctggctgcaagcgtttctgatggcgctgaccgatagcggcccgaaaaccaacattattgtggatagccagtatgtgatgggcattagcaaaccgagctttcaggaatttgtggattgggaaaacgtgagcccggaactgaacagcaccgatcagccgttttggcaagccggaatcctggccagaaatctggtgcctatggtggccacagtgcagggccagaacctgaagtaccagggtcagtcactagtcatctctgcttctatcattgtcttcaacctgctggaactggaaggtgattatcgagatgatggcaacgtgtgggtgcataccccgctgagcccgcgcaccctgaacgcgtgggtgaaagcggtggaagaaaaaaaaggtattccagttcacctagagctggccagtatgaccaacatggagctcatgagcagtattgtgcatcagcaggtcagaacatacggccccgtgttcatgtgtctcggcggactgcttacaatggtggctggtgctgtgtggctgacagtgcgagtgctcgagctgttccgggccgcgcagctggccaacgacgtggtcctccagatcatggagctttgtggtgcagcgtttcgccaggtgtgccataccaccgtgccgtggccgaacgcgagcctgaccccgaaatggaacaacgaaaccacccagccccagatcgccaactgcagcgtgtatgacttttttgtgtggctccattattattctgttcgagacacactttggccaagggtgacctaccatatgaacaaatatgcgtatcatatgctggaaagacgagccaaatataaaagaggaccaggacctggcgctaaatttgtggccgcctggacactgaaagccgctgctggtcctggacctggccagtacatcaaggccaacagcaagttcatcggcatcaccgaactcggacccggaccaggctgatgattcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - DNA, 11447 bp, Venezuelan equine encephalitis virus
  source 1..11447: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaaggagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactccatacatctcgcccgtgtcacattgtggatgggcacggttatttcctgcttgccaggtgcccggcaggggactccatcaccatggaatttaagaaagattccgtcacacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgttgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataacctttggtttcagatcagtgtcactgaaactgcaccctaagaatcccacatatctaaccacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaattaactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctcagatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 6 - DNA, 7894 bp, synthetic construct
  source 1..7894: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 7893 bp, synthetic construct
  source 1..7893: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 7927 bp, synthetic construct
  source 1..7927: mol_type = other DNA
taatacgactcactataggatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaatacgtagtttaaac

SEQ ID NO: 9 - DNA, 7926 bp, synthetic construct
  source 1..7926: mol_type = other DNA
taatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaatacgtagtttaaac

SEQ ID NO: 10-13 - skipped (4 entries)

SEQ ID NO: 14 - DNA, 8602 bp, synthetic construct
  source 1..8602: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaaagtccgccatatgaggccaccatgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggcgctgcttacagtataatcaactttgaaaaactggctgcttacggcatcctgggctttgtgtttacactggctgcctacctgctgtttggctatcctgtgtacgtggccgcttatggactgtgtaccctggtggccatgctggctgcttacaatctggtgcctatggtggccacagtggccgcctattgtcttggcggactgctgacaatggtggcagcctacagcccgagctatgcgtatcatcagtttgcagcctacggcccaggaccaggcgctaaatttgtggctgcctggacactgaaagccgccgctggaccaggtcctggacagtacatcaaggccaacagcaagttcatcggcatcaccgaactcggcccaggaccaggctatccctacgatgtgcctgattacgcctgatagtgatgattcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - DNA, 9595 bp, synthetic construct
  source 1..9595: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaaagtccgccatatgagatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - protein, 139 aa, synthetic construct
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 17 - protein, 167 aa, synthetic construct
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 18 - protein, 10 aa, synthetic construct
GFTFSSYGMH

SEQ ID NO: 19 - protein, 15 aa, synthetic construct
VIWYDGSNKYYADSV

SEQ ID NO: 20 - protein, 16 aa, synthetic construct
DPRGATLYYYYYGMDV

SEQ ID NO: 21 - protein, 11 aa, synthetic construct
RASQSINSYLD

SEQ ID NO: 22 - protein, 7 aa, synthetic construct
AASSLQS

SEQ ID NO: 23 - protein, 9 aa, synthetic construct
QQYYSTPFT

SEQ ID NO: 24 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 25 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 26 - protein, 5 aa, synthetic construct
RYWMS

SEQ ID NO: 27 - protein, 17 aa, synthetic construct
NIKQDGSEKYYVDSVKG

SEQ ID NO: 28 - protein, 12 aa, synthetic construct
EGGWFGELAFDY

SEQ ID NO: 29 - protein, 12 aa, synthetic construct
RASQRVSSSYLA

SEQ ID NO: 30 - protein, 7 aa, synthetic construct
DASSRAT

SEQ ID NO: 31 - protein, 9 aa, synthetic construct
QQYGSLPWT

SEQ ID NO: 32 - DNA, 2019 bp, synthetic construct
  source 1..2019: mol_type = other DNA
gcccgggcatttaaatgcgatcgcatcgattacgactctagaatagtctagtccgcaggccaccatgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtgccatgtttcaggcgctgagcgaaggctgcaccccgtatgatattaaccagatgctgaacgtgctgggcgatcatcaggtctcaggccttgagcagcttgagagtataatcaactttgaaaaactgactgaatggaccagttctaatgttatgcctatcctgtctcctctgacaaagggcatcctgggcttcgtgtttaccctgaccgtgccttctgagagaggacttagctgcattagcgaagcggatgcgaccaccccggaaagcgcgaacctgggcgaagaaattctgagccagctgtatctttggccaagggtgacctaccattcccctagttatgcttaccaccaatttgaaagacgagccaaatataaaagacacttccccggctttggccagagcctgctgtttggctaccctgtgtacgtgttcggcgattgcgtgcagggcgattgggatgcgattcgctttcgctattgcgcgccgccgggctatgcgctgctgcgctgcaacgataccaactatagcgctctgctggctgtgggggccctagaaggacccaggaatcaggactggcttggtgtcccaagacaacttgtaactcggatgcaggctattcagaatgccggcctgtgtaccctggtggccatgctggaagagacaatcttctggctgcaagcgtttctgatggcgctgaccgatagcggcccgaaaaccaacattattgtggatagccagtatgtgatgggcattagcaaaccgagctttcaggaatttgtggattgggaaaacgtgagcccggaactgaacagcaccgatcagccgttttggcaagccggaatcctggccagaaatctggtgcctatggtggccacagtgcagggccagaacctgaagtaccagggtcagtcactagtcatctctgcttctatcattgtcttcaacctgctggaactggaaggtgattatcgagatgatggcaacgtgtgggtgcataccccgctgagcccgcgcaccctgaacgcgtgggtgaaagcggtggaagaaaaaaaaggtattccagttcacctagagctggccagtatgaccaacatggagctcatgagcagtattgtgcatcagcaggtcagaacatacggccccgtgttcatgtgtctcggcggactgcttacaatggtggctggtgctgtgtggctgacagtgcgagtgctcgagctgttccgggccgcgcagctggccaacgacgtggtcctccagatcatggagctttgtggtgcagcgtttcgccaggtgtgccataccaccgtgccgtggccgaacgcgagcctgaccccgaaatggaacaacgaaaccacccagccccagatcgccaactgcagcgtgtatgacttttttgtgtggctccattattattctgttcgagacacactttggccaagggtgacctaccatatgaacaaatatgcgtatcatatgctggaaagacgagccaaatataaaagaggaccaggacctggcgctaaatttgtggccgcctggacactgaaagccgctgctggtcctggacctggccagtacatcaaggccaacagcaagttcatcggcatcaccgaactcggacccggaccaggctgatgatttcgaaatttaaataagcttgcggccgctagggataacagggtaattatcacgcccaaacatttacagccgcggtgtcaaaaaccgcgtgg

SEQ ID NO: 33 - protein, 619 aa, synthetic construct
MQIFVKTLTGKTITLEVEPSDTIENVKAKIQDKEGIPPDQQRLIFAGKQLEDGRTLSDYNIQKESTLHLVLRLRGAMFQALSEGCTPYDINQMLNVLGDHQVSGLEQLESIINFEKLTEWTSSNVMPILSPLTKGILGFVFTLTVPSERGLSCISEADATTPESANLGEEILSQLYLWPRVTYHSPSYAYHQFERRAKYKRHFPGFGQSLLFGYPVYVFGDCVQGDWDAIRFRYCAPPGYALLRCNDTNYSALLAVGALEGPRNQDWLGVPRQLVTRMQAIQNAGLCTLVAMLEETIFWLQAFLMALTDSGPKTNIIVDSQYVMGISKPSFQEFVDWENVSPELNSTDQPFWQAGILARNLVPMVATVQGQNLKYQGQSLVISASIIVFNLLELEGDYRDDGNVWVHTPLSPRTLNAWVKAVEEKKGIPVHLELASMTNMELMSSIVHQQVRTYGPVFMCLGGLLTMVAGAVWLTVRVLELFRAAQLANDVVLQIMELCGAAFRQVCHTTVPWPNASLTPKWNNETTQPQIANCSVYDFFVWLHYYSVRDTLWPRVTYHMNKYAYHMLERRAKYKRGPGPGAKFVAAWTLKAAAGPGPGQYIKANSKFIGITELGPGPG

SEQ ID NO: 34 - DNA, 1638 bp, synthetic construct
  source 1..1638: mol_type = other DNA
atggccgggatgttccaggcactgtccgaaggctgcacaccctatgatattaaccagatgctgaatgtcctgggagaccaccaggtctctggcctggagcagctggagagcatcatcaacttcgagaagctgaccgagtggacaagctccaatgtgatgcctatcctgtccccactgaccaagggcatcctgggcttcgtgtttaccctgacagtgccttctgagcggggcctgtcttgcatcagcgaggcagacgcaaccacaccagagtccgccaatctgggcgaggagatcctgtctcagctgtacctgtggccccgggtgacatatcactccccttcttacgcctatcaccagttcgagcggagagccaagtacaagagacacttcccaggctttggccagtctctgctgttcggctaccccgtgtacgtgttcggcgattgcgtgcagggcgactgggatgccatccggtttagatactgcgcaccacctggatatgcactgctgaggtgtaacgacaccaattattccgccctgctggcagtgggcgccctggagggccctcgcaatcaggattggctgggcgtgccaaggcagctggtgacacgcatgcaggccatccagaacgcaggcctgtgcaccctggtggcaatgctggaggagacaatcttctggctgcaggcctttctgatggccctgaccgacagcggccccaagacaaacatcatcgtggattcccagtacgtgatgggcatctccaagccttctttccaggagtttgtggactgggagaacgtgagcccagagctgaattccaccgatcagccattctggcaggcaggaatcctggcaaggaacctggtgcctatggtggccacagtgcagggccagaatctgaagtaccagggccagagcctggtcatcagcgcctccatcatcgtgtttaacctgctggagctggagggcgactatcgggacgatggcaacgtgtgggtgcacaccccactgagccccagaacactgaacgcctgggtgaaggccgtggaggagaagaagggcatcccagtgcacctggagctggcctccatgaccaatatggagctgatgtctagcatcgtgcaccagcaggtgaggacatacggacccgtgttcatgtgcctgggaggcctgctgaccatggtggcaggagccgtgtggctgacagtgcgggtgctggagctgttcagagccgcccagctggccaacgatgtggtgctgcagatcatggagctgtgcggagcagcctttcgccaggtgtgccacaccacagtgccatggcccaatgcctccctgacccccaagtggaacaatgagacaacacagcctcagatcgccaactgtagcgtgtacgacttcttcgtgtggctgcactactatagcgtgagggataccctgtggccccgcgtgacataccacatgaataagtacgcctatcacatgctggagaggcgcgccaagtataagagaggccctggcccaggcgcaaagtttgtggcagcatggaccctgaaggccgccgccggccccggccccggccagtatatcaaggctaacagtaagttcattggaatcacagagctgggacccggacctgga

SEQ ID NO: 35 - protein, 546 aa, synthetic construct
MAGMFQALSEGCTPYDINQMLNVLGDHQVSGLEQLESIINFEKLTEWTSSNVMPILSPLTKGILGFVFTLTVPSERGLSCISEADATTPESANLGEEILSQLYLWPRVTYHSPSYAYHQFERRAKYKRHFPGFGQSLLFGYPVYVFGDCVQGDWDAIRFRYCAPPGYALLRCNDTNYSALLAVGALEGPRNQDWLGVPRQLVTRMQAIQNAGLCTLVAMLEETIFWLQAFLMALTDSGPKTNIIVDSQYVMGISKPSFQEFVDWENVSPELNSTDQPFWQAGILARNLVPMVATVQGQNLKYQGQSLVISASIIVFNLLELEGDYRDDGNVWVHTPLSPRTLNAWVKAVEEKKGIPVHLELASMTNMELMSSIVHQQVRTYGPVFMCLGGLLTMVAGAVWLTVRVLELFRAAQLANDVVLQIMELCGAAFRQVCHTTVPWPNASLTPKWNNETTQPQIANCSVYDFFVWLHYYSVRDTLWPRVTYHMNKYAYHMLERRAKYKRGPGPGAKFVAAWTLKAAAGPGPGQYIKANSKFIGITELGPGPG

SEQ ID NO: 36 - DNA, 2019 bp, synthetic construct
  source 1..2019: mol_type = other DNA
gcccgggcatttaaatgcgatcgcatcgattacgactctagaatagtctagtccgcaggccaccatgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtgccatgtttcaggcgctgagcgaaggctgcaccccgtatgatattaaccagatgctgaacgtgctgggcgatcatcagtttaagcacatcaaagcctttgaccggacatttgctaacaacccaggtcccatggttgtgtttgccacacctgggcctatcctgtctcctctgacaaagggcatcctgggcttcgtgtttaccctgaccgtgccttctgagagaggacttagctgcattagcgaagcggatgcgaccaccccggaaagcgcgaacctgggcgaagaaattctgagccagctgtatctttggccaagggtgacctaccattcccctagttatgcttaccaccaatttgaaagacgagccaaatataaaagacacttccccggctttggccagagcctgctgtttggctaccctgtgtacgtgttcggcgattgcgtgcagggcgattgggatgcgattcgctttcgctattgcgcgccgccgggctatgcgctgctgcgctgcaacgataccaactatagcgctctgctggctgtgggggccctagaaggacccaggaatcaggactggcttggtgtcccaagacaacttgtaactcggatgcaggctattcagaatgccggcctgtgtaccctggtggccatgctggaagagacaatcttctggctgcaagcgtttctgatggcgctgaccgatagcggcccgaaaaccaacattattgtggatagccagtatgtgatgggcattagcaaaccgagctttcaggaatttgtggattgggaaaacgtgagcccggaactgaacagcaccgatcagccgttttggcaagccggaatcctggccagaaatctggtgcctatggtggccacagtgcagggccagaacctgaagtaccagggtcagtcactagtcatctctgcttctatcattgtcttcaacctgctggaactggaaggtgattatcgagatgatggcaacgtgtgggtgcataccccgctgagcccgcgcaccctgaacgcgtgggtgaaagcggtggaagaaaaaaaaggtattccagttcacctagagctggccagtatgaccaacatggagctcatgagcagtattgtgcatcagcaggtcagaacatacggccccgtgttcatgtgtctcggcggactgcttacaatggtggctggtgctgtgtggctgacagtgcgagtgctcgagctgttccgggccgcgcagctggccaacgacgtggtcctccagatcatggagctttgtggtgcagcgtttcgccaggtgtgccataccaccgtgccgtggccgaacgcgagcctgaccccgaaatggaacaacgaaaccacccagccccagatcgccaactgcagcgtgtatgacttttttgtgtggctccattattattctgttcgagacacactttggccaagggtgacctaccatatgaacaaatatgcgtatcatatgctggaaagacgagccaaatataaaagaggaccaggacctggcgctaaatttgtggccgcctggacactgaaagccgctgctggtcctggacctggccagtacatcaaggccaacagcaagttcatcggcatcaccgaactcggacccggaccaggctgatgatttcgaaatttaaataagcttgcggccgctagggataacagggtaattatcacgcccaaacatttacagccgcggtgtcaaaaaccgcgtgg

SEQ ID NO: 37 - protein, 619 aa, synthetic construct
MQIFVKTLTGKTITLEVEPSDTIENVKAKIQDKEGIPPDQQRLIFAGKQLEDGRTLSDYNIQKESTLHLVLRLRGAMFQALSEGCTPYDINQMLNVLGDHQFKHIKAFDRTFANNPGPMVVFATPGPILSPLTKGILGFVFTLTVPSERGLSCISEADATTPESANLGEEILSQLYLWPRVTYHSPSYAYHQFERRAKYKRHFPGFGQSLLFGYPVYVFGDCVQGDWDAIRFRYCAPPGYALLRCNDTNYSALLAVGALEGPRNQDWLGVPRQLVTRMQAIQNAGLCTLVAMLEETIFWLQAFLMALTDSGPKTNIIVDSQYVMGISKPSFQEFVDWENVSPELNSTDQPFWQAGILARNLVPMVATVQGQNLKYQGQSLVISASIIVFNLLELEGDYRDDGNVWVHTPLSPRTLNAWVKAVEEKKGIPVHLELASMTNMELMSSIVHQQVRTYGPVFMCLGGLLTMVAGAVWLTVRVLELFRAAQLANDVVLQIMELCGAAFRQVCHTTVPWPNASLTPKWNNETTQPQIANCSVYDFFVWLHYYSVRDTLWPRVTYHMNKYAYHMLERRAKYKRGPGPGAKFVAAWTLKAAAGPGPGQYIKANSKFIGITELGPGPG

SEQ ID NO: 38 - DNA, 228 bp, synthetic construct
  source 1..228: mol_type = other DNA
atgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggc

SEQ ID NO: 39 - DNA, 228 bp, synthetic construct
  source 1..228: mol_type = other DNA
atgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtgcc

SEQ ID NO: 40 - DNA, 78 bp, synthetic construct
  source 1..78: mol_type = other DNA
atggccgtcatggcgccccgaaccctcgtcctgctactctcgggggctctggccctgacccagacctgggcgggctct

SEQ ID NO: 41 - DNA, 201 bp, synthetic construct
  source 1..201: mol_type = other DNA
ccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 42 - DNA, 60 bp, synthetic construct
  source 1..60: mol_type = other DNA
atggagaccgatacactgctgctgtgggtgctgctcctgtgggtgccaggaagcacaggc

SEQ ID NO: 43 - DNA, 3178 bp, Homo sapiens
  source 1..3178: mol_type = unassigned DNA
ggcaccgattcggggcctgcccggacttcgccgcacgctgcagaacctcgcccagcgcccaccatgccccggcagctcagcgcggcggccgcgctcttcgcgtccctggccgtaattttgcacgatggcagtcaaatgagagcaaaagcatttccagaaaccagagattattctcaacctactgcagcagcaacagtacaggacataaaaaaacctgtccagcaaccagctaagcaagcacctcaccaaactttagcagcaagattcatggatggtcatatcacctttcaaacagcggccacagtaaaaattccaacaactaccccagcaactacaaaaaacactgcaaccaccagcccaattacctacaccctggtcacaacccaggccacacccaacaactcacacacagctcctccagttactgaagttacagtcggccctagcttagccccttattcactgccacccaccatcaccccaccagctcatacagctggaaccagttcatcaaccgtcagccacacaactgggaacaccactcaacccagtaaccagaccacccttccagcaactttatcgatagcactgcacaaaagcacaaccggtcagaagcctgatcaacccacccatgccccaggaacaacggcagctgcccacaataccacccgcacagctgcacctgcctccacggttcctgggcccacccttgcacctcagccatcgtcagtcaagactggaatttatcaggttctaaacggaagcagactctgtataaaagcagagatggggatacagctgattgttcaagacaaggagtcggttttttcacctcggagatacttcaacatcgaccccaacgcaacgcaagcctctgggaactgtggcacccgaaaatccaaccttctgttgaattttcagggcggatttgtgaatctcacatttaccaaggatgaagaatcatattatatcagtgaagtgggagcctatttgaccgtctcagatccagagacagtttaccaaggaatcaaacatgcggtggtgatgttccagacagcagtcgggcattccttcaagtgcgtgagtgaacagagcctccagttgtcagcccacctgcaggtgaaaacaaccgatgtccaacttcaagcctttgattttgaagatgaccactttggaaatgtggatgagtgctcgtctgactacacaattgtgcttcctgtgattggggccatcgtggttggtctctgccttatgggtatgggtgtctataaaatccgcctaaggtgtcaatcatctggataccagagaatctaattgttgcccggggggaatgaaaataatggaatttagagaactctttcatcccttccaggatggatgttgggaaattccctcagagtgtgggtccttcaaacaatgtaaaccaccatcttctattcaaatgaagtgagtcatgtgtgatttaagttcaggcagcacatcaatttctaaatactttttgtttattttatgaaagatatagtgagctgtttattttctagtttcctttagaatattttagccactcaaagtcaacatttgagatatgttgaattaacataatatatgtaaagtagaataagccttcaaattataaaccaagggtcaattgtaactaatactactgtgtgtgcattgaagattttattttacccttgatcttaacaaagcctttgctttgttatcaaatggactttcagtgcttttactatctgtgttttatggtttcatgtaacatacatattcctggtgtagcacttaactccttttccactttaaatttgtttttgttttttgagacggagtttcactcttgtcacccaggctggagtacagtggcacgatctcggcttatggcaacctccgcctcccgggttcaagtgattctcctgcttcagcttcccgagtagctgggattacaggcacacactaccacgcctggctaatttttgtatttttattatagacgggtttcaccatgttggccagactggtcttgaactcttgacctcaggtgatccacccacctcagcctcccaaagtgctgggattacaggcatgagccattgcgcccggccttaaatgttttttttaatcatcaaaaagaacaacatatctcaggttgtctaagtgtttttatgtaaaaccaacaaaaagaacaaatcagcttatattttttatcttgatgactcctgctccagaattgctagactaagaattaggtggctacagatggtagaactaaacaataagcaagagacaataataatggcccttaattattaacaaagtgccagagtctaggctaagcactttatctatatctcatttcattctcacaacttataagtgaatgagtaaactgagacttaagggaactgaatcacttaaatgtcacctggctaactgatggcagagccagagcttgaattcatgttggtctgacatcaaggtctttggtcttctccctacaccaagttacctacaagaacaatgacaccacactctgcctgaaggctcacacctcataccagcatacgctcaccttacagggaaatgggtttatccaggatcatgagacattagggtagatgaaaggagagctttgcagataacaaaatagcctatccttaataaatcctccactctctggaaggagactgaggggctttgtaaaacattagtcagttgctcatttttatgggattgcttagctgggctgtaaagatgaaggcatcaaataaactcaaagtatttttaaatttttttgataatagagaaacttcgctaaccaactgttctttcttgagtgtatagccccatcttgtggtaacttgctgcttctgcacttcatatccatatttcctattgttcactttattctgtagagcagcctgccaagaattttatttctgctgttttttttgctgctaaagaaaggaactaagtcaggatgttaacagaaaagtccacataaccctagaattcttagtcaaggaataattcaagtcagcctagagaccatgttgactttcctcatgtgtttccttatgactcagtaagttggcaaggtcctgactttagtcttaataaaacattgaattgtagtaaaggtttttgcaataaaaacttactttgg

SEQ ID NO: 44 - DNA, 1858 bp, Mus musculus
  source 1..1858: mol_type = unassigned DNA
attccggaggtgaaaaacaatggcacaacgtgtataatggccagcttctctgcctcctttctgaccacctacgagactgcgaatggttctcagatcgtgaacatttccctgccagcctctgcagaagtactgaaaaatggcagttcttgtggtaaagaaaatgtttctgaccccagcctcacaattacttttggaagaggatatttactgacactcaacttcacaaaaaatacaacacgttacagtgtccagcatatgtattttacatataacttgtcagatacagaacattttcccaatgccatcagcaaagagatctacaccatggattccacaactgacatcaaggcagacatcaacaaagcataccggtgtgtcagtgatatccgggtctacatgaagaatgtgaccgttgtgctccgggatgccactatccaggcctacctgtcgagtggcaacttcagcaaggaagagacacactgcacacaggatggaccttccccaaccactgggccacccagcccctcaccaccacttgtgcccacaaaccccactgtatccaagtacaatgttactggtaacaacggaacctgcctgctggcctctatggcactgcaactgaatatcacctacctgaaaaaggacaacaagacggtgaccagagcgttcaacatcagcccaaatgacacatctagtgggagttgcggtatcaacttggtgaccctgaaagtggagaacaagaacagagccctggaattgcagtttgggatgaatgccagctctagcctgtttttcttgcaaggagtgcgcttgaatatgactcttcctgatgccctagtgcccacattcagcatctccaaccattcactgaaagctcttcaggccactgtgggaaactcatacaagtgcaacactgaggaacacatctttgtcagcaagatgctctccctcaatgtcttcagtgtgcaggtccaggctttcaaggtggacagtgacaggtttgggtctgtggaagagtgtgttcaggatggtaacaacatgttgatccccattgctgtgggcggtgccctggcagggctgatcctcatcgtcctcattgcctacctcattggcaggaagaggagtcacgccggctatcagaccatctagcctggtgggcaggtgcaccagagatgcacaggggcctgttctcacatccccaagcttagataggtgtggaagggaggcacactttctggcaaactgttttaaaatctgctttatcaaatgtgaagttcatcttgcaacatttactatgcacaaaggaataactattgaaatgacggtgttaattttgctaactgggttaaatattgatgagaaggctccactgatttgacttttaagacttggtgtttggttcttcattcttttactcagatttaagcctatcaaagggatactctggtccagaccttggcctggcaagggtggctgatggttaggctgcacacacttaagaagcaacgggagcagggaaggcttgcacacaggcacgcacagggtcaacctctggacacttggcttgggctacctggccttgggggggctgaactctggcatctggctgggtacacacccccccaatttctgtgctctgccacccgtgagctgccactttcctaaatagaaaatggcattatttttatttacttttttgtaaagtgatttccagtcttgtgttggcgttcagggtggccctgtctctgcactgtgtacaataatagattcacactgctgacgtgtcttgcagcgtaggtgggttgtacactgggcatcagctcacgtaatgcattgcctgtaacgatgctaataaaaa

SEQ ID NO: 45 - DNA, 2339 bp, Homo sapiens
  source 1..2339: mol_type = unassigned DNA
ggcccaaccgccgcccgcgcccccgctctccgcaccgtacccggccgcctcgcgccatggcggcccccggcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacctttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcatgctgatccccatcgctgtgggtggtgccctggcggggctggtcctcatcgtcctcatcgcctacctcgtcggcaggaagaggagtcacgcaggctaccagactatctagcctggtgcacgcaggcacagcagctgcaggggcctctgttcctttctctgggcttagggtcctgtcgaaggggaggcacactttctggcaaacgtttctcaaatctgcttcatccaatgtgaagttcatcttgcagcatttactatgcacaacagagtaactatcgaaatgacggtgttaattttgctaactgggttaaatattttgctaactggttaaacattaatatttaccaaagtaggattttgagggtgggggtgctctctctgagggggtgggggtgccgctgtctctgaggggtgggggtgccgctgtctctgaggggtgggggtgccgctctctctgagggggtgggggtgccgctttctctgagggggtgggggtgccgctctctctgagggggtgggggtgctgctctctccgaggggtggaatgccgctgtctctgaggggtgggggtgccgctctaaattggctccatatcatttgagtttagggttctggtgtttggtttcttcattctttactgcactcagatttaagccttacaaagggaaagcctctggccgtcacacgtaggacgcatgaaggtcactcgtggtgaggctgacatgctcacacattacaacagtagagagggaaaatcctaagacagaggaactccagagatgagtgtctggagcgcttcagttcagctttaaaggccaggacgggccacacgtggctggcggcctcgttccagtggcggcacgtccttgggcgtctctaatgtctgcagctcaagggctggcacttttttaaatataaaaatgggtgttatttttatttttttttgtaaagtgatttttggtcttctgttgacattcggggtgatcctgttctgcgctgtgtacaatgtgagatcggtgcgttctcctgatgttttgccgtggcttggggattgtacacgggaccagctcacgtaatgcattgcctgtaacaatgtaataaaaagcctctttcttttaaaaaaaaaaaaaaaaaaaaaaaa

SEQ ID NO: 46 - DNA, 45 bp, synthetic construct
  source 1..45: mol_type = other DNA
cagtacatcaaggccaacagcaagttcatcggcatcaccgaactc

SEQ ID NO: 47 - protein, 15 aa, synthetic construct
QYIKANSKFIGITEL

SEQ ID NO: 48 - DNA, 39 bp, synthetic construct
  source 1..39: mol_type = other DNA
gctaaatttgtggctgcctggacactgaaagccgccgct

SEQ ID NO: 49 - protein, 13 aa, synthetic construct
AKFVAAWTLKAAA

SEQ ID NO: 50 - DNA, 593 bp, synthetic construct
  source 1..593: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgt

SEQ ID NO: 51 - DNA, 589 bp, synthetic construct
  source 1..589: mol_type = other DNA
tctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 52 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 53 - DNA, 1563 bp, synthetic construct
  source 1..1563: mol_type = other DNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataa

SEQ ID NO: 54 - DNA, 1653 bp, synthetic construct
  source 1..1653: mol_type = other DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 55 - DNA, 66 bp, Foot-and-Mouth disease virus
  source 1..66: mol_type = other DNA
gtaaagcaaacactgaactttgaccttctcaagttggctggagacgttgagtccaatcctgggccc

SEQ ID NO: 56 - protein, 5 aa, synthetic construct
GPGPG

SEQ ID NO: 57 - protein, 16 aa, synthetic construct
SVPAQAPKRTQAPTKA

SEQ ID NO: 58 - protein, 15 aa, synthetic construct
KLVVVGACGVGKSAL

SEQ ID NO: 59 - protein, 11 aa, synthetic construct
EYKLVVVGACG

SEQ ID NO: 60 - protein, 16 aa, synthetic construct
QCEIOWAREFLKEIGJ

SEQ ID NO: 61 - protein, 8 aa, synthetic construct
FIEUHFWI

SEQ ID NO: 62 - protein, 12 aa, synthetic construct
FEWRHRJTRUJR

SEQ ID NO: 63 - protein, 9 aa, synthetic construct
QIEJOEIJE

SEQ ID NO: 64 - protein, 9 aa, synthetic construct
QCEIOWARE

SEQ ID NO: 65 - protein, 14 aa, synthetic construct
FJELFISBOSJFIE

SEQ ID NO: 66 - protein, 10 aa, synthetic construct
NEIOREIREI

SEQ ID NO: 67 - protein, 10 aa, synthetic construct
VVVGACGVGK

SEQ ID NO: 68 - protein, 10 aa, synthetic construct
KLVVVGACGV

SEQ ID NO: 69 - protein, 9 aa, synthetic construct
VVGADGVGK

SEQ ID NO: 70 - protein, 10 aa, synthetic construct
VVVGADGVGK

SEQ ID NO: 71 - protein, 9 aa, synthetic construct
VVGAVGVGK

SEQ ID NO: 72 - protein, 10 aa, synthetic construct
VVVGAVGVGK

SEQ ID NO: 73 - protein, 9 aa, synthetic construct
AVGVGKSAL

SEQ ID NO: 74 - protein, 10 aa, synthetic construct
ILDTAGHEEY

SEQ ID NO: 75 - protein, 25 aa, synthetic construct
MTEYKLVVVGACGVGKSALTIQLIQ

SEQ ID NO: 76 - protein, 25 aa, synthetic construct
MTEYKLVVVGADGVGKSALTIQLIQ

SEQ ID NO: 77 - protein, 25 aa, synthetic construct
MTEYKLVVVGAVGVGKSALTIQLIQ

SEQ ID NO: 78 - protein, 25 aa, synthetic construct
ETCLLDILDTAGHEEYSAMRDQYMR

SEQ ID NO: 79 - protein, 189 aa, synthetic construct [sequence omitted]

SEQ ID NO: 80 - protein, 100 aa, synthetic construct
VTNTEMFVTAPDNLGYMYEVQWPGQTQPQIANCSVYDFFVWLHYYSVRDTVTNTEMFVTAPDNLGYMYEVQWPGQTQPQIANCSVYDFFVWLHYYSVRDT

SEQ ID NO: 81 - protein, 25 aa, synthetic construct
VTNTEMFVTAPDNLGYMYEVQWPGQ

SEQ ID NO: 82 - protein, 25 aa, synthetic construct
TQPQIANCSVYDFFVWLHYYSVRDT

SEQ ID NO: 83 - DNA, 19 bp, synthetic construct
  source 1..19: mol_type = other DNA
tccctatcagtgatagaga

SEQ ID NO: 84 - protein, 11 aa, synthetic construct
IEFROEIFJEF

SEQ ID NO: 85 - protein, 9 aa, synthetic construct
IEFROEIFJ

SEQ ID NO: 86 - protein, 9 aa, synthetic construct
EFROEIFJE

SEQ ID NO: 87 - protein, 9 aa, synthetic construct
FROEIFJEF

SEQ ID NO: 88 - protein, 7 aa, synthetic construct
SINFEKL

SEQ ID NO: 89 - protein, 9 aa, synthetic construct
LLLLLVVVV

SEQ ID NO: 90 - protein, 9 aa, synthetic construct
EKLAAYLLL

SEQ ID NO: 91 - DNA, 19 bp, synthetic construct
  source 1..19: mol_type = other DNA
taatacgactcactatagg

SEQ ID NO: 92 - DNA, 20 bp, synthetic construct
  source 1..20: mol_type = other DNA
taatacgactcactataggn

SEQ ID NO: 93 - DNA, 17 bp, synthetic construct
  source 1..17: mol_type = other DNA
taatacgactcactata

SEQ ID NO: 94 - DNA, 18 bp, synthetic construct
  source 1..18: mol_type = other DNA
taatacgactcactatan

SEQ ID NO: 95 - DNA, 17 bp, synthetic construct
  source 1..17: mol_type = other DNA
atttaggtgacactata

SEQ ID NO: 96 - protein, 26 aa, synthetic construct
MTEYKLVVVGACGVGKSALTIQLIQN

SEQ ID NO: 97 - protein, 27 aa, synthetic construct
MTEYKLVVVGACGVGKSALTIQLIQNH

SEQ ID NO: 98 - protein, 28 aa, synthetic construct
MTEYKLVVVGACGVGKSALTIQLIQNHF

SEQ ID NO: 99 - protein, 35 aa, synthetic construct
MTEYKLVVVGACGVGKSALTIQLIQNHFVDEYDPT

SEQ ID NO: 100 - protein, 40 aa, synthetic construct
MTEYKLVVVGACGVGKSALTIQLIQNHFVDEYDPTIEDSY

SEQ ID NO: 101 - protein, 26 aa, synthetic construct
MTEYKLVVVGADGVGKSALTIQLIQN

SEQ ID NO: 102 - protein, 27 aa, synthetic construct
MTEYKLVVVGADGVGKSALTIQLIQNH

SEQ ID NO: 103 - protein, 28 aa, synthetic construct
MTEYKLVVVGADGVGKSALTIQLIQNHF

SEQ ID NO: 104 - protein, 35 aa, synthetic construct
MTEYKLVVVGADGVGKSALTIQLIQNHFVDEYDPT

SEQ ID NO: 105 - protein, 40 aa, synthetic construct
MTEYKLVVVGADGVGKSALTIQLIQNHFVDEYDPTIEDSY

SEQ ID NO: 106 - protein, 26 aa, synthetic construct
MTEYKLVVVGAVGVGKSALTIQLIQN

SEQ ID NO: 107 - protein, 27 aa, synthetic construct
MTEYKLVVVGAVGVGKSALTIQLIQNH

SEQ ID NO: 108 - protein, 28 aa, synthetic construct
MTEYKLVVVGAVGVGKSALTIQLIQNHF

SEQ ID NO: 109 - protein, 35 aa, synthetic construct
MTEYKLVVVGAVGVGKSALTIQLIQNHFVDEYDPT

SEQ ID NO: 110 - protein, 40 aa, synthetic construct
MTEYKLVVVGAVGVGKSALTIQLIQNHFVDEYDPTIEDSY

SEQ ID NO: 111 - protein, 26 aa, synthetic construct
GETCLLDILDTAGHEEYSAMRDQYMR

SEQ ID NO: 112 - protein, 27 aa, synthetic construct
GETCLLDILDTAGHEEYSAMRDQYMRT

SEQ ID NO: 113 - protein, 28 aa, synthetic construct
DGETCLLDILDTAGHEEYSAMRDQYMRT

SEQ ID NO: 114 - protein, 35 aa, synthetic construct
VVIDGETCLLDILDTAGHEEYSAMRDQYMRTGEGF

SEQ ID NO: 115 - protein, 40 aa, synthetic construct
RKQVVIDGETCLLDILDTAGHEEYSAMRDQYMRTGEGFLC

SEQ ID NO: 116 - protein, 25 aa, synthetic construct
MTEYKLVVVGAAGVGKSALTIQLIQ

SEQ ID NO: 117 - protein, 26 aa, synthetic construct
MTEYKLVVVGAAGVGKSALTIQLIQN

SEQ ID NO: 118 - protein, 27 aa, synthetic construct
MTEYKLVVVGAAGVGKSALTIQLIQNH

SEQ ID NO: 119 - protein, 28 aa, synthetic construct
MTEYKLVVVGAAGVGKSALTIQLIQNHF

SEQ ID NO: 120 - protein, 35 aa, synthetic construct
MTEYKLVVVGAAGVGKSALTIQLIQNHFVDEYDPT

SEQ ID NO: 121 - protein, 40 aa, synthetic construct
MTEYKLVVVGAAGVGKSALTIQLIQNHFVDEYDPTIEDSY

SEQ ID NO: 122 - protein, 20 aa, synthetic construct
KLVVVGACGVGKSALTIQLI

SEQ ID NO: 123 - protein, 21 aa, synthetic construct
KLVVVGACGVGKSALTIQLIQ

SEQ ID NO: 124 - protein, 14 aa, synthetic construct
MTEYKLVVVGACGV

SEQ ID NO: 125 - protein, 16 aa, synthetic construct
MTEYKLVVVGACGVGK

SEQ ID NO: 126 - protein, 17 aa, synthetic construct
KLVVVGACGVGKSALTI

SEQ ID NO: 127 - protein, 18 aa, synthetic construct
KLVVVGACGVGKSALTIQ

SEQ ID NO: 128 - protein, 20 aa, synthetic construct
LVVVGACGVGKSALTIQLIQ

SEQ ID NO: 129 - protein, 15 aa, synthetic construct
VVGACGVGKSALTIQ

SEQ ID NO: 130 - protein, 20 aa, synthetic construct
YKLVVVGACGVGKSALTIQL

SEQ ID NO: 131 - protein, 9 aa, synthetic construct
KLVVVGACG

SEQ ID NO: 132 - protein, 8 aa, synthetic construct
VGACGVGK

SEQ ID NO: 133 - protein, 7 aa, synthetic construct
LVVVGAC

SEQ ID NO: 134 - protein, 7 aa, synthetic construct
LVVLGAC

SEQ ID NO: 135 - protein, 8 aa, synthetic construct
SIINFEKL

SEQ ID NO: 136 - DNA, 1341 bp, synthetic construct
  source 1..1341: mol_type = other DNA
atggctggcatgaccgagtataaactagtagttgtgggagcgtgtggtgtaggcaagtcggcacttacaattcagttgatacaaatgacggaatataagctcgtagtagtcggagcagacggcgtggggaaatcagcgttgactatccagttaatacaggaaacttgcctattagacatcttggatacggcaggtcatgaggaatattccgctatgagagatcagtatatgcgcatgacggagtataagcttgtggttgtcggggccgacggggtaggtaagtcagcgctcacgatacaattaattcaaatgaccgaatacaagttggtcgtggtgggggcagttggggtcggtaaatccgcgttaacgatccaacttatccaaatgacagaatataaactcgttgttgtaggtgcatgtggcgtaggaaaaagcgcattgaccatccagctaattcaggagacgtgtctccttgatatcctagacacggcggggcacgaagaatactcggctatgcgcgaccagtacatgagaatgacggaatacaaacttgttgtcgtgggtgcggatggagtagggaaaagtgctctaacaatacaactcattcagatgacagagtacaaattggtagtcgtcggtgcggtaggagttgggaagtctgcactaactattcagctcatacagatgaccgagtacaagctggtggtggtaggcgcttgcggtgtgggtaagagtgcattaaccatacagcttatacaagagacatgtctgctagatatattagataccgccgggcatgaagagtactctgccatgcgagaccaatacatgcgtatgacagagtataaattagtagtggttggggcggacggtgttggcaagagcgccttaactatacagttgatccagatgacggagtacaaactggtcgtcgttggtgcagtgggagtgggaaaatctgcgctgacgattcaactaatccaagaaacatgtttacttgacatcctcgacactgcgggtcacgaggagtattcggcgatgcgtgatcaatatatgaggatgactgagtataagttagtcgtagttggagcggtcggtgtcggaaagtccgcgctaaccattcaattgattcaaatgactgaatacaagctagtggtagtaggagcatgcggcgtcggcaaatcggctttaacaatccaactgatacagggacccggaccaggcgccaaatttgttgctgcttggacactgaaagctgctgctgggcccggaccaggccagtacatcaaggccaactctaagtttatcggcatcaccgaattgggacctggacccggctag

SEQ ID NO: 137 - protein, 10 aa, synthetic construct
ACGVGKSALT

SEQ ID NO: 138 - protein, 11 aa, synthetic construct
ACGVGKSALTI

SEQ ID NO: 139 - protein, 12 aa, synthetic construct
ACGVGKSALTIQ

SEQ ID NO: 140 - protein, 13 aa, synthetic construct
ACGVGKSALTIQL

SEQ ID NO: 141 - protein, 14 aa, synthetic construct
ACGVGKSALTIQLI

SEQ ID NO: 142 - protein, 15 aa, synthetic construct
ACGVGKSALTIQLIQ

SEQ ID NO: 143 - protein, 10 aa, synthetic construct
CGVGKSALTI

SEQ ID NO: 144 - protein, 11 aa, synthetic construct
CGVGKSALTIQ

SEQ ID NO: 145 - protein, 12 aa, synthetic construct
CGVGKSALTIQL

SEQ ID NO: 146 - protein, 13 aa, synthetic construct
CGVGKSALTIQLI

SEQ ID NO: 147 - protein, 14 aa, synthetic construct
CGVGKSALTIQLIQ

SEQ ID NO: 148 - protein, 10 aa, synthetic construct
EYKLVVVGAC

SEQ ID NO: 149 - protein, 12 aa, synthetic construct
EYKLVVVGACGV

SEQ ID NO: 150 - protein, 13 aa, synthetic construct
EYKLVVVGACGVG

SEQ ID NO: 151 - protein, 14 aa, synthetic construct
EYKLVVVGACGVGK

SEQ ID NO: 152 - protein, 15 aa, synthetic construct
EYKLVVVGACGVGKS

SEQ ID NO: 153 - protein, 16 aa, synthetic construct
EYKLVVVGACGVGKSA

SEQ ID NO: 154 - protein, 17 aa, synthetic construct
EYKLVVVGACGVGKSAL

SEQ ID NO: 155 - protein, 18 aa, synthetic construct
EYKLVVVGACGVGKSALT

SEQ ID NO: 156 - protein, 19 aa, synthetic construct
EYKLVVVGACGVGKSALTI

SEQ ID NO: 157 - protein, 20 aa, synthetic construct
EYKLVVVGACGVGKSALTIQ

SEQ ID NO: 158 - protein, 10 aa, synthetic construct
GACGVGKSAL

SEQ ID NO: 159 - protein, 11 aa, synthetic construct
GACGVGKSALT

SEQ ID NO: 160 - protein, 12 aa, synthetic construct
GACGVGKSALTI

SEQ ID NO: 161 - protein, 13 aa, synthetic construct
GACGVGKSALTIQ

SEQ ID NO: 162 - protein, 14 aa, synthetic construct
GACGVGKSALTIQL

SEQ ID NO: 163 - protein, 15 aa, synthetic construct
GACGVGKSALTIQLI

SEQ ID NO: 164 - protein, 16 aa, synthetic construct
GACGVGKSALTIQLIQ

SEQ ID NO: 165 - protein, 11 aa, synthetic construct
KLVVVGACGVG

SEQ ID NO: 166 - protein, 12 aa, synthetic construct
KLVVVGACGVGK

SEQ ID NO: 167 - protein, 13 aa, synthetic construct
KLVVVGACGVGKS

SEQ ID NO: 168 - protein, 14 aa, synthetic construct
KLVVVGACGVGKSA

SEQ ID NO: 169 - protein, 16 aa, synthetic construct
KLVVVGACGVGKSALT

SEQ ID NO: 170 - protein, 19 aa, synthetic construct
KLVVVGACGVGKSALTIQL

SEQ ID NO: 171 - protein, 10 aa, synthetic construct
LVVVGACGVG

SEQ ID NO: 172 - protein, 11 aa, synthetic construct
LVVVGACGVGK

SEQ ID NO: 173 - protein, 12 aa, synthetic construct
LVVVGACGVGKS

SEQ ID NO: 174 - protein, 13 aa, synthetic construct
LVVVGACGVGKSA

SEQ ID NO: 175 - protein, 14 aa, synthetic construct
LVVVGACGVGKSAL

SEQ ID NO: 176 - protein, 15 aa, synthetic construct
LVVVGACGVGKSALT

SEQ ID NO: 177 - protein, 16 aa, synthetic construct
LVVVGACGVGKSALTI

SEQ ID NO: 178 - protein, 17 aa, synthetic construct
LVVVGACGVGKSALTIQ

SEQ ID NO: 179 - protein, 18 aa, synthetic construct
LVVVGACGVGKSALTIQL

SEQ ID NO: 180 - protein, 19 aa, synthetic construct
LVVVGACGVGKSALTIQLI

SEQ ID NO: 181 - protein, 12 aa, synthetic construct
MTEYKLVVVGAC

SEQ ID NO: 182 - protein, 13 aa, synthetic construct
MTEYKLVVVGACG

SEQ ID NO: 183 - protein, 15 aa, synthetic construct
MTEYKLVVVGACGVG

SEQ ID NO: 184 - protein, 17 aa, synthetic construct
MTEYKLVVVGACGVGKS

SEQ ID NO: 185 - protein, 18 aa, synthetic construct
MTEYKLVVVGACGVGKSA

SEQ ID NO: 186 - protein, 19 aa, synthetic construct
MTEYKLVVVGACGVGKSAL

SEQ ID NO: 187 - protein, 20 aa, synthetic construct
MTEYKLVVVGACGVGKSALT

SEQ ID NO: 188 - protein, 11 aa, synthetic construct
TEYKLVVVGAC

SEQ ID NO: 189 - protein, 12 aa, synthetic construct
TEYKLVVVGACG

SEQ ID NO: 190 - protein, 13 aa, synthetic construct
TEYKLVVVGACGV

SEQ ID NO: 191 - protein, 14 aa, synthetic construct
TEYKLVVVGACGVG

SEQ ID NO: 192 - protein, 15 aa, synthetic construct
TEYKLVVVGACGVGK

SEQ ID NO: 193 - protein, 16 aa, synthetic construct
TEYKLVVVGACGVGKS

SEQ ID NO: 194 - protein, 17 aa, synthetic construct
TEYKLVVVGACGVGKSA

SEQ ID NO: 195 - protein, 18 aa, synthetic construct
TEYKLVVVGACGVGKSAL

SEQ ID NO: 196 - protein, 19 aa, synthetic construct
TEYKLVVVGACGVGKSALT

SEQ ID NO: 197 - protein, 20 aa, synthetic construct
TEYKLVVVGACGVGKSALTI

SEQ ID NO: 198 - protein, 10 aa, synthetic construct
VGACGVGKSA

SEQ ID NO: 199 - protein, 11 aa, synthetic construct
VGACGVGKSAL

SEQ ID NO: 200 - protein, 12 aa, synthetic construct
VGACGVGKSALT

SEQ ID NO: 201 - protein, 13 aa, synthetic construct
VGACGVGKSALTI

SEQ ID NO: 202 - protein, 14 aa, synthetic construct
VGACGVGKSALTIQ

SEQ ID NO: 203 - protein, 15 aa, synthetic construct
VGACGVGKSALTIQL

SEQ ID NO: 204 - protein, 16 aa, synthetic construct
VGACGVGKSALTIQLI

SEQ ID NO: 205 - protein, 17 aa, synthetic construct
VGACGVGKSALTIQLIQ

SEQ ID NO: 206 - protein, 10 aa, synthetic construct
VVGACGVGKS

SEQ ID NO: 207 - protein, 11 aa, synthetic construct
VVGACGVGKSA

SEQ ID NO: 208 - protein, 12 aa, synthetic construct
VVGACGVGKSAL

SEQ ID NO: 209 - protein, 13 aa, synthetic construct
VVGACGVGKSALT

SEQ ID NO: 210 - protein, 14 aa, synthetic construct
VVGACGVGKSALTI

SEQ ID NO: 211 - protein, 16 aa, synthetic construct
VVGACGVGKSALTIQL

SEQ ID NO: 212 - protein, 17 aa, synthetic construct
VVGACGVGKSALTIQLI

SEQ ID NO: 213 - protein, 18 aa, synthetic construct
VVGACGVGKSALTIQLIQ

SEQ ID NO: 214 - protein, 11 aa, synthetic construct
VVVGACGVGKS

SEQ ID NO: 215 - protein, 12 aa, synthetic construct
VVVGACGVGKSA

SEQ ID NO: 216 - protein, 13 aa, synthetic construct
VVVGACGVGKSAL

SEQ ID NO: 217 - protein, 14 aa, synthetic construct
VVVGACGVGKSALT

SEQ ID NO: 218 - protein, 15 aa, synthetic construct
VVVGACGVGKSALTI

SEQ ID NO: 219 - protein, 16 aa, synthetic construct
VVVGACGVGKSALTIQ

SEQ ID NO: 220 - protein, 17 aa, synthetic construct
VVVGACGVGKSALTIQL

SEQ ID NO: 221 - protein, 18 aa, synthetic construct
VVVGACGVGKSALTIQLI

SEQ ID NO: 222 - protein, 19 aa, synthetic construct
VVVGACGVGKSALTIQLIQ

SEQ ID NO: 223 - protein, 10 aa, synthetic construct
YKLVVVGACG

SEQ ID NO: 224 - protein, 11 aa, synthetic construct
YKLVVVGACGV

SEQ ID NO: 225 - protein, 12 aa, synthetic construct
YKLVVVGACGVG

SEQ ID NO: 226 - protein, 13 aa, synthetic construct
YKLVVVGACGVGK

SEQ ID NO: 227 - protein, 14 aa, synthetic construct
YKLVVVGACGVGKS

SEQ ID NO: 228 - protein, 15 aa, synthetic construct
YKLVVVGACGVGKSA

SEQ ID NO: 229 - protein, 16 aa, synthetic construct
YKLVVVGACGVGKSAL

SEQ ID NO: 230 - protein, 17 aa, synthetic construct
YKLVVVGACGVGKSALT

SEQ ID NO: 231 - protein, 18 aa, synthetic construct
YKLVVVGACGVGKSALTI

SEQ ID NO: 232 - protein, 19 aa, synthetic construct
YKLVVVGACGVGKSALTIQ

SEQ ID NO: 233 - protein, 24 aa, synthetic construct
SPIKLVQKVASKIPFPDRITEESV

SEQ ID NO: 234 - protein, 22 aa, synthetic construct
NNSKKKWFLFQDSKKIQVEQPQ

SEQ ID NO: 235 - protein, 25 aa, synthetic construct
DRSVLAKKLKFVTLVFRHGDRSPID

SEQ ID NO: 236 - protein, 22 aa, synthetic construct
SHTQTTLFHTFYELLIQKNKHK

SEQ ID NO: 237 - protein, 23 aa, synthetic construct
TKRQVILLHTELERFLEYLPLRF

SEQ ID NO: 238 - protein, 19 aa, synthetic construct
EDSDKLFESKAELADHQKF

SEQ ID NO: 239 - protein, 22 aa, synthetic construct
SHNELADSGIPENSFNVSSLVE

SEQ ID NO: 240 - protein, 20 aa, synthetic construct
RLVLGKFGDLTNNFSSPHAR

SEQ ID NO: 241 - protein, 16 aa, synthetic construct
RRGGALFASRPRFTPL

SEQ ID NO: 242 - protein, 20 aa, synthetic construct
LCPREEFLRLCKKIMMRSIQ

SEQ ID NO: 243 - protein, 26 aa, synthetic construct
SGSPPLRVSVGDFSQEFSPIQEAQQD

SEQ ID NO: 244 - protein, 20 aa, synthetic construct
LSPREEFLRLCKKIMMRSIQ

SEQ ID NO: 245 - protein, 27 aa, synthetic construct
RPAGRTQLLWTPAAPTAMAEVGPGHTP

SEQ ID NO: 246 - protein, 28 aa, synthetic construct
WTPAAPTAMAEVGPGHTPAHPSQGAVPP

SEQ ID NO: 247 - protein, 16 aa, synthetic construct
AAVRPEQRPAARGSRV

SEQ ID NO: 248 - protein, 23 aa, synthetic construct
PGGDSGELITDAHELGVAHPPGY

SEQ ID NO: 249 - protein, 25 aa, synthetic construct
VTSPKASPVTFPAAAFPTASPANKD

SEQ ID NO: 250 - protein, 23 aa, synthetic construct
LENNANHDETSFLLPRKESNIVD

SEQ ID NO: 251 - protein, 21 aa, synthetic construct
RGQIKLADFRLARLYSSEESR

SEQ ID NO: 252 - protein, 22 aa, synthetic construct
KHLPGVNFPGNQWNPVEGILPS

SEQ ID NO: 253 - protein, 25 aa, synthetic construct
PAPPPAVPKEHPAPPAPPPASAPTP

SEQ ID NO: 254 - protein, 20 aa, synthetic construct
LGETMGQVTEKLQPTYMEET

SEQ ID NO: 255 - protein, 24 aa, synthetic construct
PETGEIQVKTFLDREQRESYELKV

SEQ ID NO: 256 - protein, 17 aa, synthetic construct
TFPKKIQMLARDFLDEY

SEQ ID NO: 257 - protein, 23 aa, synthetic construct
EVVGGYTWPSGNIYQGYWAQGKR

SEQ ID NO: 258 - protein, 30 aa, synthetic construct
PAHPSQGAVPPSRAAAEPHLKPSPSELQTA

SEQ ID NO: 259 - protein, 30 aa, synthetic construct
DEQGREAELARSGPSAAGPVRLKPGLVPGL

SEQ ID NO: 260 - protein, 15 aa, synthetic construct
TIKNSDKNVVLEHFG

SEQ ID NO: 261 - protein, 30 aa, synthetic construct
DGGRQHSGPRRHSGAGPKPSSSEWAVCWAP

SEQ ID NO: 262 - protein, 23 aa, synthetic construct
FCGTPDYIAPKIIAYQPYGKSVD

SEQ ID NO: 263 - protein, 24 aa, synthetic construct
RGRLPAGAVRTLLSQVNKVWDQSS

SEQ ID NO: 264 - protein, 23 aa, synthetic construct
DRASFLLTDYALSPDGSIRKATG

SEQ ID NO: 265 - protein, 21 aa, synthetic construct
SPGPRTAPRPGSQKQAGKDWQ

SEQ ID NO: 266 - protein, 22 aa, synthetic construct
HASHLQEHQRIYTGEKPFKCDT

SEQ ID NO: 267 - protein, 20 aa, synthetic construct
SLPSNVLSSLVLVPLHTTPK

SEQ ID NO: 268 - protein, 25 aa, synthetic construct
GHEHQPDMQKSLLRAAFFGKCFLDR

SEQ ID NO: 269 - protein, 22 aa, synthetic construct
SSHYKFSKPALQSQSISLVQQS

SEQ ID NO: 270 - protein, 29 aa, synthetic construct
TRNSFALVPSLQRLMLRKVALKNVDSSPS

SEQ ID NO: 271 - protein, 27 aa, synthetic construct
NLKAPRLLFAPEYGPKLKLRALEDRHS

SEQ ID NO: 272 - protein, 24 aa, synthetic construct
TETVNHHYLLFQNTDLGSFHDLLR

SEQ ID NO: 273 - protein, 23 aa, synthetic construct
EDLDANLRKLNFRLFVIRGQPAD

SEQ ID NO: 274 - protein, 27 aa, synthetic construct
ERFWRNILLLSLHKGSLYPRIPGLGKE

SEQ ID NO: 275 - protein, 29 aa, synthetic construct
ELQYRGRELRFNLIANQHLLAPGFVSETR

SEQ ID NO: 276 - protein, 22 aa, synthetic construct
STLPVISDSTTKRRWSALVIGL

SEQ ID NO: 277 - protein, 25 aa, synthetic construct
KMQRRNDDKSILMHGLVSLRESSRG

SEQ ID NO: 278 - protein, 26 aa, synthetic construct
TTVTHERKQAKVVNPPIQEVGKGARK

SEQ ID NO: 279 - protein, 19 aa, synthetic construct
KGEKNGMTFSSTKDYVNNV

SEQ ID NO: 280 - protein, 23 aa, synthetic construct
SLTEESGGAVAFFPGNLSTSSSA

SEQ ID NO: 281 - protein, 24 aa, synthetic construct
DSYHLYAYHEELSATVPSQWKKIG

SEQ ID NO: 282 - protein, 26 aa, synthetic construct
GHQKLPGKIHLFEAEFTQVAKKEPDG

SEQ ID NO: 283 - protein, 28 aa, synthetic construct
GDQYKATDFVADWAGTFKMVFTPKDGSG

SEQ ID NO: 284 - protein, 23 aa, synthetic construct
TTPSGSAEYMASEVVEVFTDQAT

SEQ ID NO: 285 - protein, 23 aa, synthetic construct
PENDDLFMMPRIVDVTSLATEGG

SEQ ID NO: 286 - protein, 23 aa, synthetic construct
MSQDIKKADEQIESMTYSTERKT

SEQ ID NO: 287 - protein, 19 aa, synthetic construct
DGVSEEFWLVDLLPSTHYT

SEQ ID NO: 288 - protein, 23 aa, synthetic construct
RYNSTAATNEVSEVTVFSKSPVT

SEQ ID NO: 289 - protein, 25 aa, synthetic construct
GRMSPSQFARVPGYVGSPLAAMNPK

SEQ ID NO: 290 - protein, 25 aa, synthetic construct
SHHTHSYQRYSHPLFLPGHRLDPPI

SEQ ID NO: 291 - protein, 22 aa, synthetic construct
SHQIHSYQLYTHPLLHPWDHRD

SEQ ID NO: 292 - protein, 20 aa, synthetic construct
DKGHQFHVHPLLHSGDDLDP

SEQ ID NO: 293 - protein, 24 aa, synthetic construct
KLRTIPLSDNTIFRRICTIAKHLE

SEQ ID NO: 294 - protein, 26 aa, synthetic construct
ASATEPANDSLFSPGAANLFSTYLAR

SEQ ID NO: 295 - protein, 23 aa, synthetic construct
FPVVQSTEDVFPQGLPNEYAFVT

SEQ ID NO: 296 - protein, 27 aa, synthetic construct
AASAAAFPSQRTSWEFLQSLVSIKQEK

SEQ ID NO: 297 - protein, 26 aa, synthetic construct
GSVLQFMPFTTVSELMKVSAMSSPKV

SEQ ID NO: 298 - protein, 26 aa, synthetic construct
NQVLASRYGIRGFSTIKIFQKGESPV

SEQ ID NO: 299 - protein, 25 aa, synthetic construct
ARLQSKEYPVIFKSIMRQRLISPQL

SEQ ID NO: 300 - protein, 26 aa, synthetic construct
DVTGPHLYSIYLHGSTDKLPYVTMGS

SEQ ID NO: 301 - protein, 27 aa, synthetic construct
SHLASLKNNVSPVLRSHSFSDPSPKFA

SEQ ID NO: 302 - protein, 28 aa, synthetic construct
TAQFAPSPGQPPALSPSYPGHRLPLQQG

SEQ ID NO: 303 - protein, 19 aa, synthetic construct
PASAKSRREFDKIELAYRR

SEQ ID NO: 304 - protein, 19 aa, synthetic construct
MAGPKGFQYRALYPFRRER

SEQ ID NO: 305 - protein, 19 aa, synthetic construct
SDAFSGLTALPQSILLFGP

SEQ ID NO: 306 - protein, 23 aa, synthetic construct
STQHADLTIIDNIKEMNFLRRYK

SEQ ID NO: 307 - protein, 24 aa, synthetic construct
LHTHYDYVSALHPVSTPSKEYTSA

SEQ ID NO: 308 - protein, 26 aa, synthetic construct
SSPLGRANGRRFANPRDSFSAMGFQR

SEQ ID NO: 309 - protein, 28 aa, synthetic construct
EIHGKCENMTITSRGTTVTPTKETVSLG

SEQ ID NO: 310 - protein, 18 aa, synthetic construct
LNTGLFRIKFKEPLENLI

SEQ ID NO: 311 - protein, 30 aa, synthetic construct
SPQSGGAATLAAQARLQPVHLDVWGEHERG

SEQ ID NO: 312 - protein, 28 aa, synthetic construct
GSGSQMPAWRTRGAISASSTQKTPTTRL

SEQ ID NO: 313 - protein, 30 aa, synthetic construct
GLTRISIQRAQPLPPCLPSFRPPTALQGLS

SEQ ID NO: 314 - protein, 24 aa, synthetic construct
SRLQTRKNKKLALSSTPSNIAPSD

SEQ ID NO: 315 - protein, 22 aa, synthetic construct
WCTEMKRVFGFPVHYTDVSNMS

SEQ ID NO: 316 - protein, 26 aa, synthetic construct
GPLQLPVTRKNMPLPGVVKLPPLPGS

SEQ ID NO: 317 - protein, 21 aa, synthetic construct
ALLQNVELRRNVLVSPTPLAN

SEQ ID NO: 318 - protein, 25 aa, synthetic construct
VNGISSQPQVPFYPNLQKSQYYSTV

SEQ ID NO: 319 - protein, 22 aa, synthetic construct
YLSHTLGAASSFMRPTVPPPQF

SEQ ID NO: 320 - protein, 24 aa, synthetic construct
SLRNNMFEISDRFIGIYKTYNITK

SEQ ID NO: 321 - protein, 22 aa, synthetic construct
VTLNDMKARQKALVRERERQLA

SEQ ID NO: 322 - protein, 23 aa, synthetic construct
VKQLERGEASVVDFKKNLEYAAT

SEQ ID NO: 323 - protein, 24 aa, synthetic construct
TKLKSKAPHWTNCILHEYKNLSTS

SEQ ID NO: 324 - protein, 23 aa, synthetic construct
FAKGFRESDLNSWPVAPRPLLSV

SEQ ID NO: 325 - protein, 24 aa, synthetic construct
HLLQKQTSIQSPSLYGNSSPPLNK

SEQ ID NO: 326 - protein, 28 aa, synthetic construct
STEVEPKESPHLARHRHLMKTLVKSLST

SEQ ID NO: 327 - protein, 21 aa, synthetic construct
DGAWPVLLDKFVEWYKDKQMS

SEQ ID NO: 328 - protein, 20 aa, synthetic construct
SHKLESIKEITNFKDAKQLL

SEQ ID NO: 329 - protein, 24 aa, synthetic construct
TGKPEMDFVRLAQLFARARPMGLF

SEQ ID NO: 330 - protein, 14 aa, synthetic construct
ALTIQLIQNHFVDE

SEQ ID NO: 331 - protein, 15 aa, synthetic construct
SALTIQLIQNHFVDE

SEQ ID NO: 332 - protein, 15 aa, synthetic construct
ALTIQLIQNHFVDEY

SEQ ID NO: 333 - protein, 16 aa, synthetic construct
SALTIQLIQNHFVDEY

SEQ ID NO: 334 - protein, 13 aa, synthetic construct
ALTIQLIQNHFVD

SEQ ID NO: 335 - protein, 13 aa, synthetic construct
LTIQLIQNHFVDE

SEQ ID NO: 336 - protein, 14 aa, synthetic construct
SALTIQLIQNHFVD

SEQ ID NO: 337 - protein, 15 aa, synthetic construct
KSALTIQLIQNHFVD

SEQ ID NO: 338 - protein, 16 aa, synthetic construct
KSALTIQLIQNHFVDE

SEQ ID NO: 339 - protein, 17 aa, synthetic construct
KSALTIQLIQNHFVDEY

SEQ ID NO: 340 - protein, 13 aa, synthetic construct
TIQLIQNHFVDEY

SEQ ID NO: 341 - protein, 14 aa, synthetic construct
LTIQLIQNHFVDEY

SEQ ID NO: 342 - protein, 16 aa, synthetic construct
GKSALTIQLIQNHFVD

SEQ ID NO: 343 - protein, 17 aa, synthetic construct
GKSALTIQLIQNHFVDE

SEQ ID NO: 344 - protein, 18 aa, synthetic construct
VGKSALTIQLIQNHFVDE

SEQ ID NO: 345 - protein, 18 aa, synthetic construct
GKSALTIQLIQNHFVDEY

SEQ ID NO: 346 - protein, 19 aa, synthetic construct
VGKSALTIQLIQNHFVDEY

SEQ ID NO: 347 - protein, 9 aa, synthetic construct
IQLIQNHFV

SEQ ID NO: 348 - protein, 9 aa, synthetic construct
LTIQLIQNH

SEQ ID NO: 349 - protein, 8 aa, synthetic construct
ACGVGKSA

SEQ ID NO: 350 - protein, 9 aa, synthetic construct
ACGVGKSAL

SEQ ID NO: 351 - protein, 8 aa, synthetic construct
CGVGKSAL

SEQ ID NO: 352 - protein, 9 aa, synthetic construct
CGVGKSALT

SEQ ID NO: 353 - protein, 8 aa, synthetic construct
GACGVGKS

SEQ ID NO: 354 - protein, 9 aa, synthetic construct
GACGVGKSA

SEQ ID NO: 355 - protein, 8 aa, synthetic construct
KLVVVGAC

SEQ ID NO: 356 - protein, 8 aa, synthetic construct
LVVVGACG

SEQ ID NO: 357 - protein, 9 aa, synthetic construct
LVVVGACGV

SEQ ID NO: 358 - protein, 9 aa, synthetic construct
VGACGVGKS

SEQ ID NO: 359 - protein, 8 aa, synthetic construct
VVGACGVG

SEQ ID NO: 360 - protein, 9 aa, synthetic construct
VVGACGVGK

SEQ ID NO: 361 - protein, 8 aa, synthetic construct
VVVGACGV

SEQ ID NO: 362 - protein, 9 aa, synthetic construct
VVVGACGVG

SEQ ID NO: 363 - protein, 9 aa, synthetic construct
YKLVVVGAC

SEQ ID NO: 364 - protein, 443 aa, synthetic construct
MTEYKLVVVGACGVGKSALTIQLIQMTEYKLVVVGADGVGKSALTIQLIQETCLLDILDTAGHEEYSAMRDQYMRMTEYKLVVVGADGVGKSALTIQLIQMTEYKLVVVGAVGVGKSALTIQLIQMTEYKLVVVGACGVGKSALTIQLIQETCLLDILDTAGHEEYSAMRDQYMRMTEYKLVVVGADGVGKSALTIQLIQMTEYKLVVVGAVGVGKSALTIQLIQMTEYKLVVVGACGVGKSALTIQLIQETCLLDILDTAGHEEYSAMRDQYMRMTEYKLVVVGADGVGKSALTIQLIQMTEYKLVVVGAVGVGKSALTIQLIQETCLLDILDTAGHEEYSAMRDQYMRMTEYKLVVVGAVGVGKSALTIQLIQMTEYKLVVVGACGVGKSALTIQLIQGPGPGAKFVAAWTLKAAAGPGPGQYIKANSKFIGITELGPGPG

SEQ ID NO: 365 - protein, 9 aa, synthetic construct
FEGRKUOOI

SEQ ID NO: 366 - protein, 14 aa, synthetic construct
PJFIOEJOIJGEIO

SEQ ID NO: 367 - protein, 11 aa, synthetic construct
KLVVAGCVYYA

SEQ ID NO: 368 - protein, 25 aa, synthetic construct
VTSAVLVVLGACLALWLSGCSSPKP

SEQ ID NO: 369 - protein, 25 aa, synthetic construct
VTSAVLVVLGACLALWLSGASSPKP

SEQ ID NO: 370 - protein, 25 aa, synthetic construct
MTEYKLVVVGAGGVGKSALTIQLIQ

(19) United States
(12) Patent Application Publication
(10) Pub. No.: US 2026/0094669 A1
(43) Pub. Date: Apr. 2, 2026

(54) EPITOPE PREDICTION VIA A LEARNED GENOTYPE NETWORK ACROSS CLASS II MHC ALLELES

(71) Applicant: Seattle Project Corp., Dover, DE (US)

(72) Inventors: Daniel Sprague, Cambridge, MA (US); Ankur Dhanik, Pleasantville, NY (US); Joshua Klein, Brookline, MA (US); Christine Denise Palmer, Cambridge, MA (US); Monica Lane, Uxbridge, MA (US); Melissa Rotunno, Southborough, MA (US); Adrienne Greene, Boston, MA (US); Meghan Hart, Charlestown, MA (US)

(21) Appl. No.: 19/327,242

(22) Filed: Sep. 12, 2025

Related U.S. Application Data
(63) Continuation of application No. PCT/US2024/019762, filed on Mar. 13, 2024.
(60) Provisional application No. 63/489,944, filed on Mar. 13, 2023.
(60) Provisional application No. 63/489,888, filed on Mar. 13, 2023.
(60) Provisional application No. 63/611,654, filed on Dec. 18, 2023.

(51) Int. Cl. G16B 25/10; G16B 20/20; G16B 40/10; G16H 20/17

(52) U.S. Cl. CPC G16B 25/10; G16B 20/20; G16B 40/10; G16H 20/17

(57) ABSTRACT

Disclosed herein are presentation models useful for identifying and selecting class II neoantigens that are likely presented by a set of MHC alleles expressed by a patient. Methods disclosed herein are useful for generating personalized cancer vaccines. Specifically, the disclosed presentation models leverage protein sequence embeddings across MHC alleles of the patient genotype. A learned genotype network (“LGN”) of the presentation model aggregates embeddings from all class II HLA alleles prior to prediction. The learned genotype network generates a prediction vector that can be used to predict likelihood of presentation of epitope sequences.

### CROSS-REFERENCE TO RELATED APPLICATIONS

This is a continuation of International Patent Application Number PCT/US2024/019762 filed Mar. 13, 2023 which claims the benefit of and priority to U.S. Provisional Patent Application No. 63/489,888 filed Mar. 13, 2023, U.S. Provisional Patent Application No. 63/489,944 filed Mar. 13, 2023, and U.S. Provisional Patent Application No. 63/611,654, filed Dec. 18, 2023, the entire disclosure of each of which is hereby incorporated by reference in its entirety for all purposes.

### REFERENCE TO A SEQUENCE LISTING XML

This application contains a sequence listing which has been submitted electronically in XML format. The Sequence Listing XML is incorporated herein by reference. Said XML file is named GSO-120WO_SL.xml, created on Apr. 4, 2024, and is 495 KB in size.

### BACKGROUND

Activity of both CD8+ and CD4+ neoantigen specific T cells is critical to the success of immunotherapy. Prior efforts, such as those described in Bulik-Sullivan, B. et al. Deep learning using tumor HLA peptide mass spectrometry datasets improves neoantigen identification. Nature Biotechnology 2018 (2018), have focused on predicting presentation of HLA class I neoantigens which drive CD8+ T-cell response. Accurate prediction of immunogenic HLA Class II restricted neoantigens, which drive CD4+ T-cell response, is the next frontier.

### SUMMARY

Disclosed herein is an approach involving training/deploying multiple machine learning models in a presentation model for identifying and selecting class II neoantigens for personalized cancer vaccines. In various embodiments, the disclosed presentation model leverages protein sequence embeddings from a first machine learning model, such as a protein language model. The embeddings are input into a learned genotype network (“LGN”) which aggregates embeddings from all class II HLA alleles prior to prediction. The learned genotype network generates a prediction vector that is further provided as input into an additional machine learning model to predict likely presentation of epitope sequences. In various embodiments, immunoaffinity purified mass spectrometry data is further incorporated to refine and improve performance of the disclosed presentation model.

Disclosed herein is a method for predicting whether an epitope sequence is presented or not presented by one or more class II MHC alleles of a genotype, the method comprising: combining the epitope sequence and sequences of the one or more class II MHC alleles of the genotype to generate one or more epitope-allele encodings; providing the one or more epitope-allele encodings as input to a first machine learning model to generate one or more learned representations of the one or more epitope-allele encodings; transforming the one or more learned representations of the one or more epitope-allele encodings using a learned genotype network to generate a single prediction vector accounting for contributions of each of the one or more class II MHC alleles; and analyzing the prediction vector using a second machine learning model to generate a genotype presentation score representing a likelihood of presentation of the epitope sequence by the one or more of the class II MHC alleles of the genotype.

In various embodiments, transforming the learned representation of the one or more epitope-allele encoding using a learned genotype network comprises combining weighted combinations of the one or more learned representations. In various embodiments, the learned genotype network comprises a plurality of learned weights, wherein each learned weight is specific for a class II MHC allele. In various embodiments, combining weighted combinations of the one or more learned representations comprises: for each of the one or more learned representations, modifying the learned representation using a learned weight of the learned genotype network; and summating the one or more modified learned representations. In various embodiments, a larger value of a learned weight indicates that a corresponding class II MHC allele contributes more heavily towards presentation of the epitope sequence in comparison to a class II MHC allele corresponding to a smaller value of a learned weight. In various embodiments, a learned weight of the learned genotype network is specific for a kth class II MHC allele and is determined based on at least a non-linear transform of a learned representation an epitope-allele encoding of the kth class II MHC allele. In various embodiments, the non-linear transform influences the learned weight specific for the kth class II MHC allele based on a learned importance of the kth class II MHC allele for presentation of epitopes. In various embodiments, a learned weight of the learned genotype network is specific for a kth class II MHC allele and is determined based on at least a sigmoid transform of a learned representation an epitope-allele encoding of the kth class II MHC allele. In various embodiments, a sum of the plurality of learned weights is 1.

In various embodiments, the first machine learning model comprises a protein language model. In various embodiments, the first machine learning model comprises a neural network. In various embodiments, the one or more learned representations comprise one or more sequence embeddings. In various embodiments, combining the epitope sequence and sequences of the one or more class II MHC alleles comprises concatenating the epitope sequence and sequences of the one or more class II MHC alleles. In various embodiments, combining the epitope sequence and sequences of the one or more class II MHC alleles comprises concatenating a first instance of the epitope sequence and a sequence of a first class II MHC allele; and further concatenating a second instance of the epitope sequence and a sequence of a second class II MHC allele. In various embodiments, the one or more class II MHC alleles are expressed in the genotype of a patient. In various embodiments, the one or more class II MHC alleles comprise six class II MHC alleles expressed in the genotype of a patient. In various embodiments, the method achieves a ROC performance metric of greater than 0.9. In various embodiments, the method achieves an area under precision recall curve (AUPRC) of greater than 0.7. In various embodiments, the method achieves an AUPRC of greater than 0.7.

In various embodiments, the learned genotype network is trained to learn contributions across a plurality of class II MHC alleles. In various embodiments, one or more of the first machine learning model, the learned genotype network, or the second machine learning model are trained using training data generated by performing mass spectrometry. In various embodiments, the training data are generated from multi-allele expressing cells. In various embodiments, the training data are generated from single-allele expressing cells. In various embodiments, one or more of the first machine learning model, the learned genotype network, or the second machine learning model are trained using intermediate resolution data generated by performing HLA-DR, HLA-DQ, and HLA-DP specific pulldown of class II MHC alleles. In various embodiments, the second machine learning model comprises a classifier network. In various embodiments, the first machine learning model, the learned genotype network, and the second machine learning model are jointly trained. In various embodiments, the first machine learning model, the learned genotype network, and the second machine learning model are trained through two or more phases. In various embodiments, the first machine learning model, the learned genotype network, and the second machine learning model are trained during a first phase using single allelic training data. In various embodiments, the first machine learning model, the learned genotype network, and the second machine learning model are further trained during a second phase using intermediate resolution data comprising DR-specific, DQ-specific, and DP-specific immunoaffinity purified mass spectrometry presentation data. In various embodiments, the first machine learning model, the learned genotype network, and the second machine learning model are further trained during a third phase using multi-allelic training data. In various embodiments, the epitope sequence comprises a KRAS epitope sequence. In various embodiments, the KRAS epitope sequence comprises a G12 mutation, optionally wherein the G12 mutation is a G12C, G12V, G12D, or G12A mutation. In various embodiments, the KRAS epitope sequence comprises a Q61 mutation, optionally wherein the Q61 mutation is a Q61H mutation. In various embodiments, the epitope sequence is between 10-40 amino acids in length, optionally wherein the epitope sequence is between 10-25 amino acids in length. In various embodiments, further comprise producing or having produced the vaccine comprising the selected epitope sequence. In various embodiments, further comprise selecting the epitope sequence for inclusion in a vaccine. In various embodiments, further comprise obtaining or having obtained the vaccine comprising the selected epitope sequence; and administering the vaccine. In various embodiments, further comprise identifying one or more T-cells that are antigen-specific for the selected epitope sequence. In various embodiments, identifying the one or more T-cells comprises co-culturing the one or more T-cells with the selected epitope sequence under conditions that expand the one or more T-cells. In various embodiments, further comprise identifying one or more T-cell receptors (TCR) of the one or more identified T-cells. In various embodiments, wherein identifying the one or more T-cell receptors comprises sequencing the T-cell receptor sequences of the one or more identified T-cells. In various embodiments, a composition comprising one or more epitope sequences, wherein at least one of the one or more epitope sequences are predicted to be presented by one or more class II MHC alleles of a genotype using methods disclosed herein. In various embodiments, composition comprises a personalized cancer vaccine comprising the one or more epitope sequences. In various embodiments, wherein the one or more epitope sequences comprise a sequence identified in any one of Tables 2, 3, 6, 7, 10.

Additionally disclosed herein is a non-transitory computer readable medium for predicting whether an epitope sequence is presented or not presented by one or more class II MHC alleles of a genotype, the non-transitory computer readable medium comprising instructions that, when executed by a processor, cause the processor to: combine the epitope sequence and sequences of the one or more class II MHC alleles of the genotype to generate one or more epitope-allele encodings; provide the one or more epitope-allele encodings as input to a first machine learning model to generate one or more learned representations of the one or more epitope-allele encodings; transform the one or more learned representations of the one or more epitope-allele encodings using a learned genotype network to generate a single prediction vector accounting for contributions of each of the one or more class II MHC alleles; and analyze the prediction vector using a second machine learning model to generate a genotype presentation score representing a likelihood of presentation of the epitope sequence by the one or more of the class II MHC alleles of the genotype.

In various embodiments, the instructions that cause the processor to transform the learned representation of the one or more epitope-allele encoding using a learned genotype network further comprises instructions that, when executed by the processor, cause the processor to combine weighted combinations of the one or more learned representations. In various embodiments, the learned genotype network comprises a plurality of learned weights, wherein each learned weight is specific for a class II MHC allele. In various embodiments, the instructions that cause the processor to combine weighted combinations of the one or more learned representations further comprises instructions that, when executed by the processor, cause the processor to: for each of the one or more learned representations, modify the learned representation using a learned weight of the learned genotype network; and summate the one or more modified learned representations. In various embodiments, a larger value of a learned weight indicates that a corresponding class II MHC allele contributes more heavily towards presentation of the epitope sequence in comparison to a class II MHC allele corresponding to a smaller value of a learned weight. In various embodiments, a learned weight of the learned genotype network is specific for a kth class II MHC allele and is determined based on at least a non-linear transform of a learned representation an epitope-allele encoding of the kth class II MHC allele. In various embodiments, the non-linear transform influences the learned weight specific for the kth class II MHC allele based on a learned importance of the kth class II MHC allele for presentation of epitopes. In various embodiments, a learned weight of the learned genotype network is specific for a kth class II MHC allele and is determined based on at least a sigmoid transform of a learned representation an epitope-allele encoding of the kth class II MHC allele. In various embodiments, a sum of the plurality of learned weights is 1.

In various embodiments, the first machine learning model comprises a protein language model. In various embodiments, the first machine learning model comprises a neural network. In various embodiments, the one or more learned representations comprise one or more sequence embeddings. In various embodiments, combining the epitope sequence and sequences of the one or more class II MHC alleles comprises concatenating the epitope sequence and sequences of the one or more class II MHC alleles. In various embodiments, combining the epitope sequence and sequences of the one or more class II MHC alleles comprises concatenating a first instance of the epitope sequence and a sequence of a first class II MHC allele; and further concatenating a second instance of the epitope sequence and a sequence of a second class II MHC allele. In various embodiments, the one or more class II MHC alleles are expressed in the genotype of a patient. In various embodiments, the one or more class II MHC alleles comprise six class II MHC alleles expressed in the genotype of a patient. In various embodiments, the method achieves a ROC performance metric of greater than 0.9. In various embodiments, the method achieves an area under precision recall curve (AUPRC) of greater than 0.7. In various embodiments, the method achieves an AUPRC of greater than 0.7. In various embodiments, the learned genotype network is trained to learn contributions across a plurality of class II MHC alleles.

In various embodiments, one or more of the first machine learning model, the learned genotype network, or the second machine learning model are trained using training data generated by performing mass spectrometry. In various embodiments, the training data are generated from multi-allele expressing cells. In various embodiments, the training data are generated from single-allele expressing cells. In various embodiments, one or more of the first machine learning model, the learned genotype network, or the second machine learning model are trained using intermediate resolution data generated by performing HLA-DR, HLA-DQ, and HLA-DP specific pulldown of class II MHC alleles. In various embodiments, the second machine learning model comprises a classifier network. In various embodiments, the first machine learning model, the learned genotype network, and the second machine learning model are jointly trained. In various embodiments, the first machine learning model, the learned genotype network, and the second machine learning model are trained through two or more phases. In various embodiments, the first machine learning model, the learned genotype network, and the second machine learning model are trained during a first phase using single allelic training data. In various embodiments, the first machine learning model, the learned genotype network, and the second machine learning model are further trained during a second phase using intermediate resolution data comprising DR-specific, DQ-specific, and DP-specific immunoaffinity purified mass spectrometry presentation data. In various embodiments, the first machine learning model, the learned genotype network, and the second machine learning model are further trained during a third phase using multi-allelic training data. In various embodiments, the non-transitory computer readable medium, further comprise selecting the epitope sequence for inclusion in a vaccine.

Disclosed herein is method for patient subtyping, the method comprising: obtaining or having obtained expression levels of two or more biomarkers from a sample obtained from a patient, the two or more biomarkers selected from biomarkers involved in any of angiogenesis fibroblasts, pro-tumor immune infiltrate, anti-tumor immune infiltrate, or proliferation rate EMT signature activities; determining, based on the expression levels of the two or more biomarkers, whether to classify the patient into an immune enriched fibrotic subtype; and responsive to classifying the patient into the immune enriched fibrotic subtype, selecting the patient as a candidate for receiving a personalized cancer vaccine comprising one or more neoantigens predicted to be presented by one or more class II MHC alleles of a genotype of the patient. In various embodiments, wherein the two or more biomarkers comprise two or more of CD274, CD8A, CXCL9, GZMA, or PRF1. In various embodiments, the two or more biomarkers comprise presence or absence of somatic alterations in two or more of TP53, APC, KRAS, PIK3CA, or SMAD4. In various embodiments, a subject is a human. In various embodiments, the human is a cancer patient. In various embodiments, the sample is a tumor sample or a tumor biopsy. In various embodiments, the tumor sample or tumor biopsy comprises cells from a primary tumor or metastasized tumor. In various embodiments, obtaining or having obtained expression levels of two or more biomarkers comprises performing a histopathological characterization to determine a tumor microenvironment. In various embodiments, obtaining or having obtained expression levels of two or more biomarkers comprises performing RNA-seq to determine a tumor microenvironment. In various embodiments, determining, based on the expression levels of the two or more biomarkers, whether to classify the patient into an immune enriched fibrotic subtype comprises determining whether to classify the patient into one of the immune enriched fibrotic subtype, immune enriched non-fibrotic subtype, fibrotic subtype, or depleted subtype. In various embodiments, further comprising administering the personalized cancer vaccine to the patient. In various embodiments, the one or more neoantigens are predicted to be presented by one or more class II MHC alleles of a genotype of the patient using the method.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is an overview of an environment for identifying likelihoods of peptide presentation in patients, in accordance with an embodiment.

FIGS. 2A and 2B illustrate a method of obtaining presentation information, in accordance with an embodiment.

FIG. 3A is a high-level block diagram illustrating the computer logic components of the presentation identification system, according to one embodiment.

FIG. 3B illustrates an example set of training data, according to one embodiment.

FIG. 4A depicts an example presentation model, according to one embodiment.

FIG. 4B shows an example network architecture of a machine learning model, according to one embodiment.

FIG. 5 shows an example flow process for predicting epitope presentation, according to one embodiment.

FIG. 6 illustrates an example computer for implementing the entities shown in FIGS. 1, 2A-2B, 3A-3B, and 4-5.

FIG. 7 is an example flow process for predicting epitope presentation via a learned genotype network.

FIGS. 8A and 8B show example performances of the EDGE (max), EDGE (LGN), and EDGE (LGN+IP-MS) presentation models in comparison to BERTMHC and NetMHCIIpan4.0.

FIG. 8C shows a precision recall curve of the disclosed presentation model (referred to as “EDGE”) in comparison to BERTMHC and NetMHCIIpan4.0.

FIG. 8D shows a true positive rate v. false positive rate plot of the disclosed presentation model (EDGE (LGN+IP-MS)) in comparison to BERTMHC and NetMHCIIpan4.0.

FIG. 9 shows example increased weights of the learned genotype network (LGN) when an epitope is presented in comparison to when an epitope is not presented.

FIG. 10 shows correlation of the predicted score and immunogenic response.

FIGS. 11A and 11B show posterior distributions of logistic coefficient demonstrates that the disclosed presentation model is predictive of immunogenicity in personalized mRNA vaccines.

FIG. 12A shows differential model performances on the Reynisson et al. dataset. FIG. 12B shows gradient of each allele when calculating the EDGE-II score using either maximal score deconvolution or the LAN for the following peptide, SVPAQAPKRTQAPTKA.

FIG. 13A left shows the approach taken to evaluate the ability of EDGE-II to predict the induction of CD4+ T cell responses post vaccination. FIG. 13A right shows predicted probability of immunogenicity between the two models. FIG. 13B shows a specific motif involved in positive presentation prediction. FIG. 13C shows that five healthy donors had an allele for which the EDGE-II score exceeded p=0.5, and all 6 scores were greater than the 95^{th} percentile for that allele.

FIG. 14A shows a strong association between DQ alleles with high EDGE-II scores as compared to DP and DR alleles. FIG. 14B shows a peptide, KLVVVGACGVGKSAL, containing the motif (FIG. 13B), that was found to be presented via DQ by mass spectrometry.

FIG. 15A shows KRAS G12C-specific T cell responses. FIG. 15B shows deconvolution to single peptides ex vivo or post-IVS confirmed functional responses to EDGE-II predicted class II epitopes. FIGS. 15C and 15D show CD8+ and CD4+ T cell mediated responses to Peptide_29 as measured by IFNγ (IFN gamma).

FIGS. 16A and 16B show IFNγ, CD107α, interleukin (IL)-2, tumor necrosis factor (TNF)-α/TNFα expression profiles in CD4+ and CD8+ T cells following stimulation. FIGS. 16C and 16D show Boolean gating of quadruple-, triple-, and double-positive polyfunctional CD4+ T cells.

FIG. 17A shows CD4+ and CD8+ T cell-driven responses to G12C Peptide_91 in both donors via post-IVS ELISpot assay. FIG. 17B shows relative Peptide_91-pulsed target cells count following co-culturing with whole PBMSs from two donors. FIG. 17C shows IFNγ, TNFα, Perforin, and Granzyme B (GRZB) levels in two donors with Peptide_91-stimulation in PBMCs, CD8-depleted, and CD4-depleted conditions. FIG. 17D shows CD4+ T-cell mediated killing of target cells presenting g12C single peptide #91. FIG. 17E shows production of IFNγ, IL-2, TNFα, Perforin, and GRZB.

FIG. 18A shows EDGE-II presentation scores for HLA II genotypes. FIG. 18B shows two TCR clonotypes (TCR969 and TCR995) that elicited increased expression of activation markers CD69 and CD25 on rTCR Jurkat cells in response to KRAS G12C peptide pools and KRAS G12C single peptides. FIG. 18C shows transcriptional clustering analyses of CD4+ and CD8+ T cell phenotype clusters with varying degrees of TCR clonotype expansion across the samples. FIG. 18D shows dimensionality reduction of CEF-stimulated and G12C stimulated cell conditions. FIG. 18E shows the CD4+ cytotoxic transcriptional profiles of TCR969 and TCR995. FIG. 18F shows G12C-specific patient TCRs are CD4+ T cell derived and have a gene profile indicating cytotoxic capability.

FIG. 19A shows the average precision of identical model architectures trained and validated on the same data from Reynisson et al (2020b), with bootstrap confidence intervals.

FIG. 19B shows a histogram that summarized the amount of MS peptides as a function of the number of alleles that were used for deconvolution and to predict a given peptide. Reynisson et al.'s data is either SA or MA, whereas Gritstone's (grts) are either SA or DR/DP/DQ-specific IP-MS data.

FIG. 20 shows sampled posterior distributions for EDGE-II (left) and MARIA (right) from Bayesian logistic regression analysis. a is the intercept term and β is the regression coefficient.

FIGS. 21A through 21D show mass spectra comparing the biological sample (FIGS. 21A and 21C) to synthetic standard (FIGS. 21B and 21D), with the mass error shown in the center. Fragmentation points in each peptide are shown for N-terminal fragments (blue) and C-terminal fragments (red).

FIG. 22 shows the score distribution across alleles present in training data for KRAS G12C in EDGE-II with top scoring peptides shown.

FIG. 23A shows T cell responses to KRAS G12C class II peptide pools and individual peptides (Table 6) assessed by post-IVS IFNγ ELISpot. FIG. 23B shows T cell responses to KRAS WT peptide pool (Table 6) assessed by ex vivo IFNγ ELISpot. FIG. 23C shows a schematic of peptide sequences for KRAS WT and G12C (amino acids 1-25), and corresponding sequence from bacterial lipoprotein LppX with natural sequence (LppX-Cys_Cys) and with second Cysteine replaced by an Alanine (LppX_Cyx_Ala). In addition, EDGETM predicted core sequences for presentation in Class II are shown. FIG. 23D shows T cell responses to KRAS G12C peptide pool (Table 6) or LppX_Cys_Ala 25mer peptide (FIG. 23C) assessed by ex vivo IFNγ ELISpot. FIG. 23E shows healthy donor screening results in response to G12C minimal epitope peptides. FIG. 23F shows selected healthy donors responses to 11-25 amino acid (aa or AA) G12C class II peptides.

FIG. 24 shows representative dot plots (Donor AC16443) of post-IVS CD8+ T cell responses to vehicle control (DMSO, top row), KRAS G12C class II Pool 2 (middle row), or single peptide (bottom row; Table 6) assessed by intracellular cytokine staining (ICS).

FIG. 25A shows T cell responses to LppX_Cys_Ala 25mer and controls assessed by post-IVS IFNγ ELISpot for PBMCs, CD4^{depleted} PBMCs, and CD8^{depleted} PBMCs. FIG. 25B shows target cell killing by IVS-expanded PBMCs or depleted populations from donors AC13990 (left) and AC16443 (right) assessed by IncuCyte® assay. FIG. 25C shows cytokine secretion from killing assay LppX_Cys_Ala stimulated co-culture condition supernatants (PBMCs, CD8^{depl} PBMCs, or CD4^{depl} PBMCs) assessed by ELLA assay.

FIG. 26A shows schematic representation of patient G05-002-0122 tumor profile and therapies prior to enrollment and vaccination in study NCT03953235. FIG. 26B shows T cell responses to KRAS G12C peptide pool (Table 6) assessed by ex vivo IFNγ ELISpot. FIGS. 26C and 26D show T cell responses to KRAS G12C peptide pool and individual peptides (Table 6) or controls as assessed by ex vivo IFNγ ELISpot for PBMCs (FIG. 26C), CD4^{depl} PBMCs, and CD8^{depl} PBMCs (FIG. 26D).

FIG. 27A shows post-vaccination T cell responses to KRAS G12C peptide pool (Table 6) and controls assessed by post-IVS IFNγ ELISpot for Patient G05-002-0122. FIG. 27B shows schematic outlining single cell sequencing approach for TCR seq and digital gene expression (DGE) analyses.

FIGS. 28A and 28B show gating strategies for (28A) depletion assays and (28B) intracellular cytokine staining (ICS).

FIG. 29 shows the Jurkat functional screening gating strategy.

FIG. 30 shows ex vivo ELISpot IFNγ responses for PMBCs from healthy donors following stimulation with various KRAS neoepitopes.

FIG. 31 shows ex vivo ELISpot IFNγ responses for PMBCs from a single donor (SE-0386) following stimulation with a CD8 MHC class I peptide pool, and various single CD8 MHC class I peptides including MHC class I peptide #29 (EYKLVVVGACG).

FIG. 32 shows ex vivo ELISpot IFNγ responses for PMBCs from the indicated single donors following stimulation with a DMSO control (left column), a CD8 MHC class I peptide pool (middle column), or CD8 pool MHC class I peptide #29 (right column). Shown are responses for total PBMCs, CD8 enriched (CD4 depleted), and CD4 enriched (CD8 depleted) samples.

FIG. 33 shows ex vivo ELISpot IFNγ responses for PMBCs from a single donor (SE-0659 having HLA DRB1*07:01) following stimulation with a DMSO control (column 1 for each sample type) or KRAS G12C MHC class II peptide pools (Class II pools 1-4 for columns 2-5 for each sample type, respectively). Shown are responses for total PBMCs, CD8 enriched (CD4 depleted), and CD4 enriched (CD8 depleted) samples.

FIG. 34 shows ex vivo fluorospot IFNγ and IL-2 responses for PMBCs from a single donor (AC16443 having HLA DRB1*01:01) following stimulation with a DMSO control (columns 1-2) or KRAS G12C MHC class II peptide pools (pool 1 columns 3-4, pool 2 columns 5-6, pool 3 columns 7-8, pool 4 columns 9-10).

FIG. 35 shows ex vivo ELISpot IFNγ responses for PMBCs from single donors (top pan1 SE-0659 having HLA DRB1*07:01; bottom panel AC16443 having HLA DRB1*01:01) following stimulation with a DMSO control, indicated KRAS G12C MHC class II peptide pool, or the indicated single KRAS G12C MHC class II peptide.

FIG. 36 shows a summary of ex vivo ELISpot IFNγ responses for PMBCs from single healthy donors with either DRB1*01:01 or DRB1*07:01 when stimulated overnight with KRAS G12C class II pools or single peptide 40.

FIG. 37 shows tumor microenvironment contexture across patients based on tumor tissue utilized for neoantigen prediction. A heatmap of RNAseq from pre-treatment tumor tissue organized by tumor microenvironments based on gene expression signatures and the association with tumor mutation burden (TMB) and molecular response. Tumor RNAseq derived gene expression and tumor microenvironment contexture analyses calculated using ssGSEA and median centered and scaled.

FIG. 38A shows a heatmap organized by tumor microenvironments based on gene expression signatures relating to TMB, molecular response, and the site of tumor tissue used for neoantigen prediction. calculated using ssGSEA and median centered and scaled. FIG. 38B shows a heatmap of RNAseq expression of immune-related genes and effector T cell gene signatures relating to TMB, molecular response, and the site of tumor tissue used for neoantigen prediction. RNAseq expression was derived from DEseq2 normalized RSEM expected counts.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response.

As used herein the term “neoantigen” is an antigen that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF.

A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358. Example methods for identifying tumor specific mutations in neoantigens are described in WO2018195357, which is incorporated by reference in its entirety.

As used herein the term “tumor neoantigen” is a neoantigen present in a subject's tumor cell or tissue but not in the subject's corresponding normal cell or tissue.

As used herein the term “neoantigen-based vaccine” is a vaccine construct based on one or more neoantigens, e.g., a plurality of neoantigens.

As used herein the term “candidate neoantigen” is a mutation or other aberration giving rise to a new sequence that may represent a neoantigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon.

As used herein the term “frameshift mutation” is a mutation causing a change in the frame of the protein.

As used herein the term “indel” is an insertion or deletion of one or more nucleic acids.

As used herein, the term “percent identity”, in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and the sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to elicit an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “truncal mutation” is a mutation originating early in the development of a tumor and present in a substantial portion of the tumor's cells.

As used herein the term “subclonal mutation” is a mutation originating later in the development of a tumor and present in only a subset of the tumor's cells.

As used herein the term “exome” is a subset of the genome that codes for proteins. An exome can be the collective exons of a genome.

As used herein the term “logistic regression” is a regression model for binary data from statistics where the logit of the conditional probability that the dependent variable is equal to one is modeled as a linear function of the dependent variables.

As used herein the term “neural network” is a machine learning model for classification or regression including multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the tumor peptidome, meaning the union of the peptidomes of all cells that comprise the tumor).

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The terms “subject” and “patient” are used interchangeably and encompass a cell, tissue, or organism, human or non-human, whether in vivo, ex vivo, or in vitro, male or female.

The term subject is inclusive of mammals including humans.

The term “mammal” encompasses both humans and non-humans and includes but is not limited to humans, non-human primates, canines, felines, murines, bovines, equines, and porcines.

Abbreviations: MHC: major histocompatibility complex; HLA: human leukocyte antigen, or the human MHC gene locus; NGS: next-generation sequencing; PPV: positive predictive value; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. System Overview

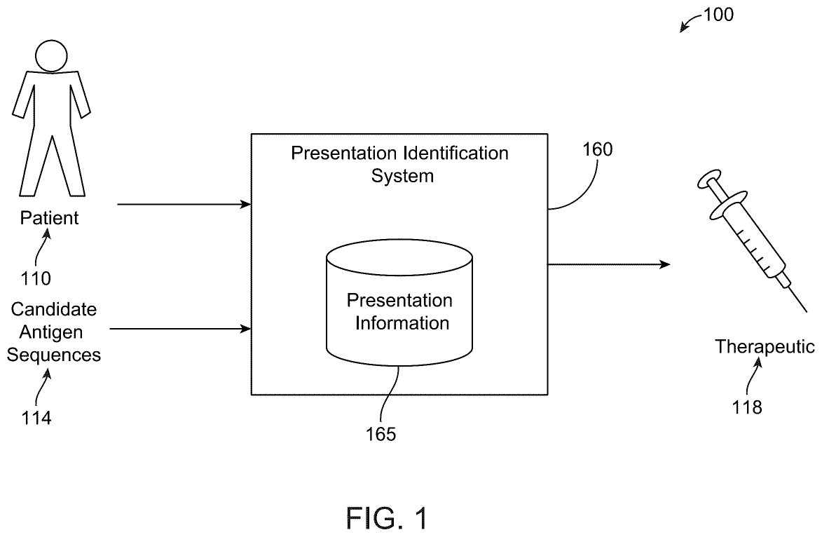

FIG. 1 is an overview of an environment for identifying likelihoods of peptide presentation in patients, in accordance with an embodiment. The environment 100 provides context in order to introduce a presentation identification system 160, itself including a presentation information store 165.

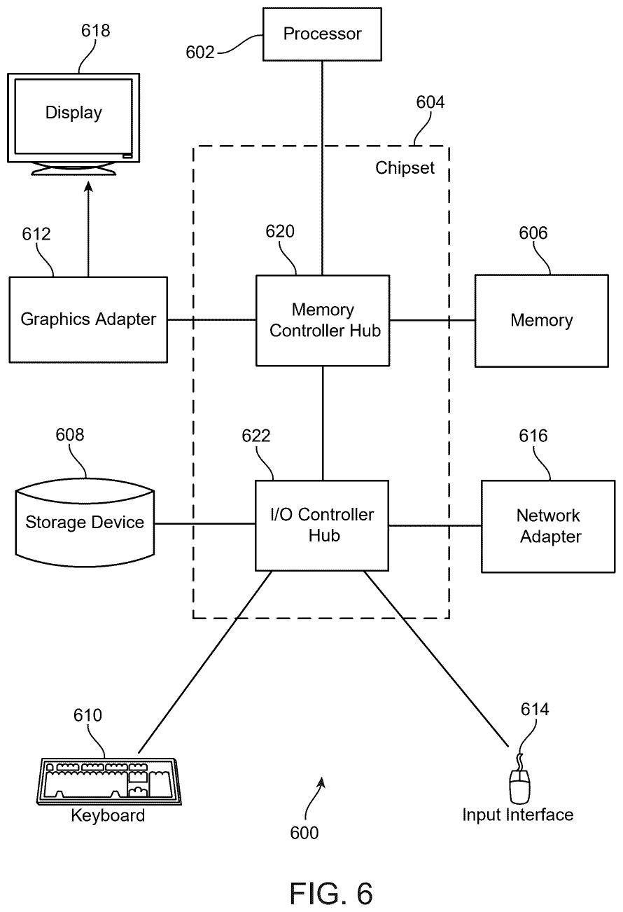

The presentation identification system 160 is one or more computer models, embodied in a computing system or device as discussed below with respect to FIG. 6, that receives peptide sequences associated with a set of MHC alleles and determines likelihoods that the peptide sequences will be presented by one or more of the set of MHC alleles. In particular embodiments, the set of MHC alleles represent a genotype of a patient, such that the patient expresses the set of MHC alleles. The presentation identification system 160 may be applied to both class I and class II MHC alleles. In particular embodiments, presentation identification system 160 is applied specifically to class II MHC alleles. Therefore, the set of alleles may represent a class II genotype of a patient.

One specific use case for the presentation identification system 160 is that it is able to receive nucleotide sequences of candidate neoantigens (e.g., shown as candidate antigen sequences 114 in FIG. 1) associated with a set of MHC alleles from tumor cells of a patient 110 and determine likelihoods that the candidate neoantigens will be presented by one or more of the associated MHC alleles of the tumor and/or induce immunogenic responses in the immune system of the patient 110. Those candidate neoantigens with high likelihoods as determined by system 160 can be selected for inclusion in a therapeutic 118, such an anti-tumor immune response can be elicited from the immune system of the patient 110 providing the tumor cells.

The presentation identification system 160 determines presentation likelihoods through one or more presentation models. Specifically, the presentation models generate likelihoods of whether given peptide sequences will be presented for a set of associated MHC alleles, and are generated based on presentation information stored in store 165. The presentation information 165 contains information on whether peptides bind to different types of MHC alleles such that those peptides are presented by MHC alleles, which in the models is determined depending on positions of amino acids in the peptide sequences. The presentation model can predict whether an unrecognized peptide sequence will be presented in association with an associated set of MHC alleles based on the presentation information 165.

### II.A. Presentation Information

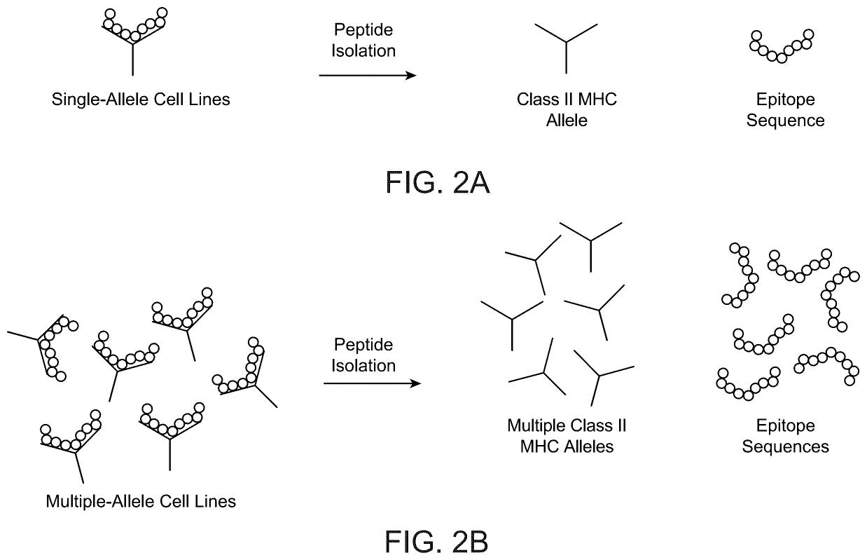

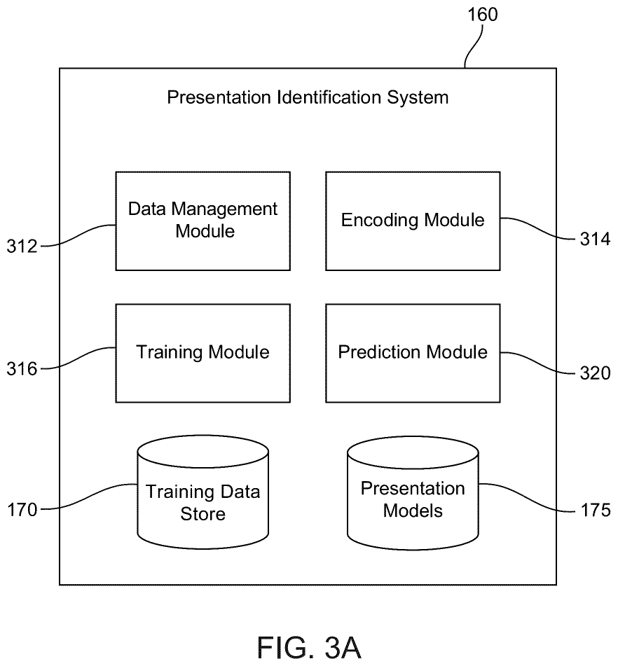

FIGS. 2A and 2B illustrate a method of obtaining presentation information, in accordance with an embodiment. The presentation information 165 includes two general categories of information: allele-interacting information and allele-noninteracting information.

Allele-interacting information includes information that influence presentation of peptide sequences that are dependent on the type of MHC allele. Allele-noninteracting information includes information that influence presentation of peptide sequences that are independent on the type of MHC allele.

Allele-interacting information primarily includes identified peptide sequences that are known to have been presented by one or more identified MHC molecules from humans, mice, etc. Notably, this may or may not include data obtained from tumor samples. In various embodiments, the presented peptide sequences may be identified from cells that express a single MHC allele. In this case the presented peptide sequences are generally collected from single-allele cell lines that are engineered to express a predetermined MHC allele and that are subsequently exposed to synthetic protein. Peptides presented on the MHC allele are isolated by techniques such as acid-elution and identified through mass spectrometry. FIG. 2A shows an example of this, where a single epitope sequence is presented on the predetermined MHC allele (e.g., class II MHC allele). The epitope is isolated and identified through mass spectrometry. Since in this situation peptides are identified through cells engineered to express a single predetermined MHC protein, the direct association between a presented peptide and the MHC protein to which it was bound to is definitively known.

The presented peptide sequences may also be collected from cells that express multiple MHC alleles. Typically in humans, 6 different types of MHC-I and up to 12 different types of MHC-II molecules are expressed for a cell. Such presented peptide sequences may be identified from multiple-allele cell lines that are engineered to express multiple predetermined MHC alleles. Such presented peptide sequences may also be identified from tissue samples, either from normal tissue samples or tumor tissue samples. In this case particularly, the MHC molecules can be immunoprecipitated from normal or tumor tissue. Peptides presented on the multiple MHC alleles can similarly be isolated by techniques such as acid-elution and identified through mass spectrometry. FIG. 2B shows an example of this, where example peptides are presented on a set of class I MHC alleles. Although FIG. 2B shows an example six alleles, there may be more alleles (e.g., up to 12 class II MHC alleles). The epitopes and are isolated and identified through mass spectrometry. In contrast to single-allele cell lines, the direct association between a presented peptide and the MHC protein to which it was bound to may be unknown since the bound peptides are isolated from the MHC molecules before being identified.

Allele-interacting information can also include mass spectrometry ion current which depends on both the concentration of peptide-MHC molecule complexes, and the ionization efficiency of peptides. The ionization efficiency varies from peptide to peptide in a sequence-dependent manner. Generally, ionization efficiency varies from peptide to peptide over approximately two orders of magnitude, while the concentration of peptide-MHC complexes varies over a larger range than that.

Allele-interacting information can also include measurements or predictions of binding affinity between a given MHC allele and a given peptide. One or more affinity models can generate such predictions. For example, presentation information 165 may include a binding affinity prediction between a peptide sequence and the MHC allele. As a specific example, presentation information 165 may include a binding affinity prediction between a peptide sequence and the class II allele HLA-DRB1:11:01.

Allele-interacting information can also include measurements or predictions of stability of the MHC complex. One or more stability models that can generate such predictions. More stable peptide-MHC complexes (i.e., complexes with longer half-lives) are more likely to be presented at high copy number on tumor cells and on antigen-presenting cells that encounter vaccine antigen. For example, presentation information 165 may include a stability prediction of a half-life of 1h for the class I molecule HLA-A*01:01. Presentation information 165 may also include a stability prediction of a half-life for the class II molecule HLA-DRB1:11:01.

Allele-interacting information can also include the measured or predicted rate of the formation reaction for the peptide-MHC complex. Complexes that form at a higher rate are more likely to be presented on the cell surface at high concentration.

Allele-interacting information can also include the sequence and length of the peptide. MHC class I molecules typically prefer to present peptides with lengths between 8 and 15 peptides. 60-80% of presented peptides have length 9. MHC class II molecules typically prefer to present peptides with lengths between 6-30 peptides.

Allele-interacting information can also include the presence of kinase sequence motifs on the neoantigen encoded peptide, and the absence or presence of specific post-translational modifications on the neoantigen encoded peptide. The presence of kinase motifs affects the probability of post-translational modification, which may enhance or interfere with MHC binding.

Allele-interacting information can also include the expression or activity levels of proteins involved in the process of post-translational modification, e.g., kinases (as measured or predicted from RNA seq, mass spectrometry, or other methods).

Allele-interacting information can also include the probability of presentation of peptides with similar sequence in cells from other individuals expressing the particular MHC allele as assessed by mass-spectrometry proteomics or other means.

Allele-interacting information can also include the expression levels of the particular MHC allele in the individual in question (e.g. as measured by RNA-seq or mass spectrometry). Peptides that bind most strongly to an MHC allele that is expressed at high levels are more likely to be presented than peptides that bind most strongly to an MHC allele that is expressed at a low level.

Allele-interacting information can also include the overall neoantigen encoded peptide-sequence-independent probability of presentation by the particular MHC allele in other individuals who express the particular MHC allele.

Allele-interacting information can also include the overall peptide-sequence-independent probability of presentation by MHC alleles in the same family of molecules (e.g., HLA-A, HLA-B, HLA-C, HLA-DQ, HLA-DR, HLA-DP) in other individuals. For example, HLA-C molecules are typically expressed at lower levels than HLA-A or HLA-B molecules, and consequently, presentation of a peptide by HLA-C is a priori less probable than presentation by HLA-A or HLA-B. For another example, HLA-DP is typically expressed at lower levels than HLA-DR or HLA-DQ; consequently, presentation of a peptide by HLA-DP is less probable than presentation by HLA-DR or HLA-DQ.

Allele-interacting information can also include the protein sequence of the particular MHC allele.

Any MHC allele-noninteracting information listed in the below section can also be modeled as an MHC allele-interacting information.

Allele-noninteracting information can include C-terminal sequences flanking the neoantigen encoded peptide within its source protein sequence. For MHC-I, C-terminal flanking sequences may impact proteasomal processing of peptides. However, the C-terminal flanking sequence is cleaved from the peptide by the proteasome before the peptide is transported to the endoplasmic reticulum and encounters MHC alleles on the surfaces of cells. Consequently, MHC molecules receive no information about the C-terminal flanking sequence, and thus, the effect of the C-terminal flanking sequence cannot vary depending on MHC allele type. For example, presentation information 165 may include the C-terminal flanking sequence of the presented peptide identified from the source protein of the peptide.

Allele-noninteracting information can also include mRNA quantification measurements. For example, mRNA quantification data can be obtained for the same samples that provide the mass spectrometry training data. RNA expression can be identified to be a strong predictor of peptide presentation. In one embodiment, the mRNA quantification measurements are identified from software tool RSEM. Detailed implementation of the RSEM software tool can be found at Bo Li and Colin N. Dewey. RSEM: accurate transcript quantification from RNA-Seq data with or without a reference genome. BMC Bioinformatics, 12:323, August 2011. In one embodiment, the mRNA quantification is measured in units of fragments per kilobase of transcript per Million mapped reads (FPKM).

Allele-noninteracting information can also include the N-terminal sequences flanking the peptide within its source protein sequence.

Allele-noninteracting information can also include the source gene of the peptide sequence. The source gene may be defined as the Ensembl protein family of the peptide sequence. In other examples, the source gene may be defined as the source DNA or the source RNA of the peptide sequence. The source gene can, for example, be represented as a string of nucleotides that encode for a protein, or alternatively be more categorically represented based on a named set of known DNA or RNA sequences that are known to encode specific proteins. In another example, allele-noninteracting information can also include the source transcript or isoform or set of potential source transcripts or isoforms of the peptide sequence drawn from a database such as Ensembl or RefSeq.

Allele-noninteracting information can also include the presence of protease cleavage motifs in the peptide, optionally weighted according to the expression of corresponding proteases in the tumor cells (as measured by RNA-seq or mass spectrometry). Peptides that contain protease cleavage motifs are less likely to be presented, because they will be more readily degraded by proteases, and will therefore be less stable within the cell.

Allele-noninteracting information can also include the turnover rate of the source protein as measured in the appropriate cell type. Faster turnover rate (i.e., lower half-life) increases the probability of presentation; however, the predictive power of this feature is low if measured in a dissimilar cell type.

Allele-noninteracting information can also include the length of the source protein, optionally considering the specific splice variants (“isoforms”) most highly expressed in the tumor cells as measured by RNA-seq or proteome mass spectrometry, or as predicted from the annotation of germline or somatic splicing mutations detected in DNA or RNA sequence data.

Allele-noninteracting information can also include the level of expression of the proteasome, immunoproteasome, thymoproteasome, or other proteases in the tumor cells (which may be measured by RNA-seq, proteome mass spectrometry, or immunohistochemistry). Different proteasomes have different cleavage site preferences. More weight will be given to the cleavage preferences of each type of proteasome in proportion to its expression level.

Allele-noninteracting information can also include the expression of the source gene of the peptide (e.g., as measured by RNA-seq or mass spectrometry). Possible optimizations include adjusting the measured expression to account for the presence of stromal cells and tumor-infiltrating lymphocytes within the tumor sample. Peptides from more highly expressed genes are more likely to be presented. Peptides from genes with undetectable levels of expression can be excluded from consideration.

Allele-noninteracting information can also include the probability that the source mRNA of the neoantigen encoded peptide will be subject to nonsense-mediated decay as predicted by a model of nonsense-mediated decay, for example, the model from Rivas et al, Science 2015.

Allele-noninteracting information can also include the typical tissue-specific expression of the source gene of the peptide during various stages of the cell cycle. Genes that are expressed at a low level overall (as measured by RNA-seq or mass spectrometry proteomics) but that are known to be expressed at a high level during specific stages of the cell cycle are likely to produce more presented peptides than genes that are stably expressed at very low levels.

Allele-noninteracting information can also include a comprehensive catalog of features of the source protein as given in e.g. uniProt or PDB http://www.rcsb.org/pdb/home/home.do. These features may include, among others: the secondary and tertiary structures of the protein, subcellular localization 11, Gene ontology (GO) terms. Specifically, this information may contain annotations that act at the level of the protein, e.g., 5′ UTR length, and annotations that act at the level of specific residues, e.g., helix motif between residues 300 and 310. These features can also include turn motifs, sheet motifs, and disordered residues.

Allele-noninteracting information can also include features describing the properties of the domain of the source protein containing the peptide, for example: secondary or tertiary structure (e.g., alpha helix vs beta sheet); Alternative splicing.

Allele-noninteracting information can also include features describing the presence or absence of a presentation hotspot at the position of the peptide in the source protein of the peptide.

Allele-noninteracting information can also include the probability of presentation of peptides from the source protein of the peptide in question in other individuals (after adjusting for the expression level of the source protein in those individuals and the influence of the different HLA types of those individuals).

Allele-noninteracting information can also include the probability that the peptide will not be detected or over-represented by mass spectrometry due to technical biases.

The expression of various gene modules/pathways as measured by a gene expression assay such as RNASeq, microarray(s), targeted panel(s) such as Nanostring, or single/multi-gene representatives of gene modules measured by assays such as RT-PCR (which need not contain the source protein of the peptide) that are informative about the state of the tumor cells, stroma, or tumor-infiltrating lymphocytes (TILs).

Allele-noninteracting information can also include the copy number of the source gene of the peptide in the tumor cells. For example, peptides from genes that are subject to homozygous deletion in tumor cells can be assigned a probability of presentation of zero.

Allele-noninteracting information can also include the probability that the peptide binds to the TAP or the measured or predicted binding affinity of the peptide to the TAP.

Peptides that are more likely to bind to the TAP, or peptides that bind the TAP with higher affinity are more likely to be presented by MHC-I.

Allele-noninteracting information can also include the expression level of TAP in the tumor cells (which may be measured by RNA-seq, proteome mass spectrometry, immunohistochemistry). For MHC-I, higher TAP expression levels increase the probability of presentation of all peptides.

Allele-noninteracting information can also include the presence or absence of tumor mutations, including, but not limited to:
i. Driver mutations in known cancer driver genes such as EGFR, KRAS, ALK, RET, ROS1, TP53, CDKN2A, CDKN2B, NTRK1, NTRK2, NTRK3
ii. In genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOB, HLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome). Peptides whose presentation relies on a component of the antigen-presentation machinery that is subject to loss-of-function mutation in the tumor have reduced probability of presentation.

Presence or absence of functional germline polymorphisms, including, but not limited to: In genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOB, HLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome)

Allele-noninteracting information can also include tumor type (e.g., NSCLC, melanoma).

Allele-noninteracting information can also include known functionality of HLA alleles, as reflected by, for instance HLA allele suffixes. For example, the N suffix in the allele name HLA-A*24:09N indicates a null allele that is not expressed and is therefore unlikely to present epitopes; the full HLA allele suffix nomenclature is described at https://www.ebi.ac.uk/ipd/imgt/hla/nomenclature/suffixes.html.

Allele-noninteracting information can also include clinical tumor subtype (e.g., squamous lung cancer vs. non-squamous).

Allele-noninteracting information can also include smoking history.

Allele-noninteracting information can also include history of sunburn, sun exposure, or exposure to other mutagens.

Allele-noninteracting information can also include the typical expression of the source gene of the peptide in the relevant tumor type or clinical subtype, optionally stratified by driver mutation. Genes that are typically expressed at high levels in the relevant tumor type are more likely to be presented.

Allele-noninteracting information can also include the frequency of the mutation in all tumors, or in tumors of the same type, or in tumors from individuals with at least one shared MHC allele, or in tumors of the same type in individuals with at least one shared MHC allele.

In the case of a mutated tumor-specific peptide, the list of features used to predict a probability of presentation may also include the annotation of the mutation (e.g., missense, read-through, frameshift, fusion, etc.) or whether the mutation is predicted to result in nonsense-mediated decay (NMD). For example, peptides from protein segments that are not translated in tumor cells due to homozygous early-stop mutations can be assigned a probability of presentation of zero. NMD results in decreased mRNA translation, which decreases the probability of presentation.

### II.B. Presentation Identification System

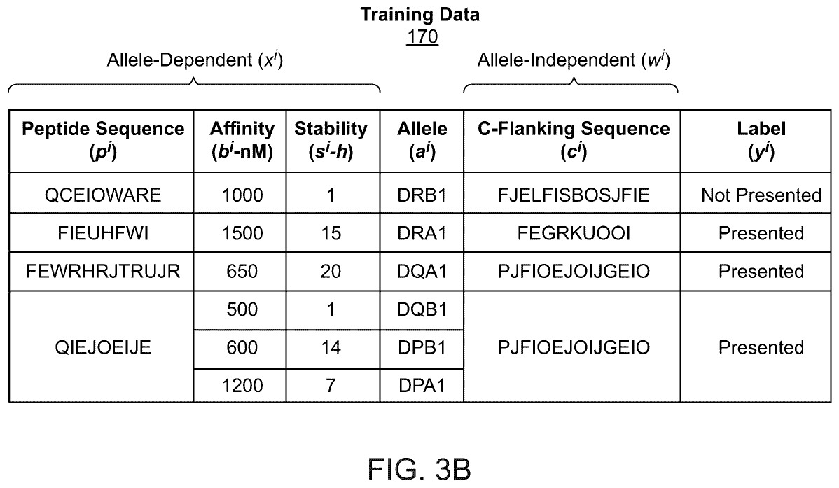

FIG. 3A is a high-level block diagram illustrating the computer logic components of the presentation identification system 160, according to one embodiment. In this example embodiment, the presentation identification system 160 includes a data management module 312, an encoding module 314, a training module 316, and a prediction module 320. The presentation identification system 160 is also comprised of a training data store 170 and a presentation models store 175. Some embodiments of the presentation identification system 160 have different modules than those described here. Similarly, the functions can be distributed among the modules in a different manner than is described here.

The data management module 312 generates sets of training data 170 from the presentation information 165. Each set of training data contains a plurality of data instances, in which each data instance i contains a set of independent variables zⁱ that include one or more of a presented or non-presented peptide sequence pⁱ, one or more associated MHC alleles aⁱ associated with the peptide sequence pⁱ, and a dependent variable yⁱ that represents information that the presentation identification system 160 is interested in predicting for new values of independent variables.

In one particular implementation, the dependent variable yⁱ is a binary label indicating whether peptide pⁱ was presented by the one or more associated MHC alleles aⁱ (e.g., MHC alleles representing a genotype of a patient) However, it is appreciated that in other implementations, the dependent variable yⁱ can represent any other kind of information that the presentation identification system 160 is interested in predicting dependent on the independent variables zⁱ. For example, in another implementation, the dependent variable yⁱ may also be a numerical value indicating the mass spectrometry ion current identified for the data instance.

The peptide sequence pⁱ for data instance i is a sequence of kᵢ amino acids, in which kᵢ may vary between data instances i within a range. For example, that range may be 8-15 for MHC class I or 6-30 for MHC class II. In one specific implementation of presentation identification system 160, all peptide sequences pⁱ in a training data set may have the same length, e.g. 9. The number of amino acids in a peptide sequence may vary depending on the type of MHC alleles (e.g., MHC alleles in humans, etc.). The MHC alleles aⁱ for data instance i indicate which MHC alleles were present in association with the corresponding peptide sequence pⁱ.

The data management module 312 may also include additional allele-interacting variables, such as binding affinity bⁱ and stability sⁱ predictions in conjunction with the peptide sequences pⁱ and associated MHC alleles aⁱ contained in the training data 170. For example, the training data 170 may contain binding affinity predictions bⁱ between a peptide pⁱ and each of the associated MHC molecules indicated in aⁱ. As another example, the training data 170 may contain stability predictions sⁱ for each of the MHC alleles indicated in aⁱ.

The data management module 312 may also include allele-noninteracting variables wⁱ, such as C-terminal flanking sequences and mRNA quantification measurements in conjunction with the peptide sequences pⁱ.

The data management module 312 also identifies peptide sequences that are not presented by MHC alleles to generate the training data 170. Generally, this involves identifying the “longer” sequences of source protein that include presented peptide sequences prior to presentation. When the presentation information contains engineered cell lines, the data management module 312 identifies a series of peptide sequences in the synthetic protein to which the cells were exposed to that were not presented on MHC alleles of the cells. When the presentation information contains tissue samples, the data management module 312 identifies source proteins from which presented peptide sequences originated from, and identifies a series of peptide sequences in the source protein that were not presented on MHC alleles of the tissue sample cells.

The data management module 312 may also artificially generate peptides with random sequences of amino acids and identify the generated sequences as peptides not presented on MHC alleles. This can be accomplished by randomly generating peptide sequences allows the data management module 312 to easily generate large amounts of synthetic data for peptides not presented on MHC alleles. Since in reality, a small percentage of peptide sequences are presented by MHC alleles, the synthetically generated peptide sequences are highly likely not to have been presented by MHC alleles even if they were included in proteins processed by cells.

FIG. 3B illustrates an example set of training data 170, according to one embodiment. Specifically, the first data instance in the training data 170 indicates peptide presentation information from a single-allele cell line involving the allele HLA-DRB1 and peptide sequence QCEIOWAREFLKEIGJ (SEQ ID NO: XX). The second data instance in the training data 170 indicates peptide presentation information from a single-allele cell line involving the allele HLA-DRA1 and peptide sequence FIEUHFWI (SEQ ID NO: XX).

The third data instance in the training data 170 indicates peptide presentation information from a single-allele cell line involving the allele HLA-DQA1 and FEWRHRJTRUJR (SEQ ID NO: XX). The fourth data instance in the training data 170 indicates peptide information from a multiple-allele cell line involving the alleles HLA-DQB1, HLA-DPB1, and HLA-DPA1 and a peptide sequence QIEJOEIJE (SEQ ID NO: XX). In various embodiments, instead of training data indicating whether a particular allele presented a particular epitope sequence, the training data 170 may include intermediate resolution data. As used herein, “intermediate resolution data” refers to data that is higher resolution than full, multi-allele data, but lower resolution than single-allele data. The “resolution” of a data refers to the ability to extract which MHC allele presented an epitope. Thus, single-allele data is the highest resolution data in which presentation of an epitope can be directly attributed to a single allele. Full, multi-allele data (e.g., multi-allele data of six or more MHC alleles) is lower resolution given that presentation of an epitope can only be attributed to a set of MHC alleles. Intermediate resolution data may be include presentation of epitopes by two or more MHC alleles. Therefore, intermediate resolution data is lower resolution than single-allele data in that presentation of an epitope cannot be directly attributed to an allele. Given that single-allele data can be difficult and expensive to produce, intermediate resolution data may be more preferable. Intermediate resolution data can be higher resolution than full, multi-allele data. As an example, intermediate resolution data may indicate whether a family of alleles presented a particular epitope sequence. For example, each data instance may identify whether a peptide sequence was presented or not presented by a family of alleles, such as one of HLA-DR, HLA-DQ, or HLA-DP class II alleles. For example, the HLA-DR family of alleles can include, but is not limited to HLA-DPA1 and HLA-DPB1. The HLA-DQ family of alleles can include, but is not limited to HLA-DQA1, HLA-DQA2, HLA-DQB1, and HLA-DQB2. The HLA-DR family of alleles can include, but is not limited to HLA-DRA, HLA-DRB1, HLA-DRB3, HLA-DRB4, and HLA-DRB5. As described in further detail herein, methods can involve isolating specific HLA-peptide molecules and more specifically, isolating specific families of HLA alleles.

In various embodiments, isolation of HLA-peptide molecules can be performed using classic immunoprecipitation (IP) methods after lysis and solubilization of tissue sample. A clarified lysate can be used for HLA specific IP. Immunoprecipitation may be performed using antibodies coupled to beads where the antibody is specific for HLA molecules. For a pan-Class I HLA immunoprecipitation, a pan-Class I CR antibody is used, for Class II HLA-DR, an HLA-DR antibody is used. The antibody is covalently attached to NHS-sepharose beads during overnight incubation. After covalent attachment, the beads are washed and aliquoted for IP.

Immunoprecipitations can also be performed with antibodies that are not covalently attached to beads. This may be accomplished using sepharose or magnetic beads coated with Protein A and/or Protein G to hold the antibody to the column. Example antibodies that can be used to selectively enrich MHC/peptide complex are listed below.

| Antibody Name | Specificity |
|---|---|
| W6/32 | Class I HLA-A, B, C |
| L243 | Class II - HLA-DR |
| Tu36 | Class II - HLA-DR |
| LN3 | Class II - HLA-DR |
| Tu39 | Class II - HLA-DR, DP, DQ |
| WR18 (Bio-Rad, | Class II - HLA-DR, DP, DQ |
| Catalog # MCA477) | |
| B7/21 (Leinco Technologies, | Class II - HLA-DP |
| Catalog# H260) | |
| HLA-DQA1/2866R (MyBioSource, | Class II - HLA-DQ |
| Catalog# MBS4380589) | |

In particular embodiments, sub-samples (sub-samples split from a common sample) can undergo separate enrichment processes using different antibodies. For example, a sub-sample can undergo enrichment using an antibody exhibiting specificity for class II—HLA-DR alleles. As another example, a sub-sample can undergo enrichment using an antibody exhibiting specificity for class II—HLA-DP alleles. As another example, a sub-sample can undergo enrichment using an antibody exhibiting specificity for class II—HLA-DQ alleles. As another example, a first sub-sample can undergo enrichment using an antibody exhibiting specificity for class II—HLA-DP alleles, a second sub-sample can undergo enrichment using an antibody exhibiting specificity for class II—HLA-DR alleles, and a third sub-sample can undergo enrichment using an antibody exhibiting specificity for class II—HLA-DQ alleles. By selectively enriching MHC/peptide complexes for each of DR, DP, and DQ alleles, the intermediate resolution training data can be generated (e.g., lower resolution than single allelic training data but higher resolution than pan-Class II immunoprecipitation) indicating which family of alleles presented a particular epitope sequence.

Returning to FIG. 3B, the first data instance indicates that peptide sequence QCEIOWARE (SEQ ID NO: XX) was not presented by the allele HLA-DRB1. In various embodiments, the negatively-labeled peptide sequences may be randomly generated by the data management module 312 or identified from source protein of presented peptides. The training data 170 may also include a binding affinity prediction of 1000 nM and a stability prediction of a half-life of 1h for the peptide sequence-allele pair. The training data 170 also includes allele-noninteracting variables, such as the C-terminal flanking sequence of the peptide FJELFISBOSJFIE (SEQ ID NO: XX) and a mRNA quantification measurement of 10² TPM. The fourth data instance indicates that peptide sequence QIEJOEIJE (SEQ ID NO: XX) was presented by one of the alleles DQB1, DPB1, or DPA1. The training data 170 also includes binding affinity predictions and stability predictions for each of the alleles, as well as the C-terminal flanking sequence of the peptide and the mRNA quantification measurement for the peptide.

Referring next to the encoding module 314, it encodes information contained in the training data 170 into a numerical representation that can be used to generate the one or more presentation models. In one implementation, the encoding module 314 one-hot encodes sequences (e.g., peptide sequences or C-terminal flanking sequences) over a predetermined 20-letter amino acid alphabet. Specifically, a peptide sequence pⁱ with kᵢ amino acids is represented as a row vector of 20·kᵢ elements, where a single element among pⁱ_{20·(j-)+1}, pⁱ_{20·(j-1)+2}, . . . , pⁱ_{20·j} that corresponds to the alphabet of the amino acid at the j-th position of the peptide sequence has a value of 1. Otherwise, the remaining elements have a value of 0. As an example, for a given alphabet {A, C, D, E, F, G, H, I, K, L, M, N, P, Q, R, S, T, V, W, Y}, the peptide sequence EAF of 3 amino acids for data instance i may be represented by the row vector of 60 elements pⁱ=[0 0 0 1 0 0 0 0 0 0 0 0 0 0 0 0 0 0 0 0 1 0 0 0 0 0 0 0 0 0 0 0 0 0 0 0 0 0 0 0 0 0 0 0 1 0 0 0 0 0 0 0 0 0 0 0 0 0 0 0]. The C-terminal flanking sequence cⁱ can be similarly encoded as described above, as well as the protein sequence dh for MHC alleles, and other sequence data in the presentation information.

When the training data 170 contains sequences of differing lengths of amino acids, the encoding module 314 may further encode the peptides into equal-length vectors by adding a PAD character to extend the predetermined alphabet. For example, this may be performed by left-padding the peptide sequences with the PAD character until the length of the peptide sequence reaches the peptide sequence with the greatest length in the training data 170. Thus, when the peptide sequence with the greatest length has kₘₐₓ amino acids, the encoding module 314 numerically represents each sequence as a row vector of (20+1)·kₘₐₓ elements. As an example, for the extended alphabet {PAD, A, C, D, E, F, G, H, I, K, L, M, N, P, Q, R, S, T, V, W, Y} and a maximum amino acid length of kₘₐₓ=5, the same example peptide sequence EAF of 3 amino acids may be represented by the row vector of 105 elements pⁱ=[1 0 0 0 0 0 0 0 0 0 0 0 0 0 0 0 0 0 0 0 0 1 0 0 0 0 0 0 0 0 0 0 0 0 0 0 0 0 0 0 0 0 0 0 0 0 10 0 0 0 0 0 0 0 0 0 0 0 0 0 0 0 0 1 0 0 0 0 0 0 0 0 0 0 0 0 0 0 0 0 0 0 0 0 0 0 0 0 1 0 0 0 0 0 0 0 0 0 0 0 0 0 0 0]. The C-terminal flanking sequence cⁱ or other sequence data can be similarly encoded as described above. Thus, each independent variable or column in the peptide sequence pⁱ or cⁱ represents presence of a particular amino acid at a particular position of the sequence.

In one implementation, the encoding module 314 performs label encoding to map a particular amino acid to another value. Here, label encoding can involve the function of a lookup table that stores embeddings of a fixed dictionary and size. For example, the encoding module 314 maps a particular amino acid to an integer, and a trained embedding layer is used to convert the integer to a d-dimensional continuous embedding. In this situation, a particular integer value (e.g., “1”) corresponds to a particular learned vector. Thus, given a particular amino acid “A,” the encoding module 314 maps the amino acid “A” to an integer (e.g., “1”) which then queried against the look-up table to identify the corresponding learned embedding.

Although the above method of encoding sequence data was described in reference to sequences having amino acid sequences, the method can similarly be extended to other types of sequence data, such as DNA or RNA sequence data, and the like.

In various embodiments, the encoding module 314 also encodes the one or more MHC alleles aⁱ for data instance i as a row vector of m elements, in which each element h=1, 2, . . . , m corresponds to a unique identified MHC allele. The elements corresponding to the MHC alleles identified for the data instance i have a value of 1. Otherwise, the remaining elements have a value of 0. As an example, the alleles HLA-B*07:02 and HLA-DRB1*10:01 for a data instance i corresponding to a multiple-allele cell line among m=4 unique identified MHC allele types {HLA-A*01:01, HLA-C*01:08, HLA-B*07:02, HLA-DRB1*10:01} may be represented by the row vector of 4 elements aⁱ=[0 0 1 1], in which a₃ⁱ=1 and a₄ⁱ=1. Although the example is described herein with 4 identified MHC allele types, the number of MHC allele types can be hundreds or thousands in practice. As previously discussed, each data instance i typically contains at most 6 different MHC class I allele types in association with the peptide sequence pᵢ and/or at most 4 different MHC class II DR allele types in association with the peptide sequence pᵢ, and/or at most 12 different MHC class II allele types in association with the peptide sequence pᵢ.

In various embodiments, the encoding module 314 encodes MHC alleles by concatenating MHC allele sequences to a corresponding epitope sequence (e.g., sequence of an epitope for prediction of presentation). In various embodiments, the encoding module 314 encodes MHC alleles by concatenating a threshold number of amino acids of an MHC allele sequence that are likely in contact with the epitope sequence during binding. In various embodiments, the threshold number of amino acids of an MHC allele is between 10 and 50 amino acids. In various embodiments, the threshold number of amino acids of an MHC allele is between 15 and 45 amino acids, between 25 and 40 amino acids, or between 30 and 35 amino acids. In particular embodiments, the threshold number of amino acids of an MHC allele is 30 amino acids, 31 amino acids, 32 amino acids, 33 amino acids, 34 amino acids, or 35 amino acids. In particular embodiments, the threshold number of amino acids of an MHC allele is 34 amino acids.

In various embodiments, the encoding module 314 also encodes the label yⁱ for each data instance i as a binary variable having values from the set of {0, 1}, in which a value of 1 indicates that peptide xⁱ was presented by one of the associated MHC alleles aⁱ, and a value of 0 indicates that peptide xⁱ was not presented by any of the associated MHC alleles aⁱ. When the dependent variable yⁱ represents the mass spectrometry ion current, the encoding module 314 may additionally scale the values using various functions, such as the log function having a range of (−∞, ∞) for ion current values between [0, ∞).

The encoding module 314 may represent a pair of allele-interacting variables

$${x}_{h}^{i}$$

for peptide pᵢ and an associated MHC allele h as a row vector in which numerical representations of allele-interacting variables are concatenated one after the other. For example, the encoding module 314 may represent

$${x}_{h}^{i}$$

as a row vector equal to [pⁱ],

$$[ {p}^{i} {b}_{h}^{i} ] ,$$

$$[ {p}^{i} {s}_{h}^{i} ] , or [ {p}^{i} {b}_{h}^{i} {s}_{h}^{i} ] ,$$

where

$${b}_{h}^{i}$$

is the binding affinity prediction for peptide pᵢ and associated MHC allele h, and similarly for

$${s}_{h}^{i}$$

for stability. Alternatively, one or more combination of allele-interacting variables may be stored individually (e.g., as individual vectors or matrices). Further example encodings by the encoding module 314 is described in WO2018195357, which is hereby incorporated by reference in its entirety.

### II.C. Example Presentation Model

Embodiments disclosed herein involve training and/or deploying presentation models for predicting whether an epitope sequence is presented or not presented (e.g., by class II MHC alleles of a patient genotype). In various embodiments, an example presentation model includes multiple machine learning models that are trained and deployed to generate presentation predictions representing whether an epitope sequence is presented or not presented. In various embodiments, an example presentation model includes one machine learning model, two machine learning models, or three machine learning models.

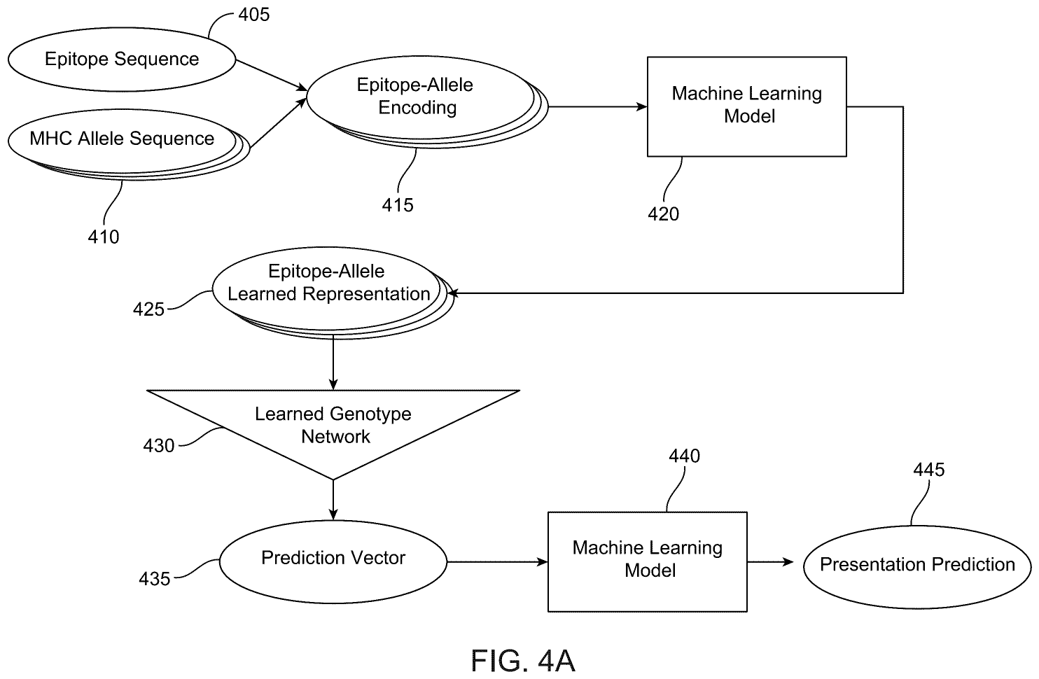

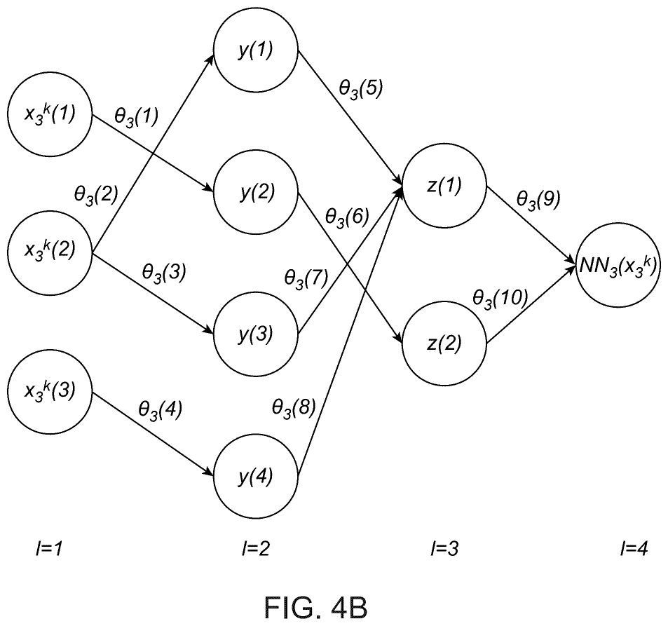

Reference is now made to FIG. 4A, which depicts an example presentation model, according to one embodiment. FIG. 4A introduces an example presentation model that includes three machine learning models, including a machine learning model 420, a learned genotype network 430, and a machine learning model 440. The process in FIG. 4A begins with an epitope sequence 405 (e.g., a sequence of an epitope for predicting whether the epitope is likely to be presented) and a set of MHC allele sequences 410. The set of MHC allele sequences 410 may be a set of class I MHC alleles or a set of class II MHC alleles. The set of MHC allele sequences 410 represent the MHC allele sequences expressed by a patient (e.g., a genotype of a patient). In particular embodiments, the set of MHC allele sequences 410 include six class II MHC allele sequences.

FIG. 4A further shows a step of combining the epitope sequence 405 and sequences of the MHC alleles 410 of the genotype to generate epitope-allele encodings 415. In various embodiments, an epitope-allele encoding 415 is generated for each MHC allele sequence 410. Therefore, in embodiments in which there are six MHC allele sequences 410 (e.g., corresponding to six MHC alleles expressed in a patient genotype), then combining the epitope sequence 405 and sequences of the MHC alleles 410 results in six epitope-allele encodings 415.

In various embodiments, combining the epitope sequence 405 and sequences of the MHC alleles 410 of the genotype involves performing a concatenation of the sequences. For example, a first epitope-allele encoding 415 may represent a concatenation of the epitope sequence 405 and a first MHC allele sequence 410. Additionally, a second epitope-allele encoding may represent a concatenation of the epitope sequence 405 and a second MHC allele sequence. Thus, each epitope-allele encoding 415 includes relevant sequence information of both the epitope and corresponding MHC allele. In some embodiments, an epitope-allele encoding 415 comprises a linear peptide sequence consisting of an epitope and its flanking amino acids, concatenated with structurally relevant amino acids from the corresponding MHC allele.

Referring to the machine learning model 420, it receives the epitope-allele encodings 415 as input and generates learned representations of the one or more epitope-allele encodings (also referred to as epitope-allele learned representations 425). In various embodiments, the epitope-allele learned representations 425 comprise sequence embeddings. Such sequence embeddings may represent embedded protein sequences with rich structural information. In various embodiments, the machine learning model 420 comprises a neural network. In various embodiments, the machine learning model 420 comprises a protein language model. In particular embodiments, the machine learning model 420 comprises an evolutionary scale model (ESM) language model. Further details of ESM language models are described in Rives, A. et al., “Biological structure and function emerge from scaling unsupervised learning to 250 million protein sequences.” PNAS 2021, 118(15) e2016239118 and Lin, Z. et al., “Evolutionary-scale prediction of atomic level protein structure with a language model.” bioRxiv 2022.07.20.500902, each of which is hereby incorporated by reference in its entirety.

The epitope-allele learned representations 425 are transformed using a learned genotype network 425. Here, the learned genotype network 425 aggregates embeddings from all class II HLA alleles and accounts for contributions across the class II MHC alleles of the genotype. The learned genotype network generates a single prediction vector 435.

Generally, the learned genotype network 425 pools the epitope-allele learned representations 425, thereby learning from all the class II MHC alleles present in a sample as opposed to merely the most likely class II MHC allele that presents the epitope. Conventional methodologies (e.g., BERTMHC) merely treat the MHC alleles separately and take the MHC allele with the maximum probability of presenting the epitope. In various embodiments, the learned genotype network 430 calculates a learned weighted average of each representation. More weight (“attention”) is given to alleles that are valuable for making the correct classification. Thus, in the learned genotype network 430, each class II MHC allele of the genotype competes with the other class II MHC alleles present in the genotype for attention from the model. Thus, the outputted prediction vector 435 from the learned genotype network 430 is dependent on the particular genotype of alleles.

In various embodiments, the learned genotype network 425 combines weighted combinations of epitope-allele learned representations. For example, the learned genotype network 430 can include a plurality of learned weights (e.g., weight that are learned during training of the learned genotype network 430). In various embodiments, each learned weight is specific for a class II MHC allele. In various embodiments, there may be more than one learned weight assigned to a class II MHC allele. In various embodiments, a sum of the learned weights of the learned genotype network 430 is 1. Thus, each MHC allele competes with the other MHC alleles as a less valuable MHC allele would be associated with a lower weight whereas a more valuable MHC allele would be associated with a higher weight. For example, a larger value of a learned weight indicates that a corresponding MHC allele contributes more heavily towards presentation of the epitope sequence in comparison to a MHC allele corresponding to a smaller value of a learned weight. In various embodiments, combining the weighted combinations of the epitope-allele learned representations 425 comprises: for each of the one or more learned representations, modifying the learned representation using a learned weight of the learned genotype network; and summating the one or more modified learned representations.

In various embodiments, a learned weight of the learned genotype network is specific for a kth class II MHC allele and is determined based on at least a non-linear transform of a learned representation an epitope-allele encoding of the kth class II MHC allele. For example, the non-linear transform influences the learned weight specific for the kth class II MHC allele based on a learned importance of the kth class II MHC allele for presentation of epitopes. In various embodiments, the learned importance is a learned weight matrix. In various embodiments, a learned weight of the learned genotype network is specific for a kth class II MHC allele and is determined based on at least a softmax transform of an epitope-allele learned representation of the kth class II MHC allele. In various embodiments, a learned weight of the learned genotype network is specific for a kth class II MHC allele and is determined based on both 1) a non-linear transform of a learned representation an epitope-allele encoding of the kth class II MHC allele and 2) a sigmoid transform of an epitope-allele learned representation of the kth class II MHC allele. In various embodiments, a learned weight of the learned genotype network is specific for a kth class II MHC allele and is determined based on (1) a non-linear transform of a learned representation an epitope-allele encoding of the kth class II MHC allele (2) a sigmoid transform of an epitope-allele learned representation of the kth class II MHC allele, and (3) a softmax transform of the elementwise multiplication of (1) and (2).

An exemplary pooling operation of the learned genotype network is described below. Specifically, the pooling operation can be expressed as shown in Equation (la)

$$z = {\sum }_{k = 1}^{K} {a}_{k} {h}_{k} \qquad ( 1 a )$$

where z=single classification vector representing weighted sum of each epitope-allele learned representation 425, over each allele in a sample, where K=the number of alleles in the sample/genotype (e.g., ranges from 1 (single allele) to 12 (full HLA class II haplotype), where hₖ represents an epitope-allele learned representation 425 epitope i joined with allele k. Specifically, hₖ can be represented as hₖ=ESM(x_{i,k})=learned representation from an ESM (protein language model), where h=(h₁, . . . , h_{K})=the set of all learned representations for a given epitope over all alleles in a sample/patient.

Furthermore, the learned genotype network calculates aₖ shown in Equation (1b)

$${a}_{k} = \frac{exp \{ {v}^{T} tanh ( {W}_{1} {h}_{k}^{T} ) ⊙ σ ( {W}_{2} {h}_{k}^{T} ) \}}{{\sum }_{j = 1}^{K} exp \{ {v}^{T} tanh ( {W}_{1} {h}_{j}^{T} ) ⊙ σ ( {W}_{2} {h}_{j}^{T} ) \}} \qquad ( 1 b )$$

W₁, W₂, v=weight matrices learned by the learned genotype network
T=transpose operation
σ(⋅)=sigmoid function
tan h(⋅)=non-linear hyperbolic tangent function
⊙=elementwise multiplication, e.g. [1,1]⊙[0,1]=[0,1]
tanh

$${W}_{1} {h}_{k}^{T} = ( nonlinear )$$

transform of epitope-allele learned representation that identifies alleles most useful to the final prediction. The weight matrix W₁ will learn to push alleles important for presentation k towards 1, and less important alleles towards −1.

$$σ ( {W}_{2} {h}_{k}^{T} ) = called$$

a “gate” in neural networks that controls influence of valuable and less valuable information and introduces additional non-linearity.

Returning to FIG. 4A, a machine learning model 440 analyzes the prediction vector 435 to generate a presentation prediction 445. In various embodiments, the machine learning model 440 comprises a classifier network, and therefore, the presentation prediction 445 is a classification (presented or not presented). Here, the presentation prediction 445 is indicative of whether the epitope is presented or not presented by one or more MHC alleles of the patient genotype. In various embodiments, the presentation prediction 445 is a score, also referred to herein as a “genotype presentation score.” Here, the score can represent a likelihood of presentation of the epitope sequence by the one or more of the class II MHC alleles of the genotype. In various embodiments, the score is further correlated with a resulting immunogenic response likely caused by the presentation of the epitope by an allele of the class II MHC alleles of the genotype.

Generally, a machine learning model, such as machine learning model 420, learned genotype network 430, or machine learning model 440 described in FIG. 4A, is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks).

A machine learning model (e.g., any of machine learning model 420, learned genotype network 430, or machine learning model 440) can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, the machine learning model (e.g., any of machine learning model 420, learned genotype network 430, or machine learning model 440) has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

FIG. 4B shows an example network architecture of a machine learning model, according to one embodiment. In various embodiments, the example machine learning model shown in FIG. 4B may be the machine learning model 420 shown in FIG. 4A, or a portion of the machine learning model 420 shown in FIG. 4A. Specifically, FIG. 4B illustrates an example network model NN₃(⋅) in association with an arbitrary MHC allele h=3.

As shown in FIG. 4B, the network model NN₃(⋅) for MHC allele h=3 includes three input nodes at layer 1=1, four nodes at layer 1=2, two nodes at layer 1=3, and one output node at layer 1=4. The network model NN₃(⋅) is associated with a set of ten parameters θ₃(1), θ₃(2), . . . , θ₃(10). The network model NN₃(⋅) receives input values (e.g., epitope-allele encodings 315 shown in FIG. 4A) for three allele-interacting variables x₃^{k}(1), x₃^{k}(2), and x₃^{k}(3) for MHC allele h=3 and outputs the value NN₃(x₃^{k}). The network function may also include one or more network models each taking different allele interacting variables as input.

In another instance, the identified MHC alleles h=1, 2, . . . , m are associated with a single network model NN_{H}(⋅), and NNₕ(⋅) denotes one or more outputs of the single network model associated with MHC allele h. In such an instance, the set of parameters θₕ may correspond to a set of parameters for the single network model, and thus, the set of parameters θₕ may be shared by all MHC alleles.

### II.D. Training the Presentation Model

The training module 316 constructs one or more presentation models that generates a prediction of whether peptide sequences will be presented by MHC alleles associated with the peptide sequences. In various embodiments, the one or more presentation models generate presentation likelihoods representing whether peptide sequences will be presented by MHC alleles associated with the peptide sequences. Specifically, given a peptide sequence p^{k} and a set of MHC alleles a^{k} associated with the peptide sequence p^{k}, each presentation model generates an estimate uₖ indicating a likelihood that the peptide sequence p^{k} will be presented by one or more of the associated MHC alleles a^{k}.

The training module 316 constructs the one more presentation models based on the training data sets stored in store 170 generated from the presentation information stored in 165. In various embodiments, regardless of the specific type of presentation model, the presentation models capture the dependence between independent variables and dependent variables in the training data 170 such that a loss function is minimized. Specifically, the loss function [symbol] (y_{i∈S}, u_{i∈S}; 0) represents discrepancies between values of dependent variables y_{i∈S} for one or more data instances S in the training data 170 and the estimated likelihoods u_{i∈S} for the data instances S generated by the presentation model. In one particular implementation referred throughout the remainder of the specification, the loss function (y_{i∈S}, u_{i∈S}; 0) is the negative log likelihood function given by equation (2a) as follows:

$$ℓ ( {y}_{i ∈ S} , {u}_{i ∈ S} ; θ ) = {\sum }_{i ∈ S} ( {y}_{i} log {u}_{i} + ( 1 - {y}_{i} ) log ( 1 - {u}_{i} ) ) . \qquad ( 2 a )$$

However, in practice, another loss function may be used. For example, when predictions are made for the mass spectrometry ion current, the loss function is the mean squared loss given by equation 2b as follows:

$$ℓ ( {y}_{i ∈ S} , {u}_{i ∈ S} ; θ ) = {\sum }_{i ∈ S} ( {‖ {y}_{i} - {u}_{i} ‖}_{2}^{2} ) . \qquad ( 2 b )$$

The presentation model may be a parametric model in which one or more parameters θ mathematically specify the dependence between the independent variables and dependent variables. Typically, various parameters of parametric-type presentation models that minimize the loss function (y_{i∈S}, u_{i∈S}; 0) are determined through gradient-based numerical optimization algorithms, such as batch gradient algorithms, stochastic gradient algorithms, and the like. Alternatively, the presentation model may be a non-parametric model in which the model structure is determined from the training data 170 and is not strictly based on a fixed set of parameters.

In various embodiments, the training module 316 constructs presentation models comprising one or more per-allele models. In various embodiments, the training module 316 constructs presentation models to predict presentation likelihoods of peptides in a multiple-allele setting where two or more MHC alleles are present. Example per-allele models and presentation models for multiple allele settings are described in further detail in WO2018195357, which is incorporated by reference in its entirety. In various embodiments, the training module 316 incorporates allele-noninteracting variables or allele noninteracting variables according to dependency functions. Example dependency functions for allele interacting variables or allele noninteracting variables are described in further detail in WO2018195357, which is incorporated by reference in its entirety.

Generally, the training module 316 trains a presentation model using training data, such as mass spectrometry data in which presented peptides have been isolated and identified. In various embodiments, the training module 316 trains a presentation model using single allelic training data (e.g., training data obtained from single-allele expressing cells). In this situation, the presentation model can be trained to recognize the direct association between a presented peptide and the corresponding MHC allele that presented the peptide.

In various embodiments, the training module 316 trains a presentation model using multi-allelic training data (e.g., training data obtained from multi-allele expressing cells). For example, multi-allele expressing cells can express two MHC class II alleles, three MHC class II alleles, four MHC class II alleles, five MHC class II alleles, six MHC class II alleles, seven MHC class II alleles, eight MHC class II alleles, nine MHC class II alleles, ten MHC class II alleles, eleven MHC class II alleles, or twelve MHC class II alleles. In various embodiments, such multi-allele expressing cells can be engineered to express the desired number of MHC class II alleles. In some embodiments, multi-allele expressing cells can be obtained from patient samples. Therefore, the particular set of alleles expressed by the multi-allele expressing cells obtained from a patient can represent a genotype of the patient. Here, the direct association between a presented peptide and the MHC allele that presented the peptide may be unknown. Thus, the presentation model may learn associations between certain peptides and certain sets of MHC alleles that present or do not present the peptides.

In various embodiments, the training module 316 trains a presentation model using both single allelic training data and multi-allelic training data. Thus, the presentation model can be trained to learn direct association between a presented peptide and the corresponding MHC allele that presented the peptide, and can further be trained to learn associations between certain peptides and certain sets of MHC alleles that present or do not present the peptides.

In various embodiments, the training module 316 trains a presentation model using intermediate resolution data that indicates whether a family of alleles presented a particular epitope sequence. Here, intermediate resolution data represents data of lower resolution in comparison to single-allele data and represents data of higher resolution in comparison to multi-allele data. An example of such intermediate resolution data includes immune affinity purified mass spectrometry data using antibodies specific to DR, DP, or DQ alleles. For example, in the context of class II MHC alleles, intermediate resolution data can include DR-specific immunoaffinity purified mass spectrometry presentation data. DR-specific immunoaffinity purified mass spectrometry presentation data can indicate whether an epitope was presented or not presented by the HLA-DR family of alleles (e.g., including HLA-DRA, HLA-DRB1, HLA-DRB3, HLA-DRB4, and HLA-DRB5). As another example, intermediate resolution data can include DQ-specific immunoaffinity purified mass spectrometry presentation data. DQ-specific immunoaffinity purified mass spectrometry presentation data can indicate whether an epitope was presented or not presented by the HLA-DQ family of alleles (e.g., including HLA-DQA1, HLA-DQA2, HLA-DQB1, and HLA-DQB2). As another example, intermediate resolution data can include DP-specific immunoaffinity purified mass spectrometry presentation data. DP-specific immunoaffinity purified mass spectrometry presentation data can indicate whether an epitope was presented or not presented by the HLA-DP family of alleles (e.g., including HLA-DPA1 and HLA-DPB1). In various embodiments, intermediate resolution data includes each of DR-specific, DQ-specific, and DP-specific immunoaffinity purified mass spectrometry presentation data.

In various embodiments, the training module 316 trains a presentation model using single allelic training data and multi-allelic training data. In various embodiments, the training module 316 trains a presentation model using single allelic training data and intermediate resolution data including each of DR-specific, DQ-specific, and DP-specific immunoaffinity purified mass spectrometry presentation data. In various embodiments, the training module 316 trains a presentation model using multi-allelic training data and intermediate resolution data including each of DR-specific, DQ-specific, and DP-specific immunoaffinity purified mass spectrometry presentation data. In various embodiments, the training module 316 trains a presentation model using each of single allelic training data, multi-allelic training data, and intermediate resolution data including each of DR-specific, DQ-specific, and DP-specific immunoaffinity purified mass spectrometry presentation data.

In various embodiments, the training module 316 trains a presentation model using different training through multiple phases. In various embodiments, the training module 316 may train a presentation model during a first phase using single allelic training data. Thus, during this first phase, the training module 316 trains the presentation model to recognize direct associations between a presented peptide and corresponding MHC alleles that presented the peptide. In particular embodiments, the training module 316 may train a presentation model during a first phase using both single allelic and multi-allelic training data.

The training module 316 may further train a presentation model during a second phase using intermediate resolution data, such as intermediate resolution data including each of DR-specific, DQ-specific, and DP-specific immunoaffinity purified mass spectrometry presentation data. Here, the presentation model further learns relationships between families of class II MHC alleles (e.g., DR, DQ, and DP families) and epitopes. This further supplements the direct associations between presented peptides and corresponding MHC alleles that were learned from the single-allelic training data.

In various embodiments, the training module 316 may further train a presentation model during a third phase using multi-allelic training data. Thus, the training module 316 trains the presentation model to learn associations between peptides and certain sets of MHC alleles that present or do not present the peptides. This third phase may additionally reduce or eliminate biases that arise in the first and/or second phases of training (e.g., when using single allelic training data and/or intermediate resolution data).

Referring again to FIG. 4A, in various embodiments, the various machine learning models (e.g., machine learning model 420, machine learning model 440) and the learned genotype network 430 of the presentation model are jointly trained using the training data. Thus, the parameters of the machine learning models and learned genotype network 430 are adjusted together during training. In various embodiments, the machine learning models (e.g., machine learning model 420, machine learning model 440) and the learned genotype network 430 may undergo separate training. For example, the training module 316 may deactivate the learned genotype network 430 during certain phases of training and activate the learned genotype network 430 during other phases of training. In one example, the training module 316 may deactivate the learned genotype network 430 during a first phase of training when using single allelic training data. In one example, the training module 316 may deactivate the learned genotype network 430 during a second phase of training when using intermediate resolution data comprising DR-specific, DQ-specific, and DP-specific immunoaffinity purified mass spectrometry presentation data. In one example, the training module 316 may deactivate the learned genotype network 430 during both a first phase of training when using single allelic training data and during a second phase of training when using intermediate resolution data comprising DR-specific, DQ-specific, and DP-specific immunoaffinity purified mass spectrometry presentation data. The training module 316 can then activate the learned genotype network 430 for training during the third phase using multi-allelic training data. This pattern of deactivation/activation of the learned genotype network 430 during different phases of training can be beneficial to avoid biases that arise due to different proportions of training data (e.g., different proportions of DP/DQ/DR data).

### II.E. Deploying the Presentation Model

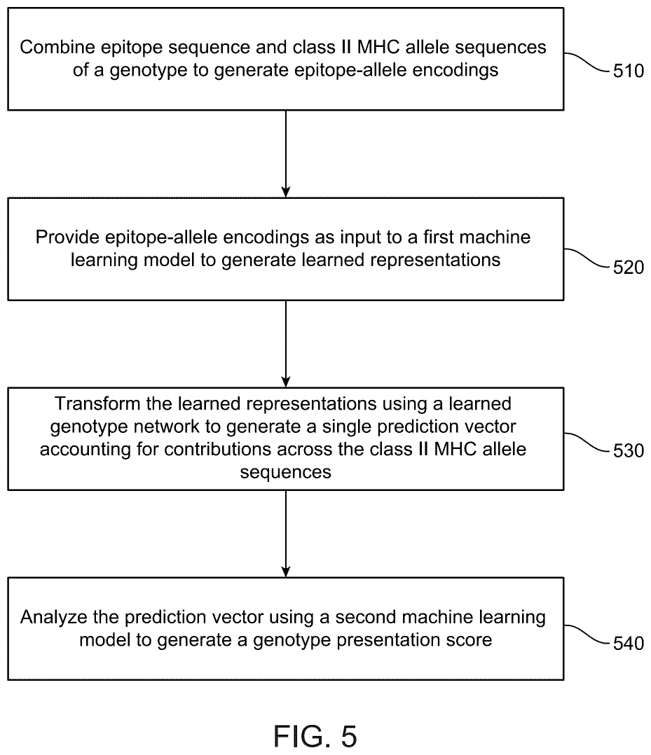

FIG. 5 shows an example flow process for predicting epitope presentation, according to one embodiment. In particular embodiments, the flow process shown in FIG. 5 is useful for predicting epitope presentation by a plurality of class II MHC alleles of a patient genotype. Generally, the patient genotype refers to the set of class II MHC alleles expressed by the patient. In various embodiments, the set of class II MHC alleles expressed by the patient includes six class II MHC alleles. In various embodiments, the set of class II MHC alleles expressed by the patient includes two class II MHC alleles, three class II MHC alleles, four class II MHC alleles, five class II MHC alleles, six class II MHC alleles, seven class II MHC alleles, eight class II MHC alleles, nine class II MHC alleles, ten class II MHC alleles, eleven class II MHC alleles, or twelve class II MHC alleles. In various embodiments, the example flow process of FIG. 5 shows the analysis of the presentation model shown in FIG. 4A, which involves deploying at least a machine learning model 420, a learned genotype network 430, and a machine learning model 440 for generating a presentation prediction.

As shown in FIG. 5, step 510 involves combining an epitope sequence and one or more class II MHC allele sequences of a genotype (e.g., genotype of a patient). Here, step 510 involves generating epitope-allele encodings representing the combination of the epitope sequence and class II allele sequences. In various embodiments, combining the epitope sequence and class II MHC allele sequences involves concatenating the epitope sequence and class II MHC allele sequences to generate the epitope-allele encodings.

Step 520 involves providing the epitope-allele encodings as input to a first machine learning model to generate learned representations. In various embodiments, the first machine learning model is a protein language model, such as an Evolutionary Scale Model (ESM2) language model. Thus, the first machine learning model outputs learned representations, examples of which include sequence embeddings.

Step 530 involves transforming the learned representations using a learned genotype network to generate a single prediction vector. Here, the learned genotype network aggregates the learned representations across the class II MHC alleles of the genotype, thereby accounting for contributions across the class II MHC alleles of the genotype. Thus, in contrast to current approaches that merely take the single class II MHC allele that is most likely to present the epitope, the inclusion of the learned genotype network learns contributions from all of the class II MHC alleles of the genotype.

Step 540 involves analyzing the single prediction vector using a second machine learning model to generate a genotype presentation score. In various embodiments, the genotype presentation score is indicative of whether the epitope is presented or not presented by an allele of the class II MHC alleles of the genotype. In various embodiments, the genotype presentation score is correlated with a resulting immunogenic response likely caused by the presentation of the epitope by an allele of the class II MHC alleles of the genotype.

### II.F. Cassette Design Module

A cassette design module can be used to generate a vaccine cassette sequence based on selected candidate peptides for injection into a patient. For example, a cassette design module can be used to generate a sequence encoding concatenated epitope sequences, such as concatenated T cell epitopes. Various cassette design modules are known to those skilled in the art, for example the cassette design modules described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

A set of therapeutic epitopes may be generated based on the selected peptides determined by a prediction module associated with presentation likelihoods above a predetermined threshold, where the presentation likelihoods are determined by the presentation models. However it is appreciated that in other embodiments, the set of therapeutic epitopes may be generated based on any one or more of a number of methods (alone or in combination), for example, based on binding affinity or predicted binding affinity to HLA class I or class II alleles of the patient, binding stability or predicted binding stability to HLA class I or class II alleles of the patient, random sampling, and the like.

Therapeutic epitopes may correspond to selected peptides themselves. Therapeutic epitopes may also include C- and/or N-terminal flanking sequences in addition to the selected peptides. N- and C-terminal flanking sequences can be the native N- and C-terminal flanking sequences of the therapeutic vaccine epitope in the context of its source protein. Therapeutic epitopes can represent a fixed-length epitope Therapeutic epitopes can represent a variable-length epitope, in which the length of the epitope can be varied depending on, for example, the length of the C- or N-flanking sequence. For example, the C-terminal flanking sequence and the N-terminal flanking sequence can each have varying lengths of 2-5 residues, resulting in 16 possible choices for the epitope.

A cassette design module can also generate cassette sequences by taking into account presentation of junction epitopes that span the junction between a pair of therapeutic epitopes in the cassette. Junction epitopes are novel non-self but irrelevant epitope sequences that arise in the cassette due to the process of concatenating therapeutic epitopes and linker sequences in the cassette. The novel sequences of junction epitopes are different from the therapeutic epitopes of the cassette themselves.

A cassette design module can generate a cassette sequence that reduces the likelihood that junction epitopes are presented in the patient. Specifically, when the cassette is injected into the patient, junction epitopes have the potential to be presented by HLA class I or HLA class II alleles of the patient, and stimulate a CD8 or CD4 T-cell response, respectively. Such reactions are often times undesirable because T-cells reactive to the junction epitopes have no therapeutic benefit, and may diminish the immune response to the selected therapeutic epitopes in the cassette by antigenic competition (Janetzki, S., Price, L., Schroeder, H., Britten, C. M., Welters, M. J. P., and Hoos, A. (2015). Guidelines for the automated evaluation of Elispot assays. Nat Protoc 10, 1098-1115).

A cassette design module can iterate through one or more candidate cassettes, and determine a cassette sequence for which a presentation score of junction epitopes associated with that cassette sequence is below a numerical threshold. The junction epitope presentation score is a quantity associated with presentation likelihoods of the junction epitopes in the cassette, and a higher value of the junction epitope presentation score indicates a higher likelihood that junction epitopes of the cassette will be presented by HLA class I or HLA class II or both.

In one embodiment, a cassette design module may determine a cassette sequence associated with the lowest junction epitope presentation score among the candidate cassette sequences.

A cassette design module may iterate through one or more candidate cassette sequences, determine the junction epitope presentation score for the candidate cassettes, and identify an optimal cassette sequence associated with a junction epitope presentation score below the threshold.

A cassette design module may further check the one or more candidate cassette sequences to identify if any of the junction epitopes in the candidate cassette sequences are self-epitopes for a given patient for whom the vaccine is being designed. To accomplish this, the cassette design module checks the junction epitopes against a known database such as BLAST. In one embodiment, the cassette design module may be configured to design cassettes that avoid junction self-epitopes.

A cassette design module can perform a brute force approach and iterate through all or most possible candidate cassette sequences to select the sequence with the smallest junction epitope presentation score. However, the number of such candidate cassettes can be prohibitively large as the capacity of the vaccine increases. For example, for a vaccine capacity of 20 epitopes, the cassette design module has to iterate through ˜10¹⁸ possible candidate cassettes to determine the cassette with the lowest junction epitope presentation score. This determination may be computationally burdensome (in terms of computational processing resources required), and sometimes intractable, for the cassette design module to complete within a reasonable amount of time to generate the vaccine for the patient. Moreover, accounting for the possible junction epitopes for each candidate cassette can be even more burdensome. Thus, a cassette design module may select a cassette sequence based on ways of iterating through a number of candidate cassette sequences that are significantly smaller than the number of candidate cassette sequences for the brute force approach.

A cassette design module can generate a subset of randomly or at least pseudo-randomly generated candidate cassettes, and selects the candidate cassette associated with a junction epitope presentation score below a predetermined threshold as the cassette sequence.

Additionally, the cassette design module may select the candidate cassette from the subset with the lowest junction epitope presentation score as the cassette sequence. For example, the cassette design module may generate a subset of ˜1 million candidate cassettes for a set of 20 selected epitopes, and select the candidate cassette with the smallest junction epitope presentation score. Although generating a subset of random cassette sequences and selecting a cassette sequence with a low junction epitope presentation score out of the subset may be sub-optimal relative to the brute force approach, it requires significantly less computational resources thereby making its implementation technically feasible. Further, performing the brute force method as opposed to this more efficient technique may only result in a minor or even negligible improvement in junction epitope presentation score, thus making it not worthwhile from a resource allocation perspective. A cassette design module can determine an improved cassette configuration by formulating the epitope sequence for the cassette as an asymmetric traveling salesman problem (TSP). Given a list of nodes and distances between each pair of nodes, the TSP determines a sequence of nodes associated with the shortest total distance to visit each node exactly once and return to the original node. For example, given cities A, B, and C with known distances between each other, the solution of the TSP generates a closed sequence of cities, for which the total distance traveled to visit each city exactly once is the smallest among possible routes. The asymmetric version of the TSP determines the optimal sequence of nodes when the distance between a pair of nodes are asymmetric. For example, the “distance” for traveling from node A to node B may be different from the “distance” for traveling from node B to node A. By solving for an improved optimal cassette using an asymmetric TSP, the cassette design module can find a cassette sequence that results in a reduced presentation score across the junctions between epitopes of the cassette. The solution of the asymmetric TSP indicates a sequence of therapeutic epitopes that correspond to the order in which the epitopes should be concatenated in a cassette to minimize the junction epitope presentation score across the junctions of the cassette. A cassette sequence determined through this approach can result in a sequence with significantly less presentation of junction epitopes while potentially requiring significantly less computational resources than the random sampling approach, especially when the number of generated candidate cassette sequences is large. Illustrative examples of different computational approaches and comparisons for optimizing cassette design are described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

An illustrative non-limiting cassette of concantenated KRAS-associated MHC class I neoepitopes that are linked through their native flanking sequences, includes 4 iterations for each of the KRAS neoepitopes having the mutations KRAS G12C, KRAS G12D, KRAS G12V, and KRAS Q61H, and has been ordered to minimize potential junctional epitopes is represented by the amino acid sequence NEIOREIREI and having the order of KRAS-associated neoepitopes: G12C G12D Q61H G12D G12V G12C Q61H G12D G12V G12C Q61H G12D G12V Q61H G12V G12C.

Shared (neo)antigen sequences for inclusion in a shared antigen vaccine and appropriate patients for treatment with such vaccine can be chosen by one of skill in the art, e.g., as described in U.S. application Ser. No. 17/058,128, herein incorporated by reference for all purposes. Mass spectrometry (MS) validation of candidate shared (neo)antigens can performed as part of the selection process.

A cassette design module can also generate cassette sequences by taking into account additional protein sequences encoded in the vaccine. For example, a cassette design module used to generate a sequence encoding concatenated T cell epitopes can take into account T cell epitopes already encoded by additional protein sequences present in the vaccine (e.g., full-length protein sequences), such as by removing T cell epitopes already encoded by the additional protein sequences from the list of candidate sequences.

A cassette design module can also generate cassette sequences by taking into account the size of the sequences. Without wishing to be bound by theory, in general, increased cassette size can negatively impact vaccine aspects, such as vaccine production and/or vaccine efficacy. In one example, the cassette design module can take into account overlapping sequences, such as overlapping T cell epitope sequences. In general, a single sequence containing overlapping T cell epitope sequences (also referred to as a “frame”) is more efficient than separately linking individual T cell epitope sequences as it reduces the sequence size needed to encode the multiple peptides. Accordingly, in an illustrative example, a cassette design module used to generate a sequence encoding concatenated T cell epitopes can take into account the cost/benefit of extending a candidate T cell epitope to encode one or more additional T cell epitopes, such as determining the benefit gained in additional population coverage for an MHC presenting the additional T cell epitope versus the cost of increasing the size of the sequence.

A cassette design module can also generate cassette sequences by taking into account the magnitude of stimulation of an immune response generated by validated epitopes.

A cassette design module can also generate cassette sequences by taking into account presentation of encoded epitopes across a population, for example that at least one immunogenic epitope is presented by at least one HLA across a proportion of a population, for example by at least 85%, 90%, or 95% of a population (e.g., HLA-A, HLA-B and HLA-C genes over four major ethnic groups, namely European (EUR), African American (AFA), Asian and Pacific Islander (APA) and Hispanic (HIS)). As an illustrative non-limiting example, a cassette design module can also generate cassette sequences such that at least one HLA is present at least across 85%, 90%, or 95% of a population that presents at least one validated epitope or presents at least 4, 5, 6, or 7 predicted epitopes.

A cassette design module can also generate cassette sequences by taking into account other aspects that improve potential safety, such as limiting encoding or the potential to encode a functional protein, functional protein domain, functional protein subunit, or functional protein fragment potentially presenting a safety risk. In some cases, a cassette design module can limit sequence size of encoded peptides such that they are less than 50%, less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length protein. In some cases, a cassette design module can limit sequence size of encoded peptides such that a single contiguous sequence is less than 50% of the translated, corresponding full-length protein, but more than one sequence may be derived from the same translated, corresponding full-length protein and together encode more than 50%. In an illustrative example, if a single sequence containing overlapping T cell epitope sequences (“frame”) is larger than 50% of the translated, corresponding full-length protein, the frame can be split into multiple frames (e.g., f1, f2 etc.) such that each frame is less than 50% of the translated, corresponding full-length protein. A cassette design module can also limit sequence size of encoded peptides such that a single contiguous sequence is less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length protein. Where multiple frames from the same gene are encoded, the multiple frames can have overlapping sequences with each other, in other words each separately encode the same sequence. Where multiple frames from the same gene are encoded, the two or more nucleic acid sequences derived from the same gene can be ordered such that a first nucleic acid sequence cannot be immediately followed by or linked to a second nucleic acid sequence if the second nucleic acid sequence follows, immediately or not, the first nucleic acid sequence in the corresponding gene. For example, if there are 3 frames within the same gene (f1,f2,f3 in increasing order of amino acid position):

The following cassette orderings are not allowed:
f1 immediately followed by f2
f2 immediately followed by f3
f1 immediately followed by f3
   The following cassette orderings are allowed:
f3 immediately followed by f2
f2 immediately followed by f1

### III. Neoantigens

Neoantigens can include nucleotides or polypeptides. For example, a neoantigen can be an RNA sequence that encodes for a polypeptide sequence. Neoantigens useful in vaccines can therefore include nucleotide sequences or polypeptide sequences.

Disclosed herein are isolated peptides that comprise tumor specific mutations identified by the methods disclosed herein, peptides that comprise known tumor specific mutations, and mutant polypeptides or fragments thereof identified by methods disclosed herein.

Neoantigen peptides can be described in the context of their coding sequence where a neoantigen includes the nucleotide sequence (e.g., DNA or RNA) that codes for the related polypeptide sequence.

One or more polypeptides encoded by a neoantigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more neoantigens can be presented on the surface of a tumor.

One or more neoantigens can be is immunogenic in a subject having a tumor, e.g., capable of eliciting a T cell response or a B cell response in the subject.

One or more neoantigens that induce an autoimmune response in a subject can be excluded from consideration in the context of vaccine generation for a subject having a tumor.

The size of at least one neoantigenic peptide molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the neoantigenic peptide molecules are equal to or less than 50 amino acids.

Neoantigenic peptides and polypeptides can be: for MHC Class 115 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would consist of: (3) the entire stretch of novel tumor-specific amino acids—thus bypassing the need for computational or in vitro test-based selection of the strongest HLA-presented shorter peptide. In both cases, use of a longer peptide allows endogenous processing by patient cells and may lead to more effective antigen presentation and induction of T cell responses.

Neoantigenic peptides and polypeptides can be presented on an HLA protein. In some aspects neoantigenic peptides and polypeptides are presented on an HLA protein with greater affinity than a wild-type peptide. In some aspects, a neoantigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In various embodiments, neoantigenic peptides include KRAS peptide sequences. Kristen rat sarcoma viral oncogene (KRAS) refers to the proto-oncogene that encodes a small GTPase involved in the Ras/mitogen-activated protein kinase (MAPK) pathway. As a small GTPase, KRAS functions as molecular switch that exists in binary states (on or off). These on and off states are regulated by binding of guanine triphosphate or guanine diphosphate, respectively. KRAS holds a critical role in the signal transduction from receptor tyrosine kinases involved in recognizing and processing extracellular signaling molecules (ligands). Subsequent KRAS interactions with Raf, mitogen-activated extracellular signal-regulated kinase (ERK) (MEK) 1/2, and ERK 1/2 makes up the complex intracellular signaling events leading to modulation of key biological processes such as gene expression, cell growth/survival, cellular division, and cellular differentiation. In clinical settings, RAS mutations remain the most common gene mutations in human cancers. KRAS mutations/overexpression results in altered intracellular signaling that can result in pathogenic cellular proliferation and survival, in the context of multiple cancer subtypes.

In various embodiments, neoantigenic peptides include KRAS G12C peptide sequences. KRAS G12C peptide sequences refer to a single point mutation in codon 12 of KRAS resulting in a transition from a glycine to cysteine. This mutation results in a protein product that favors the active state of the GTPase. KRAS G12C mutations are common in non-small cell lung cancer.

In some aspects, neoantigenic peptides and polypeptides do not induce an autoimmune response and/or invoke immunological tolerance when administered to a subject.

Also provided are compositions comprising at least two or more neoantigenic peptides. In some embodiments the composition contains at least two distinct peptides. At least two distinct peptides can be derived from the same polypeptide. By distinct polypeptides is meant that the peptide vary by length, amino acid sequence, or both. The peptides are derived from any polypeptide known to or have been found to contain a tumor specific mutation. Suitable polypeptides from which the neoantigenic peptides can be derived can be found for example in the COSMIC database. COSMIC curates comprehensive information on somatic mutations in human cancer. The peptide contains the tumor specific mutation. In some aspects the tumor specific mutation is a driver mutation for a particular cancer type.

Neoantigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, neoantigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to induce CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

A neoantigenic peptide can be linked to the T helper peptide either directly or via a spacer either at the amino or carboxy terminus of the peptide. The amino terminus of either the neoantigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect a neoantigen includes a nucleic acid (e.g. polynucleotide) that encodes a neoantigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothiate backbone, or combinations thereof and it may or may not contain introns. A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

Further disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Specifically, disclosed herein are cassettes including a KRAS-associated MHC class II neoepitope. KRAS-associated MHC class II neoepitopes include, but are not limited to, neoepitopes having KRAS G12 mutations and/or KRAS Q61 mutations. Cassettes can include KRAS-associated MHC class II neoepitopes having a KRAS G12 mutation. Cassettes can include KRAS-associated MHC class II neoepitopes having a KRAS Q61 mutation. Cassettes can include KRAS-associated MHC class II neoepitopes having KRAS G12C, KRAS G12V, KRAS G12D, KRAS G12A, and/or KRAS Q61H mutations. Cassettes can include KRAS-associated MHC class II neoepitopes having a KRAS G12C mutation. Cassettes can include KRAS-associated MHC class II neoepitopes having a KRAS G12V mutation. Cassettes can include KRAS-associated MHC class II neoepitopes having a KRAS G12D mutation. Cassettes can include KRAS-associated MHC class II neoepitopes having a KRAS G12A mutation. Cassettes can include KRAS-associated MHC class II neoepitopes having a KRAS Q61H mutation.

Cassettes can also include KRAS-associated MHC class I neoepitopes, e.g., a cassette can encode both a KRAS-associated MHC class II neoepitope and a KRAS-associated MHC class I neoepitope. In some instances, a KRAS-associated MHC class I neoepitope may be a fragment of (e.g., embedded within) a longer KRAS-associated MHC class II neoepitope sequence. Cassettes can include KRAS-associated MHC class I neoepitopes having a KRAS G12 mutation. Cassettes can include KRAS-associated MHC class I neoepitopes having a KRAS Q61 mutation. Cassettes can include KRAS-associated MHC class I neoepitopes having KRAS G12C, KRAS G12V, KRAS G12D, KRAS G12A, and/or KRAS Q61H mutations. Cassettes can include KRAS-associated MHC class I neoepitopes having a KRAS G12C mutation. Cassettes can include KRAS-associated MHC class I neoepitopes having a KRAS G12V mutation. Cassettes can include KRAS-associated MHC class I neoepitopes having a KRAS G12D mutation. Cassettes can include KRAS-associated MHC class I neoepitopes having a KRAS G12A mutation. Cassettes can include KRAS-associated MHC class I neoepitopes having a KRAS Q61H mutation.

Cassettes can also include iterations of KRAS-associated MHC class I and/or MHC class II neoepitopes. KRAS-associated MHC class I and/or MHC class II neoepitopes include, but are not limited to, neoepitopes having KRAS G12 mutations and/or KRAS Q61 mutations. Cassettes can include iterations of KRAS-associated MHC class I and/or MHC class II neoepitopes having a KRAS G12 mutation. Cassettes can include iterations of KRAS-associated MHC class I and/or MHC class II neoepitopes having a KRAS Q61 mutation. Cassettes can include iterations of KRAS-associated MHC class I and/or MHC class II neoepitopes having KRAS G12C, KRAS G12V, KRAS G12D, KRAS G12A, and/or KRAS Q61H mutations. Cassettes can include iterations of KRAS-associated MHC class I and/or MHC class II neoepitopes having a KRAS G12C mutation. Cassettes can include iterations of KRAS-associated MHC class I and/or MHC class II neoepitopes having a KRAS G12V mutation. Cassettes can include iterations of KRAS-associated MHC class I and/or MHC class II neoepitopes having a KRAS G12D mutation. Cassettes can include iterations of KRAS-associated MHC class I and/or MHC class II neoepitopes having a KRAS G12A mutation. Cassettes can include iterations of KRAS-associated MHC class I and/or MHC class II neoepitopes having a KRAS Q61H mutation. Cassettes can include iterations of each of KRAS-associated MHC class I and/or MHC class II neoepitopes having a KRAS G12C, KRAS G12V, KRAS G12D, and KRAS Q61H mutation. Cassettes can include iterations of at least two distinct KRAS-associated MHC class I and/or MHC class II neoepitopes selected from the group consisting of: a KRAS G12C, KRAS G12V, KRAS G12D, KRAS G12A, and KRAS Q61H mutation. Cassettes can include iterations of at least three distinct KRAS-associated MHC class I and/or MHC class II neoepitopes selected from the group consisting of: a KRAS G12C, KRAS G12V, KRAS G12D, KRAS G12A, and KRAS Q61H mutation. Cassettes can include iterations only of a single distinct KRAS-associated MHC class I and/or MHC class II neoepitope. Cassettes can include iterations only of a single distinct KRAS-associated MHC class I and/or MHC class II neoepitope having a KRAS G12C mutation. Cassettes can include iterations only of a single distinct KRAS-associated MHC class I and/or MHC class II neoepitope having a KRAS G12D mutation. Cassettes can include iterations only of a single distinct KRAS-associated MHC class I and/or MHC class II neoepitope having a KRAS G12V mutation. Cassettes can include iterations only of a single distinct KRAS-associated MHC class I and/or MHC class II neoepitope having a KRAS G12A mutation. Cassettes can include iterations only of a single distinct KRAS-associated MHC class I and/or MHC class II neoepitope having a KRAS Q61H mutation.

KRAS-associated MHC class I neoepitopes having a KRAS G12C mutation include VVVGACGVGK or KLVVVGACGV. KRAS-associated MHC class I neoepitopes having a KRAS G12D mutation include VVGADGVGK or VVVGADGVGK, KRAS-associated MHC class I neoepitopes having a KRAS G12V mutation include VVGAVGVGK, VVVGAVGVGK, or AVGVGKSAL.

Cassettes can include iterations of each of KRAS-associated MHC class I neoepitopes having the amino acid sequences VVVGACGVGK, VVVGADGVGK, VVGAVGVGK, and ILDTAGHEEY. Cassettes can include iterations of at least two distinct KRAS-associated MHC class I neoepitopes having the amino acid sequences selected from the group consisting of: VVVGACGVGK, VVVGADGVGK, VVGAVGVGK, and ILDTAGHEEY. Cassettes can include iterations of at least three distinct KRAS-associated MHC class I neoepitopes having the amino acid sequences selected from the group consisting of: VVVGACGVGK, VVVGADGVGK, VVGAVGVGK, and ILDTAGHEEY. Cassettes can include iterations of at least one of KRAS-associated MHC class I neoepitopes having the amino acid sequences VVVGACGVGK, VVVGADGVGK, VVGAVGVGK, and ILDTAGHEEY.

KRAS-associated MHC class I and/or MHC class II neoepitopes can include native N- and/or C-terminal flanking sequences of the therapeutic vaccine epitope in the context of the native KRAS protein. KRAS-associated MHC class I and/or MHC class II neoepitopes that include native flanking sequences can be linked (concatenated) to other neoepitopes encoded in a cassette, including other neoepitopes (e.g., other KRAS-associated MHC class I and/or MHC class II neoepitopes) that include their respective native flanking sequences.

Illustrative non-limiting examples of KRAS-associated MHC class I neoantigens that encode MHC class I neoepitopes having native linkers are the 25mers MTEYKLVVVGACGVGKSALTIQLIQ for KRAS G12C, MTEYKLVVVGADGVGKSALTIQLIQ for KRAS G12D, MTEYKLVVVGAVGVGKSALTIQLIQ for KRAS G12V, and ETCLLDILDTAGHEEYSAMRDQYMR for KRAS Q61H. An illustrative non-limiting cassette of concantenated KRAS-associated MHC class I neoepitopes that are linked through their native flanking sequences and that includes 4 iterations for each of the KRAS neoepitopes having the mutations KRAS G12C, KRAS G12D, KRAS G12V, and KRAS Q61H is represented by the amino acid sequence of NEIOREIREI.

Epitope-encoding nucleic acid sequences that encode KRAS-associated MHC class I and/or MHC class II neoepitopes, such as those that include native N- and/or C-terminal flanking sequences, can encode multiple known and/or predicted KRAS-associated MHC class I and/or MHC class II neoepitopes. As an illustrative example, the KRAS G12V 25mer MTEYKLVVVGAVGVGKSALTIQLIQ encodes each of the known and/or predicted KRAS-associated MHC class I neoepitopes VVGAVGVGK, VVVGAVGVGK, and AVGVGKSAL.

Epitope-encoding nucleic acid sequences, including those that encode KRAS-associated MHC class I and/or MHC class II neoepitopes, can be in any order in a cassette. Epitope-encoding nucleic acid sequences, including those that encode KRAS-associated MHC class I and/or MHC class II neoepitopes, can be in an order that minimizes junctional epitopes, as described further herein. As an illustrative non-limiting example, concantenated KRAS-associated MHC class I neoepitopes linked together to minimize junctional epitopes is represented by the amino acid sequence of NEIOREIREI and has the order: G12C G12D Q61H G12D G12V G12C Q61H G12D G12V G12C Q61H G12D G12V Q61H G12V G12C.

Also disclosed herein are peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database. COSMIC curates comprehensive information on somatic mutations in human cancer. Tumor antigens (e.g., shared tumor antigens and tumor neoantigens) can include, but are not limited to, those described in U.S. application Ser. No. 17/058,128, herein incorporated by reference for all purposes. Antigen peptides can be described in the context of their coding sequence where an antigen includes the nucleotide sequence (e.g., DNA or RNA) that codes for the related polypeptide sequence.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. The peptides are derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue.

Antigen peptides can be described in the context of their coding sequence where an antigen includes the nucleotide sequence (e.g., DNA or RNA) that codes for the related polypeptide sequence.

Antigens can be selected that are predicted to be presented on the cell surface of a cell, such as a tumor cell, an infected cell, or an immune cell, including professional antigen presenting cells such as dendritic cells. Antigens can be selected that are predicted to be immunogenic.

Research methods for NGS analysis of tumor and normal exome and transcriptomes have been described and applied in the antigen identification space. Certain optimizations for greater sensitivity and specificity for antigen identification in the clinical setting can be considered. These optimizations can be grouped into two areas, those related to laboratory processes and those related to the NGS data analysis. The research methods described can also be applied to identification of antigens in other settings, such as identification of identifying antigens from an infectious disease organism, an infection in a subject, or an infected cell of a subject. Examples of optimizations are known to those skilled in the art, for example the methods described in more detail in U.S. Pat. No. 10,055,540, US Application Pub. No. US20200010849A1, U.S. application Ser. No. 16/606,577, and international patent application publications WO2020181240A1, WO/2018/195357 and WO/2018/208856, each herein incorporated by reference, in their entirety, for all purposes.

Methods for identifying antigens (e.g., antigens derived from a tumor or an infectious disease organism) include identifying antigens that are likely to be presented on a cell surface (e.g., presented by MHC on a tumor cell, an infected cell, or an immune cell, including professional antigen presenting cells such as dendritic cells), and/or are likely to be immunogenic. As an example, one such method may comprise the steps of: obtaining at least one of exome, transcriptome or whole genome nucleotide sequencing and/or expression data from a tumor, an infected cell, or an infectious disease organism, wherein the nucleotide sequencing data and/or expression data is used to obtain data representing peptide sequences of each of a set of antigens (e.g., antigens derived from a tumor or an infectious disease organism); inputting the peptide sequence of each antigen into one or more presentation models to generate a set of numerical likelihoods that each of the antigens is presented by one or more MHC alleles on a cell surface, such as a tumor cell or an infected cell of the subject, the set of numerical likelihoods having been identified at least based on received mass spectrometry data; and selecting a subset of the set of antigens based on the set of numerical likelihoods to generate a set of selected antigens.

### III.A. Identification of Tumor Specific Mutations in Neoantigens

Also disclosed herein are methods for the identification of certain mutations (e.g., the variants or alleles that are present in cancer cells). In particular, these mutations can be present in the genome, transcriptome, proteome, or exome of cancer cells of a subject having cancer but not in normal tissue from the subject. Specific methods for identifying neoantigens, including shared neoantigens, that are specific to tumors are known to those skilled in the art, for example the methods described in more detail in U.S. Pat. No. 10,055,540, US Application Pub. No. US20200010849A1, and international patent application publications WO/2018/195357 and WO/2018/208856, each herein incorporated by reference, in their entirety, for all purposes. Examples of shared neoantigens that are specific to tumors are described in more detail in international patent application publication WO2019226941A1, herein incorporated by reference in its entirety, for all purposes. Shared neoantigens include, but are not limited to, KRAS-associated mutations (e.g., KRAS G12C, KRAS G12V, KRAS G12D, KRAS G12A, and/or KRAS Q61H mutations). For example, KRAS-associated MHC class I and/or MHC class II neoepitopes can include those mutations with reference to wild-type (WT) human KRAS, such as with reference to the following exemplary amino acid sequence:

MTEYKLVVVGAGGVGKSALTIQLIQNHFVDEYDPTIEDSYRKQVV
IDGETCLLDILDTAGQEEYSAMRDQYMRTGEGFLCVFAINNTKSF
EDIHHYREQIKRVKDSEDVPMVLVGNKCDLPSRTVDTKQAQDLAR
SYGIPFIETSAKTRQRVEDAFYTLVREIRQYRLKKISKEEKTPGC
VKIKKCIIM.

Genetic mutations in tumors can be considered useful for the immunological targeting of tumors if they lead to changes in the amino acid sequence of a protein exclusively in the tumor. Useful mutations include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence. Mutations can also include one or more of non-frameshift indel, missense or nonsense substitution, splice site alteration, genomic rearrangement or gene fusion, or any genomic or expression alteration giving rise to a neoORF.

Peptides with mutations or mutated polypeptides arising from for example, splice-site, frameshift, readthrough, or gene fusion mutations in tumor cells can be identified by sequencing DNA, RNA or protein in tumor versus normal cells.

Also mutations can include previously identified tumor specific mutations. Known tumor mutations can be found at the Catalogue of Somatic Mutations in Cancer (COSMIC) database.

A variety of methods are available for detecting the presence of a particular mutation or allele in an individual's DNA or RNA. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling-circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. can be a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they utilize incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

A number of initiatives obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle includes adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to identify mutations. As described above, four major sequencing-by-synthesis platforms are currently available: the Genome Sequencers from Roche/454 Life Sciences, the 1G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., US Patent Application No. 2006/0252077) can be linked to each fragment to be captured on a surface coated with a respective second member of that coupling pair.

Subsequent to the capture, the sequence can be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies.

Any cell type or tissue can be utilized to obtain nucleic acid samples for use in methods described herein. For example, a DNA or RNA sample can be obtained from a tumor or a bodily fluid, e.g., blood, obtained by known techniques (e.g. venipuncture) or saliva. Alternatively, nucleic acid tests can be performed on dry samples (e.g. hair or skin). In addition, a sample can be obtained for sequencing from a tumor and another sample can be obtained from normal tissue for sequencing where the normal tissue is of the same tissue type as the tumor. A sample can be obtained for sequencing from a tumor and another sample can be obtained from normal tissue for sequencing where the normal tissue is of a distinct tissue type relative to the tumor.

Tumors can include one or more of lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer.

Alternatively, protein mass spectrometry can be used to identify or validate the presence of mutated peptides bound to MHC proteins on tumor cells. Peptides can be acid-eluted from tumor cells or from HLA molecules that are immunoprecipitated from tumor, and then identified using mass spectrometry.

### IV. Vaccine Compositions

Also disclosed herein is an immunogenic composition, e.g., a vaccine composition, capable of raising a specific immune response, e.g., a tumor-specific immune response. Vaccine compositions typically comprise a plurality of neoantigens, e.g., selected using a method described herein. Vaccine compositions can also be referred to as vaccines.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 neoantigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different neoantigen sequences, 6, 7, 8, 9, 10 11, 12, 13, or 14 different neoantigen sequences, or 12, 13 or 14 different neoantigen sequences.

A vaccine can contain between 1 and 30 antigen-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen-encoding nucleic acid sequences, 6, 7, 8, 9, 10 11, 12, 13, or 14 different antigen-encoding nucleic acid sequences, or 12, 13 or 14 different antigen-encoding nucleic acid sequences.

Antigen-encoding nucleic acid sequences can refer to the antigen encoding portion of an antigen “cassette.” Features of an antigen cassette are described in greater detail herein. A cassette can contain two or more antigen-encoding nucleic acid sequences linked together in a cassette (e.g., concatenated antigen-encoding nucleic acid sequence encoding concatenated antigens that each include a T cell epitope, such as an antigen including both a T cell epitope and linkers or in some instances an antigen simply refers to the T cell epitope).

A vaccine can contain between 1 and 30 distinct epitope-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct epitope-encoding nucleic acid sequences, 6, 7, 8, 9, 10 11, 12, 13, or 14 distinct epitope-encoding nucleic acid sequences, or 12, 13 or 14 distinct epitope-encoding nucleic acid sequences. Epitope-encoding nucleic acid sequences can refer to sequences for individual epitope sequences, such as each of the concatenated T cell epitopes of two or more antigen-encoding nucleic acid sequences linked together in a cassette.

A vaccine can contain at two repeats of an epitope-encoding nucleic acid sequence. A used herein, an “iteration” (or interchangeably a “repeat”) refers to two or more iterations of an identical nucleic acid epitope-encoding nucleic acid sequences (inclusive of the optional 5′ linker sequence and/or the optional 3′ linker sequences described herein) within an antigen-encoding nucleic acid sequence. In one example, the antigen-encoding nucleic acid sequence portion of a cassette encodes at least two iterations of an epitope-encoding nucleic acid sequence. In further non-limiting examples, the antigen-encoding nucleic acid sequence portion of a cassette encodes more than one distinct epitope, and at least one of the distinct epitopes is encoded by at least two iterations of the nucleic acid sequence encoding the distinct epitope (i.e., at least two distinct epitope-encoding nucleic acid sequences). In illustrative non-limiting examples, an antigen-encoding nucleic acid sequence encodes epitopes A, B, and C encoded by epitope-encoding nucleic acid sequences epitope-encoding sequence A (E_{A}), epitope-encoding sequence B (E_{B}), and epitope-encoding sequence C (E_{C}), and exemplary antigen-encoding nucleic acid sequences having iterations of at least one of the distinct epitopes are illustrated by, but is not limited to, the formulas below:
Iteration of one distinct epitope (iteration of epitope A):

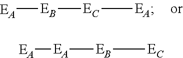

Iteration of multiple distinct epitopes (iterations of epitopes A, B, and C):

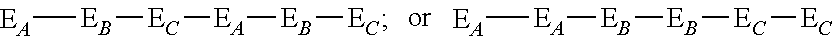

Multiple iterations of multiple distinct epitopes (iterations of epitopes A, B, and C):

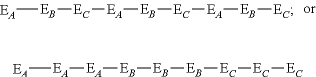

The above examples are not limiting and the antigen-encoding nucleic acid sequences having iterations of at least one of the distinct epitopes can encode each of the distinct epitopes in any order or frequency. For example, the order and frequency can be a random arrangement of the distinct epitopes, e.g., in an example with epitopes A, B, and C, by the formula E_{A}-E_{B}-E_{C}-E_{C}-E_{A}-E_{B}-E_{A}-E_{C}-E_{A}-E_{C}-E_{C}-E_{B}.

Also provided for herein is an antigen-encoding cassette, the antigen-encoding cassette having at least one antigen-encoding nucleic acid sequence described, from 5′ to 3′, by the formula:

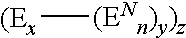

where E represents a nucleotide sequence including a distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof. In some aspects, at least one of the distinct epitope-encoding nucleic acid sequences with the at least two iterations encodes a KRAS-associated MHC class I and/or MHC class II neoepitope.

Each E or E^{N} can independently comprise any epitope-encoding nucleic acid sequence described herein (e.g., a peptide encoding an infectious disease T cell epitope and/or a neoantigen epitope). For example, Each E or E^{N} can independently comprises a nucleotide sequence described, from 5′ to 3′, by the formula (L5_{b}-N_{c}-L3_{d}), where N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. Epitopes and linkers that can be used are further described herein.

Iterations of an epitope-encoding nucleic acid sequences (inclusive of optional 5′ linker sequence and/or the optional 3′ linker sequences) can be linearly linked directly to one another (e.g., E_{A}-E_{A}- . . . as illustrated above). Iterations of an epitope-encoding nucleic acid sequences can be separated by one or more additional nucleotides sequences. In general, iterations of an epitope-encoding nucleic acid sequences can be separated by any size nucleotide sequence applicable for the compositions described herein. In one example, iterations of an epitope-encoding nucleic acid sequences can be separated by a separate distinct epitope-encoding nucleic acid sequence (e.g., E_{A}-E_{B}-E_{C}-E_{A} . . . , as illustrated above). In examples where iterations are separated by a single separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 75 nucleotides, such as in antigen-encoding nucleic acid represented by E_{A}-E_{B}-E_{A} . . . , E_{A} is separated by 75 nucleotides. In an illustrative example, an antigen-encoding nucleic acid having the sequence VTNTEMFVTAPDNLGYMYEVQWPGQTQPQIANCSVYDFFVWLHYYSVRDTVTNTEMF VTAPDNLGYMYEVQWPGQTQPQIANCSVYDFFVWLHYYSVRDT encoding iterations of 25mer antigens Trp1 (VTNTEMFVTAPDNLGYMYEVQWPGQ) and Trp2 (TQPQIANCSVYDFFVWLHYYSVRDT), the iterations of Trp1 are separated by the 25mer Trp2 and thus the repeats of the Trp1 epitope-encoding nucleic acid sequences are separated the 75 nucleotide Trp2 epitope-encoding nucleic acid sequence. In examples where iterations are separated by 2, 3, 4, 5, 6, 7, 8, or 9 separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 150, 225, 300, 375, 450, 525, 600, or 675 nucleotides, respectively.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or MHC class II molecules. Hence, vaccine compositions can comprise different fragments capable of associating with at least 2 preferred, at least 3 preferred, or at least 4 preferred MHC class I molecules and/or MHC class II molecules.

The vaccine composition can be capable of raising a specific cytotoxic T-cells response and/or a specific helper T-cell response.

A vaccine composition can further comprise an adjuvant and/or a carrier. Examples of useful adjuvants and carriers are given herein below. A composition can be associated with a carrier such as e.g. a protein or an antigen-presenting cell such as e.g. a dendritic cell (DC) capable of presenting the peptide to a T-cell.

Adjuvants are any substance whose admixture into a vaccine composition increases or otherwise modifies the immune response to a neoantigen. Carriers can be scaffold structures, for example a polypeptide or a polysaccharide, to which a neoantigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diptheria toxoid are suitable carriers. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Neoantigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more neoantigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the neoantigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of neoantigens, e.g., Salmonella typhi vectors, and the like will be apparent to those skilled in the art from the description herein.

### IV.A. Additional Considerations for Vaccine Design and Manufacture

IV.A.1. Determination of a Set of Peptides that Cover all Tumor Subclones

Truncal peptides, meaning those presented by all or most tumor subclones, will be prioritized for inclusion into the vaccine. Optionally, if there are no truncal peptides predicted to be presented and immunogenic with high probability, or if the number of truncal peptides predicted to be presented and immunogenic with high probability is small enough that additional non-truncal peptides can be included in the vaccine, then further peptides can be prioritized by estimating the number and identity of tumor subclones and choosing peptides so as to maximize the number of tumor subclones covered by the vaccine.

### IV.A.2. Neoantigen/Antigen Prioritization

After all of the above neoantigen filters are applied, more candidate neoantigens may still be available for vaccine inclusion than the vaccine technology can support. Additionally, uncertainty about various aspects of the neoantigen analysis may remain and tradeoffs may exist between different properties of candidate vaccine neoantigens. Thus, in place of predetermined filters at each step of the selection process, an integrated multi-dimensional model can be considered that places candidate neoantigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of neoantigens may lower the probability that a tumor will escape immune attack via downregulation or mutation of HLA molecules)
   Coverage of HLA classes (covering both HLA-I and HLA-II may increase the probability of therapeutic response and decrease the probability of tumor escape)

Additionally, optionally, neoantigens can be deprioritized (e.g., excluded) from the vaccination if they are predicted to be presented by HLA alleles lost or inactivated in either all or part of the patient's tumor. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010).

### IV.B. Antigen Cassette

The methods employed for the selection of one or more antigens, the cloning and construction of an “antigen cassette” and its insertion into a viral vector are within the skill in the art given the teachings provided herein. By “antigen cassette” or “cassette” is meant the combination of a selected antigen or plurality of antigens (e.g., antigen-encoding nucleic acid sequences) and the other regulatory elements necessary to transcribe the antigen(s) and express the transcribed product. The selected antigen or plurality of antigens can refer to distinct epitope sequences, e.g., an antigen-encoding nucleic acid sequence in the cassette can encode an epitope-encoding nucleic acid sequence (or plurality of epitope-encoding nucleic acid sequences) such that the epitopes are transcribed and expressed. An antigen or plurality of antigens can be operatively linked to regulatory components in a manner which permits transcription. Such components include conventional regulatory elements that can drive expression of the antigen(s) in a cell transfected with the viral vector. Thus the antigen cassette can also contain a selected promoter which is linked to the antigen(s) and located, with other, optional regulatory elements, within the selected viral sequences of the recombinant vector. A cassette can include one or more antigens, such as one or more pathogen-derived peptides, virus-derived peptides, bacteria-derived peptides, fungus-derived peptides, parasite-derived peptides, and/or tumor-derived peptides. A cassette can have one or more antigen-encoding nucleic acid sequences, such as a cassette containing multiple antigen-encoding nucleic acid sequences each independently operably linked to separate promoters and/or linked together using other multicistonic systems, such as 2A ribosome skipping sequence elements (e.g., E2A, P2A, F2A, or T2A sequences) or Internal Ribosome Entry Site (IRES) sequence elements. A linker can also have a cleavage site, such as a TEV or furin cleavage site. Linkers with cleavage sites can be used in combination with other elements, such as those in a multicistronic system. In a non-limiting illustrative example, a furin protease cleavage site can be used in conjunction with a 2A ribosome skipping sequence element such that the furin protease cleavage site is configured to facilitate removal of the 2A sequence following translation. In a cassette containing more than one antigen-encoding nucleic acid sequences, each antigen-encoding nucleic acid sequence can contain one or more epitope-encoding nucleic acid sequences (e.g., an antigen-encoding nucleic acid sequence encoding concatenated T cell epitopes).

Useful promoters can be constitutive promoters or regulated (inducible) promoters, which will enable control of the amount of antigen(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

Also disclosed herein is a viral vector comprising a cassette with at least one payload sequence operably linked to a regulatable promoter that is a TET promoter system, such as a TET-On system or TET-Off system. Without wishing to be bound by theory, a TET promoter system can be used to minimize transcription of payload nucleic acids encoded in a cassette, such as antigens encoded in a vaccine cassette, during viral production. TET promoter systems are described in detail in international patent application publication WO2020/243719, herein incorporated by reference for all purposes.

A TET promoter system can include a tetracycline (TET) repressor protein (TETr) controlled promoter. Accordingly, also disclosed herein is a viral vector comprising a cassette with at least one payload sequence operably linked to a tetracycline (TET) repressor protein (TETr) controlled promoter. A TETr controlled promoter can include the 19 bp TET operator (TETo) sequence TCCCTATCAGTGATAGAGA. A TETr controlled promoter can include 2, 3, 4, 5, 6, 7, 8, 9, or 10 or more TETo nucleic acid sequences. In TETr controlled promoter have 2 or more TETo nucleic acid sequences, the TETo sequences can be linked together. In TETr controlled promoter have 2 or more TETo nucleic acid sequences, the TETo sequences can be directly linked together. In TETr controlled promoter have 2 or more TETo nucleic acid sequences, the TETo sequences can be linked together with a linker sequence, such as a linker sequence having 1, 2, 3, 4, 5, 6, 7, 8, 9, 10, 11, 12, 13, 14, 15, 16, 17, 18, 19, or 20 or more nucleotides. In general, a TETr controlled promoter can use any promoter sequence desired, such as a SV40, EF-1, RSV, PGK, HSA, MCK or EBV promoter sequence. A TETr controlled promoter can use a CMV promoter sequence. A TETr controlled promoter can use a minimal CMV promoter sequence. TETo sequences can be upstream (5′) of a promoter sequence region where RNA polymerase binds. In an illustrative example, 7 TETo sequences are upstream (5′) of a promoter sequence. A TETr controlled promoter operably linked to the at least one payload nucleic acid sequence with TETo sequence upstream of the promoter sequence region can have an ordered sequence described in the formula, from 5′ to 3′:

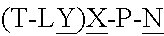

where N is a payload nucleic acid sequence, P is a RNA polymerase binding sequence of the promoter sequence operably linked to payload nucleic acid sequence, T is a TETo nucleic acid sequences comprising the nucleotide sequence JFKSIFEMMSJDSSU is a linker sequence, where Y=0 or 1 for each X, and wherein X=1, 2, 3, 4, 5, 6, 7, 8, 9, 10, 11, 12, 13, 14, 15, 16,
17, 18, 19, or 20. In an illustrative example, X=7 and Y=1 for each X describes where 7 TETo sequences are upstream (5′) of the promoter sequence and each TETo sequence is separated by a linker.

A TETo sequences can be downstream (3′) of a promoter sequence region where RNA polymerase binds. In another illustrative example, 2 TETo sequences are downstream (3′) of a promoter sequence. A TETr controlled promoter operably linked to the at least one payload nucleic acid sequence with TETo sequence downstream of the promoter sequence region can have an ordered sequence described in the formula, from 5′ to 3′:

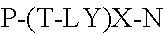

where N is a payload nucleic acid sequence, P is a RNA polymerase binding sequence of the promoter sequence operably linked to payload nucleic acid sequence, T is a TETo nucleic acid sequences comprising the nucleotide sequence JFKSIFEMMSJDSSU, L is a linker sequence, where Y=0 or 1 for each X, and wherein X=1, 2, 3, 4, 5, 6, 7, 8, 9, 10, 11, 12, 13, 14, 15, 16, 17, 18, 19, or 20. In an illustrative example, X=2 and Y=1 for each X describes where 2 TETo sequences are downstream (3′) of the promoter sequence and each TETo sequence is separated by a linker.

Viral production of vectors with TETr controlled promoters can use any viral production cell line engineered to express a TETr sequence (tTS), such as a 293 cell line or its derivatives (e.g., a 293F cell line) engineered to express tTS. Viral production of vectors with TETr controlled promoters in tTS-expressing cell can improve viral production. Viral production of vectors with TETr controlled promoters in tTS-expressing cell can improve viral infectivity defined as viral particles (VP) per infectious unit (IU). Viral production of vectors with TETr controlled promoters in tTS-expressing cell can improve viral production and/or viral infectivity by at least 1.5, at least 2, at least 2.5, at least 3, at least 3.5, at least 4, at least 4.5, at least 5, at least 6, at least 7, at least 8, at least 9, or at least 10-fold relative to production in a non-tTS-expressing cell. Viral production of vectors with TETr controlled promoters in tTS-expressing cell can improve viral production and/or viral infectivity by at least 15, at least 20, at least 25, at least 30, at least 35, at least 40, at least 45, at least 50, at least 60, at least 70, at least 80, at least 90, or at least 100-fold relative to production in a non-tTS-expressing cell. Viral production of vectors with TETr controlled promoters in tTS-expressing cell can improve viral production and/or viral infectivity by at least 1.5, at least 2, at least 2.5, at least 3, at least 3.5, at least 4, at least 4.5, at least 5, at least 6, at least 7, at least 8, at least 9, or at least 10-fold relative to production of a vector not having a TETr controlled promoter. Viral production of vectors with TETr controlled promoters in tTS-expressing cell can improve viral production and/or viral infectivity by at least 15, at least 20, at least 25, at least 30, at least 35, at least 40, at least 45, at least 50, at least 60, at least 70, at least 80, at least 90, or at least 100-fold relative to production of a vector not having a TETr controlled promoter.

The antigen cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. The poly-A sequence generally can be inserted in the cassette following the antigen-based sequences and before the viral vector sequences. A common intron sequence can also be derived from SV-40, and is referred to as the SV-40 T intron sequence. An antigen cassette can also contain such an intron, located between the promoter/enhancer sequence and the antigen(s). Selection of these and other common vector elements are conventional [see, e.g., Sambrook et al, “Molecular Cloning. A Laboratory Manual.”, 2d edit., Cold Spring Harbor Laboratory, New York (1989) and references cited therein] and many such sequences are available from commercial and industrial sources as well as from Genbank.

An antigen cassette can have one or more antigens. For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more antigens. Antigens can be linked directly to one another. Antigens can also be linked to one another with linkers. Antigens can be in any orientation relative to one another including N to C or C to N.

As described elsewhere herein, the antigen cassette can be located in the site of any selected deletion in a viral vector, such as the deleted structural proteins of a VEE backbone or the site of the E1 gene region deletion or E3 gene region deletion of a ChAd-based vector, among others which may be selected.

The antigen cassette can be described using the following formula to describe the ordered sequence of each element, from 5′ to 3′:

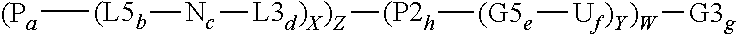

wherein P and P2 comprise promoter nucleotide sequences, N comprises an MHC class I epitope-encoding nucleic acid sequence, L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II antigen-encoding nucleic acid sequence, where for each X the corresponding Nc is an epitope encoding nucleic acid sequence, where for each Y the corresponding Uf is a MHC class II epitope-encoding nucleic acid sequence (e.g., universal MHC class II epitope-encoding nucleic acid sequence). A universal sequence can comprise at least one of Tetanus toxoid and PADRE. A universal sequence can comprise a Tetanus toxoid peptide. A universal sequence can comprise a PADRE peptide. A universal sequence can comprise a Tetanus toxoid and PADRE peptides. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4 or 5, Z=1 to 400, and W=0, 1, 2, 3, 4 or 5.

In one example, elements present include where a=0, b=1, d=1, e=1, g=1, h=0, X=10, Y=2, Z=1, and W=1, describing where no additional promoter is present (e.g. only the promoter nucleotide sequence provided by a vector backbone, such as an RNA alphavirus backbone is present), 10 MHC class I epitopes are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to the to a vector backbone (e.g., an RNA alphavirus backbone). Examples of linking the 3′ end of the antigen cassette to a vector backbone (e.g., an RNA alphavirus backbone) include linking directly to the 3′ UTR elements provided by the vector backbone, such as a 3′ 19-nt CSE. Examples of linking the 5′ end of the antigen cassette to a vector backbone (e.g., an RNA alphavirus backbone) include linking directly to a promoter or 5′ UTR element of the vector backbone, such as a subgenomic promoter sequence (e.g., a 26S subgenomic promoter sequence), an alphavirus 5′ UTR, a 51-nt CSE, or a 24-nt CSE.

Other examples include: where a=1 describing where a promoter other than the promoter nucleotide sequence provided by a vector backbone (e.g., an RNA alphavirus backbone) is present; where a=1 and Z is greater than 1 where multiple promoters other than the promoter nucleotide sequence provided by the vector backbone are present each driving expression of 1 or more distinct MHC class I epitope encoding nucleic acid sequences; where h=1 describing where a separate promoter is present to drive expression of the MHC class II epitope-encoding nucleic acid sequences; and where g=0 describing the MHC class II epitope-encoding nucleic acid sequence, if present, is directly linked to a vector backbone (e.g., an RNA alphavirus backbone).

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

Other examples include where each antigen that is present can have a 5′ linker, a 3′ linker, neither, or both. In examples where more than one antigen is present in the same antigen cassette, some antigens may have both a 5′ linker and a 3′ linker, while other antigens may have either a 5′ linker, a 3′ linker, or neither. In other examples where more than one antigen is present in the same antigen cassette, some antigens may have either a 5′ linker or a 3′ linker, while other antigens may have either a 5′ linker, a 3′ linker, or neither.

The promoter nucleotide sequences P and/or P2 can be the same as a promoter nucleotide sequence provided by a vector backbone, such as an RNA alphavirus backbone. For example, the promoter sequence provided by the vector backbone, Pn and P2, can each comprise a subgenomic promoter sequence (e.g., a 26S subgenomic promoter sequence) or a CMV promoter. The promoter nucleotide sequences P and/or P2 can be different from the promoter nucleotide sequence provided by a vector backbone (e.g., an RNA alphavirus backbone), as well as can be different from each other.

The 5′ linker L5 can be a native sequence or a non-natural sequence. Non-natural sequence include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

For each X, each N can encode a MHC class I epitope, a MHC class II epitope, an epitope/antigen capable of stimulating a B cell response, or a combination thereof. For each X, each N can encode a combination of a MHC class I epitope, a MHC class II epitope, and an epitope/antigen capable of stimulating a B cell response. For each X, each N can encode a combination of a MHC class I epitope and a MHC class II epitope. For each X, each N can encode a combination of a MHC class I epitope and an epitope/antigen capable of stimulating a B cell response. For each X, each N can encode a combination of a MHC class II epitope and an epitope/antigen capable of stimulating a B cell response. For each X, each N can encode a MHC class II epitope. For each X, each N can encode an epitope/antigen capable of stimulating a B cell response. For each X, each N can encode a MHC class I epitope 7-15 amino acids in length. For each X, each N can also encodes a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can also encodes a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. For each X, each N can encode a MHC class II epitope. For each X, each N can encode a MHC class II epitope 6-30, 6-35, or 6-40 amino acids in length. For each X, each N can encode a MHC class II epitope 10-30, 10-35, or 10-40 amino acids in length. For each X, each N can also encodes a MHC class II epitope at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 amino acids in length. For each X, each N can also encodes a MHC class II epitope at least 10 amino acids in length. For each X, each N can also encodes a MHC class II epitope at least 10, at least 11, at least 12, at least 13, at least 14, at least 15, at least 16, at least 17, at least 18, at least 19, or at least 20 amino acids in length. For each X, each N can also encodes a MHC class II epitope at least 10, at least 15, at least 20, at least 25, at least 30, at least 35, or at least 40 amino acids in length. For each X, each N can encode an epitope capable of stimulating a B cell response.

A cassette, including each cassette respectively in a multicistronic system, can be at least 100, 200, 300, 400, 500, 600, 700, 800, or 900 nucleotides in length. A cassette can be at least 1000, 2000, 3000, 4000, 5000, 6000, 7000, 8000, 9000, or 10000 nucleotides in length. A cassette can be at least 1000 nucleotides in length. A cassette can be at least 2000 nucleotides in length. A cassette can be at least 3000 nucleotides in length. A cassette can be at least 4000 nucleotides in length. A cassette can be at least 5000 nucleotides in length. A cassette can be at least 6000 nucleotides in length. A cassette can be at least 7000 nucleotides in length. A cassette can be at least 8000 nucleotides in length. A cassette can be at least 9000 nucleotides in length. A cassette can be between 100-1000, 100-2000, 100-3000, 100-4000, 100-5000, 100-6000, 100-7000, 100-8000, 100-9000, or 100-10000 nucleotides in length. A cassette can be between 500-1000, 500-2000, 500-3000, 500-4000, 500-5000, 500-6000, 500-7000, 500-8000, 500-9000, or 500-10000 nucleotides in length. A cassette can be between 1000-2000, 1000-3000, 1000-4000, 1000-5000, 1000-6000, 1000-7000, 1000-8000, 1000-9000, or 1000-10000 nucleotides in length.

A cassette can be about the length deleted from an alphavirus (e.g., the length of deleted structural proteins in a VEE backbone). A cassette can be less than the length deleted from an alphavirus. A cassette can be more than the length deleted from an alphavirus.

For vectors including multiple cassettes, the total length of all cassettes combined can be at least 100, 200, 300, 400, 500, 600, 700, 800, or 900 nucleotides in length. For vectors including multiple cassettes, the total length of all cassettes combined can be at least 1000, 2000, 3000, 4000, 5000, 6000, 7000, 8000, 9000, or 10000 nucleotides in length. For vectors including multiple cassettes, the total length of all cassettes combined can be between 100-1000, 100-2000, 100-3000, 100-4000, 100-5000, 100-6000, 100-7000, 100-8000, 100-9000, or 100-10000 nucleotides in length. For vectors including multiple cassettes, the total length of all cassettes combined can be between 500-1000, 500-2000, 500-3000, 500-4000, 500-5000, 500-6000, 500-7000, 500-8000, 500-9000, or 500-10000 nucleotides in length. For vectors including multiple cassettes, the total length of all cassettes combined can be between 1000-2000, 1000-3000, 1000-4000, 1000-5000, 1000-6000, 1000-7000, 1000-8000, 1000-9000, or 1000-10000 nucleotides in length.

The cassette encoding the one or more antigens can be 700 nucleotides or less. The cassette encoding the one or more antigens can be 700 nucleotides or less and encode 2 distinct epitope-encoding nucleic acid sequences (e.g., encode 2 distinct infectious disease or tumor derived nucleic acid sequences encoding an immunogenic polypeptide). The cassette encoding the one or more antigens can be 700 nucleotides or less and encode at least 2 distinct epitope-encoding nucleic acid sequences. The cassette encoding the one or more antigens can be 700 nucleotides or less and encode 3 distinct epitope-encoding nucleic acid sequences. The cassette encoding the one or more antigens can be 700 nucleotides or less and encode at least 3 distinct epitope-encoding nucleic acid sequences. The cassette encoding the one or more antigens can be 700 nucleotides or less and include 1-10, 1-5, 1, 2, 3, 4, 5, 6, 7, 8, 9, 10, or more antigens.

The cassette encoding the one or more antigens can be between 375-700 nucleotides in length. The cassette encoding the one or more antigens can be between 375-700 nucleotides in length and encode 2 distinct epitope-encoding nucleic acid sequences (e.g., encode 2 distinct infectious disease or tumor derived nucleic acid sequences encoding an immunogenic polypeptide). The cassette encoding the one or more antigens can be between 375-700 nucleotides in length and encode at least 2 distinct epitope-encoding nucleic acid sequences. The cassette encoding the one or more antigens can be between 375-700 nucleotides in length and encode 3 distinct epitope-encoding nucleic acid sequences. The cassette encoding the one or more antigens be between 375-700 nucleotides in length and encode at least 3 distinct epitope-encoding nucleic acid sequences. The cassette encoding the one or more antigens can be between 375-700 nucleotides in length and include 1-10, 1-5, 1, 2, 3, 4, 5, 6, 7, 8, 9, 10, or more antigens.

The cassette encoding the one or more antigens can be 600, 500, 400, 300, 200, or 100 nucleotides in length or less. The cassette encoding the one or more antigens can be 600, 500, 400, 300, 200, or 100 nucleotides in length or less and encode 2 distinct epitope-encoding nucleic acid sequences. The cassette encoding the one or more antigens can be 600, 500, 400, 300, 200, or 100 nucleotides in length or less and encode at least 2 distinct epitope-encoding nucleic acid sequences. The cassette encoding the one or more antigens can be 600, 500, 400, 300, 200, or 100 nucleotides in length or less and encode 3 distinct epitope-encoding nucleic acid sequences. The cassette encoding the one or more antigens can be 600, 500, 400, 300, 200, or 100 nucleotides in length or less and encode at least 3 distinct epitope-encoding nucleic acid sequences. The cassette encoding the one or more antigens can be 600, 500, 400, 300, 200, or 100 nucleotides in length or less and include 1-10, 1-5, 1, 2, 3, 4, 5, 6, 7, 8, 9, 10, or more antigens.

The cassette encoding the one or more antigens can be between 375-600, between 375-500, or between 375-400 nucleotides in length. The cassette encoding the one or more antigens can be between 375-600, between 375-500, or between 375-400 nucleotides in length and encode 2 distinct epitope-encoding nucleic acid sequences. The cassette encoding the one or more antigens can be between 375-600, between 375-500, or between 375-400 nucleotides in length and encode at least 2 distinct epitope-encoding nucleic acid sequences. The cassette encoding the one or more antigens can be between 375-600, between 375-500, or between 375-400 nucleotides in length and encode 3 distinct epitope-encoding nucleic acid sequences. The cassette encoding the one or more antigens can be between 375-600, between 375-500, or between 375-400 nucleotides in length and encode at least 3 distinct epitope-encoding nucleic acid sequences. The cassette encoding the one or more antigens can be between 375-600, between 375-500, or between 375-400 nucleotides in length and include 1-10, 1-5, 1, 2, 3, 4, 5, 6, 7, 8, 9, 10, or more antigens.

In some instances, an antigen or epitope in a cassette encoding additional antigens and/or epitopes may be an immunodominant epitope relative to the others encoded.

Immunodominance, in general, is the skewing of an immune response towards only one or a few specific immunogenic peptides. Immunodominance can be assessed as part of an immune monitoring protocol. For example, immunodominance can be assessed through evaluating T cell and/or B cell responses to the encoded antigens.

Immunodominance can be assessed as the impact of an immunodominant antigen's presence on the immune response to one or more other antigens. For example, an immunodominant antigen and its respective immune response (e.g., an immunodominant MHC class I epitope) can reduce the immune response of another antigen relative to the immune response in the absence of the immunodominant antigen. This reduction can be such that the immune response in the presence of the immunodominant antigen is not considered a therapeutically effective response. For example, an MHC class I epitope would generally be considered immunodominant if T cell responses to other antigens are no longer considered therapeutically effective responses compared to responses elicited in the absence of the immunodominant MHC class I epitope. An immune response can also be reduced to below a limit of detection or near the limit of detection. relative to the response in the absence of the immunodominant antigen. For example, an MHC class I epitope would generally be considered immunodominant if T cell responses to other antigens are at or below the limit of detection compared to responses elicited in the absence of the immunodominant MHC class I epitope. In general, the assessment of immunodominance is between two antigens both capable of stimulating an immune response, e.g., between two T cell epitopes in a vaccine composition administered to a subject possessing a cognate MHC allele known or predicted to present each epitope, respectively. Immunodominance can be assessed through evaluating relative immune responses to other antigens in the presence and absence of the suspected immunodominant antigen.

Immunodominance can be assessed as a relative difference in the immune responses between two or more antigens. Immunodominance can refer to a 5-fold, 10-fold, 20-fold, 30-fold, 40-fold, or 50-fold immune response of a specific antigen relative to another antigen encoded in the same cassette. Immunodominance can refer to a 100-fold, 200-fold, 300-fold, 400-fold, or 500-fold immune response of a specific antigen relative to another antigen encoded in the same cassette. Immunodominance can refer to a 1000-fold, 2000-fold, 3000-fold, 4000-fold, or 5000-fold immune response of a specific antigen relative to another antigen encoded in the same cassette. Immunodominance can refer to a 10,000-fold immune response of a specific antigen relative to another antigen encoded in the same cassette.

In some instances, it may be desired to avoid vaccine compositions containing an immunodominant epitope. For example, it may be desired to avoid designing a vaccine cassette encoding an immunodominant epitope. Without wishing to be bound by theory, administering and/or encoding an immunodominant epitope together with additional epitope may reduce the immune response to the additional epitopes, including potentially ultimately reducing vaccine efficacy against the additional epitopes. As an illustrative non-limiting example, vaccine compositions including TP53-associated neoepitopes may have the immune response, e.g., a T cell response, skewed towards the TP53-associated neoepitope negatively impacting (e.g., reducing the immune response to where the immune response is not a therapeutically effective response and/or to below a limit of detection) the immune response to other antigens or epitopes in the vaccine composition (e.g., one or more KRAS-associated neoepitopes in the vaccine composition, such as any of the KRAS-associated neoepitopes QCEIOWAREFLKEIGJ, IEFROEIFJEF, IEFROEIFJ, EFROEIFJE, FROEIFJEF, SINFEKL, LLLLLVVVV, and EKLAAYLLL (shown in SEQ ID NOs:). Accordingly, vaccine compositions can be designed to not contain an immunodominant epitope, such as designing a vaccine cassette (e.g., a (neo)antigen-encoding cassette) to not encode an immunodominant epitope. For example, the cassette does not encode an epitope that reduces an immune response to another epitope encoded in the cassette when administered in a vaccine composition to a subject relative to an immune response when the other epitope is administered in the absence of the immunodominant MHC class I epitope. In another example, the cassette does not encode an epitope that reduces an immune response to another epitope encoded in the cassette to below a limit of detection when administered in a vaccine composition to a subject relative to an immune response when the other epitope is administered in the absence of the immunodominant MHC class I epitope. In another example, the cassette does not encode an epitope that reduces an immune response to another epitope encoded in the cassette, wherein the immune response is not a therapeutically effective response, when administered in a vaccine composition to a subject relative to an immune response when the other epitope is administered in the absence of the immunodominant MHC class I epitope. In another example, the cassette does not encode an epitope that stimulates a 5-fold, 10-fold, 20-fold, 30-fold, 40-fold, or 50-fold or greater immune response relative to another epitope encoded in the same cassette in a vaccine composition administered to a subject, where each antigen is capable of stimulating an immune response in the subject. In another example, the cassette does not encode an epitope that stimulates a 100-fold, 200-fold, 300-fold, 400-fold, or 500-fold or greater immune response relative to another epitope encoded in the same cassette in a vaccine composition administered to a subject, where each antigen is capable of stimulating an immune response in the subject. In another example, the cassette does not encode an epitope that stimulates a 1000-fold, 2000-fold, 3000-fold, 4000-fold, or 5000-fold or greater immune response relative to another epitope encoded in the same cassette in a vaccine composition administered to a subject, where each antigen is capable of stimulating an immune response in the subject. In another example, the cassette does not encode an epitope that results in a 10,000-fold or greater immune response relative to another epitope encoded in the same cassette in a vaccine composition administered to a subject, where each antigen is capable of stimulating an immune response in the subject.

### IV.C. Immune Modulators

Vectors described herein, such as C68 vectors described herein or alphavirus vectors described herein, can comprise a nucleic acid which encodes at least one antigen and the same or a separate vector can comprise a nucleic acid which encodes at least one immune modulator. An immune modulator can include a binding molecule (e.g., an antibody such as an scFv) which binds to and blocks the activity of an immune checkpoint molecule. An immune modulator can include a cytokine, such as IL-2, IL-7, IL-12 (including IL-12 p35, p40, p70, and/or p70-fusion constructs), IL-15, or IL-21. An immune modulator can include a modified cytokine (e.g., pegIL-2). Vectors can comprise an antigen cassette and one or more nucleic acid molecules encoding an immune modulator.

Illustrative immune checkpoint molecules that can be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+ (αβ) T cells), CD160 (also referred to as BY55), and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, and CGEN-15049. Illustrative immune checkpoint inhibitors include Tremelimumab (CTLA-4 blocking antibody), anti-OX40, PD-L1 monoclonal Antibody (Anti-B7-H1; MEDI4736), ipilimumab, MK-3475 (PD-1 blocker), Nivolumamb (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Antibody-encoding sequences can be engineered into vectors such as C68 using ordinary skill in the art. An exemplary method is described in Fang et al., Stable antibody expression at therapeutic levels using the 2A peptide. Nat Biotechnol. 2005 May; 23(5):584-90. Epub 2005 Apr. 17; herein incorporated by reference for all purposes.

### IV.D. Self-Amplifying RNA Vectors

In general, all self-amplifying RNA (SAM) vectors contain a self-amplifying backbone derived from a self-replicating virus. The term “self-amplifying backbone” refers to minimal sequence(s) of a self-replicating virus that allows for self-replication of the viral genome. For example, minimal sequences that allow for self-replication of an alphavirus can include conserved sequences for nonstructural protein-mediated amplification (e.g., a nonstructural protein 1 (nsP1) gene, a nsP2 gene, a nsP3 gene, a nsP4 gene, and/or a polyA sequence). A self-amplifying backbone can also include sequences for expression of subgenomic viral RNA (e.g., a 26S promoter element for an alphavirus). SAM vectors can be positive-sense RNA polynucleotides or negative-sense RNA polynucleotides, such as vectors with backbones derived from positive-sense or negative-sense self-replicating viruses. Self-replicating viruses include, but are not limited to, alphaviruses, flaviviruses (e.g., Kunjin virus), measles viruses, and rhabdoviruses (e.g., rabies virus and vesicular stomatitis virus). Examples of SAM vector systems derived from self-replicating viruses are described in greater detail in Lundstrom (Molecules. 2018 Dec. 13; 23(12). pii: E3310. doi: 10.3390/molecules23123310), herein incorporated by reference for all purposes.

### IV.D.1. Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbial Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes.

Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template. Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

### IV.D.2. Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, or self-amplifying mRNA (SAM) vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly stimulate an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, stimulates a robust immune response to antigen, does not stimulate an immune response to the vector itself, and can be used in a safe manner. Furthermore, the antigen expression cassette can be designed to stimulate different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, a alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

### IV.D.3. Self-Amplifying Virus Production In Vitro

A convenient technique well-known in the art for RNA production is in vitro transcription (IVT). In this technique, a DNA template of the desired vector is first produced by techniques well-known to those in the art, including standard molecular biology techniques such as cloning, restriction digestion, ligation, gene synthesis (e.g., chemical and/or enzymatic synthesis), and polymerase chain reaction (PCR).

The DNA template contains a RNA polymerase promoter at the 5′ end of the sequence desired to be transcribed into RNA (e.g., SAM). Promoters include, but are not limited to, bacteriophage polymerase promoters such as T3, T7, K11, or SP6. Depending on the specific RNA polymerase promoter sequence chosen, additional 5′ nucleotides can transcribed in addition to the desired sequence. For example, the canonical T7 promoter can be referred to by the sequence TAATACGACTCACTATAGG, in which an IVT reaction using the DNA template TAATACGACTCACTATAGGN for the production of desired sequence N will result in the mRNA sequence GG-N. In general, and without wishing to be bound by theory, T7 polymerase more efficiently transcribes RNA transcripts beginning with guanosine. In instances where additional 5′ nucleotides are not desired (e.g., no additional GG), the RNA polymerase promoter contained in the DNA template can be a sequence the results in transcripts containing only the 5′ nucleotides of the desired sequence, e.g., a SAM having the native 5′ sequence of the self-replicating virus from which the SAM vector is derived. For example, a minimal T7 promoter can be referred to by the sequence TAATACGACTCACTATA, in which an IVT reaction using the DNA template TAATACGACTCACTATAN for the production of desired sequence N will result in the mRNA sequence N. Likewise, a minimal SP6 promoter referred to by the sequence ATTTAGGTGACACTATA can be used to generate transcripts without additional 5′ nucleotides. In a typical IVT reaction, the DNA template is incubated with the appropriate RNA polymerase enzyme, buffer agents, and nucleotides (NTPs).

The resulting RNA polynucleotide can optionally be further modified including, but limited to, addition of a 5′ cap structure such as 7-methylguanosine or a related structure, and optionally modifying the 3′ end to include a polyadenylate (polyA) tail. In a modified IVT reaction, RNA is capped with a 5′ cap structure co-transcriptionally through the addition of cap analogues during IVT. Cap analogues can include dinucleotide (m⁷G-ppp-N) cap analogues or trinucleotide (m⁷G-ppp-N-N) cap analogues, where N represents a nucleotide or modified nucleotide (e.g., ribonucleosides including, but not limited to, adenosine, guanosine, cytidine, and uradine). Exemplary cap analogues and their use in IVT reactions are also described in greater detail in U.S. Pat. No. 10,519,189, herein incorporated by reference for all purposes. As discussed, T7 polymerase more efficiently transcribes RNA transcripts beginning with guanosine. To improve transcription efficiency in templates that do not begin with guanosine, a trinucleotide cap analogue (m⁷G-ppp-N-N) can be used. The trinucleotide cap analogue can increase transcription efficiency 2, 3, 4, 5, 6, 7, 8, 9, 10, 11, 12, 13, 14, 15, 16, 17, 18, 19, 20-fold or more relative to an IVT reaction using a dinucleotide cap analogue (m⁷G-ppp-N).

A 5′ cap structure can also be added following transcription, such as using a vaccinia capping system (e.g., NEB Cat. No. M2080) containing mRNA 2′-O-methyltransferase and S-Adenosyl methionine.

The resulting RNA polynucleotide can optionally be further modified separately from or in addition to the capping techniques described including, but limited to, modifying the 3′ end to include a polyadenylate (polyA) tail.

The RNA can then be purified using techniques well-known in the field, such as phenol-chloroform extraction or column purification (e.g., chromatography-based purification).

### IV.D.4. Delivery Via Lipid Nanoparticle

An aspect to consider in vaccine vector design is immunity against the vector itself. This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes.

In the case of alphavirus vectors, the standard delivery method is the previously discussed helper virus system that provides capsid, E1, and E2 proteins in trans to produce infectious viral particles. However, it is important to note that the E1 and E2 proteins are often major targets of neutralizing antibodies (Strauss 1994). Thus, the efficacy of using alphavirus vectors to deliver antigens of interest to target cells may be reduced if infectious particles are targeted by neutralizing antibodies.

An alternative to viral particle mediated gene delivery is the use of nanomaterials to deliver expression vectors (Riley 2017). Nanomaterial vehicles, importantly, can be made of non-immunogenic materials and generally avoid stimulating immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target effects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

IV.E. Chimpanzee adenovirus (ChAd)
IV.E.1. Viral delivery with chimpanzee adenovirus

Vaccine compositions for delivery of one or more antigens (e.g., via an antigen cassette) can be created by providing adenovirus nucleotide sequences of chimpanzee origin, a variety of novel vectors, and cell lines expressing chimpanzee adenovirus genes. A nucleotide sequence of a chimpanzee C68 adenovirus (also referred to herein as ChAdV68) can be used in a vaccine composition for antigen delivery (See SEQ ID NO: 1). Use of C68 adenovirus derived vectors is described in further detail in U.S. Pat. No. 6,083,716, which is herein incorporated by reference in its entirety, for all purposes. ChAdV68-based vectors and delivery systems are described in detail in US App. Pub. No. US20200197500A1 and international patent application publication WO2020243719A1, each of which is herein incorporated by reference for all purposes.

In a further aspect, provided herein is a recombinant adenovirus comprising the DNA sequence of a chimpanzee adenovirus such as C68 and an antigen cassette operatively linked to regulatory sequences directing its expression. The recombinant virus is capable of infecting a mammalian, preferably a human, cell and capable of expressing the antigen cassette product in the cell. In this vector, the native chimpanzee E1 gene, and/or E3 gene, and/or E4 gene can be deleted. An antigen cassette can be inserted into any of these sites of gene deletion. The antigen cassette can include an antigen against which a primed immune response is desired.

In another aspect, provided herein is a mammalian cell infected with a chimpanzee adenovirus such as C68.

In still a further aspect, a novel mammalian cell line is provided which expresses a chimpanzee adenovirus gene (e.g., from C68) or functional fragment thereof.

In still a further aspect, provided herein is a method for delivering an antigen cassette into a mammalian cell comprising the step of introducing into the cell an effective amount of a chimpanzee adenovirus, such as C68, that has been engineered to express the antigen cassette.

Still another aspect provides a method for stimulating an immune response in a mammalian host to treat cancer. The method can comprise the step of administering to the host an effective amount of a recombinant chimpanzee adenovirus, such as C68, comprising an antigen cassette that encodes one or more antigens from the tumor against which the immune response is targeted.

Still another aspect provides a method for stimulating an immune response in a mammalian host to treat or prevent a disease in a subject, such as an infectious disease. The method can comprise the step of administering to the host an effective amount of a recombinant chimpanzee adenovirus, such as C68, comprising an antigen cassette that encodes one or more antigens, such as from the infectious disease against which the immune response is targeted.

Also disclosed is a non-simian mammalian cell that expresses a chimpanzee adenovirus gene obtained from the sequence of SEQ ID NO: 1. The gene can be selected from the group consisting of the adenovirus E1A, E1B, E2A, E2B, E3, E4, L1, L2, L3, L4 and L5 of SEQ ID NO: 1.

Also disclosed is a nucleic acid molecule comprising a chimpanzee adenovirus DNA sequence comprising a gene obtained from the sequence of SEQ ID NO: 1. The gene can be selected from the group consisting of said chimpanzee adenovirus E1A, E1B, E2A, E2B, E3, E4, L1, L2, L3, L4 and L5 genes of SEQ ID NO: 1. In some aspects the nucleic acid molecule comprises SEQ ID NO: 1. In some aspects the nucleic acid molecule comprises the sequence of SEQ ID NO: 1, lacking at least one gene selected from the group consisting of E1A, E1B, E2A, E2B, E3, E4, L1, L2, L3, L4 and L5 genes of SEQ ID NO: 1.

Also disclosed is a vector comprising a chimpanzee adenovirus DNA sequence obtained from SEQ ID NO: 1 and an antigen cassette operatively linked to one or more regulatory sequences which direct expression of the cassette in a heterologous host cell, optionally wherein the chimpanzee adenovirus DNA sequence comprises at least the cis-elements necessary for replication and virion encapsidation, the cis-elements flanking the antigen cassette and regulatory sequences. In some aspects, the chimpanzee adenovirus DNA sequence comprises a gene selected from the group consisting of E1A, E1B, E2A, E2B, E3, E4, L1, L2, L3, L4 and L5 gene sequences of SEQ ID NO: 1. In some aspects the vector can lack the E1A and/or E1B gene.

Also disclosed herein is a adenovirus vector comprising: a partially deleted E4 gene comprising a deleted or partially-deleted E4orf2 region and a deleted or partially-deleted E4orf3 region, and optionally a deleted or partially-deleted E4orf4 region. The partially deleted E4 can comprise an E4 deletion of at least nucleotides 34,916 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of nucleotides 34,916 to 34,942 of the sequence shown in SEQ ID NO:1, at least a partial deletion of nucleotides 34,952 to 35,305 of the sequence shown in SEQ ID NO:1, and at least a partial deletion of nucleotides 35,302 to 35,642 of the sequence shown in SEQ ID NO:1, and wherein the vector comprises at least nucleotides 2 to 36,518 of the sequence set forth in SEQ ID NO:1 The partially deleted E4 can comprise an E4 deletion of at least nucleotides 34,980 to 36,516 of the sequence shown in SEQ ID NO:1, and wherein the vector comprises at least nucleotides 2 to 36,518 of the sequence set forth in SEQ ID NO:1. The partially deleted E4 can comprise an E4 deletion of at least nucleotides 34,979 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of E4Orf2, a fully deleted E4Orf3, and at least a partial deletion of E4Orf4. The partially deleted E4 can comprise an E4 deletion of at least a partial deletion of E4Orf2, at least a partial deletion of E4Orf3, and at least a partial deletion of E4Orf4. The partially deleted E4 can comprise an E4 deletion of at least a partial deletion of E4Orf1, a fully deleted E4Orf2, and at least a partial deletion of E4Orf3. The partially deleted E4 can comprise an E4 deletion of at least a partial deletion of E4Orf2 and at least a partial deletion of E4Orf3. The partially deleted E4 can comprise an E4 deletion between the start site of E4Orf1 to the start site of E4Orf5. The partially deleted E4 can be an E4 deletion adjacent to the start site of E4Orf1. The partially deleted E4 can be an E4 deletion adjacent to the start site of E4Orf2. The partially deleted E4 can be an E4 deletion adjacent to the start site of E4Orf3. The partially deleted E4 can be an E4 deletion adjacent to the start site of E4Orf4. The E4 deletion can be at least 50, at least 100, at least 200, at least 300, at least 400, at least 500, at least 600, at least 700, at least 800, at least 900, at least
1000, at least 1100, at least 1200, at least 1300, at least 1400, at least 1500, at least 1600, at least 1700, at least 1800, at least 1900, or at least 2000 nucleotides. The E4 deletion can be at least 700 nucleotides. The E4 deletion can be at least 1500 nucleotides. The E4 deletion can be 50 or less, 100 or less, 200 or less, 300 or less, 400 or less, 500 or less, 600 or less, 700 or less, 800 or
less, 900 or less, 1000 or less, 1100 or less, 1200 or less, 1300 or less, 1400 or less, 1500 or less, 1600 or less, 1700 or less, 1800 or less, 1900 or less, or 2000 or less nucleotides. The E4 deletion can be 750 nucleotides or less. The E4 deletion can be at least 1550 nucleotides or less. that lacks at least nucleotides 34,916 to 35,642 of the sequence shown in SEQ ID NO:1. The partially deleted E4 gene can be the E4 gene sequence shown in SEQ ID NO:1 that lacks the E4 gene sequence shown in SEQ ID NO:1 and that lacks at least nucleotides 34,916 to 34,942, nucleotides 34,952 to 35,305 of the sequence shown in SEQ ID NO:1, and nucleotides 35,302 to 35,642 of the sequence shown in SEQ ID NO:1. The partially deleted E4 gene can be the E4 gene sequence shown in SEQ ID NO:1 and that lacks at least nucleotides 34,980 to 36,516 of the sequence shown in SEQ ID NO:1. The partially deleted E4 gene can be the E4 gene sequence shown in SEQ ID NO:1 and that lacks at least nucleotides 34,979 to 35,642 of the sequence shown in SEQ ID NO:1. The adenovirus vector having the partially deleted E4 gene can have a cassette, wherein the cassette comprises at least one payload nucleic acid sequence, and wherein the cassette comprises at least one promoter sequence operably linked to the at least one payload nucleic acid sequence. The adenovirus vector having the partially deleted E4 gene can have one or more genes or regulatory sequences of the ChAdV68 sequence shown in SEQ ID NO: 1, optionally wherein the one or more genes or regulatory sequences comprise at least one of the chimpanzee adenovirus inverted terminal repeat (ITR), E1A, E1B, E2A, E2B, E3, E4, L1, L2, L3, L4, and L5 genes of the sequence shown in SEQ ID NO: 1. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, and optionally the nucleotides 2 to 34,916 additionally lack nucleotides 577 to 3403 of the sequence shown in SEQ ID NO:1 corresponding to an E1 deletion and/or lack nucleotides 27,125 to 31,825 of the sequence shown in SEQ ID NO:1 corresponding to an E3 deletion. The adenovirus vector having the partially deleted E4 gene can have nucleotides 35,643 to 36,518 of the sequence shown in SEQ ID NO:1, and wherein the partially deleted E4 gene is 5′ of the nucleotides 35,643 to 36,518. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein the partially deleted E4 gene is 5′ of the nucleotides 35,643 to 36,518.

The partially deleted E4 gene can be the E4 gene sequence shown in SEQ ID NO:1 that lacks at least nucleotides 34,916 to 35,642 of the sequence shown in SEQ ID NO:1, nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein the partially deleted E4 gene is 5′ of the nucleotides 35,643 to 36,518.

Also disclosed herein is a host cell transfected with a vector disclosed herein such as a C68 vector engineered to expression an antigen cassette. Also disclosed herein is a human cell that expresses a selected gene introduced therein through introduction of a vector disclosed herein into the cell.

Also disclosed herein is a method for delivering an antigen cassette to a mammalian cell comprising introducing into said cell an effective amount of a vector disclosed herein such as a C68 vector engineered to expression the antigen cassette.

Also disclosed herein is a method for producing an antigen comprising introducing a vector disclosed herein into a mammalian cell, culturing the cell under suitable conditions and producing the antigen.

### IV.E.2. E1-Expressing Complementation Cell Lines

To generate recombinant chimpanzee adenoviruses (Ad) deleted in any of the genes described herein, the function of the deleted gene region, if essential to the replication and infectivity of the virus, can be supplied to the recombinant virus by a helper virus or cell line, i.e., a complementation or packaging cell line. For example, to generate a replication-defective chimpanzee adenovirus vector, a cell line can be used which expresses the E1 gene products of the human or chimpanzee adenovirus; such a cell line can include HEK293 or variants thereof. The protocol for the generation of the cell lines expressing the chimpanzee E1 gene products (Examples 3 and 4 of U.S. Pat. No. 6,083,716) can be followed to generate a cell line which expresses any selected chimpanzee adenovirus gene.

An AAV augmentation assay can be used to identify a chimpanzee adenovirus E1-expressing cell line. This assay is useful to identify E1 function in cell lines made by using the E1 genes of other uncharacterized adenoviruses, e.g., from other species. That assay is described in Example 4B of U.S. Pat. No. 6,083,716.

A selected chimpanzee adenovirus gene, e.g., E1, can be under the transcriptional control of a promoter for expression in a selected parent cell line. Inducible or constitutive promoters can be employed for this purpose. Among inducible promoters are included the sheep metallothionine promoter, inducible by zinc, or the mouse mammary tumor virus (MMTV) promoter, inducible by a glucocorticoid, particularly, dexamethasone. Other inducible promoters, such as those identified in International patent application WO95/13392, incorporated by reference herein can also be used in the production of packaging cell lines. Constitutive promoters in control of the expression of the chimpanzee adenovirus gene can be employed also.

A parent cell can be selected for the generation of a novel cell line expressing any desired C68 gene. Without limitation, such a parent cell line can be HeLa [ATCC Accession No. CCL 2], A549 [ATCC Accession No. CCL 185], KB [CCL 17], Detroit [e.g., Detroit 510, CCL and WI-38 [CCL 75] cells. Other suitable parent cell lines can be obtained from other sources. Parent cell lines can include CHO, HEK293 or variants thereof, 911, HeLa, A549, LP-293, PER.C6, or AE1-2a.

An E1-expressing cell line can be useful in the generation of recombinant chimpanzee adenovirus E1 deleted vectors. Cell lines constructed using essentially the same procedures that express one or more other chimpanzee adenoviral gene products are useful in the generation of recombinant chimpanzee adenovirus vectors deleted in the genes that encode those
products. Further, cell lines which express other human Ad E1 gene products are also useful in generating chimpanzee recombinant Ads.

### IV.E.3. Recombinant Viral Particles as Vectors

The compositions disclosed herein can comprise viral vectors, that deliver at least one antigen to cells. Such vectors comprise a chimpanzee adenovirus DNA sequence such as C68 and an antigen cassette operatively linked to regulatory sequences which direct expression of the cassette. The C68 vector is capable of expressing the cassette in an infected mammalian cell. The C68 vector can be functionally deleted in one or more viral genes. An antigen cassette comprises at least one antigen under the control of one or more regulatory sequences such as a promoter.

Optional helper viruses and/or packaging cell lines can supply to the chimpanzee viral vector any necessary products of deleted adenoviral genes.

The term “functionally deleted” means that a sufficient amount of the gene region is removed or otherwise altered, e.g., by mutation or modification, so that the gene region is no longer capable of producing one or more functional products of gene expression. Mutations or modifications that can result in functional deletions include, but are not limited to, nonsense mutations such as introduction of premature stop codons and removal of canonical and non-canonical start codons, mutations that alter mRNA splicing or other transcriptional processing, or combinations thereof. If desired, the entire gene region can be removed.

Modifications of the nucleic acid sequences forming the vectors disclosed herein, including sequence deletions, insertions, and other mutations may be generated using standard molecular biological techniques and are within the scope of this invention.

### IV.E.4. Construction of the Viral Plasmid Vector

The chimpanzee adenovirus C68 vectors useful in this invention include recombinant, defective adenoviruses, that is, chimpanzee adenovirus sequences functionally deleted in the E1a or E1b genes, and optionally bearing other mutations, e.g., temperature-sensitive mutations or deletions in other genes. It is anticipated that these chimpanzee sequences are also useful in forming hybrid vectors from other adenovirus and/or adeno-associated virus sequences.

Homologous adenovirus vectors prepared from human adenoviruses are described in the published literature [see, for example, Kozarsky I and II, cited above, and references cited therein, U.S. Pat. No. 5,240,846].

In the construction of useful chimpanzee adenovirus C68 vectors for delivery of an antigen cassette to a human (or other mammalian) cell, a range of adenovirus nucleic acid sequences can be employed in the vectors. A vector comprising minimal chimpanzee C68 adenovirus sequences can be used in conjunction with a helper virus to produce an infectious recombinant virus particle. The helper virus provides essential gene products required for viral infectivity and propagation of the minimal chimpanzee adenoviral vector. When only one or more selected deletions of chimpanzee adenovirus genes are made in an otherwise functional viral vector, the deleted gene products can be supplied in the viral vector production process by propagating the virus in a selected packaging cell line that provides the deleted gene functions in trans.

### IV.E.5. Recombinant Minimal Adenovirus

A minimal chimpanzee Ad C68 virus is a viral particle containing just the adenovirus cis-elements necessary for replication and virion encapsidation. That is, the vector contains the cis-acting 5′ and 3′ inverted terminal repeat (ITR) sequences of the adenoviruses (which function as origins of replication) and the native 5′ packaging/enhancer domains (that contain sequences necessary for packaging linear Ad genomes and enhancer elements for the E1 promoter). See, for example, the techniques described for preparation of a “minimal” human Ad vector in International Patent Application WO96/13597 and incorporated herein by reference.

### IV.E.6. Other Defective Adenoviruses

Recombinant, replication-deficient adenoviruses can also contain more than the minimal chimpanzee adenovirus sequences. These other Ad vectors can be characterized by deletions of various portions of gene regions of the virus, and infectious virus particles formed by the optional use of helper viruses and/or packaging cell lines.

As one example, suitable vectors may be formed by deleting all or a sufficient portion of the C68 adenoviral immediate early gene E1a and delayed early gene E1b, so as to eliminate their normal biological functions. Replication-defective E1-deleted viruses are capable of replicating and producing infectious virus when grown on a chimpanzee adenovirus-transformed, complementation cell line containing functional adenovirus E1a and E1b genes which provide the corresponding gene products in trans. Based on the homologies to known adenovirus sequences, it is anticipated that, as is true for the human recombinant E1-deleted adenoviruses of the art, the resulting recombinant chimpanzee adenovirus is capable of infecting many cell types and can express antigen(s), but cannot replicate in most cells that do not carry the chimpanzee E1 region DNA unless the cell is infected at a very high multiplicity of infection.

As another example, all or a portion of the C68 adenovirus delayed early gene E3 can be eliminated from the chimpanzee adenovirus sequence which forms a part of the recombinant virus.

Chimpanzee adenovirus C68 vectors can also be constructed having a deletion of the E4 gene. Still another vector can contain a deletion in the delayed early gene E2a.

Deletions can also be made in any of the late genes L1 through L5 of the chimpanzee C68 adenovirus genome. Similarly, deletions in the intermediate genes IX and IVa2 can be useful for some purposes. Other deletions may be made in the other structural or non-structural adenovirus genes.

The above discussed deletions can be used individually, i.e., an adenovirus sequence can contain deletions of E1 only. Alternatively, deletions of entire genes or portions thereof effective to destroy or reduce their biological activity can be used in any combination. For example, in one exemplary vector, the adenovirus C68 sequence can have deletions of the E1 genes and the E4 gene, or of the E1, E2a and E3 genes, or of the E1 and E3 genes, or of E1, E2a and E4 genes, with or without deletion of E3, and so on. As discussed above, such deletions can be used in combination with other mutations, such as temperature-sensitive mutations, to achieve a desired result.

The cassette comprising antigen(s) be inserted optionally into any deleted region of the chimpanzee C68 Ad virus. Alternatively, the cassette can be inserted into an existing gene region to disrupt the function of that region, if desired.

### IV.E.7. Helper Viruses

Depending upon the chimpanzee adenovirus gene content of the viral vectors employed to carry the antigen cassette, a helper adenovirus or non-replicating virus fragment can be used to provide sufficient chimpanzee adenovirus gene sequences to produce an infective recombinant viral particle containing the cassette.

Useful helper viruses contain selected adenovirus gene sequences not present in the adenovirus vector construct and/or not expressed by the packaging cell line in which the vector is transfected. A helper virus can be replication-defective and contain a variety of adenovirus genes in addition to the sequences described above. The helper virus can be used in combination with the E1-expressing cell lines described herein.

For C68, the “helper” virus can be a fragment formed by clipping the C terminal end of the C68 genome with SspI, which removes about 1300 bp from the left end of the virus. This clipped virus is then co-transfected into an E1-expressing cell line with the plasmid DNA, thereby forming the recombinant virus by homologous recombination with the C68 sequences in the plasmid.

Helper viruses can also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus can optionally contain a reporter gene. A number of such reporter genes are known to the art. The presence of a reporter gene on the helper virus which is different from the antigen cassette on the adenovirus vector allows both the Ad vector and the helper virus to be independently monitored. This second reporter is used to enable separation between the resulting recombinant virus and the helper virus upon purification.

### IV.E.8. Assembly of Viral Particle and Infection of a Cell Line

Assembly of the selected DNA sequences of the adenovirus, the antigen cassette, and other vector elements into various intermediate plasmids and shuttle vectors, and the use of the plasmids and vectors to produce a recombinant viral particle can all be achieved using conventional techniques. Such techniques include conventional cloning techniques of cDNA, in vitro recombination techniques (e.g., Gibson assembly), use of overlapping oligonucleotide sequences of the adenovirus genomes, polymerase chain reaction, and any suitable method which provides the desired nucleotide sequence. Standard transfection and co-transfection techniques are employed, e.g., CaPO4 precipitation techniques or liposome-mediated transfection methods such as lipofectamine. Other conventional methods employed include homologous recombination of the viral genomes, plaquing of viruses in agar overlay, methods of measuring signal generation, and the like.

For example, following the construction and assembly of the desired antigen cassette-containing viral vector, the vector can be transfected in vitro in the presence of a helper virus into the packaging cell line. Homologous recombination occurs between the helper and the vector
sequences, which permits the adenovirus-antigen sequences in the vector to be replicated and packaged into virion capsids, resulting in the recombinant viral vector particles.

The resulting recombinant chimpanzee C68 adenoviruses are useful in transferring an antigen cassette to a selected cell. In in vivo experiments with the recombinant virus grown in the packaging cell lines, the E1-deleted recombinant chimpanzee adenovirus demonstrates utility in transferring a cassette to a non-chimpanzee, preferably a human, cell.

### IV.E.9. Use of the Recombinant Virus Vectors

The resulting recombinant chimpanzee C68 adenovirus containing the antigen cassette (produced by cooperation of the adenovirus vector and helper virus or adenoviral vector and packaging cell line, as described above) thus provides an efficient gene transfer vehicle which can deliver antigen(s) to a subject in vivo or ex vivo.

The above-described recombinant vectors are administered to humans according to published methods for gene therapy. A chimpanzee viral vector bearing an antigen cassette can be administered to a patient, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The chimpanzee adenoviral vectors are administered in sufficient amounts to transduce the human cells and to provide sufficient levels of antigen transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the liver, intranasal, intravenous, intramuscular, subcutaneous, intradermal, oral and other parental routes of administration. Routes of administration may be combined, if desired.

Dosages of the viral vector will depend primarily on factors such as the condition being treated, the age, weight and health of the patient, and may thus vary among patients. The dosage will be adjusted to balance the therapeutic benefit against any side effects and such dosages may vary depending upon the therapeutic application for which the recombinant vector is employed. The levels of expression of antigen(s) can be monitored to determine the frequency of dosage administration.

Recombinant, replication defective adenoviruses can be administered in a “pharmaceutically effective amount”, that is, an amount of recombinant adenovirus that is effective in a route of administration to transfect the desired cells and provide sufficient levels of expression of the selected gene to provide a vaccinal benefit, i.e., some measurable level of protective immunity. C68 vectors comprising an antigen cassette can be co-administered with adjuvant. Adjuvant can be separate from the vector (e.g., alum) or encoded within the vector, in particular if the adjuvant is a protein. Adjuvants are well known in the art.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired.

### V. Therapeutic and Manufacturing Methods

Also provided is a method of inducing a tumor specific immune response in a subject, vaccinating against a tumor, treating and or alleviating a symptom of cancer in a subject by administering to the subject one or more neoantigens such as a plurality of neoantigens identified using methods disclosed herein.

In some aspects, a subject has been diagnosed with cancer or is at risk of developing cancer. A subject can be a human, dog, cat, horse or any animal in which a tumor specific immune response is desired. A tumor can be any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

A neoantigen can be administered in an amount sufficient to induce a CTL response.

A neoantigen can be administered alone or in combination with other therapeutic agents. The therapeutic agent is for example, a chemotherapeutic agent, radiation, or immunotherapy. Any suitable therapeutic treatment for a particular cancer can be administered.

In addition, a subject can be further administered an anti-immunosuppressive/immunostimulatory agent such as a checkpoint inhibitor. For example, the subject can be further administered an anti-CTLA antibody or anti-PD-1 or anti-PD-L1. Blockade of CTLA-4 or PD-L1 by antibodies can enhance the immune response to cancerous cells in the patient. In particular, CTLA-4 blockade has been shown effective when following a vaccination protocol.

The optimum amount of each neoantigen to be included in a vaccine composition and the optimum dosing regimen can be determined. For example, a neoantigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of neoantigens present in the composition is/are tissue, cancer, and/or patient-specific. For instance, the exact selection of peptides can be guided by expression patterns of the parent proteins in a given tissue. The selection can be dependent on the specific type of cancer, the status of the disease, earlier treatment regimens, the immune status of the patient, and, of course, the HLA-haplotype of the patient. Furthermore, a vaccine can contain individualized components, according to personal needs of the particular patient. Examples include varying the selection of neoantigens according to the expression of the neoantigen in the particular patient or adjustments for secondary treatments following a first round or scheme of treatment.

For a composition to be used as a vaccine for cancer, neoantigens with similar normal self-peptides that are expressed in high amounts in normal tissues can be avoided or be present in low amounts in a composition described herein. On the other hand, if it is known that the tumor of a patient expresses high amounts of a certain neoantigen, the respective pharmaceutical composition for treatment of this cancer can be present in high amounts and/or more than one neoantigen specific for this particularly neoantigen or pathway of this neoantigen can be included.

Compositions comprising a neoantigen can be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when the cancer has metastasized. In such cases, in view of the minimization of extraneous substances and the relative nontoxic nature of a neoantigen, it is possible and can be felt desirable by the treating physician to administer substantial excesses of these compositions.

For therapeutic use, administration can begin at the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered at the site of surgical exiscion to induce a local immune response to the tumor. Disclosed herein are compositions for parenteral administration which comprise a solution of the neoantigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Neoantigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the neoantigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired neoantigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9106309WOAWO 91/06309; and Felgner et al., Proc. Natl. Acad. Sci. USA 84: 7413-7414 (1987).

Neoantigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more neoantigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the neoantigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of neoantigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a tumor vaccine, comprising performing the steps of a method disclosed herein; and producing a tumor vaccine comprising a plurality of neoantigens or a subset of the plurality of neoantigens.

Neoantigens disclosed herein can be manufactured using methods known in the art. For example, a method of producing a neoantigen or a vector (e.g., a vector including at least one sequence encoding one or more neoantigens) disclosed herein can include culturing a host cell under conditions suitable for expressing the neoantigen or vector wherein the host cell comprises at least one polynucleotide encoding the neoantigen or vector, and purifying the neoantigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes a neoantigen or vector disclosed herein, optionally wherein the isolated polynucleotide further comprises a promoter sequence operably linked to the at least one nucleic acid sequence that encodes the neoantigen or vector. In certain embodiments the isolated polynucleotide can be cDNA.

V.A. Identification of MHC/peptide target-reactive T cells and TCRs

T cells can be isolated from blood, lymph nodes, or tumors of patients. T cells can be enriched for antigen-specific T cells, e.g., by sorting antigen-MHC tetramer binding cells or by sorting activated cells stimulated in an in vitro co-culture of T cells and antigen-pulsed antigen presenting cells. Various reagents are known in the art for antigen-specific T cell identification including antigen-loaded tetramers and other MHC-based reagents.

Antigen-relevant alpha-beta (or gamma-delta) TCR dimers can be identified by single cell sequencing of TCRs of antigen-specific T cells. Alternatively, bulk TCR sequencing of antigen-specific T cells can be performed and alpha-beta pairs with a high probability of matching can be determined using a TCR pairing method known in the art.

Alternatively or in addition, antigen-specific T cells can be obtained through in vitro priming of naïve T cells from healthy donors. T cells obtained from PBMCs, lymph nodes, or cord blood can be repeatedly stimulated by antigen-pulsed antigen presenting cells to prime differentiation of antigen-experienced T cells. TCRs can then be identified similarly as described above for antigen-specific T cells from patients.

### VI. Antigen Use and Administration

Vaccination methods, protocols, and schedules that can also be used include, but are not limited to, those described in international application publication WO2021092095, herein incorporated by reference for all purposes.

Each vector in a prime/boost strategy typically includes a cassette that includes antigens. Cassettes can include about 1-50 antigens, separated by linkers such as the natural sequence that normally surrounds each antigen or other non-natural linker sequences such as AAY. Cassettes can also include MHCII antigens such a tetanus toxoid antigen and PADRE antigen, which can be considered universal class II antigens. Cassettes can also include a targeting sequence such as a ubiquitin targeting sequence. In addition, each vaccine dose can be administered to the subject in conjunction with (e.g., concurrently, before, or after) an immune modulator. Each vaccine dose can be administered to the subject in conjunction with (e.g., concurrently, before, or after) a checkpoint inhibitor (CPI). CPI's can include those that inhibit CTLA4, PD1, and/or PDL1 such as antibodies or antigen-binding portions thereof. Such antibodies can include tremelimumab or durvalumab. Each vaccine dose can be administered to the subject in conjunction with (e.g., concurrently, before, or after) a cytokine, such as IL-2, IL-7, IL-12 (including IL-12 p35, p40, p70, and/or p70-fusion constructs), IL-15, or IL-21.

Each vaccine dose can be administered to the subject in conjunction with (e.g., concurrently, before, or after) a modified cytokine (e.g., pegIL-2).

A vaccination protocol can be used to dose a subject with one or more antigens. A priming vaccine and a boosting vaccine can be used to dose the subject. The priming vaccine can be with any of the antigen encoding vectors described herein, such as vectors based on ChAdV68 (e.g., the sequences shown in SEQ ID NO:1 or 2). The boosting dose can be with any of the antigen encoding vectors described herein, such as vectors based on ChAdV68 (e.g., the sequences shown in SEQ ID NO:1 or 2) or SAM-based vectors (e.g., the sequences shown in SEQ ID NO:3 or 4). One or more boosting doses can be administered and can be serial administration of the same boosting vaccine (e.g., serial administration of the same Chad68-based vectors or serial administration of the same SAM-based vectors) or can be serial administration of different boosting vaccines (e.g., administration of a SAM-based vector followed by administration of a ChAdV68-based vector). Serial administration of different vaccines can include any combination of different vaccines. For example, a vaccine strategy can use a ChAdV68-based prime, followed by one or more SAM-based boosts, and the SAM-based boosts followed by a ChAdV68-based boost. Illustrative non-limiting vaccine strategies include, but are not limited to: ChAdV prime-SAM boost-SAM boost-ChAdV boost; or ChAdV prime-SAM boost-SAM boost-SAM boost-SAM boost-ChAdV boost.

ChAdV68-based vaccines can be administered at a dose ranging from 1×10¹¹ viral particles to 1×10¹² viral particles. ChAdV68-based vaccines can be administered at a dose of 1×10¹¹ viral particles. ChAdV68-based vaccines can be administered at a dose of 5×10¹¹ viral particles. ChAdV68-based vaccines can be administered at a dose of 1×10¹² viral particles. The selected dosage for ChAdV68-based vaccines will depend on, e.g., the composition, the manner of administration, the stage and severity of the disease being treated, the weight and general state of health of the patient, and the judgment of the prescribing physician.

SAM-based vaccines can be administered at a dose ranging 10-300 μg RNA. SAM-based vaccines can be administered at a dose ranging 100-300 μg RNA. SAM-based vaccines can be administered at a dose of 100 μg RNA. SAM-based vaccines can be administered at a dose of 300 μg RNA. The selected dosage for SAM-based vaccines will depend on, e.g., the composition, the manner of administration, the stage and severity of the disease being treated, the weight and general state of health of the patient, and the judgment of the prescribing physician.

A priming vaccine can be injected (e.g., intramuscularly) in a subject. Bilateral injections per dose can be used. For example, one or more injections of ChAdV68 (C68) can be used (e.g., total dose 1×10¹² viral particles); one or more injections of SAM vectors at low vaccine dose selected from the range 0.001 to 1 ug RNA, in particular 0.1 or 1 ug can be used; or one or more injections of SAM vectors at high vaccine dose selected from the range 1 to 300 ug RNA, in particular 10, 100, or 300 ug can be used.

A vaccine boost (boosting vaccine) can be injected (e.g., intramuscularly) after prime vaccination. Bilateral injections per dose can be used. For example, one or more injections of ChAdV68 (C68) can be used (e.g., total dose 1×10¹² viral particles); one or more injections of SAM vectors at low vaccine dose selected from the range 0.001 to 1 ug RNA, in particular 0.1 or 1 ug can be used; or one or more injections of SAM vectors at high vaccine dose selected from the range 1 to 300 ug RNA, in particular 10, 100 or 300 ug can be used.

A boosting vaccine can be administered about every 1, 2, 3, 4, 5, 6, 7, 8, 9, or 10 weeks, e.g., every 4 weeks and/or 8 weeks after the prime. A boosting vaccine can be administered every 4 weeks after the prime. A boosting vaccine can be administered every 6 weeks after the prime. A boosting vaccine can be administered every 12 weeks after the prime.

Boosting doses can be administered at different intervals during the course of a vaccination protocol. For example, illustrative non-limiting examples include prime-4 w-boost-12 w-boost-12 w-boost; or prime-4 w-boost-6 w-boost-6 w-boost-6 w-boost-6 w-boost, where “w” represents weeks.

One or more of the vaccine administrations can include co-administration of one or more checkpoint inhibitors. Illustrative immune checkpoint inhibitors include Tremelimumab (CTLA-4 blocking antibody), anti-OX40, PD-L1 monoclonal Antibody (Anti-B7-H1; MEDI4736), ipilimumab, MK-3475 (PD-1 blocker), Nivolumamb (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). In illustrative non-limiting examples, Nivolumamb, Yervoy/ipilimumab, or a combination thereof.

Anti-CTLA-4 (e.g., tremelimumab) can also be administered to the subject. For example, anti-CTLA4 can be administered subcutaneously near the site of the intramuscular vaccine injection (ChAdV68 prime or SAM low doses) to ensure drainage into the same lymph node. Tremelimumab is a selective human IgG2 mAb inhibitor of CTLA-4. Target Anti-CTLA-4 (tremelimumab) subcutaneous dose is typically 70-75 mg (in particular 75 mg) with a dose range of, e.g., 1-100 mg or 5-420 mg.

In certain instances an anti-PD-L1 antibody can be used such as durvalumab (MEDI 4736). Durvalumab is a selective, high affinity human IgGI mAb that blocks PD-L1 binding to PD-1 and CD80. Durvalumab is generally administered at 20 mg/kg i.v. every 4 weeks.

Immune monitoring can be performed before, during, and/or after vaccine administration. Such monitoring can inform safety and efficacy, among other parameters.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

Immune responses, such as T cell responses and B cells responses, can be assessed as part of an immune monitoring protocol. For example, the ability of a vaccine composition described herein to stimulate an immune response can be monitored and/or assessed. As used herein, “stimulate an immune response” refers to any increase in a immune response, such as initiating an immune response (e.g., a priming vaccine stimulating the initiation of an immune response in a naïve subject) or enhancement of an immune response (e.g., a boosting vaccine stimulating the enhancement of an immune response in a subject having a pre-existing immune response to an antigen, such as a pre-existing immune response initiated by a priming vaccine). Tcell responses can be measured using one or more methods known in the art such as ELISpot, intracellular cytokine staining, cytokine secretion and cell surface capture, T cell proliferation, MHC multimer staining, or by cytotoxicity assay. T cell responses to epitopes encoded in vaccines can be monitored from PBMCs by measuring induction of cytokines, such as IFNγ, using an ELISpot assay. Specific CD4 or CD8 T cell responses to epitopes encoded in vaccines can be monitored from PBMCs by measuring induction of cytokines captured intracellularly or extracellularly, such as IFNγ, using flow cytometry. Specific CD4 or CD8 T cell responses to epitopes encoded in the vaccines can be monitored from PBMCs by measuring T cell populations expressing T cell receptors specific for epitope/MHC class I complexes using MHC multimer staining. Specific CD4 or CD8 T cell responses to epitopes encoded in the vaccines can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific for epitopes encoded in vaccines can be assessed functionally by chromium release assay or alternative colorimetric cytotoxicity assays.

B cell responses can be measured using one or more methods known in the art such as assays used to determine B cell differentiation (e.g., differentiation into plasma cells), B cell or plasma cell proliferation, B cell or plasma cell activation (e.g., upregulation of costimulatory markers such as CD80 or CD86), antibody class switching, and/or antibody production (e.g., an ELISA).

Disease status of a subject can be monitored following administration of any of the vaccine compositions described herein. For example, disease status may be monitored using isolated cell-free DNA (cfDNA) from a subject. In addition, the efficacy of a vaccine therapy may be monitored using isolated cfDNA from a subject. cfDNA monitoring can include the steps of: a. isolating or having isolated cfDNA from a subject; b. sequencing or having sequenced the isolated cfDNA; c. determining or having determined a frequency of one or more mutations in the cfDNA relative to a wild-type germline nucleic acid sequence of the subject, and d. assessing or having assessed from step (c) the status of a disease in the subject. The method can also include, following step (c) above, d. performing more than one iteration of steps (a)-(c) for the given subject and comparing the frequency of the one or more mutations determined in the more than one iterations; and f. assessing or having assessed from step (d) the status of a disease in the subject. The more than one iterations can be performed at different time points, such as a first iteration of steps (a)-(c) performed prior to administration of the vaccine composition and a second iteration of steps (a)-(c) is performed subsequent to administration of the vaccine composition. Step (c) can include comparing: the frequency of the one or more mutations determined in the more than one iterations, or the frequency of the one or more mutations determined in the first iteration to the frequency of the one or more mutations determined in the second iteration. An increase in the frequency of the one or more mutations determined in subsequent iterations or the second iteration can be assessed as disease progression. A decrease in the frequency of the one or more mutations determined in subsequent iterations or the second iteration can be assessed as a response. In some aspects, the response is a Complete Response (CR) or a Partial Response (PR). A therapy can be administered to a subject following an assessment step, such as where assessment of the frequency of the one or more mutations in the cfDNA indicates the subject has the disease. The cfDNA isolation step can use centrifugation to separate cfDNA from cells or cellular debris. cfDNA can be isolated from whole blood, such as by separating the plasma layer, buffy coat, and red bloods. cfDNA sequencing can use next generation sequencing (NGS), Sanger sequencing, duplex sequencing, whole-exome sequencing, whole-genome sequencing, de novo sequencing, phased sequencing, targeted amplicon sequencing, shotgun sequencing, or combinations thereof, and may include enriching the cfDNA for one or more polynucleotide regions of interest prior to sequencing (e.g., polynucleotides known or suspected to encode the one or more mutations, coding regions, and/or tumor exome polynucleotides). Enriching the cfDNA may include hybridizing one or more polynucleotide probes, which may be modified (e.g., biotinylated), to the one or more polynucleotide regions of interest. In general, any number of mutations may be monitored simultaneously or in parallel.

Homologous vaccination regimens can include an interval between homologous doses to improve efficacy of the second dose. For example, a ChAdV68-based vaccine can be administered as an initial dose and include an interval prior to re-administration of the ChAdV68-based vaccine as a boosting dose to improve efficacy, such as reducing the impact of ChAdV-specific neutralizing antibody titers on the efficacy of the boosting dose. For example, an initial dose may induce production of neutralizing antibodies which then subsequently wane over time. In illustrative non-limiting examples for ChAdV68-based vaccines described herein, the interval is at least 27 weeks. The interval can be 27 weeks. The interval can be 28 weeks. The interval can be 29 weeks. The interval can be 30 weeks. The interval can be 31 weeks. The interval can be 32 weeks. The interval can be 33 weeks. The interval can be at least 27 weeks.

The interval can be at least 28 weeks. The interval can be at least 29 weeks. The interval can be at least 30 weeks. The interval can be at least 31 weeks. The interval can be at least 32 weeks. The interval can be at least 33 weeks.

The interval between ChAdV68-based vaccine administrations in a homologous prime-boost strategy can be as few as 8 weeks. The interval can be 8 weeks. The interval can be 9 weeks. The interval can be 10 weeks. The interval can be 11 weeks. The interval can be 12 weeks. The interval can be 13 weeks. The interval can be 14 weeks. The interval can be 15 weeks. The interval can be 16 weeks. The interval can be 17 weeks. The interval can be 18 weeks. The interval can be 19 weeks. The interval can be 20 weeks. The interval can be 21 weeks. The interval can be 23 weeks. The interval can be 24 weeks. The interval can be 25 weeks. The interval can be 26 weeks.

The interval between ChAdV68-based vaccine administrations in a homologous prime-boost strategy can be at least 8 weeks. The interval can be at least 9 weeks. The interval can be at least 10 weeks. The interval can be at least 11 weeks. The interval can be at least 12 weeks. The interval can be at least 13 weeks. The interval can be at least 14 weeks. The interval can be at least 15 weeks. The interval can be at least 16 weeks. The interval can be at least 17 weeks. The interval can be at least 18 weeks. The interval can be at least 19 weeks. The interval can be at least 20 weeks. The interval can be at least 21 weeks. The interval can be at least 23 weeks. The interval can be at least 24 weeks. The interval can be at least 25 weeks. The interval can be at least 26 weeks.

The interval between ChAdV68-based vaccine administrations in a homologous prime-boost strategy can be 2 months. The interval can be 2.5 months. The interval can be 3 months. The interval can be 3.5 months. The interval can be 4 months. The interval can be 4.5 months. The interval can be 5 months. The interval can be 5.5 months. The interval can be 6 months. The interval can be 6.5 months. The interval can be 7 months. The interval can be 7.5 months. The interval can be 8 months. The interval can be 8.5 months. The interval can be at least 2 months. The interval can be at least 2.5 months. The interval can be at least 3 months. The interval can be at least 3.5 months. The interval can be at least 4 months. The interval can be at least 4.5 months. The interval can be at least 5 months. The interval can be at least 5.5 months.

The interval can be at least 6 months. The interval can be at least 6.5 months. The interval can be at least 7 months. The interval can be at least 7.5 months. The interval can be at least 8 months. The interval can be at least 8.5 months.

### VII. Example Computing Device

FIG. 6 illustrates an example computer for implementing the entities shown in FIGS. 1, 2A-2B, 3A-3B, and 4-5. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

In some embodiments, the computing device 600 includes at least one processor 602 coupled to a chipset 604. The chipset 604 includes a memory controller hub 620 and an input/output (I/O) controller hub 622. A memory 606 and a graphics adapter 612 are coupled to the memory controller hub 620, and a display 618 is coupled to the graphics adapter 612. A storage device 608, an input interface 614, and network adapter 616 are coupled to the I/O controller hub 622. Other embodiments of the computing device 600 have different architectures.

The storage device 608 is a non-transitory computer-readable storage medium such as a hard drive, compact disk read-only memory (CD-ROM), DVD, or a solid-state memory device. The memory 606 holds instructions and data used by the processor 602. The input interface 614 is a touch-screen interface, a mouse, track ball, or other type of input interface, a keyboard, or some combination thereof, and is used to input data into the computing device 600. In some embodiments, the computing device 600 may be configured to receive input (e.g., commands) from the input interface 614 via gestures from the user. The graphics adapter 612 displays images and other information on the display 618. For example, the display 618 can show an indication of a predicted cell trajectory. The network adapter 616 couples the computing device 600 to one or more computer networks.

The computing device 600 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 608, loaded into the memory 606, and executed by the processor 602.

The types of computing devices 600 can vary from the embodiments described herein. For example, the computing device 600 can lack some of the components described above, such as graphics adapters 612, input interface 614, and displays 618. In some embodiments, a computing device 600 can include a processor 602 for executing instructions stored on a memory 606.

In various embodiments, methods described herein can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a cell trajectory of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer.

Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### VIII. Patient Subtyping

Disclosed herein are methods for performing patient subtyping. Cancer is known to be heterogenous, with one or more genetic drivers or signatures driving clinical presentation and progression, treatment responsivity, and other clinical outcomes. Progress in tumor profiling has enabled the potential to implement patient subtyping in clinical practice, with the aim to deliver precision medicine. For example, patient subtyping can be beneficial for identifying a subset of patients who are likely to respond favorably to a treatment, such as a disclosed personalized cancer vaccine containing one or more neoantigens. The one or more neoantigens can be predicted to be presented by one or more class II MHC alleles of a genotype of the patient.

In various embodiments, patient subtyping involves evaluating a tumor subtype of a patient. Evaluating a tumor subtype can occur through various methods, including but not limited to histopathological and molecular characterization. In various embodiments, molecular characterization can include assessment of molecular markers of genomic, epigenetic, transcriptomic, and/or proteomic data. Assessment is driven by methods of analysis that examine and report changes in presence/expression of one or more molecular markers (e.g., change in presence/expression of one or more molecular markers across different tumor subtypes). For example, RNA-sequencing involves collection of RNA molecules from a biological source and conversion of RNA molecules into a sequencing library (DNA) that is amenable for contemporary sequencing methods. In various embodiments, methods involve performing a histopathological characterization to determine a tumor microenvironment. In various embodiments, methods involve performing RNA-seq to determine a tumor microenvironment.

The large stream of sequencing reads or data that are generated are analyzed for enrichment and/or functional pathway analyses. Enrichment based analyses reveal the relative up or down regulation of one or more genes in a given sample and can be informative for patient subtyping. Additionally, functional analysis of RNA-sequencing data from a sample can provide up/down regulated insights into particular biological pathways or processes through analysis of multiple gene expression signatures. Whether independently or together, the RNA-sequencing data generated from patient tumor samples allows for stratification of an initial heterogenous pool of clinical samples and next enables selection of treatments that uniquely fit each patient subclass. Overall, these methods provide an empirical basis for processing patients into subtypes through examination of their tumor properties and match them with appropriate treatments.

In various embodiments, evaluating a tumor subtype involves assessing for an origin of a tumor (e.g., primary or metastasized tumor). In various embodiments, evaluating a tumor subtype involves determining a tumor microenvironment (TME) of the tumor. Example TME of a tumor can include a fibrotic, immune-depleted, immune enriched/non-fibrotic, or immune-enriched/fibrotic microenvironments.

In various embodiments, performing patient subtyping involves determining a TME of a tumor, which involves obtaining or having obtained expression levels of two or more biomarkers from a sample obtained from a patient, the two or more biomarkers selected from biomarkers involved in any of angiogenesis fibroblasts, pro-tumor immune infiltrate, anti-tumor immune infiltrate, or proliferation rate EMT signature activities; determining, based on the expression levels of the two or more biomarkers, whether to classify the patient into an immune enriched fibrotic subtype; and responsive to classifying the patient into the immune enriched fibrotic subtype, selecting the patient as a candidate for receiving a personalized cancer vaccine comprising one or more neoantigens predicted to be presented by one or more class II MHC alleles of a genotype of the patient.

In various embodiments, the two or more biomarkers comprise two or more of CD274, CD8A, CXCL9, GZMA, or PRF1. Other biomarkers can include gene expression signatures involved in any of cytolytic (CYT), cytotoxic T lymphocytes (CTL), and IFNγ. gene expression signatures. In various embodiments, the two or more biomarkers comprise presence or absence of somatic alterations in two or more of TP53, APC, KRAS, PIK3CA, or SMAD4. The biomarkers and corresponding UniProt identifiers are shown in Table 1.

In various embodiments, the expression levels of the two or more biomarkers are informative for determining whether to classify the patient into any of an immune enriched fibrotic subtype, immune enriched non-fibrotic subtype, fibrotic subtype, or depleted subtype. In particular embodiments, if the patient is classified into an immune enriched fibrotic subtype, the patient is as a candidate for receiving a personalized cancer vaccine comprising one or more neoantigens predicted to be presented by one or more class II MHC alleles of a genotype of the patient. Disclosed in further details herein are methods for identifying one or more neoantigens predicted to be presented by one or more class II MHC alleles of a genotype of the patient (e.g., using disclosed methodologies and/or disclosed presentation models). In various embodiments, methods disclosed herein further comprise administering the personalized cancer vaccine to the patient.

In various embodiments, if the patient is classified into any of immune enriched non-fibrotic subtype, fibrotic subtype, or depleted subtype, the patient is not selected as a candidate. Thus, the patient does not receive a personalized cancer vaccine.

### IX. Additional Embodiments

Additionally disclosed herein are methods for identifying neoantigens from a tumor of a subject that are likely to be presented on the cell surface of the tumor or immune cells, including professional antigen presenting cells such as dendritic cells, and/or are likely to be immunogenic. As an example, one such method may comprise the steps of: obtaining at least one of exome, transcriptome or whole genome tumor nucleotide sequencing data from the tumor cell of the subject, wherein the tumor nucleotide sequencing data is used to obtain data representing peptide sequences of each of a set of neoantigens, and wherein the peptide sequence of each neoantigen comprises at least one alteration that makes it distinct from the corresponding wild-type, parental peptide sequence; inputting the peptide sequence of each neoantigen into one or more presentation models to generate a set of numerical likelihoods that each of the neoantigens is presented by one or more MHC alleles on the tumor cell surface of the tumor cell of the subject or cells present in the tumor, the set of numerical likelihoods having been identified at least based on received mass spectrometry data; and selecting a subset of the set of neoantigens based on the set of numerical likelihoods to generate a set of selected neoantigens.

The presentation model can comprise a statistical regression or a machine learning (e.g., deep learning) model trained on a set of reference data (also referred to as a training data set) comprising a set of corresponding labels, wherein the set of reference data is obtained from each of a plurality of distinct subjects where optionally some subjects can have a tumor, and wherein the set of reference data comprises at least one of: data representing exome nucleotide sequences from tumor tissue, data representing exome nucleotide sequences from normal tissue, data representing transcriptome nucleotide sequences from tumor tissue, data representing proteome sequences from tumor tissue, and data representing MHC peptidome sequences from tumor tissue, and data representing MHC peptidome sequences from normal tissue. The reference data can further comprise mass spectrometry data, sequencing data, RNA sequencing data, and proteomics data for single-allele cell lines engineered to express a predetermined MHC allele that are subsequently exposed to synthetic protein, normal and tumor human cell lines, and fresh and frozen primary samples, and T cell assays (e.g., ELISPOT). In certain aspects, the set of reference data includes each form of reference data.

The presentation model can comprise a set of features derived at least in part from the set of reference data, and wherein the set of features comprises at least one of allele dependent-features and allele-independent features. In certain aspects each feature is included.

Also disclosed herein are methods for generating an output for constructing a personalized cancer vaccine by identifying one or more neoantigens from one or more tumor cells of a subject that are likely to be presented on a surface of the tumor cells. As an example, one such method may comprise the steps of: obtaining at least one of exome, transcriptome, or whole genome nucleotide sequencing data from the tumor cells and normal cells of the subject, wherein the nucleotide sequencing data is used to obtain data representing peptide sequences of each of a set of neoantigens identified by comparing the nucleotide sequencing data from the tumor cells and the nucleotide sequencing data from the normal cells, and wherein the peptide sequence of each neoantigen comprises at least one alteration that makes it distinct from the corresponding wild-type, peptide sequence identified from the normal cells of the subject; encoding the peptide sequences of each of the neoantigens into a corresponding numerical vector, each numerical vector including information regarding a plurality of amino acids that make up the peptide sequence and a set of positions of the amino acids in the peptide sequence; inputting the numerical vectors, using a computer processor, into a deep learning presentation model to generate a set of presentation likelihoods for the set of neoantigens, each presentation likelihood in the set representing the likelihood that a corresponding neoantigen is presented by one or more class II MHC alleles on the surface of the tumor cells of the subject, the deep learning presentation model; selecting a subset of the set of neoantigens based on the set of presentation likelihoods to generate a set of selected neoantigens; and generating the output for constructing the personalized cancer vaccine based on the set of selected neoantigens.

In some embodiments, the presentation model comprises a plurality of parameters identified at least based on a training data set and a function representing a relation between the numerical vector received as an input and the presentation likelihood generated as output based on the numerical vector and the parameters. In certain embodiments, the training data set comprises labels obtained by mass spectrometry measuring presence of peptides bound to at least one class II MHC allele identified as present in at least one of a plurality of samples, training peptide sequences encoded as numerical vectors including information regarding a plurality of amino acids that make up the peptide sequence and a set of positions of the amino acids in the peptide sequence, and at least one HLA allele associated with the training peptide sequences.

Dendritic cell presentation to naïve T cell features can comprise at least one of: A feature described above. The dose and type of antigen in the vaccine. (e.g., peptide, mRNA, virus, etc.): (1) The route by which dendritic cells (DCs) take up the antigen type (e.g., endocytosis, micropinocytosis); and/or (2) The efficacy with which the antigen is taken up by DCs. The dose and type of adjuvant in the vaccine. The length of the vaccine antigen sequence. The number and sites of vaccine administration. Baseline patient immune functioning (e.g., as measured by history of recent infections, blood counts, etc). For RNA vaccines: (1) the turnover rate of the mRNA protein product in the dendritic cell; (2) the rate of translation of the mRNA after uptake by dendritic cells as measured in in vitro or in vivo experiments; and/or (3) the number or rounds of translation of the mRNA after uptake by dendritic cells as measured by in vivo or in vitro experiments. The presence of protease cleavage motifs in the peptide, optionally giving additional weight to proteases typically expressed in dendritic cells (as measured by RNA-seq or mass spectrometry). The level of expression of the proteasome and immunoproteasome in typical activated dendritic cells (which may be measured by RNA-seq, mass spectrometry, immunohistochemistry, or other standard techniques). The expression levels of the particular MHC allele in the individual in question (e.g., as measured by RNA-seq or mass spectrometry), optionally measured specifically in activated dendritic cells or other immune cells. The probability of peptide presentation by the particular MHC allele in other individuals who express the particular MHC allele, optionally measured specifically in activated dendritic cells or other immune cells. The probability of peptide presentation by MHC alleles in the same family of molecules (e.g., HLA-A, HLA-B, HLA-C, HLA-DQ, HLA-DR, HLA-DP) in other individuals, optionally measured specifically in activated dendritic cells or other immune cells.

Immune tolerance escape features can comprise at least one of: Direct measurement of the self-peptidome via protein mass spectrometry performed on one or several cell types. Estimation of the self-peptidome by taking the union of all k-mer (e.g. 5-25) substrings of self-proteins. Estimation of the self-peptidome using a model of presentation similar to the presentation model described above applied to all non-mutation self-proteins, optionally accounting for germline variants.

Ranking can be performed using the plurality of neoantigens provided by at least one model based at least in part on the numerical likelihoods. Following the ranking a selecting can be performed to select a subset of the ranked neoantigens according to a selection criteria. After selecting a subset of the ranked peptides can be provided as an output.

A number of the set of selected neoantigens may be 20.

The presentation model may represent dependence between presence of a pair of a particular one of the MHC alleles and a particular amino acid at a particular position of a peptide sequence; and likelihood of presentation on the tumor cell surface, by the particular one of the MHC alleles of the pair, of such a peptide sequence comprising the particular amino acid at the particular position.

A method disclosed herein can also include applying the one or more presentation models to the peptide sequence of the corresponding neoantigen to generate a dependency score for each of the one or more MHC alleles indicating whether the MHC allele will present the corresponding neoantigen based on at least positions of amino acids of the peptide sequence of the corresponding neoantigen.

A method disclosed herein can also include transforming the dependency scores to generate a corresponding per-allele likelihood for each MHC allele indicating a likelihood that the corresponding MHC allele will present the corresponding neoantigen; and combining the per-allele likelihoods to generate the numerical likelihood.

The step of transforming the dependency scores can model the presentation of the peptide sequence of the corresponding neoantigen as mutually exclusive.

A method disclosed herein can also include transforming a combination of the dependency scores to generate the numerical likelihood.

The step of transforming the combination of the dependency scores can model the presentation of the peptide sequence of the corresponding neoantigen as interfering between MHC alleles.

The set of numerical likelihoods can be further identified by at least an allele noninteracting feature, and a method disclosed herein can also include applying an allele noninteracting one of the one or more presentation models to the allele noninteracting features to generate a dependency score for the allele noninteracting features indicating whether the peptide sequence of the corresponding neoantigen will be presented based on the allele noninteracting features.

A method disclosed herein can also include combining the dependency score for each MHC allele in the one or more MHC alleles with the dependency score for the allele noninteracting feature; transforming the combined dependency scores for each MHC allele to generate a corresponding per-allele likelihood for the MHC allele indicating a likelihood that the corresponding MHC allele will present the corresponding neoantigen; and combining the per-allele likelihoods to generate the numerical likelihood.

A method disclosed herein can also include transforming a combination of the dependency scores for each of the MHC alleles and the dependency score for the allele noninteracting features to generate the numerical likelihood.

A set of numerical parameters for the presentation model can be trained based on a training data set including at least a set of training peptide sequences identified as present in a plurality of samples and one or more MHC alleles associated with each training peptide sequence, wherein the training peptide sequences are identified through mass spectrometry on isolated peptides eluted from MHC alleles derived from the plurality of samples.

The samples can also include cell lines engineered to express a single MHC class I or class II allele.

The samples can also include cell lines engineered to express a plurality of MHC class I or class II alleles.

The samples can also include human cell lines obtained or derived from a plurality of patients.

The samples can also include fresh or frozen tumor samples obtained from a plurality of patients.

The samples can also include fresh or frozen tissue samples obtained from a plurality of patients.

The samples can also include peptides identified using T-cell assays.

The training data set can further include data associated with: peptide abundance of the set of training peptides present in the samples; peptide length of the set of training peptides in the samples.

The training data set may be generated by comparing the set of training peptide sequences via alignment to a database comprising a set of known protein sequences, wherein the set of training protein sequences are longer than and include the training peptide sequences.

The training data set may be generated based on performing or having performed nucleotide sequencing on a cell line to obtain at least one of exome, transcriptome, or whole genome sequencing data from the cell line, the sequencing data including at least one nucleotide sequence including an alteration.

The training data set may be generated based on obtaining at least one of exome, transcriptome, and whole genome normal nucleotide sequencing data from normal tissue samples.

The training data set may further include data associated with proteome sequences associated with the samples.

The training data set may further include data associated with MHC peptidome sequences associated with the samples.

The training data set may further include data associated with peptide-MHC binding affinity measurements for at least one of the isolated peptides.

The training data set may further include data associated with peptide-MHC binding stability measurements for at least one of the isolated peptides.

The training data set may further include data associated with transcriptomes associated with the samples.

The training data set may further include data associated with genomes associated with the samples.

The training peptide sequences may be of lengths within a range of k-mers where k is between 8-15, inclusive for MHC class I or 6-30 inclusive for MHC class II.

A method disclosed herein can also include encoding the peptide sequence using a one-hot encoding scheme.

A method disclosed herein can also include encoding the training peptide sequences using a left-padded one-hot encoding scheme.

A method of treating a subject having a tumor, comprising performing the steps of claim 1, and further comprising obtaining a tumor vaccine comprising the set of selected neoantigens, and administering the tumor vaccine to the subject.

A method disclosed herein can also include identifying one or more T cells that are antigen-specific for at least one of the neoantigens in the subset. In some embodiments, the identification comprises co-culturing the one or more T cells with one or more of the neoantigens in the subset under conditions that expand the one or more antigen-specific T cells. In further embodiments, the identification comprises contacting the one or more T cells with a tetramer comprising one or more of the neoantigens in the subset under conditions that allow binding between the T cell and the tetramer. In even further embodiments, the method disclosed herein can also include identifying one or more T cell receptors (TCR) of the one or more identified T cells. In certain embodiments, identifying the one or more T cell receptors comprises sequencing the T cell receptor sequences of the one or more identified T cells. The method disclosed herein can further comprise genetically engineering a plurality of T cells to express at least one of the one or more identified T cell receptors; culturing the plurality of T cells under conditions that expand the plurality of T cells; and infusing the expanded T cells into the subject. In some embodiments, genetically engineering the plurality of T cells to express at least one of the one or more identified T cell receptors comprises cloning the T cell receptor sequences of the one or more identified T cells into an expression vector; and transfecting each of the plurality of T cells with the expression vector. In some embodiments, the method disclosed herein further comprises culturing the one or more identified T cells under conditions that expand the one or more identified T cells; and infusing the expanded T cells into the subject.

Also disclosed herein is an isolated T cell that is antigen-specific for at least one selected neoantigen in the subset.

Also disclosed herein is a methods for manufacturing a tumor vaccine, comprising the steps of: obtaining at least one of exome, transcriptome or whole genome tumor nucleotide sequencing data from the tumor cell of the subject, wherein the tumor nucleotide sequencing data is used to obtain data representing peptide sequences of each of a set of neoantigens, and wherein the peptide sequence of each neoantigen comprises at least one mutation that makes it distinct from the corresponding wild-type, parental peptide sequence; inputting the peptide sequence of each neoantigen into one or more presentation models to generate a set of numerical likelihoods that each of the neoantigens is presented by one or more MHC alleles on the tumor cell surface of the tumor cell of the subject, the set of numerical likelihoods having been identified at least based on received mass spectrometry data; and selecting a subset of the set of neoantigens based on the set of numerical likelihoods to generate a set of selected neoantigens; and producing or having produced a tumor vaccine comprising the set of selected neoantigens.

Also disclosed herein is a tumor vaccine including a set of selected neoantigens selected by performing the method comprising the steps of: obtaining at least one of exome, transcriptome or whole genome tumor nucleotide sequencing data from the tumor cell of the subject, wherein the tumor nucleotide sequencing data is used to obtain data representing peptide sequences of each of a set of neoantigens, and wherein the peptide sequence of each neoantigen comprises at least one mutation that makes it distinct from the corresponding wild-type, parental peptide sequence; inputting the peptide sequence of each neoantigen into one or more presentation models to generate a set of numerical likelihoods that each of the neoantigens is presented by one or more MHC alleles on the tumor cell surface of the tumor cell of the subject, the set of numerical likelihoods having been identified at least based on received mass spectrometry data; and selecting a subset of the set of neoantigens based on the set of numerical likelihoods to generate a set of selected neoantigens; and producing or having produced a tumor vaccine comprising the set of selected neoantigens.

The tumor vaccine may include one or more of a nucleotide sequence, a polypeptide sequence, RNA, DNA, a cell, a plasmid, or a vector.

The tumor vaccine may include one or more neoantigens presented on the tumor cell surface.

The tumor vaccine may include one or more neoantigens that is immunogenic in the subject.

The tumor vaccine may not include one or more neoantigens that induce an autoimmune response against normal tissue in the subject.

The tumor vaccine may include an adjuvant.

The tumor vaccine may include an excipient.

A method disclosed herein may also include selecting neoantigens that have an increased likelihood of being presented on the tumor cell surface relative to unselected neoantigens based on the presentation model.

A method disclosed herein may also include selecting neoantigens that have an increased likelihood of being capable of inducing a tumor-specific immune response in the subject relative to unselected neoantigens based on the presentation model.

A method disclosed herein may also include selecting neoantigens that have an increased likelihood of being capable of being presented to naïve T cells by professional antigen presenting cells (APCs) relative to unselected neoantigens based on the presentation model, optionally wherein the APC is a dendritic cell (DC).

A method disclosed herein may also include selecting neoantigens that have a decreased likelihood of being subject to inhibition via central or peripheral tolerance relative to unselected neoantigens based on the presentation model.

A method disclosed herein may also include selecting neoantigens that have a decreased likelihood of being capable of inducing an autoimmune response to normal tissue in the subject relative to unselected neoantigens based on the presentation model.

The exome or transcriptome nucleotide sequencing data may be obtained by performing sequencing on the tumor tissue.

The sequencing may be next generation sequencing (NGS) or any massively parallel sequencing approach.

The set of numerical likelihoods may be further identified by at least MHC-allele interacting features comprising at least one of: the predicted affinity with which the MHC allele and the neoantigen encoded peptide bind; the predicted stability of the neoantigen encoded peptide-MHC complex; the sequence and length of the neoantigen encoded peptide; the probability of presentation of neoantigen encoded peptides with similar sequence in cells from other individuals expressing the particular MHC allele as assessed by mass-spectrometry proteomics or other means; the expression levels of the particular MHC allele in the subject in question (e.g. as measured by RNA-seq or mass spectrometry); the overall neoantigen encoded peptide-sequence-independent probability of presentation by the particular MHC allele in other distinct subjects who express the particular MHC allele; the overall neoantigen encoded peptide-sequence-independent probability of presentation by MHC alleles in the same family of molecules (e.g., HLA-A, HLA-B, HLA-C, HLA-DQ, HLA-DR, HLA-DP) in other distinct subjects.

The set of numerical likelihoods are further identified by at least MHC-allele noninteracting features comprising at least one of: the C- and N-terminal sequences flanking the neoantigen encoded peptide within its source protein sequence; the presence of protease cleavage motifs in the neoantigen encoded peptide, optionally weighted according to the expression of corresponding proteases in the tumor cells (as measured by RNA-seq or mass spectrometry); the turnover rate of the source protein as measured in the appropriate cell type; the length of the source protein, optionally considering the specific splice variants (“isoforms”) most highly expressed in the tumor cells as measured by RNA-seq or proteome mass spectrometry, or as predicted from the annotation of germline or somatic splicing mutations detected in DNA or RNA sequence data; the level of expression of the proteasome, immunoproteasome, thymoproteasome, or other proteases in the tumor cells (which may be measured by RNA-seq, proteome mass spectrometry, or immunohistochemistry); the expression of the source gene of the neoantigen encoded peptide (e.g., as measured by RNA-seq or mass spectrometry); the typical tissue-specific expression of the source gene of the neoantigen encoded peptide during various stages of the cell cycle; a comprehensive catalog of features of the source protein and/or its domains as can be found in e.g. uniProt or PDB http://www.rcsb.org/pdb/home/home.do; features describing the properties of the domain of the source protein containing the peptide, for example: secondary or tertiary structure (e.g., alpha helix vs beta sheet); alternative splicing; the probability of presentation of peptides from the source protein of the neoantigen encoded peptide in question in other distinct subjects; the probability that the peptide will not be detected or over-represented by mass spectrometry due to technical biases; the expression of various gene modules/pathways as measured by RNASeq (which need not contain the source protein of the peptide) that are informative about the state of the tumor cells, stroma, or tumor-infiltrating lymphocytes (TILs); the copy number of the source gene of the neoantigen encoded peptide in the tumor cells; the probability that the peptide binds to the TAP or the measured or predicted binding affinity of the peptide to the TAP; the expression level of TAP in the tumor cells (which may be measured by RNA-seq, proteome mass spectrometry, immunohistochemistry); presence or absence of tumor mutations, including, but not limited to: driver mutations in known cancer driver genes such as EGFR, KRAS, ALK, RET, ROS1, TP53, CDKN2A, CDKN2B, NTRK1, NTRK2, NTRK3, and in genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOB, HLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome). Peptides whose presentation relies on a component of the antigen-presentation machinery that is subject to loss-of-function mutation in the tumor have reduced probability of presentation; presence or absence of functional germline polymorphisms, including, but not limited to: in genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOB, HLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome); tumor type (e.g., NSCLC, melanoma); clinical tumor subtype (e.g., squamous lung cancer vs. non-squamous); smoking history; the typical expression of the source gene of the peptide in the relevant tumor type or clinical subtype, optionally stratified by driver mutation.

The at least one mutation may be a frameshift or nonframeshift indel, missense or nonsense substitution, splice site alteration, genomic rearrangement or gene fusion, or any genomic or expression alteration giving rise to a neoORF.

The tumor cell may be selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer.

A method disclosed herein may also include obtaining a tumor vaccine comprising the set of selected neoantigens or a subset thereof, optionally further comprising administering the tumor vaccine to the subject.

At least one of neoantigens in the set of selected neoantigens, when in polypeptide form, may include at least one of: a binding affinity with MHC with an IC50 value of less than 1000 nM, for MHC Class I polypeptides a length of 8-15, 8, 9, 10, 11, 12, 13, 14, or 15 amino acids, for MHC Class II polypeptides a length of 6-30, 6, 7, 8, 9, 10, 11, 12, 13, 14, 15, 16, 17, 18,19, 20, 21, 22, 23, 24, 25, 26, 27, 28, 29, or 30 amino acids, presence of sequence motifs within or near the polypeptide in the parent protein sequence promoting proteasome cleavage, and presence of sequence motifs promoting TAP transport. For MHC Class II, presence of sequence motifs within or near the peptide promoting cleavage by extracellular or lysosomal proteases (e.g., cathepsins) or HLA-DM catalyzed HLA binding.

Also disclosed herein is a methods for generating a model for identifying one or more neoantigens that are likely to be presented on a tumor cell surface of a tumor cell, comprising the steps of: receiving mass spectrometry data comprising data associated with a plurality of isolated peptides eluted from major histocompatibility complex (MHC) derived from a plurality of samples; obtaining a training data set by at least identifying a set of training peptide sequences present in the samples and one or more MHCs associated with each training peptide sequence; training a set of numerical parameters of a presentation model using the training data set comprising the training peptide sequences, the presentation model providing a plurality of numerical likelihoods that peptide sequences from the tumor cell are presented by one or more MHC alleles on the tumor cell surface.

The presentation model may represent dependence between: presence of a particular amino acid at a particular position of a peptide sequence; and likelihood of presentation, by one of the MHC alleles on the tumor cell, of the peptide sequence containing the particular amino acid at the particular position.

The samples can also include cell lines engineered to express a single MHC class I or class II allele.

The samples can also include cell lines engineered to express a plurality of MHC class I or class II alleles.

The samples can also include human cell lines obtained or derived from a plurality of patients.

The samples can also include fresh or frozen tumor samples obtained from a plurality of patients.

The samples can also include peptides identified using T-cell assays.

The training data set may further include data associated with: peptide abundance of the set of training peptides present in the samples; peptide length of the set of training peptides in the samples.

A method disclosed herein can also include obtaining a set of training protein sequences based on the training peptide sequences by comparing the set of training peptide sequences via alignment to a database comprising a set of known protein sequences, wherein the set of training protein sequences are longer than and include the training peptide sequences.

A method disclosed herein can also include performing or having performed mass spectrometry on a cell line to obtain at least one of exome, transcriptome, or whole genome nucleotide sequencing data from the cell line, the nucleotide sequencing data including at least one protein sequence including a mutation.

A method disclosed herein can also include: encoding the training peptide sequences using a one-hot encoding scheme.

A method disclosed herein can also include obtaining at least one of exome, transcriptome, and whole genome normal nucleotide sequencing data from normal tissue samples; and training the set of parameters of the presentation model using the normal nucleotide sequencing data.

The training data set may further include data associated with proteome sequences associated with the samples.

The training data set may further include data associated with MHC peptidome sequences associated with the samples.

The training data set may further include data associated with peptide-MHC binding affinity measurements for at least one of the isolated peptides.

The training data set may further include data associated with peptide-MHC binding stability measurements for at least one of the isolated peptides.

The training data set may further include data associated with transcriptomes associated with the samples.

The training data set may further include data associated with genomes associated with the samples.

A method disclosed herein may also include logistically regressing the set of parameters.

The training peptide sequences may be lengths within a range of k-mers where k is between 8-15, inclusive for MHC class I or 6-30, inclusive for MHC class II.

A method disclosed herein may also include encoding the training peptide sequences using a left-padded one-hot encoding scheme.

A method disclosed herein may also include determining values for the set of parameters using a deep learning algorithm.

Disclosed herein is are methods for identifying one or more neoantigens that are likely to be presented on a tumor cell surface of a tumor cell, comprising executing the steps of: receiving mass spectrometry data comprising data associated with a plurality of isolated peptides eluted from major histocompatibility complex (MHC) derived from a plurality of fresh or frozen tumor samples; obtaining a training data set by at least identifying a set of training peptide sequences present in the tumor samples and presented on one or more MHC alleles associated with each training peptide sequence; obtaining a set of training protein sequences based on the training peptide sequences; and training a set of numerical parameters of a presentation model using the training protein sequences and the training peptide sequences, the presentation model providing a plurality of numerical likelihoods that peptide sequences from the tumor cell are presented by one or more MHC alleles on the tumor cell surface.

The presentation model may represent dependence between: presence of a pair of a particular one of the MHC alleles and a particular amino acid at a particular position of a peptide sequence; and likelihood of presentation on the tumor cell surface, by the particular one of the MHC alleles of the pair, of such a peptide sequence comprising the particular amino acid at the particular position.

A method disclosed herein can also include selecting a subset of neoantigens, wherein the subset of neoantigens is selected because each has an increased likelihood that it is presented on the cell surface of the tumor relative to one or more distinct tumor neoantigens.

A method disclosed herein can also include selecting a subset of neoantigens, wherein the subset of neoantigens is selected because each has an increased likelihood that it is capable of inducing a tumor-specific immune response in the subject relative to one or more distinct tumor neoantigens.

A method disclosed herein can also include selecting a subset of neoantigens, wherein the subset of neoantigens is selected because each has an increased likelihood that it is capable of being presented to naïve T cells by professional antigen presenting cells (APCs) relative to one or more distinct tumor neoantigens, optionally wherein the APC is a dendritic cell (DC).

A method disclosed herein can also include selecting a subset of neoantigens, wherein the subset of neoantigens is selected because each has a decreased likelihood that it is subject to inhibition via central or peripheral tolerance relative to one or more distinct tumor neoantigens.

A method disclosed herein can also include selecting a subset of neoantigens, wherein the subset of neoantigens is selected because each has a decreased likelihood that it is capable of inducing an autoimmune response to normal tissue in the subject relative to one or more distinct tumor neoantigens.

A method disclosed herein can also include selecting a subset of neoantigens, wherein the subset of neoantigens is selected because each has a decreased likelihood that it will be differentially post-translationally modified in tumor cells versus APCs, optionally wherein the APC is a dendritic cell (DC).

Described herein are compositions for delivery of KRAS-associated MHC class II neoepitopes and associated methods of use.

A composition for delivery of KRAS-associated MHC class II neoepitopes can include an antigen expression system. A composition for delivery of an antigen expression system can include an antigen expression system, wherein the antigen expression system includes one or more vectors that have (a) a vector backbone including (i) at least one promoter nucleotide sequence, and (ii) optionally, at least one polyadenylation (poly(A)) sequence; and (b) a cassette, wherein the cassette includes: (i) at least one antigen-encoding nucleic acid sequence including an epitope-encoding nucleic acid sequence encoding a KRAS-associated MHC class II neoepitope.

The at least one antigen-encoding nucleic acid sequence can also include a 5′ linker sequence and/or a 3′ linker sequence. A 5′ linker sequence and/or a 3′ linker sequence can include native linker sequences.

The cassette can also include a second promoter nucleotide sequence operably linked to the antigen-encoding nucleic acid sequence. The cassette can also include at least one MHC class I epitope-encoding nucleic acid sequence. The cassette can also include at least one nucleic acid sequence encoding a GPGPG amino acid linker sequence. The cassette can also include at least one second poly(A) sequence, wherein the second poly(A) sequence is a native poly(A) sequence or an exogenous poly(A) sequence to the vector backbone, and wherein if the second promoter nucleotide sequence is absent, the antigen-encoding nucleic acid sequence is operably linked to the at least one promoter nucleotide sequence.

Also described herein is a KRAS-associated MHC class II neoepitope-based vaccine. A KRAS-associated MHC class II neoepitope-based vaccine can include at least one KRAS-associated MHC class II neoepitope. A KRAS-associated MHC class II neoepitope-based vaccine can include a KRAS-associated MHC class II neoepitope-encoding nucleic acid sequence encoding the at least one KRAS-associated MHC class II neoepitope. A KRAS-associated MHC class II neoepitope-encoding nucleic acid sequence can include a viral vector, an mRNA vector, or a DNA vector. A KRAS-associated MHC class II neoepitope-encoding nucleic acid sequence can include an mRNA vector.

KRAS-associated MHC class II neoepitopes can include a KRAS G12C mutation, a KRAS G12V mutation, a KRAS G12D mutation, a KRAS G12A mutation, and a KRAS Q61H mutation.

KRAS-associated MHC class II neoepitopes
having a KRAS G12C mutation can
include
MTEYKLVVVGACGVGKSALTIQLIQ,
MTEYKLVVVGACGVGKSALTIQLIQN,
MTEYKLVVVGACGVGKSALTIQLIQNH,
MTEYKLVVVGACGVGKSALTIQLIQNHF,
MTEYKLVVVGACGVGKSALTIQLIQNHFVDEYDPT,
or
MTEYKLVVVGACGVGKSALTIQLIQNHFVDEYDPTIEDSY.
KRAS-associated MHC class II neoepitopes
having a KRAS G12D mutation can include
MTEYKLVVVGADGVGKSALTIQLIQ,
MTEYKLVVVGADGVGKSALTIQLION,
MTEYKLVVVGADGVGKSALTIQLIQNH,
MTEYKLVVVGADGVGKSALTIQLIQNHF,
MTEYKLVVVGADGVGKSALTIQLIQNHFVDEYDPT,
or
MTEYKLVVVGADGVGKSALTIQLIQNHFVDEYDPTIEDSY.
KRAS-associated MHC class II neoepitopes
having a KRAS G12V mutation can include
MTEYKLVVVGAVGVGKSALTIQLIQ,
MTEYKLVVVGAVGVGKSALTIQLIQN,
MTEYKLVVVGAVGVGKSALTIQLIQNH,
MTEYKLVVVGAVGVGKSALTIQLIQNHF,
MTEYKLVVVGAVGVGKSALTIQLIQNHFVDEYDPT,
or
MTEYKLVVVGAVGVGKSALTIQLIQNHFVDEYDPTIEDSY.
KRAS-associated MHC class II neoepitopes
having a KRAS Q61H mutation caninclude
ETCLLDILDTAGHEEYSAMRDQYMR,
GETCLLDILDTAGHEEYSAMRDQYMR,
GETCLLDILDTAGHEEYSAMRDQYMRT,
DGETCLLDILDTAGHEEYSAMRDQYMRT,
VVIDGETCLLDILDTAGHEEYSAMRDQYMRTGEGF,
or
RKQVVIDGETCLLDILDTAGHEEYSAMRDQYMRTGEGFLC.
KRAS-associated MHC class II neoepitopes
having a KRAS G12A mutation can include
MTEYKLVVVGAAGVGKSALTIQLIQ,
MTEYKLVVVGAAGVGKSALTIQLION,
MTEYKLVVVGAAGVGKSALTIQLIQNH,
MTEYKLVVVGAAGVGKSALTIQLIQNHF,
MTEYKLVVVGAAGVGKSALTIQLIQNHFVDEYDPT,
or
MTEYKLVVVGAAGVGKSALTIQLIQNHFVDEYDPTIEDSY.

A composition for delivery of an antigen expression system can include an antigen expression system, wherein the antigen expression system includes one or more vectors, including: (a) a vector backbone including (i) at least one promoter nucleotide sequence, and (ii) optionally, at least one polyadenylation (poly(A)) sequence; and (b) a cassette, wherein the cassette including: (i) at least one antigen-encoding nucleic acid sequence, including: an epitope-encoding nucleic acid sequence encoding a KRAS-associated MHC class II neoepitope, wherein the a KRAS-associated MHC class II neoepitope includes a KRAS G12C mutation.

The at least one antigen-encoding nucleic acid sequence can also include a 5′ linker sequence and/or a 3′ linker sequence. A 5′ linker sequence and/or a 3′ linker sequence can include native linker sequences.

The cassette can also include a second promoter nucleotide sequence operably linked to the antigen-encoding nucleic acid sequence. The cassette can also include at least one MHC class I epitope-encoding nucleic acid sequence. The cassette can also include at least one nucleic acid sequence encoding a GPGPG amino acid linker sequence. The cassette can also include at least one second poly(A) sequence, wherein the second poly(A) sequence is a native poly(A) sequence or an exogenous poly(A) sequence to the vector backbone, and wherein if the second promoter nucleotide sequence is absent, the antigen-encoding nucleic acid sequence is operably linked to the at least one promoter nucleotide sequence.

KRAS-associated MHC class II neoepitopes having a KRAS G12C mutation can be any of the amino acid sequences shown in Table 2.

KRAS-associated MHC class II neoepitopes
having a KRAS G12C mutation can be
EYKLVVVGACG,
KLVVVGACGVGKSALTIQLI,
KLVVVGACGVGKSALTIQLIQ,
MTEYKLVVVGACGV,
MTEYKLVVVGACGVGK,
KLVVVGACGVGKSALTI,
KLVVVGACGVGKSALTIQ,
LVVVGACGVGKSALTIQLIQ,
MTEYKLVVVGACGVGK,
VVGACGVGKSALTIQ,
or
YKLVVVGACGVGKSALTIQL.
KRAS-associated MHC class II neoepitopes
having a KRAS G12C mutation can be
EYKLVVVGACG,
KLVVVGACGVGKSALTIQLI,
KLVVVGACGVGKSALTIQLIQ,
MTEYKLVVVGACGV,
or
MTEYKLVVVGACGVGK.
KRAS-associated MHC class II neoepitopes
having a KRAS G12C mutation can be
KLVVVGACGVGKSALTI,
KLVVVGACGVGKSALTIQ,
LVVVGACGVGKSALTIQLIQ,
MTEYKLVVVGACGVGK,
VVGACGVGKSALTIQ,
or
YKLVVVGACGVGKSALTIQL.
KRAS-associated MHC class II neoepitopes
having a KRAS G12C mutation can be
KLVVVGACG.
KRAS-associated MHC class II neoepitopes
having a KRAS G12C mutation can be
EYKLVVVGACG.
KRAS-associated MHC class II neoepitopes
having a KRAS G12C mutation can be
KLVVVGACGVGKSALTIQLI
or
KLVVVGACGVGKSALTIQLIQ.
KRAS-associated MHC class II neoepitopes
having a KRAS G12C mutation can be
MTEYKLVVVGACGV
or
MTEYKLVVVGACGVGK.
KRAS-associated MHC class II neoepitopes
having a KRAS G12C mutation can be
MTEYKLVVVGACGVGKSALTIQLIQ.

An antigen-encoding nucleic acid sequence can encode a peptide comprising the amino acid sequence MTEYKLVVVGACGVGKSALTIQLIQ, such as encoding an antigen having the sequence MTEYKLVVVGACGVGKSALTIQLIQ that is further processed to produce a KRAS-associated MHC class II neoepitopes having a KRAS G12C mutation.

KRAS-associated MHC class II neoepitopes
having a KRAS G12C mutation can be
MTEYKLVVVGACGVGKSALTIQLIQ
MTEYKLVVVGACGVGKSALTIQLIQN,
MTEYKLVVVGACGVGKSALTIQLIQNH,
MTEYKLVVVGACGVGKSALTIQLIQNHF,
MTEYKLVVVGACGVGKSALTIQLIQNHFVDEYDPT,
or
MTEYKLVVVGACGVGKSALTIQLIQNHFVDEYDPTIEDSY.
An antigen-encoding nucleic acid sequence
can encode a peptide comprising the
amino acid sequence
MTEYKLVVVGACGVGKSALTIQLIQ,
MTEYKLVVVGACGVGKSALTIQLIQN,
MTEYKLVVVGACGVGKSALTIQLIQNH,
MTEYKLVVVGACGVGKSALTIQLIQNHF,
MTEYKLVVVGACGVGKSALTIQLIQNHFVDEYDPT,
or
MTEYKLVVVGACGVGKSALTIQLIQNHFVDEYDPTIEDSY.

An epitope-encoding nucleic acid sequence can further encode a KRAS-associated MHC class I neoepitope (e.g., a KRAS G12C MHC class I neoepitope). An epitope-encoding nucleic acid sequence can further encode a KRAS-associated MHC class I neoepitope where the KRAS-associated MHC class I neoepitope is a fragment of a KRAS-associated MHC class II neoepitope, e.g., where a MHC class I epitope is embedded within an encoded MHC class II neoepitope sequence and/or embedded within an antigen that includes a MHC class II neoepitope sequence (e.g., an antigen that is capable of being further processed into an MHC class I and/or MHC class II neoepitope).

A KRAS-associated MHC class II neoepitope can be predicted to be capable of presentation by at least one HLA allele. A KRAS-associated MHC class II neoepitope can be validated to be capable of presentation by at least one HLA allele.

HLA alleles can include a DRB1 allele, such as DRB1*01:01 and/or DRB1*07:01.

A KRAS-associated MHC class II neoepitope and HLA pair can be KLVVVGACGVGKSALTIQLI paired with DRB1*01:01. A KRAS-associated MHC class II neoepitope can be KLVVVGACGVGKSALTIQ and the at least one HLA allele comprises DRB1*07:01. A KRAS-associated MHC class II neoepitope and HLA pair can be MTEYKLVVVGACGVGK paired with DRB1*07:01. A KRAS-associated MHC class II neoepitope and HLA pair can be MTEYKLVVVGACGV paired with DRB1*07:01.

HLA alleles can include a DPB1 allele, such as DPB1*04:01, DPB1*104:01, DPB1*01:01, or DPB1*13:01.

A KRAS-associated MHC class II neoepitope and HLA pair can be KLVVVGACGVGKSALTI paired with DPB1*04:01. A KRAS-associated MHC class II neoepitope and HLA pair can be LVVVGACGVGKSALTIQLIQ paired with DPB1*04:01 or DPB1*104:01. A KRAS-associated MHC class II neoepitope and HLA pair can be VVGACGVGKSALTIQ paired with DPB1*01:01. A KRAS-associated MHC class II neoepitope and HLA pair can be YKLVVVGACGVGKSALTIQL paired with DPB1*13:01.

HLA alleles can include a DPA1 allele, such as DPA1*01:03, DPA1*02:02, or DPA1*02:01.

A KRAS-associated MHC class II neoepitope and HLA pair can be KLVVVGACGVGKSALTI paired with DPA1*01:03. A KRAS-associated MHC class II neoepitope and HLA pair can be LVVVGACGVGKSALTIQLIQ paired with DPA1*01:03. A KRAS-associated MHC class II neoepitope and HLA pair can be VVGACGVGKSALTIQ paired with DPA1*02:02. A KRAS-associated MHC class II neoepitope and HLA pair can be YKLVVVGACGVGKSALTIQL paired with DPA1*02:01.

Any of the compositions described herein, when administered to a subject and the epitope-encoding nucleic acid sequence is expressed and translated, the expressed KRAS-associated MHC class II neoepitope can stimulate an immune response. Stimulating an immune response can include stimulating CD4 T cells. Stimulating an immune response can include production of a cytokine, such as IL2 and/or IFNγ. Stimulating an immune response can include increasing the frequency of CD25+ and/or CD69+ CD4 T cells.

KRAS-associated MHC class II neoepitopes can be 10-25 amino acids in length.

Antigen-encoding nucleic acid sequences (e.g., an antigen that encompasses the epitope, such as an antigen that can be further processed into an epitope, and/or an antigen that in some instances is the epitope) can encode a peptide that is between 10-35 amino acids in length. Antigen-encoding nucleic acid sequences can encode a peptide that is at least 10-35 amino acids in length. Antigen-encoding nucleic acid sequences can encode a peptide that is between 10-40 amino acids in length. Antigen-encoding nucleic acid sequences can encode a peptide that is at least 10-40 amino acids in length.

Also described herein are methods of stimulating a T cell responses. Methods of stimulating a T cell responses can include delivering or having delivered any of the compositions (e.g., any of the antigen expression systems) described herein to a cell, where an epitope-encoding nucleic acid sequence is capable of being expressed and translated by the cell, and a KRAS-associated MHC class II neoepitope is capable of presentation by at least one HLA allele on the cell surface, and wherein the cell is contact with or comes in contact with a cognate T cell thereby stimulating the T cell response.

Delivering can include administering or having administered the antigen expression system to a subject.

Methods of stimulating a T cell responses can include administering or having administered any of the compositions (e.g., any of the antigen expression systems) described herein to a subject, wherein an epitope-encoding nucleic acid sequence is capable of being expressed and translated by a cell of the subject, and a KRAS-associated MHC class II neoepitope is capable of presentation by at least one HLA allele on the cell surface.

Methods herein can include determining or having determined an HLA-haplotype of a subject (“haplotyping a subject”) prior to administering or having administered an antigen expression system.

Haplotyping can include determining or having determined whether the HLA-haplotype of a subject includes DRB1*01:01 and/or DRB1*07:01 prior to administering or having administered the antigen expression system. Haplotyping can include determining or having determined whether the HLA-haplotype of a subject includes DRB1*01:01, DRB1*07:01, DPB1*04:01, DPB1*104:01, DPB1*01:01, DPB1*13:01, DPA1*01:03, DPA1*02:02, or DPA1*02:01 prior to administering or having administered the antigen expression system.

Also described herein are methods for stimulating an immune response in a subject, the method comprising the method comprising administering to the subject a KRAS-associated MHC class II neoepitope-based vaccine to the subject. A KRAS-associated MHC class II neoepitope-based vaccine can include at least one KRAS-associated MHC class II neoepitope. A KRAS-associated MHC class II neoepitope-based vaccine can include a KRAS-associated MHC class II neoepitope-encoding nucleic acid sequence encoding at least one KRAS-associated MHC class II neoepitope. A KRAS-associated MHC class II neoepitope-encoding nucleic acid sequence can include a viral vector, an mRNA vector, or a DNA vector. A KRAS-associated MHC class II neoepitope-encoding nucleic acid sequence can include an mRNA vector.

Provided for herein is a composition for delivery of an antigen expression system, comprising: the antigen expression system, wherein the antigen expression system comprises one or more vectors, the one or more vectors comprising: (a) a vector backbone, wherein the vector backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding a KRAS-associated MHC class II neoepitope, and (II) optionally, a 5′ linker sequence, and (III) optionally, a 3′ linker sequence; (ii) optionally, a second promoter nucleotide sequence operably linked to the antigen-encoding nucleic acid sequence; and (iii) optionally, at least one MHC class I epitope-encoding nucleic acid sequence; (iv) optionally, at least one nucleic acid sequence encoding a GPGPG amino acid linker sequence; and (v) optionally, at least one second poly(A) sequence, wherein the second poly(A) sequence is a native poly(A) sequence or an exogenous poly(A) sequence to the vector backbone, and wherein if the second promoter nucleotide sequence is absent, the antigen-encoding nucleic acid sequence is operably linked to the at least one promoter nucleotide sequence.

In some aspects, the KRAS-associated MHC class II neoepitope is selected from the group consisting of: a KRAS G12C mutation, a KRAS G12V mutation, a KRAS G12D mutation, a KRAS G12A mutation, and a KRAS Q61H mutation. In some aspects, the at least one antigen-encoding nucleic acid sequence comprising the KRAS G12C mutation comprises an amino acid sequence selected from the group consisting of: MTEYKLVVVGACGVGKSALTIQLIQ, MTEYKLVVVGACGVGKSALTIQLIQN, MTEYKLVVVGACGVGKSALTIQLIQNH, MTEYKLVVVGACGVGKSALTIQLIQNHF, MTEYKLVVVGACGVGKSALTIQLIQNHFVDEYDPT, and MTEYKLVVVGACGVGKSALTIQLIQNHFVDEYDPTIEDSY. In some aspects, the at least one antigen-encoding nucleic acid sequence comprising the KRAS G12D mutation comprises an amino acid sequence selected from the group consisting of: MTEYKLVVVGADGVGKSALTIQLIQ, MTEYKLVVVGADGVGKSALTIQLIQN, MTEYKLVVVGADGVGKSALTIQLIQNH, MTEYKLVVVGADGVGKSALTIQLIQNHF, MTEYKLVVVGADGVGKSALTIQLIQNHFVDEYDPT, and MTEYKLVVVGADGVGKSALTIQLIQNHFVDEYDPTIEDSY. In some aspects, the at least one antigen-encoding nucleic acid sequence comprising the KRAS G12V mutation comprises an amino acid sequence selected from the group consisting of: MTEYKLVVVGAVGVGKSALTIQLIQ, MTEYKLVVVGAVGVGKSALTIQLIQN, MTEYKLVVVGAVGVGKSALTIQLIQNH, MTEYKLVVVGAVGVGKSALTIQLIQNHF, MTEYKLVVVGAVGVGKSALTIQLIQNHFVDEYDPT, and MTEYKLVVVGAVGVGKSALTIQLIQNHFVDEYDPTIEDSY. In some aspects, the at least one antigen-encoding nucleic acid sequence comprising the KRAS Q61H mutation comprises an amino acid sequence selected from the group consisting of: ETCLLDILDTAGHEEYSAMRDQYMR, GETCLLDILDTAGHEEYSAMRDQYMR, GETCLLDILDTAGHEEYSAMRDQYMRT, DGETCLLDILDTAGHEEYSAMRDQYMRT, VVIDGETCLLDILDTAGHEEYSAMRDQYMRTGEGF, and RKQVVIDGETCLLDILDTAGHEEYSAMRDQYMRTGEGFLC. In some aspects, the at least one antigen-encoding nucleic acid sequence comprising the KRAS G12A mutation comprises an amino acid sequence selected from the group consisting of: MTEYKLVVVGAAGVGKSALTIQLIQ, MTEYKLVVVGAAGVGKSALTIQLIQN, MTEYKLVVVGAAGVGKSALTIQLIQNH, MTEYKLVVVGAAGVGKSALTIQLIQNHF, MTEYKLVVVGAAGVGKSALTIQLIQNHFVDEYDPT, and MTEYKLVVVGAAGVGKSALTIQLIQNHFVDEYDPTIEDSY. Also provided for herein is a composition for delivery of an antigen expression system, comprising: the antigen expression system, wherein the antigen expression system comprises one or more vectors, the one or more vectors comprising: (a) a vector backbone, wherein the vector backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding a KRAS-associated MHC class II neoepitope, wherein the a KRAS-associated MHC class II neoepitope comprises a KRAS G12C mutation, and (II) optionally, a 5′ linker sequence, and (III) optionally, a 3′ linker sequence; (ii) optionally, a second promoter nucleotide sequence operably linked to the antigen-encoding nucleic acid sequence; and (iii) optionally, at least one MHC class I epitope-encoding nucleic acid sequence; (iv) optionally, at least one nucleic acid sequence encoding a GPGPG amino acid linker sequence; and (v) optionally, at least one second poly(A) sequence, wherein the second poly(A) sequence is a native poly(A) sequence or an exogenous poly(A) sequence to the vector backbone, and wherein if the second promoter nucleotide sequence is absent, the antigen-encoding nucleic acid sequence is operably linked to the at least one promoter nucleotide sequence. In some aspects, the KRAS-associated MHC class II neoepitope comprises any of the amino acid sequences shown in Table 2. In some aspects, the KRAS-associated MHC class II neoepitope comprises any of the amino acid sequences selected from the group consisting of: EYKLVVVGACG, KLVVVGACGVGKSALTIQLI, KLVVVGACGVGKSALTIQLIQ, MTEYKLVVVGACGV, MTEYKLVVVGACGVGK, KLVVVGACGVGKSALTI, KLVVVGACGVGKSALTIQ, LVVVGACGVGKSALTIQLIQ, MTEYKLVVVGACGVGK, VVGACGVGKSALTIQ, and YKLVVVGACGVGKSALTIQL. In some aspects, the KRAS associated MHC class II neoepitope comprises any of the amino acid sequences selected from the group consisting of: EYKLVVVGACG, KLVVVGACGVGKSALTIQLI, KLVVVGACGVGKSALTIQLIQ, MTEYKLVVVGACGV, and MTEYKLVVVGACGVGK. In some aspects, the KRAS-associated MHC class II neoepitope comprises any of the amino acid sequences selected from the group consisting of: KLVVVGACGVGKSALTI, KLVVVGACGVGKSALTIQ, LVVVGACGVGKSALTIQLIQ, MTEYKLVVVGACGVGK, VVGACGVGKSALTIQ, and YKLVVVGACGVGKSALTIQL. In some aspects, the KRAS-associated MHC class II neoepitope comprises the amino acid sequence KLVVVGACG. In some aspects, the KRAS-associated MHC class II neoepitope comprises the amino acid sequence EYKLVVVGACG. In some aspects, the KRAS-associated MHC class II neoepitope comprises the amino acid sequence KLVVVGACGVGKSALTIQLI or KLVVVGACGVGKSALTIQLIQ. In some aspects, the KRAS-associated MHC class II neoepitope comprises the amino acid sequence MTEYKLVVVGACGV or MTEYKLVVVGACGVGK. In some aspects, the KRAS-associated MHC class II neoepitope comprises the amino acid sequence MTEYKLVVVGACGVGKSALTIQLIQ. In some aspects, the at least one antigen-encoding nucleic acid sequence encodes a peptide comprising the amino acid sequence MTEYKLVVVGACGVGKSALTIQLIQ. In some aspects, the KRAS-associated MHC class II neoepitope comprises any of the amino acid sequences selected from the group consisting of: MTEYKLVVVGACGVGKSALTIQLIQ, MTEYKLVVVGACGVGKSALTIQLIQN, MTEYKLVVVGACGVGKSALTIQLIQNH, MTEYKLVVVGACGVGKSALTIQLIQNHF, MTEYKLVVVGACGVGKSALTIQLIQNHFVDEYDPT, and MTEYKLVVVGACGVGKSALTIQLIQNHFVDEYDPTIEDSY. In some aspects, the at least one antigen-encoding nucleic acid sequence comprises an amino acid sequence selected from the group consisting of: MTEYKLVVVGACGVGKSALTIQLIQ, MTEYKLVVVGACGVGKSALTIQLIQN, MTEYKLVVVGACGVGKSALTIQLIQNH, MTEYKLVVVGACGVGKSALTIQLIQNHF, MTEYKLVVVGACGVGKSALTIQLIQNHFVDEYDPT, and MTEYKLVVVGACGVGKSALTIQLIQNHFVDEYDPTIEDSY.

In some aspects, the epitope-encoding nucleic acid sequence further encodes a KRAS-associated MHC class I neoepitope. In some aspects, the KRAS-associated MHC class I neoepitope comprises a fragment of the KRAS-associated MHC class II neoepitope. In some aspects, the KRAS-associated MHC class II neoepitope is predicted to be capable of presentation by at least one HLA allele. In some aspects, the KRAS-associated MHC class II neoepitope is validated to be capable of presentation by at least one HLA allele. In some aspects, the at least one HLA allele comprises a DRB1 allele. In some aspects, the DRB1 allele comprises DRB1*01:01 and/or DRB1*07:01. In some aspects, the KRAS-associated MHC class II neoepitope comprises the amino acid sequence KLVVVGACGVGKSALTIQLI and the at least one HLA allele comprises DRB1*01:01. In some aspects, the KRAS-associated MHC class II neoepitope comprises the amino acid sequence KLVVVGACGVGKSALTIQ and the at least one HLA allele comprises DRB1*07:01. In some aspects, the KRAS-associated MHC class II neoepitope comprises the amino acid sequence MTEYKLVVVGACGVGK and the at least one HLA allele comprises DRB1*07:01. In some aspects, the KRAS-associated MHC class II neoepitope comprises the amino acid sequence MTEYKLVVVGACGV and the at least one HLA allele comprises DRB1*07:01. In some aspects, the at least one HLA allele comprises a DPB1 allele. In some aspects, the DPB1 allele is DPB1*04:01, DPB1*104:01, DPB1*01:01, or DPB1*13:01. In some aspects, the KRAS-associated MHC class II neoepitope comprises the amino acid sequence KLVVVGACGVGKSALTI and the at least one HLA allele comprises DPB1*04:01. In some aspects, the KRAS-associated MHC class II neoepitope comprises the amino acid sequence LVVVGACGVGKSALTIQLIQ and the at least one HLA allele comprises DPB1*04:01 or DPB1*104:01. In some aspects, the KRAS-associated MHC class II neoepitope comprises the amino acid sequence VVGACGVGKSALTIQ and the at least one HLA allele comprises DPB1*01:01. In some aspects, the KRAS-associated MHC class II neoepitope comprises the amino acid sequence YKLVVVGACGVGKSALTIQL and the at least one HLA allele comprises DPB1*13:01. In some aspects, the at least one HLA allele comprises a DPA1 allele. In some aspects, the DPA1 allele is DPA1*01:03, DPA1*02:02, or DPA1*02:01. In some aspects, the KRAS-associated MHC class II neoepitope comprises the amino acid sequence KLVVVGACGVGKSALTI and the at least one HLA allele comprises DPA1*01:03. In some aspects, the KRAS-associated MHC class II neoepitope comprises the amino acid sequence LVVVGACGVGKSALTIQLIQ and the at least one HLA allele comprises DPA1*01:03. In some aspects, the KRAS-associated MHC class II neoepitope comprises the amino acid sequence VVGACGVGKSALTIQ and the at least one HLA allele comprises DPA1*02:02. In some aspects, the KRAS-associated MHC class II neoepitope comprises the amino acid sequence YKLVVVGACGVGKSALTIQL and the at least one HLA allele comprises DPA1*02:01. In some aspects, when the composition is administered to a subject and the epitope-encoding nucleic acid sequence is expressed and translated, the KRAS-associated MHC class II neoepitope is capable of stimulating an immune response. In some aspects, the immune response comprises stimulating CD4 T cells. In some aspects, the immune response comprises production of a cytokine. In some aspects, the cytokine comprises IL2 and/or IFNγ. In some aspects, the immune response comprises an increase in the frequency of CD25+ and/or CD69+CD4 T cells. In some aspects, the KRAS-associated MHC class II neoepitope is 10-25 amino acids in length. In some aspects, the antigen-encoding nucleic acid sequence encodes a peptide that is between 10-35 amino acids in length. In some aspects, the antigen-encoding nucleic acid sequence encodes a peptide that is at least 10-35 amino acids in length. In some aspects, the antigen-encoding nucleic acid sequence encodes a peptide that is between 10-40 amino acids in length. In some aspects, the antigen-encoding nucleic acid sequence encodes a peptide that is at least 10-40 amino acids in length.

Also provided herein is a method of stimulating a T cell response, the method comprising delivering or having delivered any one of the above antigen expression systems to a cell, wherein the epitope-encoding nucleic acid sequence is capable of being expressed and translated by the cell, and the KRAS-associated MHC class II neoepitope is capable of presentation by at least one HLA allele on the cell surface, and wherein the cell is is contact with or comes in contact with a cognate T cell thereby stimulating the T cell response. In some aspects, the delivering comprises administering or having administered the antigen expression system to a subject.

Also provided herein is a method of stimulating an immune response, the method comprising administering or having administered any one of the above antigen expression systems to a subject, wherein the epitope-encoding nucleic acid sequence is capable of being expressed and translated by a cell of the subject, and the KRAS-associated MHC class II neoepitope is capable of presentation by at least one HLA allele on the cell surface. In some aspects, the method comprises determining or having determined an HLA-haplotype of the subject prior to administering or having administered the antigen expression system. In some aspects, the method comprises determining or having determined the HLA-haplotype of the subject comprises DRB1*01:01 and/or DRB1*07:01 prior to administering or having administered the antigen expression system. In some aspects, the method comprises determining or having determined the HLA-haplotype of the subject comprises DRB1*01:01, DRB1*07:01, DPB1*04:01, DPB1*104:01, DPB1*01:01, DPB1*13:01, DPA1*01:03, DPA1*02:02, or DPA1*02:01 prior to administering or having administered the antigen expression system.

Also provided for herein is a KRAS-associated MHC class II neoepitope-based vaccine comprising: 1) at least one KRAS-associated MHC class II neoepitope, or 2) a KRAS-associated MHC class II neoepitope-encoding nucleic acid sequence encoding the at least one KRAS-associated MHC class II neoepitope.

In some aspects, the KRAS-associated MHC class II neoepitope comprises any of the amino acid sequences shown in Table 2. In some aspects, the KRAS-associated MHC class II neoepitope comprises any of the amino acid sequences selected from the group consisting of: EYKLVVVGACG, KLVVVGACGVGKSALTIQLI, KLVVVGACGVGKSALTIQLIQ, MTEYKLVVVGACGV, MTEYKLVVVGACGVGK, KLVVVGACGVGKSALTI, KLVVVGACGVGKSALTIQ, LVVVGACGVGKSALTIQLIQ, MTEYKLVVVGACGVGK, VVGACGVGKSALTIQ, and YKLVVVGACGVGKSALTIQL. In some aspects, the KRAS-associated MHC class II neoepitope comprises any of the amino acid sequences selected from the group consisting of: EYKLVVVGACG, KLVVVGACGVGKSALTIQLI, KLVVVGACGVGKSALTIQLIQ, MTEYKLVVVGACGV, and MTEYKLVVVGACGVGK. In some aspects, the KRAS-associated MHC class II neoepitope comprises any of the amino acid sequences selected from the group consisting of: KLVVVGACGVGKSALTI, KLVVVGACGVGKSALTIQ, LVVVGACGVGKSALTIQLIQ, MTEYKLVVVGACGVGK, VVGACGVGKSALTIQ, and YKLVVVGACGVGKSALTIQL. In some aspects, the KRAS-associated MHC class II neoepitope comprises the amino acid sequence KLVVVGACG. In some aspects, the KRAS-associated MHC class II neoepitope comprises the amino acid sequence EYKLVVVGACG. In some aspects, the KRAS-associated MHC class II neoepitope comprises the amino acid sequence KLVVVGACGVGKSALTIQLI or KLVVVGACGVGKSALTIQLIQ. In some aspects, the KRAS-associated MHC class II neoepitope comprises the amino acid sequence MTEYKLVVVGACGV or MTEYKLVVVGACGVGK. In some aspects, the KRAS-associated MHC class II neoepitope comprises the amino acid sequence MTEYKLVVVGACGVGKSALTIQLIQ.

In some aspects, the KRAS-associated MHC class II neoepitope-encoding nucleic acid sequence comprises a viral vector, an mRNA vector, or a DNA vector. In some aspects, the KRAS-associated MHC class II neoepitope-encoding nucleic acid sequence comprises an mRNA vector. Also provided for herein is a method for stimulating an immune response in a subject, the method comprising administering to the subject a KRAS-associated MHC class II neoepitope-based vaccine to the subject, wherein the KRAS-associated MHC class II neoepitope-based vaccine comprises: 1) at least one KRAS-associated MHC class II neoepitope, or 2) a KRAS-associated MHC class II neoepitope-encoding nucleic acid sequence encoding the at least one KRAS-associated MHC class II neoepitope. In some aspects, the KRAS-associated MHC class II neoepitope comprises any of the amino acid sequences shown in Table 2. In some aspects, the KRAS-associated MHC class II neoepitope comprises any of the amino acid sequences selected from the group consisting of: EYKLVVVGACG, KLVVVGACGVGKSALTIQLI, KLVVVGACGVGKSALTIQLIQ, MTEYKLVVVGACGV, MTEYKLVVVGACGVGK, KLVVVGACGVGKSALTI, KLVVVGACGVGKSALTIQ, LVVVGACGVGKSALTIQLIQ, MTEYKLVVVGACGVGK, VVGACGVGKSALTIQ, and YKLVVVGACGVGKSALTIQL. In some aspects, the KRAS-associated MHC class II neoepitope comprises any of the amino acid sequences selected from the group consisting of: EYKLVVVGACG, KLVVVGACGVGKSALTIQLI, KLVVVGACGVGKSALTIQLIQ, MTEYKLVVVGACGV, and MTEYKLVVVGACGVGK. In some aspects, the KRAS-associated MHC class II neoepitope comprises any of the amino acid sequences selected from the group consisting of: KLVVVGACGVGKSALTI, KLVVVGACGVGKSALTIQ, LVVVGACGVGKSALTIQLIQ, MTEYKLVVVGACGVGK, VVGACGVGKSALTIQ, and YKLVVVGACGVGKSALTIQL. In some aspects, the KRAS-associated MHC class II neoepitope comprises the amino acid sequence KLVVVGACG. In some aspects, the KRAS-associated MHC class II neoepitope comprises the amino acid sequence EYKLVVVGACG. In some aspects, the KRAS-associated MHC class II neoepitope comprises the amino acid sequence KLVVVGACGVGKSALTIQLI or KLVVVGACGVGKSALTIQLIQ. In some aspects, the KRAS-associated MHC class II neoepitope comprises the amino acid sequence MTEYKLVVVGACGV or MTEYKLVVVGACGVGK. In some aspects, the KRAS-associated MHC class II neoepitope comprises the amino acid sequence MTEYKLVVVGACGVGKSALTIQLIQ. In some aspects, the KRAS-associated MHC class II neoepitope-encoding nucleic acid sequence comprises a viral vector, an mRNA vector, or a DNA vector. In some aspects, the KRAS-associated MHC class II neoepitope-encoding nucleic acid sequence comprises an mRNA vector.

In some aspects, the method comprises determining or having determined an HLA-haplotype of the subject prior to administering or having administered the antigen expression system. In some aspects, the method comprises determining or having determined the HLA-haplotype of the subject comprises DRB1*01:01 and/or DRB1*07:01 prior to administering or having administered the antigen expression system. In some aspects, the method comprises determining or having determined the HLA-haplotype of the subject comprises DRB1*01:01, DRB1*07:01, DPB1*04:01, DPB1*104:01, DPB1*01:01, DPB1*13:01, DPA1*01:03, DPA1*02:02, or DPA1*02:01 prior to administering or having administered the antigen expression system. In some aspects, any of the above compositions further comprise a nanoparticulate delivery vehicle. The nanoparticulate delivery vehicle, in some aspects, may be a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system. In some aspects, any of the above compositions further comprise a plurality of LNPs, wherein the LNPs comprise: the antigen expression system; a cationic lipid; a non-cationic lipid; and a conjugated lipid that inhibits aggregation of the LNPs, wherein at least about 95% of the LNPs in the plurality of LNPs either: have a non-lamellar morphology; or are electron-dense. In some aspects, the non-cationic lipid is a mixture of (1) a phospholipid and (2) cholesterol or a cholesterol derivative. In some aspects, the conjugated lipid that inhibits aggregation of the LNPs is a polyethyleneglycol (PEG)-lipid conjugate. In some aspects, the PEG-lipid conjugate is selected from the group consisting of: a PEG-diacylglycerol (PEG-DAG) conjugate, a PEG dialkyloxypropyl (PEG-DAA) conjugate, a PEG-phospholipid conjugate, a PEG-ceramide (PEG-Cer) conjugate, and a mixture thereof. In some aspects the PEG-DAA conjugate is a member selected from the group consisting of: a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, and a mixture thereof. In some aspects, the antigen expression system is fully encapsulated in the LNPs.

In some aspects, the non-lamellar morphology of the LNPs comprises an inverse hexagonal (H_{II}) or cubic phase structure. In some aspects, the cationic lipid comprises from about 10 mol % to about 50 mol % of the total lipid present in the LNPs. In some aspects, the cationic lipid comprises from about 20 mol % to about 50 mol % of the total lipid present in the LNPs. In some aspects, the cationic lipid comprises from about 20 mol % to about 40 mol % of the total lipid present in the LNPs. In some aspects, the non-cationic lipid comprises from about 10 mol % to about 60 mol % of the total lipid present in the LNPs. In some aspects, the non-cationic lipid comprises from about 20 mol % to about 55 mol % of the total lipid present in the LNPs. In some aspects, the non-cationic lipid comprises from about 25 mol % to about 50 mol % of the total lipid present in the LNPs. In some aspects, the conjugated lipid comprises from about 0.5 mol % to about 20 mol % of the total lipid present in the LNPs. In some aspects, the conjugated lipid comprises from about 2 mol % to about 20 mol % of the total lipid present in the LNPs. In some aspects, the conjugated lipid comprises from about 1.5 mol % to about 18 mol % of the total lipid present in the LNPs. In some aspects, greater than 95% of the LNPs have a non-lamellar morphology. In some aspects, greater than 95% of the LNPs are electron dense. In some aspects, any of the above compositions further comprise a plurality of LNPs, wherein the LNPs comprise: a cationic lipid comprising from 50 mol % to 65 mol % of the total lipid present in the LNPs; a conjugated lipid that inhibits aggregation of LNPs comprising from 0.5 mol % to 2 mol % of the total lipid present in the LNPs; and a non-cationic lipid comprising either: a mixture of a phospholipid and cholesterol or a derivative thereof, wherein the phospholipid comprises from 4 mol % to 10 mol % of the total lipid present in the LNPs and the cholesterol or derivative thereof comprises from 30 mol % to 40 mol % of the total lipid present in the LNPs; a mixture of a phospholipid and cholesterol or a derivative thereof, wherein the phospholipid comprises from 3 mol % to 15 mol % of the total lipid present in the LNPs and the cholesterol or derivative thereof comprises from 30 mol % to 40 mol % of the total lipid present in the LNPs; or up to 49.5 mol % of the total lipid present in the LNPs and comprising a mixture of a phospholipid and cholesterol or a derivative thereof, wherein the cholesterol or derivative thereof comprises from 30 mol % to 40 mol % of the total lipid present in the LNPs. In some aspects, any of the above compositions further comprise a plurality of LNPs, wherein the LNPs comprise: a cationic lipid comprising from 50 mol % to 85 mol % of the total lipid present in the LNPs; a conjugated lipid that inhibits aggregation of LNPs comprising from 0.5 mol % to 2 mol % of the total lipid present in the LNPs; and a non-cationic lipid comprising from 13 mol % to 49.5 mol % of the total lipid present in the LNPs. In some aspects, the phospholipid comprises dipalmitoylphosphatidylcholine (DPPC), distearoylphosphatidylcholine (DSPC), or a mixture thereof. In some aspects, the conjugated lipid comprises a polyethyleneglycol (PEG)-lipid conjugate. In some aspects, the PEG-lipid conjugate comprises a PEG-diacylglycerol (PEG-DAG) conjugate, a PEG-dialkyloxypropyl (PEG-DAA) conjugate, or a mixture thereof. In some aspects, the PEG-DAA conjugate comprises a PEG-dimyristyloxypropyl (PEG-DMA) conjugate, a PEG-distearyloxypropyl (PEG-DSA) conjugate, or a mixture thereof. In some aspects, the PEG portion of the conjugate has an average molecular weight of about 2,000 daltons.

In some aspects, the conjugated lipid comprises from 1 mol % to 2 mol % of the total lipid present in the LNPs. In some aspects, the LNP comprises a compound having a structure of Formula I:

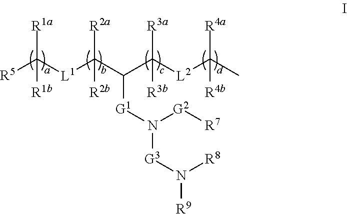

or a pharmaceutically acceptable salt, tautomer, prodrug or stereoisomer thereof, wherein: L¹ and L² are each independently -0(C=0)-, —(C=0)0-, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond; G¹ is C₁-C₂ alkylene,-(C=0)-, -0(C=0)-, —SC(═O)—, —R^{a}C(═O)— or a direct bond: —C(═O)—, —(C=0)0-, —C(═O)S—, —C(═O)R^{a}— or a direct bond; G is C₁-C₆ alkylene; R^{a} is H or C1-C12 alkyl; R^{1a} and R^{1b} are, at each occurrence, independently either: (a) H or C1-C12 alkyl; or (b) R^{1a} is H or C₁-C12 alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either: (a) H or C1-C12 alkyl; or (b) R^{2a} is H or C1-C12 alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a): H or C1-C12 alkyl; or (b) R^{3a} is H or C1-C12 alkyl, and R^{3b} together with the carbon atom to which it is bound is taken together with an adjacent R and the carbon atom to which it is bound to form a carbon-carbon double bond; R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond; R⁵ and R⁶ are each independently H or methyl; R⁷ is C4-C20 alkyl; R⁸ and R⁹ are each independently C1-C12 alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

In some aspects, the LNP comprises a compound having a structure of Formula II:

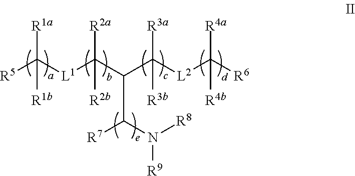

or a pharmaceutically acceptable salt, tautomer, prodrug or stereoisomer thereof, wherein: L¹ and L² are each independently -0(C=0)-, —(C=0)0- or a carbon-carbon double bond; R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond; R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently methyl or cycloalkyl; R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, provided that: at least one of Ri, R^{2a}, R^{3a} or R^{4a} is C₁-C₁₂ alkyl, or at least one of L¹ or L² is -0(C=0)- or —(C=0)0-; and R^{1a} and R^{1b} are not isopropyl when a is 6 or n-butyl when a is 8.

In some aspects, any of the above compositions further comprise one or more excipients comprising a neutral lipid, a steroid, and a polymer conjugated lipid. In some aspects, the neutral lipid comprises at least one of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and 1,2-Dioleoyl-sn-glycero-3-phosphoethanolamine (DOPE). In some aspects, the neutral lipid is DSPC. In some aspects, the molar ratio of the compound to the neutral lipid ranges from about 2:1 to about 8:1. In some aspects, the steroid is cholesterol. In some aspects, the molar ratio of the compound to cholesterol ranges from about 2:1 to 1:1. In some aspects, the polymer conjugated lipid is a pegylated lipid. In some aspects, the molar ratio of the compound to the pegylated lipid ranges from about 100:1 to about 25:1. In some aspects, the pegylated lipid is PEG-DAG, a PEG polyethylene (PEG-PE), a PEG-succinoyl-diacylglycerol (PEG-S-DAG), PEG-cer or a PEG dialkyoxypropylcarbamate. In some aspects, the pegylated lipid has the following structure III:

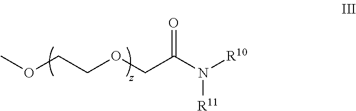

or a pharmaceutically acceptable salt, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60. In some aspects, R¹⁰ and R¹¹ are each independently straight, saturated alkyl chains having 12 to 16 carbon atoms. In some aspects, the average z is about 45.

In some aspects, the LNP self-assembles into non-bilayer structures when mixed with polyanionic nucleic acid. In some aspects, the non-bilayer structures have a diameter between 60 nm and 120 nm. In some aspects, the non-bilayer structures have a diameter of about 70 nm, about 80 nm, about 90 nm, or about 100 nm. In some aspects, wherein the nanoparticulate delivery vehicle has a diameter of about 100 nm.

The practice of the methods herein will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### EXAMPLES

### Example 1: Disclosed EDGE Presentation Models Outperform BERTMHC and NetMHCIIpan4.0

Disclosed herein is an EDGE presentation model that leverages structural information of putative epitopes and HLA class II alleles from their in-situ context to predict presentation of peptides by HLA class II. This presentation model leverages the Evolutionary Scale Model (ESM) pre-trained protein language model (LM), which has been demonstrated to embed protein sequences with rich structural information. The input to the model is a linear peptide consisting of an epitope and its flanking amino acids, concatenated with structurally relevant amino acids from each HLA allele. This allows the model to treat the modeling problem entirely as a natural language processing task, which minimizes imputation of covariates found in prior approaches when performing inference in the context of vaccine design, while maximizing the richness of the LM embeddings on longer linear peptides. This also allows the model to generalize to any allele that has a known sequence. Additionally, DR-, DP-, and DQ-specific immunoaffinity purified mass spectrometry multi-allelic (MA) presentation data were generated per tumor or cell line sample, spanning 89 alleles in aggregate. Incrementally decreasing the HLA class II allele MA resolution during training results in substantially improved predictions for situations where MA presentation data has completely ambiguous epitope presentation across DR/DP/DQ alleles. Overall, the presentation model achieves an Average Precision (AP) of 0.92 and ROC-AUC of 0.98 on the same benchmark validation data as the current state-of-the-art model BERTMHC, which achieved an AP of 0.81 and ROC-AUC of 0.95. These are the best AP and ROC-AUC for an HLA Class II presentation model on this benchmark dataset to the best of our knowledge. The presentation model is a significant advancement in HLA class II epitope prediction and brings neoantigen vaccine design optimized for both class I and class II presentation within reach.

Generally, the disclosed EDGE presentation model addresses shortcomings of prior methods (e.g., including BERTMHC described in Cheng, J., Bendjama, K., Rittner, K. & Malone, B. BERTMHC: improved MHC-peptide class II interaction prediction with transformer and multiple instance learning. Bioinformatics 37, 4172-4179 (2021), which is hereby incorporated by reference in its entirety). In particular, BERTMHC generates predictions across a set of alleles that are independent from each other. Thus, BERTMHC's methodology does not learn from other alleles present in a genotype of a sample. In contrast, the EDGE presentation model disclosed herein implements at least a learned genotype network that aggregates contributions from across alleles of a genotype. In other words, unlike BERTMHC, the EDGE presentation model allows the network to learn from all the class II MHC alleles present in a sample, not just the most likely class II MHC allele. Here, the pooling is conducted on the embeddings of each epitope:allele combination from ESM. This is done by calculating a learned weighted average of each representation. More weight (“attention”) is given to alleles important for making the correct classification. Thus, in the EDGE presentation model, each class II MHC allele must compete with the other alleles present in the genotype for attention from the model, because the network restricts the total amount of attention given over all the alleles. Therefore, the output of the network is dependent on the genotype of alleles.

Specifically, three different presentation models (referred to as EDGE presentation models) were constructed and compared to two previously documented pipelines.

EDGE (max): A first presentation model, referred to in this example as “EDGE (max)” includes a first machine learning model (e.g., an Evolutionary Scale Model (ESM2) language model), a second machine learning model (e.g., a classifier), but does not include a pooling network. Thus, the EDGE (max) presentation model does not aggregate embeddings across all class II HLA alleles and instead, merely takes the single class II HLA allele that most likely presents the epitope.

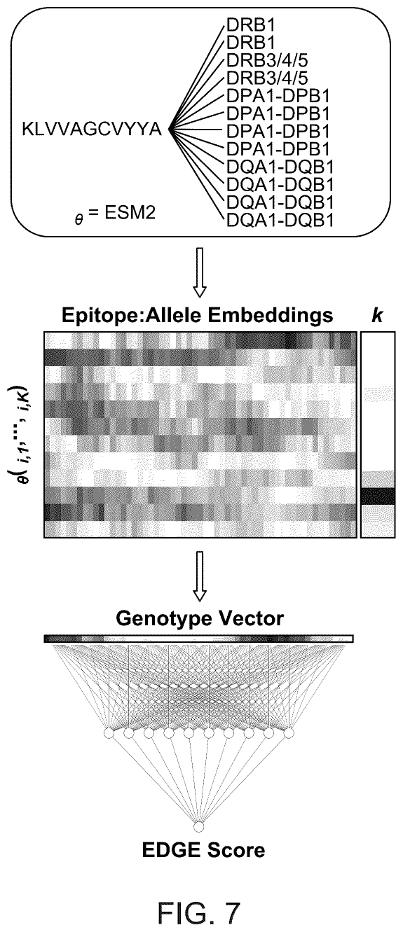

EDGE (LGN): A second presentation model, referred to in this example as “EDGE (LGN)” includes the example presentation model described in FIG. 4A and further shown in FIG. 7. Specifically, EDGE (LGN) includes a first machine learning model (e.g., an Evolutionary Scale Model (ESM2) language model), a learned genotype network (e.g., the pooling network 430 shown in FIG. 4A), and a second machine learning model (e.g., a classifier). Thus, EDGE (LGN) aggregates embeddings from all class II HLA alleles prior to prediction. Here, EDGE (LGN) is trained using only a publicly available dataset (e.g., Reynisson class II dataset obtained from Reynisson, B., Alvarez, B., Paul, S., Peters, B. & Nielsen, M. NetMHCpan-4.1 and NetMHCIIpan-4.0: Improved predictions of MHC antigen presentation by concurrent motif deconvolution and integration of MS MHC eluted ligand data. Nucleic Acids Res 48, W449-W454 (2021), which is incorporated by reference in its entirety). Here, the Reynisson class II dataset includes presentation data of single allelic samples.

EDGE (LGN+IP-MS): A third presentation model, referred to in this example as “EDGE (LGN+IP-MS)” includes the presentation model described in FIG. 4A and further shown in FIG. 7. Specifically, EDGE (LGN+IP-MS) includes a first machine learning model (e.g., an Evolutionary Scale Model (ESM2) language model), a learned genotype network (e.g., the pooling network 430 shown in FIG. 4A), and a second machine learning model (e.g., a classifier). Thus, EDGE (LGN+IP-MS) aggregates embeddings from all class II HLA alleles prior to prediction. Here, the EDGE (LGN+IP-MS) presentation model differs from the aforementioned EDGE (LGN) presentation model according to the underlying training data that is used. Specifically, the EDGE (LGN+IP-MS) presentation model is trained using both the Reynisson class II dataset as well as an additional dataset. The additional dataset includes both 1) presentation data of multi-allelic samples and 2) presentation data of intermediate resolution DP/DQ/DR class II alleles. The training of the EDGE (LGN+IP-MS) is as follows:
Train on the class II single allele data (e.g., publicly available Reynisson class II single allele data), which trains the models to build concrete relationships between class II single alleles and epitope sequences
Next, train exclusively on intermediate resolution DP/DQ/DR class II allele data, which allows the model to strengthen its learned relationship between epitope sequence and allele sequence
Train on full multi-allele class II data (e.g., Reynisson class II data including primarily full class II genotypes)

The two previously documented pipelines include BERTMHC (further described in Cheng, J., Bendjama, K., Rittner, K. & Malone, B. BERTMHC: improved MHC-peptide class II interaction prediction with transformer and multiple instance learning. Bioinformatics 37, 4172-4179 (2021), which is hereby incorporated by reference in its entirety) and NetMHCIIpan4.0 (further described in Reynisson, B., Alvarez, B., Paul, S., Peters, B. & Nielsen, M. NetMHCpan-4.1 and NetMHCIIpan-4.0: Improved predictions of MHC antigen presentation by concurrent motif deconvolution and integration of MS MHC eluted ligand data. Nucleic Acids Res 48, W449-W454 (2021), which is hereby incorporated by reference in its entirety).

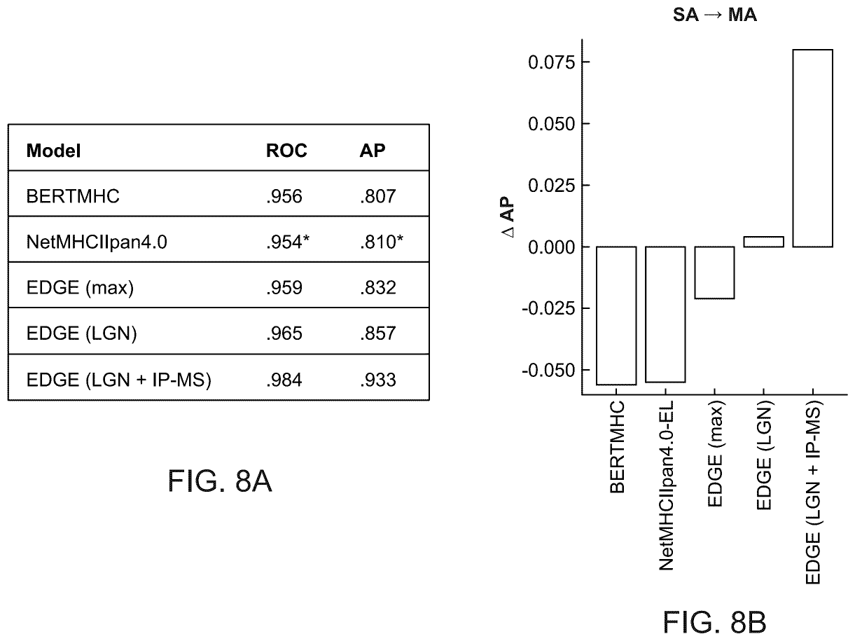

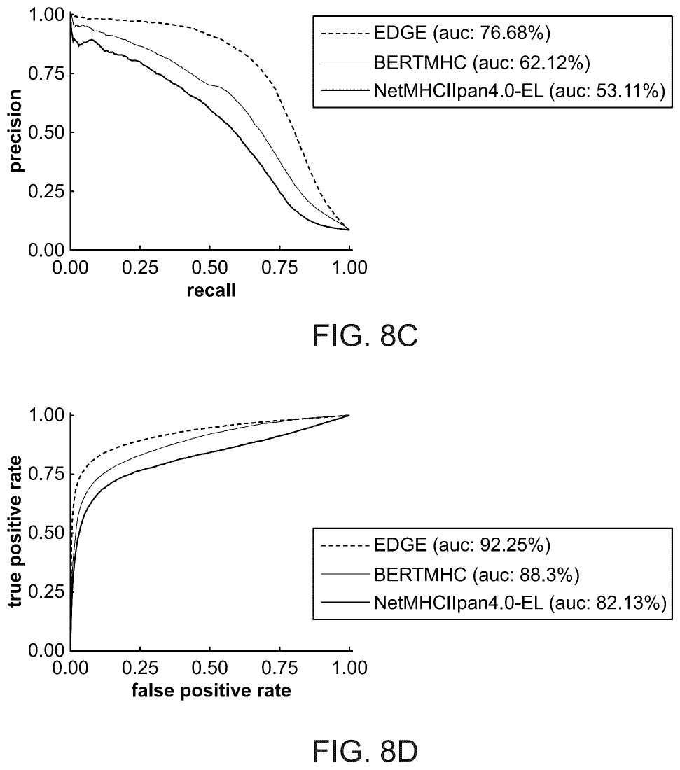

Reference is now made to FIGS. 8A and 8B, which show example performances of the EDGE (max), EDGE (LGN), and EDGE (LGN+IP-MS) presentation models in comparison to BERTMHC and NetMHCIIpan4.0. Specifically, FIGS. 8A and 8B show the performance metrics of 1) area under receiver operating characteristic (“ROC” or “ROC-AUC”) and 2) average precision (AP). As evident in FIGS. 8A and 8B, each of the three EDGE presentation models (EDGE (max), EDGE (LGN), and EDGE (LGN+IP-MS)) achieved substantially improved ROC-AUC and AP performance metrics over the existing methods of BERTMHC and NetMHCIIpan4.0.

Additionally, EDGE (LGN) achieved improved performance metrics in comparison to EDGE (max), which demonstrates the value of including the learned genotype network for aggregating embeddings from all class II HLA alleles prior to prediction. The LGN allows all alleles in a genotype to contribute to the gradient during backpropagation, as opposed to the max operator. In other words, by accounting for contributions across multiple class II MHC alleles, the EDGE (LGN) achieves improved performance. Furthermore, EDGE (LGN+IP-MS) achieved additional improved performance metrics in comparison to EDGE (LGN). This demonstrates the value of further training using immunoaffinity purified mass spectrometry data (e.g., DP/DQ/DR intermediate resolution data). Here, the immunoaffinity purified mass spectrometry data likely enabled the models to further learn relationships between epitope sequences with less difficulty than directly attempting to predict over the full genotype, thereby enabling improved predictions.

FIG. 8B shows the change in average precision (AP) when predicting for single allelic (SA) data in comparison to multi-allelic (MA) data. In particular, each of BERTMHC, NetMHCIIpan4.0 and EDGE (max) performed worse when predicting multi-allelic data. In contrast, the EDGE (LGN) and EDGE (LGN+IP-MS) presentation models exhibited improved average precision when predicting multi-allelic data. Thus, inclusion of the LGN in the presentation model improves predictive power on multi-allelic data.

Reference is now made to FIGS. 8C and 8D, which respectively show a precision recall curve and a true positive rate v. false positive rate plot of the EDGE (LGN+IP-MS) presentation model in comparison to BERTMHC and NetMHCIIpan4.0. Specifically, FIGS. 8C and 8D both show that the EDGE (LGN+IP-MS) presentation model outperforms both the BERTMHC and NetMHCIIpan4.0 pipelines. Specifically, the EDGE (LGN+IP-MS) presentation model achieved an area under the precision-recall curve of 76.68%, whereas BERTMHC and NetmHCIIpan4.0 each achieved lower area under the precision-recall curve values of 62.12% and 53.11%, respectively. Additionally, the EDGE (LGN+IP-MS) presentation model achieved an area under the true positive rate v. false positive rate curve of 92.25%, whereas BERTMHC and NetMHCIIpan4.0 each achieved lower AUC values of 88.3% and 82.13%, respectively.

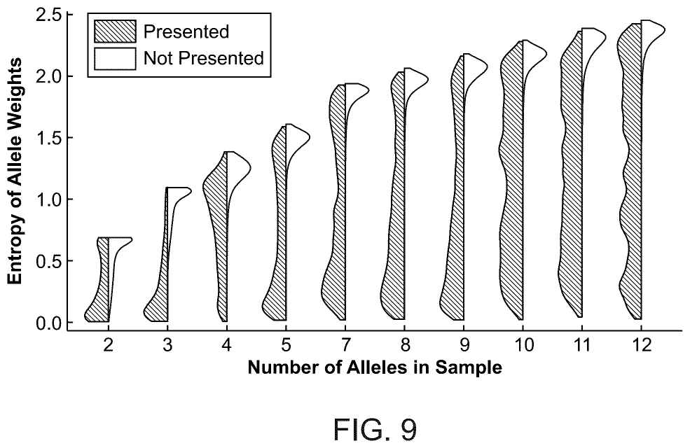

FIG. 9 shows how uniform the allele weights of the learned genotype network (LGN) are in presented and unpresented data for all allele counts. In particular, when an epitope is presented, the LGN places increased weight on individual alleles in comparison to when an epitope is not presented. This is illustrated in FIG. 9 as a decrease in entropy, which is a measure of how uniform a distribution is, in presented samples compared to nonpresented samples indicates that the attention layer is learning useful information. Entropy is calculated as:

$$Entropy = - {\sum }_{x ∈ X} p ( x ) log p ( x )$$

Here, entropy is maximized on a uniform distribution whereas a biased situation would result in low entropy.

### Example 2: EDGE Presentation Model Generates Predictions that are Correlated with Immnogenicity

Publicly available data were collected from two personalized mRNA vaccine studies with class II restricted epitopes and ELISPOT response data. Immunogenic response in the two personalized mRNA vaccine studies was correlated with the predicted score of the EDGE presentation model (referred to herein as the “EDGE score”).

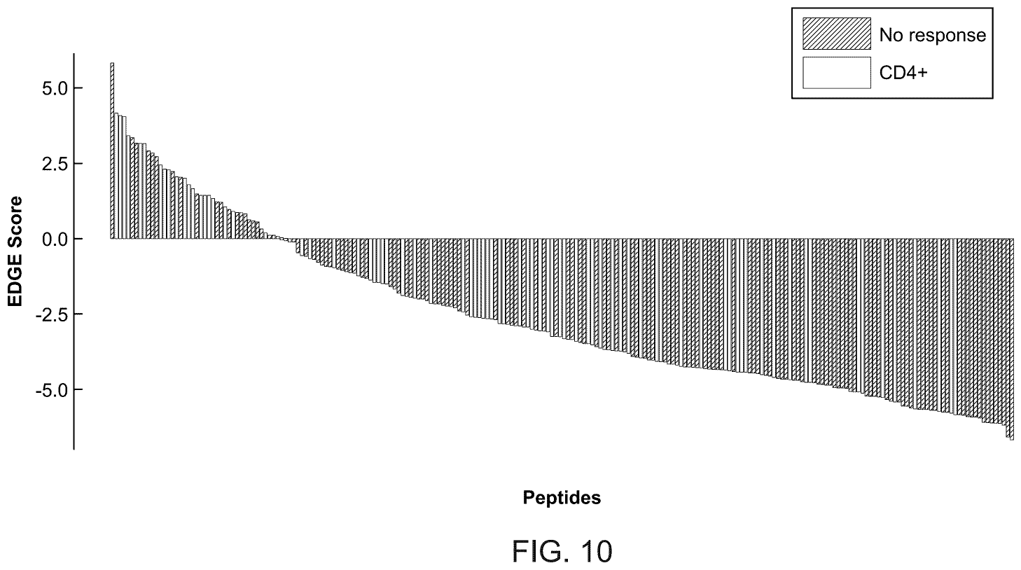

Reference is now made to FIG. 10, which shows correlation of the predicted score and immunogenic response. Specifically, as shown in FIG. 10, the EDGE score was significantly predictive of immunogenic response, particularly for scores greater than zero. This indicates that the disclosed EDGE presentation model is not merely predictive for presentation of epitopes, but is further correlated with an immunogenic response likely triggered by the presentation of the presented epitopes.

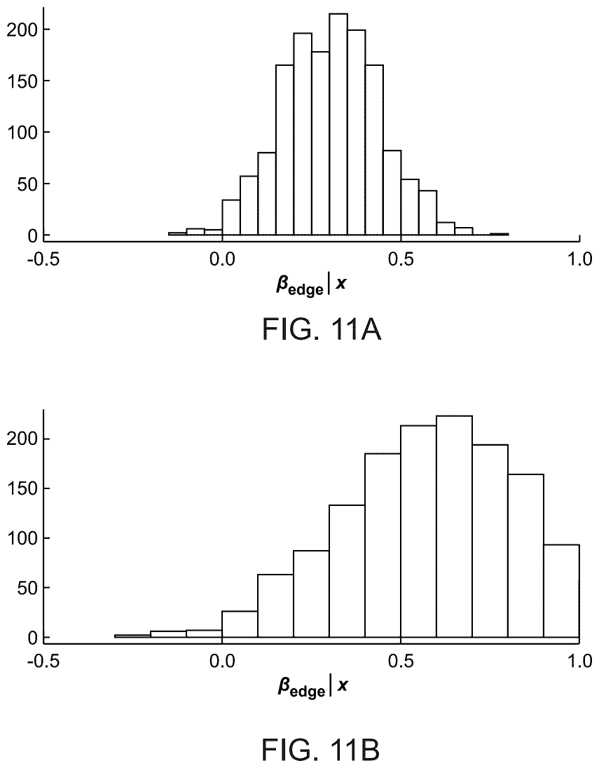

FIGS. 11A and 11B further show posterior distributions of logistic coefficients. These demonstrate that the disclosed pipeline is predictive of immunogenicity in personalized mRNA vaccines. In particular, the posterior predictive distribution allows us to assess the uncertainty in the relationship between EDGE score and immunogenic potential and determine plausible thresholds for relevant EDGE scores.

### Example 3: EDGE-II Delivers State-of-the-Art HLA Class II Presentation Predictions

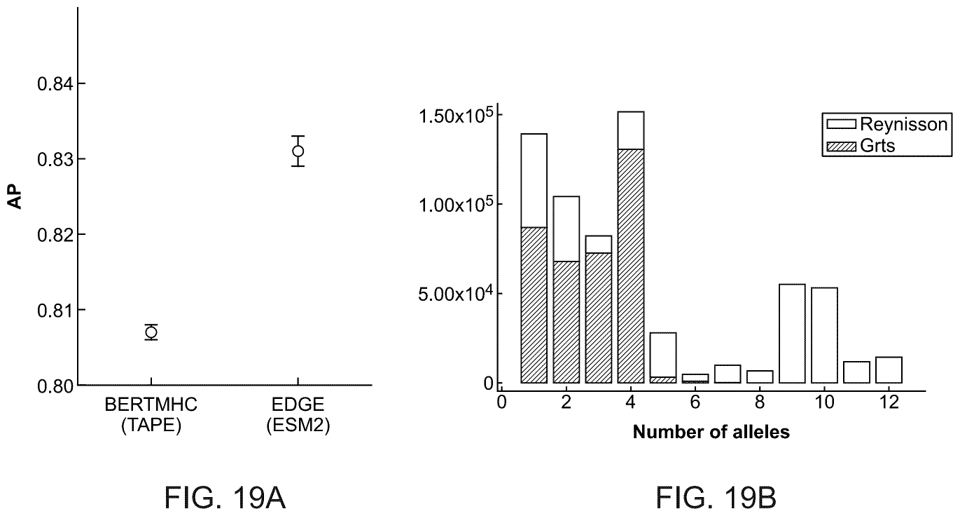

Recent advancements in machine learning have enabled development and use of pre-trained protein large language models (PLLM) for a variety of biological inference tasks because these models embed the complex physicochemical and structural information of protein sequences^{3,4}. Fine-tuning the parameters of a PLLM for inference tasks in protein biology, rather than starting from random initialization, generally increases performance of predictive models⁵. BERTMHC² is a HLA class II presentation model that utilizes a prior PLLM, TAPE⁶, and demonstrated improved predictions over NetMHCIIpan4.0¹. Herein describes a sequence-only class II algorithm, named EDGE-II, using the ESM2 PLLM⁷. ESM2 was benchmarked for class II presentation prediction performance compared to the TAPE model used in BERTMHC. A model using either TAPE (38M parameters, 512 hidden dimensions) or the closest version of ESM2 (35M parameters, 480 hidden dimensions) was built and trained. Evaluation occurred on the well-known Reynisson et al. class II presentation data^{1,8}. The predictive model trained on ESM2 significantly outperformed TAPE (FIG. 19A).

After integration of ESM2, improvement of the deconvolution of multi-allelic immunopeptidomics data, typically derived from HLA genotyped human tissues or cell lines, was pursued. Multi-allelic data contributes to the of training HLA presentation algorithms for class I and class II, and such data utilizes a mathematical method to produce a single presentation probability across the multiple alleles. Within the context of HLA presentation, the technology described herein refers to this process as allele deconvolution. Prior class II methods enforced heuristics such as iterative pseudo-labeling⁸ or per-allele maximal score deconvolution². To determine if EDGE-II could learn to deconvolute multiallelic data based off the alleles present in the sample and the putative peptide being considered, a Learned Allele-weighting Network (LAN) was implemented. The LAN operates on the latent epitope:allele embeddings from ESM2 rather than on the final output of prediction probabilities, as in BERTMHC, NetMHCIIpan4.0, and existing class I models (FIG. 7). In the LAN, the allele weights, a, are based on the complete genotype present in a sample and were calculated at inference time by incorporating a specialized auxiliary attention network into EDGE-II. Since EDGE-II can predict for any HLA allele with a known sequence, unsupervised manifold regularization was implemented to discourage the LAN from overfitting to HLA sequences in the training dataset.

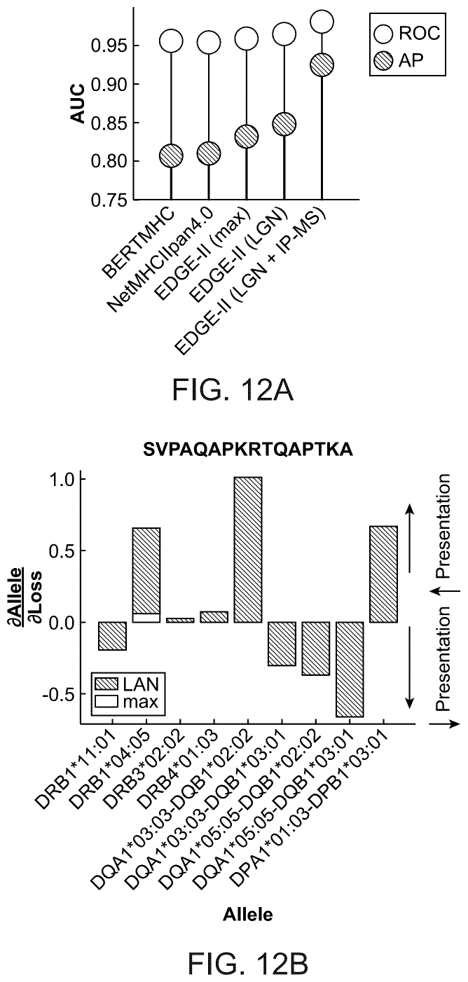

Inclusion of the LAN in EDGE-II resulted in Average Precision (AP)=0.848±0.001, ROC-AUC=0.963±0.003 when trained and validated on the Reynisson et al. dataset, which was a substantial gain in performance over the maximal output probability deconvolution approach in BERTMHC (AP=0.807±0.001, ROC-AUC=0.954±0.0004), NetMHCIIpan4.1 (AP=0.810±0.001, ROC-AUC=0.956±0.0004), or EDGE-II with maximal output probability deconvolution instead of LAN (AP=0.831±0.002, ROC-AUC=0.958±0.0003; FIG. 12A). Since standard MA data is generated using pan-specific antibodies, experiments leveraging immunoprecipitation (IP) with HLA-DR/DP/DQ-specific antibodies to generate mass spectrometry (MS) presentation data (referred to herein as “IP-MS data”) were conducted to determine if this would lead to performance improvements in EDGE-II. Inclusion of these data as an intermediate training step between SA training and MA deconvolution training led to substantial improvements in ROC-AUC and AP compared to the same model architecture that did not include these data, with EDGE-II achieving a final validation AP=0.925±0.005 and ROC-AUC=0.981±0.004 (FIG. 12A). Next, EDGE-II was applied to an independent test set of mass spectrometry data curated by Cheng, J., Bendjama, K., Rittner, K. & Malone, B. BERTMHC: improved MHC-peptide class II interaction prediction with transformer and multiple instance learning. Bioinformatics 37, 4172-4179 (2021), when evaluating BERTMHC against NetMHCIIpan4.0. Results indicate that EDGE-II (AP=0.684±0.007, ROC-AUC=0.898±0.004), trained on the same dataset as BERTMHC and NetMHCIIpan4.0 and excluding the aforementioned IP-MS data, substantially outperformed BERTMHC (AP=0.621±0.006, ROC-AUC=0.883±0.002) and NetMHCIIpan4.0 (AP=0.531±0.003, ROC-AUC=0.821±0.003). With the inclusion of the IP-MS data while training, EDGE-II achieved a test AP of 0.713±0.004 and ROC-AUC of 0.900±0.002 (FIG. 8C).

The LAN was further characterized to understand its behavior during training and prediction. A feature of the LAN was that all alleles present in a sample contributed to parameter updates, whereas only the allele with maximal probability did in prior methods. To demonstrate this, random sampling of a positively labeled peptide, SVPAQAPKRTQAPTKA, from the validation data and isolation of the gradient of each allele when calculating the EDGE-II score using either maximal score deconvolution or the LAN was performed (FIG. 12B). The gradient and its direction (positive or negative) indicated if a peptide:allele is associated with presentation or not. Notably, the LAN allowed for alleles in a MA sample to have both positive gradients (peptide:allele more likely to present) and negative gradients (peptide:allele less likely to present) during training. Therefore, the LAN of EDGE-II updated the presentation likelihood in either direction for each allele in a MA sample even while predicting a single presentation likelihood for the full class II genotype.

Furthermore, it was found that the deconvolution function learned by EDGE-II was more complex than standard aggregation techniques such as maximum, mean, or sum functions. A measure of distribution uniformity, entropy, was leveraged to quantify the behavior of attention weights over the alleles in a sample after training EDGE-II with the LAN. Results indicated that the a, distribution entropy for presented multiallelic samples was significantly lower than non-presented multiallelic samples for any number of alleles present in a sample (p<<0.05; T-Test), indicating that the LAN placed increased weight on specific alleles when predicting true positive peptides from a set of alleles (FIG. 9). However, the distribution of entropy values was more diffuse as the number of alleles in a sample increased. Conversely, the a, distributions for negatively presented samples were approximately uniform (FIG. 9). This indicated that each allele in a negative sample contributed approximately equally to EDGE-II's final prediction.

### Example 4: EDGE-II Predicts Immunogenic Neoepitopes Presented by HLA Class II in a Personalized Neoantigen Cancer Vaccine

To determine if EDGE-II scores based only on sequence data are predictive of immunogenicity for class II presented epitopes, a series of experiments were conducted. Incorporation of additional covariates such as gene expression and cleavage signatures generally improved prediction performance on immunopeptidomics data. However, given the inherent biases present in immunopeptidomics training data, it was less clear how much this benefit would extend to the prediction of immunogenicity of the presented epitopes. For HLA class II, the MARIA algorithm⁹ demonstrated significant association between high scoring class II peptides and immunogenicity post vaccination and underscored that existing sequence-only models performed relatively poorly on this task. Limiting the types of data required for making predictions, however, makes the algorithm more widely applicable. In addition, it has been demonstrated that a non-trivial proportion of class II restricted peptides have no detectable RNA expression, of which a significant amount were extracellular proteins⁹.

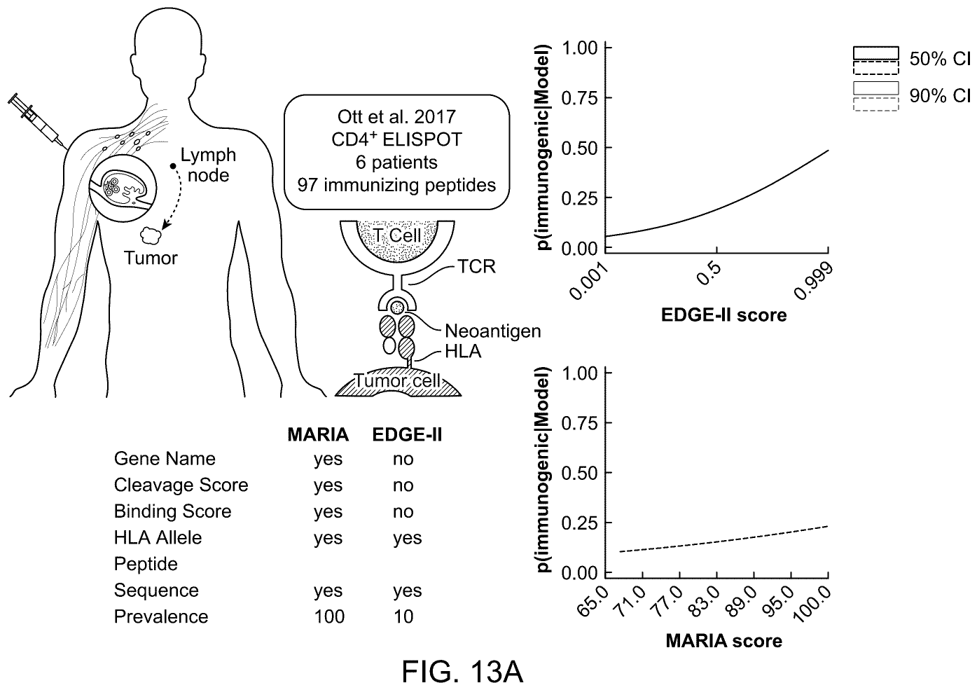

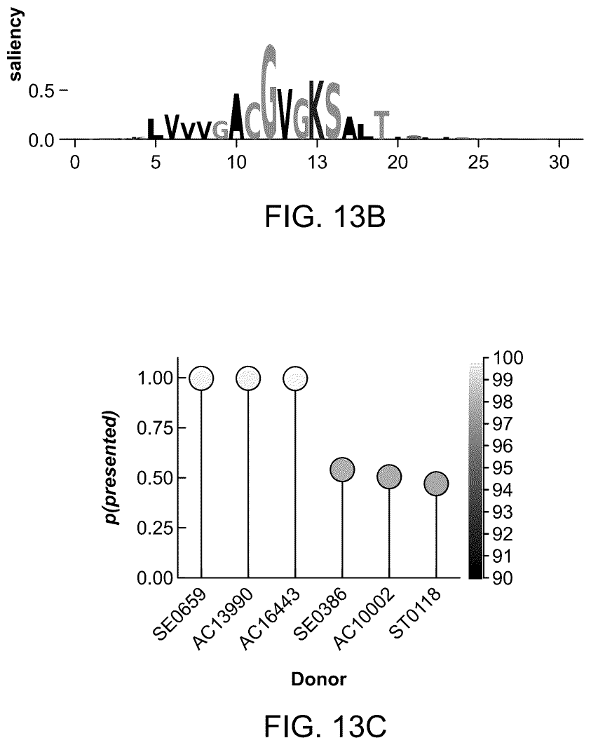

ELISpot response data were collected with full HLA class II genotypes from Ott et al.¹⁰ to determine if sequence only EDGE-II scores were positively associated with immunogenicity in the context of a personalized cancer vaccine. Ott and colleagues generated personalized mutanome targeted peptide vaccines with the goal of inducing CD4⁺ and CD8⁺ T cell responses in melanoma patients, from which ELISpot T cell response data were collected. As such, evaluation of the ability of EDGE-II to predict the induction of CD4⁺ T cell responses post vaccination was examined (FIG. 13A, left). For each peptide in the ELISpot response data, a sliding 15 amino acid (aa) window was implemented across the peptide and an EDGE-II score was calculated. The maximum score across the windows was then saved as the final presentation prediction for that peptide (Table 3). Next, a comparison of the predictive ability of sequence-only EDGE-II scores (logits) to MARIA scores (percentiles) using Bayesian explanatory logistic regression to calculate the conditional probability of CD4⁺ immunogenicity given the score from each model, prob(CD4⁺|score, Model), was performed.

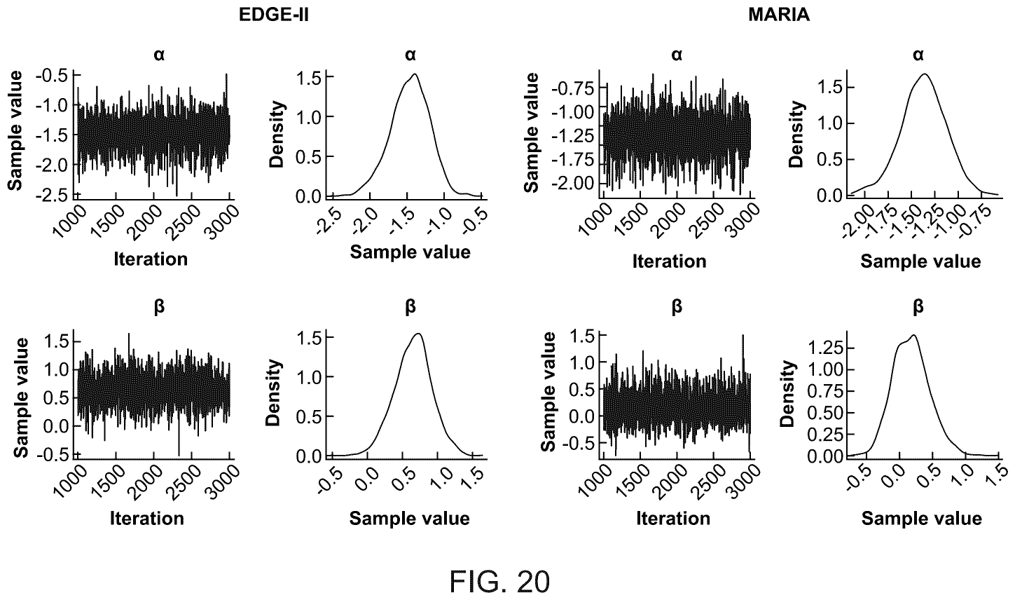

EDGE-II scores were positively associated with immunogenicity over the full dataset, with mean regression coefficient β_{edge}=0.65 and 99.1% certainty of positive association, whereas MARIA had a mean regression coefficient βₘₐᵣᵢₐ=0.18 and 72.0% certainty of positive association (FIG. 20). This difference is reflected in the predicted probability of immunogenicity between the two models (FIG. 13A, top right). EDGE-II gave 9/97 peptides a presentation probability greater than 0.99, for which the expected probability of CD4⁺ immunogenicity was 0.41±0.12 90% CI (FIG. 13A, top right). In contrast, the lowest scoring 10% of peptides in the dataset had a predicted probability of immunogenicity of 0.05±0.04 90% CI (FIG. 13A, top right). MARIA yielded a lower range between the highest and lowest 10% of peptides in the dataset, and with greater uncertainty for low scoring peptides (FIG. 13A, bottom right). The top 10% of peptides as scored by MARIA had an expected probability of generating a CD4⁺ T cell response of 0.23±0.07 90% CI while the lowest 10% had an expected probability of 0.11 with a wide asymmetric 90% credible interval of (−0.06, +0.39). On the high quality PCV CD4⁺ immunogenicity data, EDGE-II demonstrated superior specificity despite using only sequence features. Furthermore, this analysis revealed that low scoring EDGE-II peptides have low likelihood of generating a CD4+ T cell response, whereas the highest scoring peptides were on average 8× more likely to generate a CD4+ T cell response.

### Example 5: EDGE-II Addresses the Gap in the Prediction of KRAS G12C Class II-Presented Peptides

Driver mutations in the KRAS gene are prevalent in cancer and targeting KRAS G12C mutations with small molecule inhibitors had provided clear therapeutic benefit to patients, leading to the approval of sotorasib and adagrasib^{11,12}. Acquired resistance mutations are, however, observed within months of treatment initiation and pose a challenge for providing long-term clinical benefit to patients with cancer. An “off-the-shelf” cancer vaccine targeting KRAS mutations could potentially offer an alternative therapeutic approach for providing more durable benefit to these patients^{13,14}. KRAS G12 mutants, including KRAS G12C, have recently been demonstrated to generate a class II-dependent immune response. Despite these observations, previous publicly available HLA class II models have yielded low presentation scores for KRAS G12C epitopes across all alleles. NetMHCIIpan4.0, for example, predicted no binders for any epitope containing the G12C mutation (Table 4). Further explanation of the headers of Table 4 are as follows, “Pos” refers to residue number (starting from 0), “MHC” refers to the MHC molecule name, “Peptide” refers to the amino acid sequence, “Of” refers to the starting position offset of the optimal binding core (starting from 0), “Core” refers to the binding core register, “Core_Rel” refers to the reliability of the binding core, expressed as the fraction of networks in the ensemble selecting the optimal core, “Identity” refers to the annotation of the input sequence, if specified, “Score_EL” refers to the eluted ligand prediction score, “% Rank_EL” refers to the Percentile rank of eluted ligand prediction score, “Exp_bind” refers to if the input was given in PEPTIDE format with an annotated affinity value (for benchmarking purposes), and “BindLevel” which refers to strong binder (SB) and weak binder (WB). A peptide was identified as a strong binder if the % Rank was below the specified threshold for the strong binders and the peptide will be identified as a weak binder if the % Rank was above the threshold of the strong binders but below the specified threshold for the weak binders.

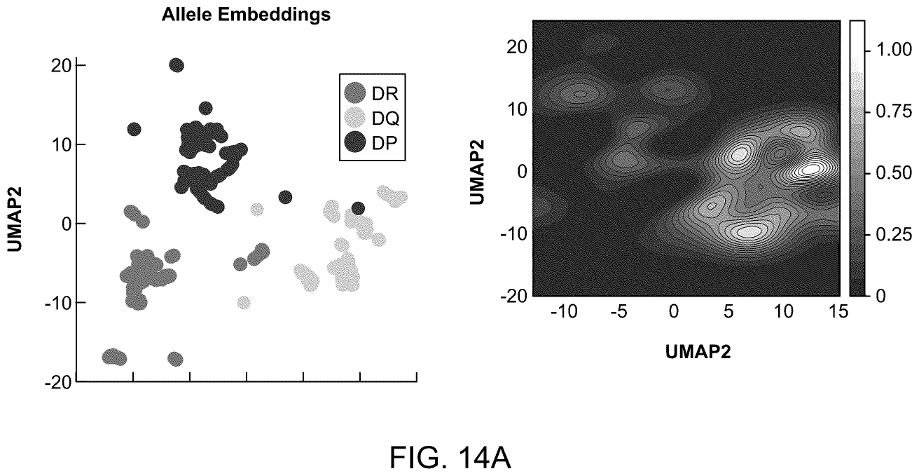

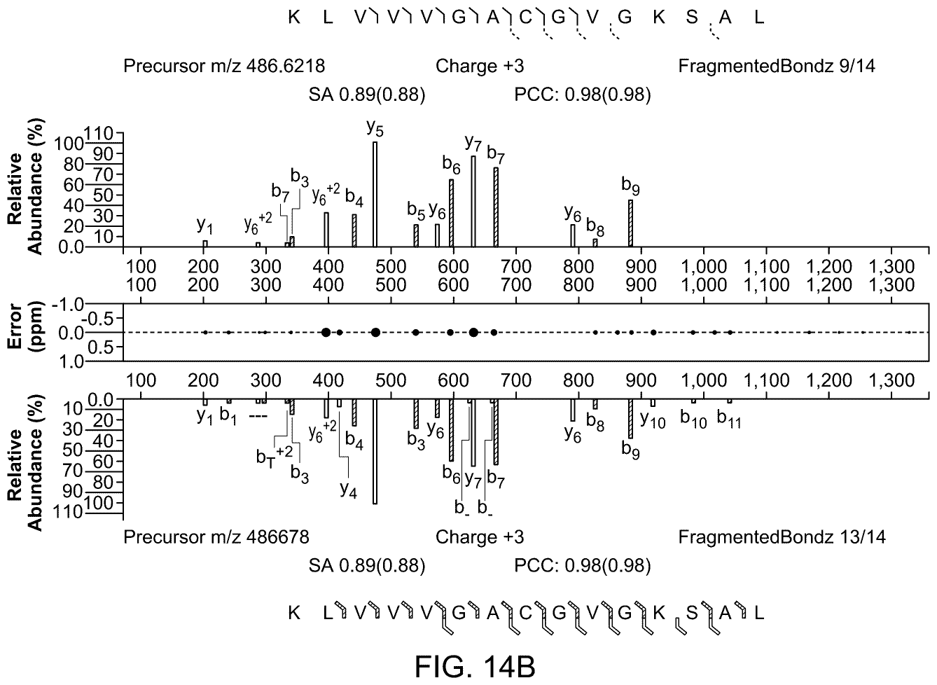

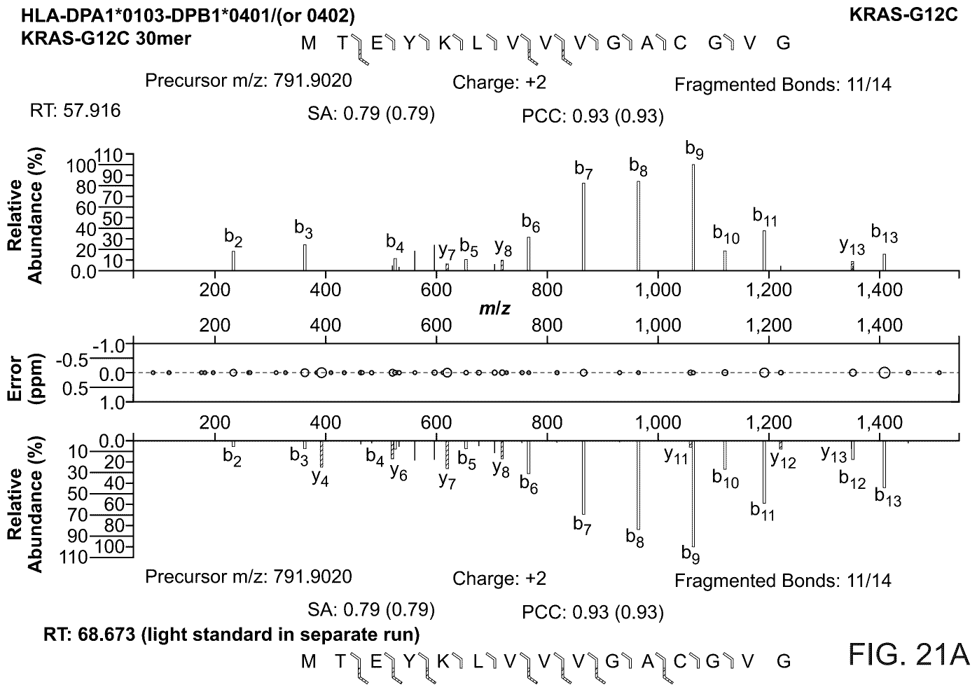

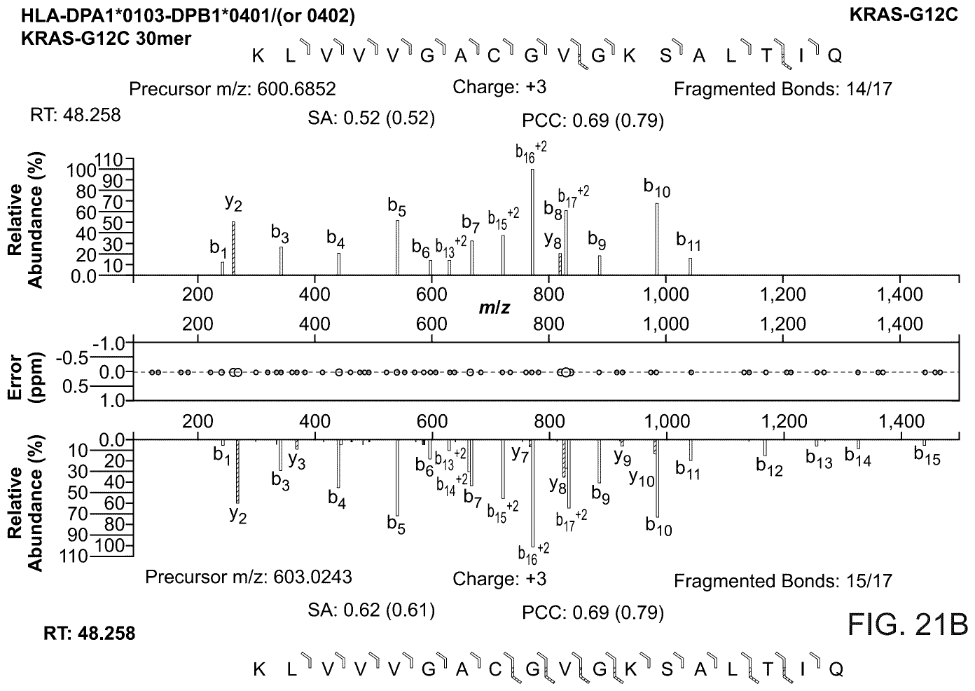

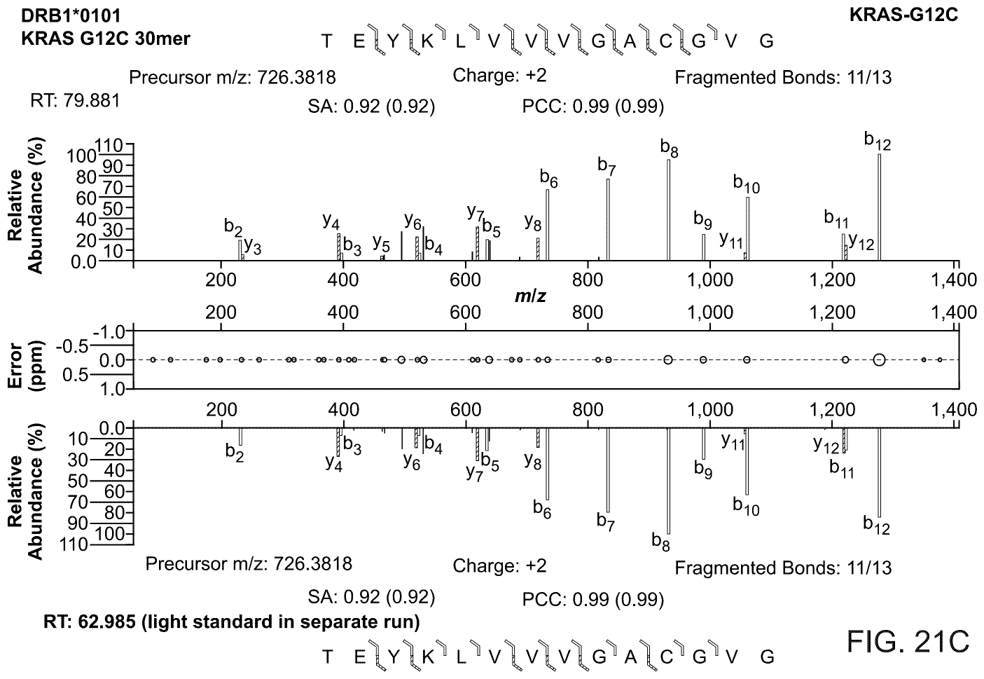

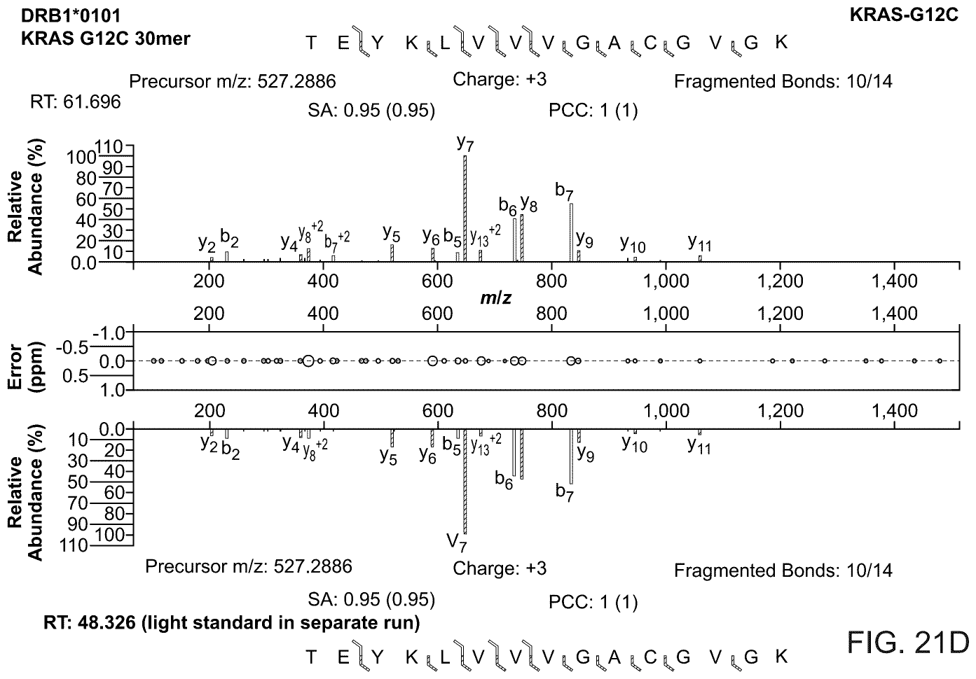

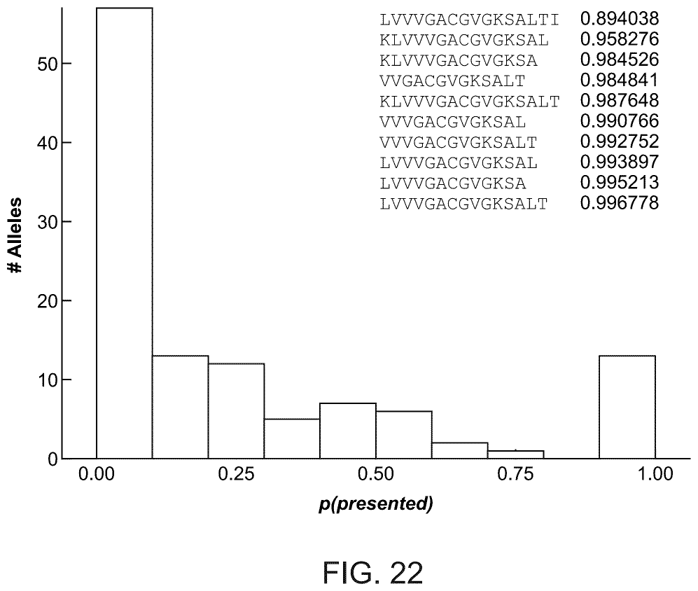

Prediction of the presentation of 15-mer KRAS G12C peptides across 1,067 unique HLA alleles by EDGE-II revealed a specific motif found in positive presentation prediction (FIG. 13B). DQ alleles were identified to be more strongly associated with high EDGE-II scores as compared to DP and DR alleles (FIG. 14A). A peptide, KLVVVGACGVGKSAL, containing the motif (FIG. 13B), was found to be presented via DQ by mass spectrometry (FIG. 14B). Peptides were also found to be presented by DP and DR alleles (FIG. 21A, FIG. 21B, FIG. 21C, and FIG. 21D). Previously published work shows that naïve TCR repertoires from healthy donors recognize KRAS G12 mutant class I epitopes (Bear, Blanchard et al., 2021)¹⁹. EDGE-II was leveraged for the prediction of KRAS G12C peptide presentation from 6 healthy donors. Five healthy donors had an allele for which the EDGE-II score exceeded p=0.5, and all 6 scores were greater than the 95^{th} percentile for that allele (FIG. 13C). Therefore, whether EDGE-II predicted peptides could be recognized by naïve T cells in these 6 healthy donors was investigated.

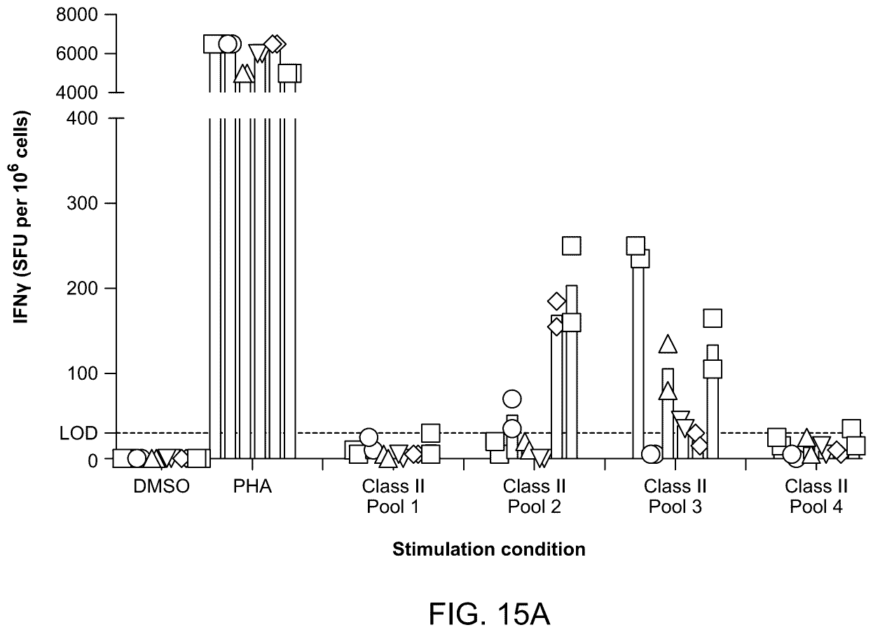

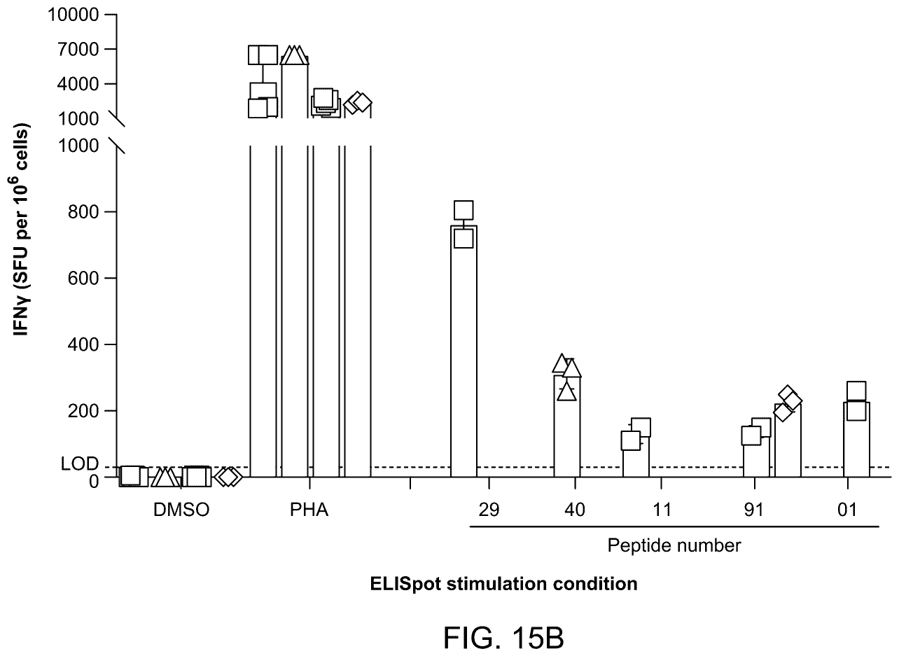

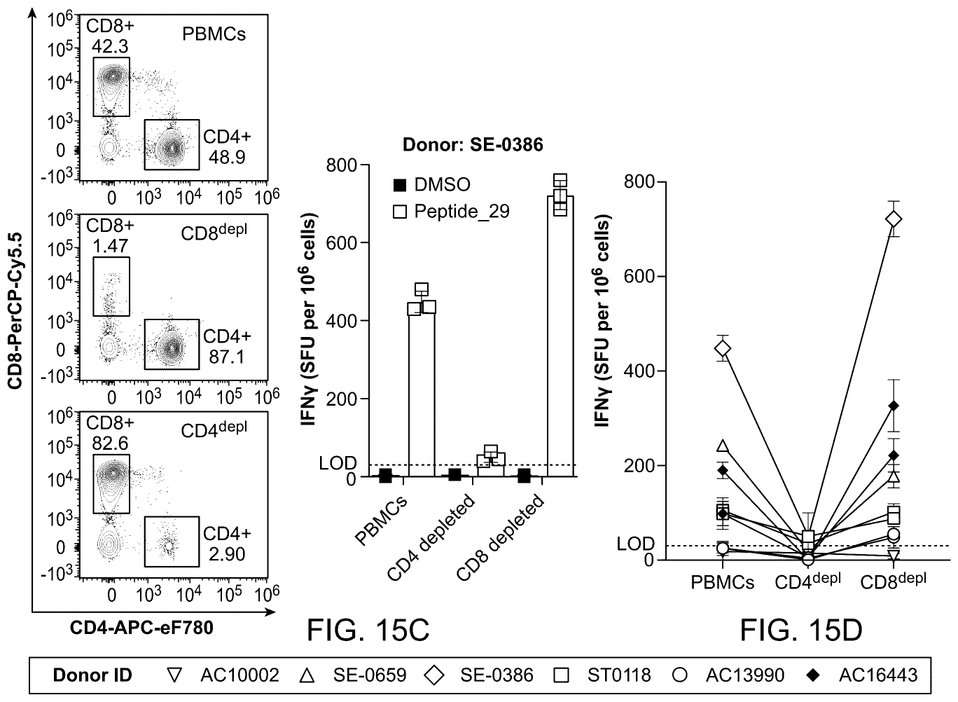

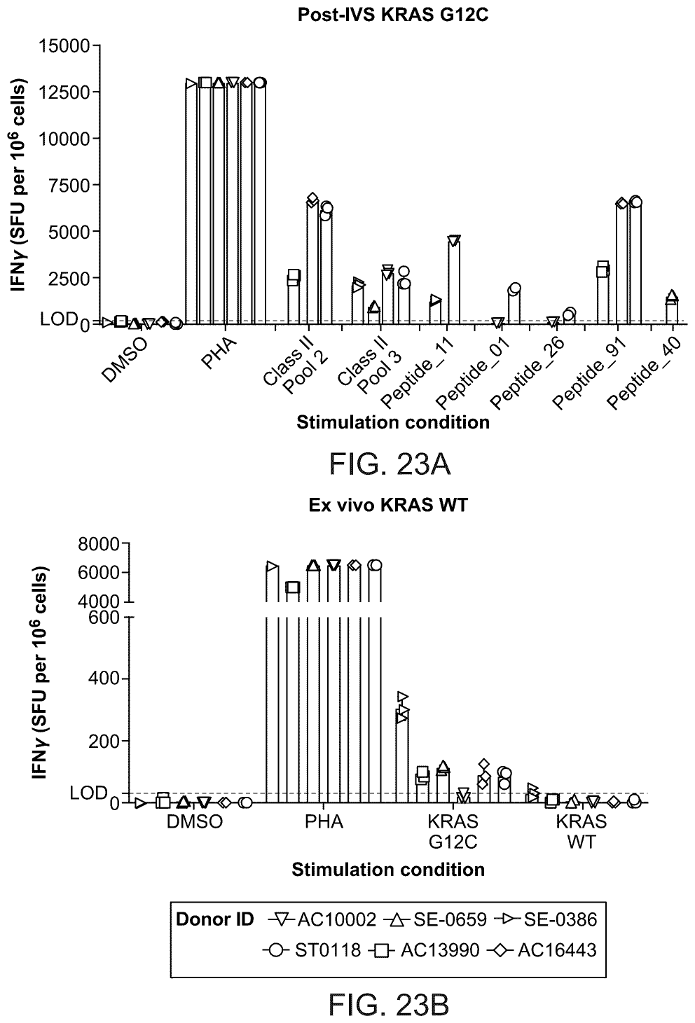

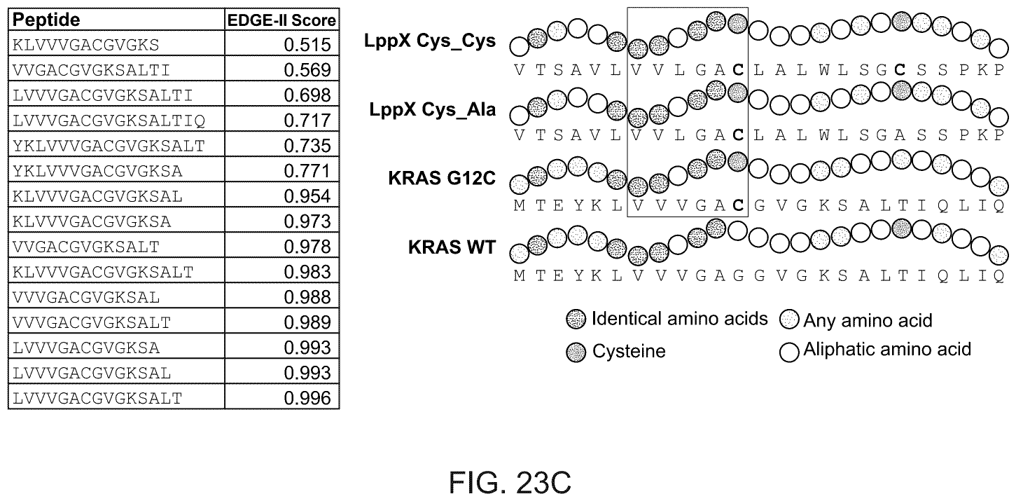

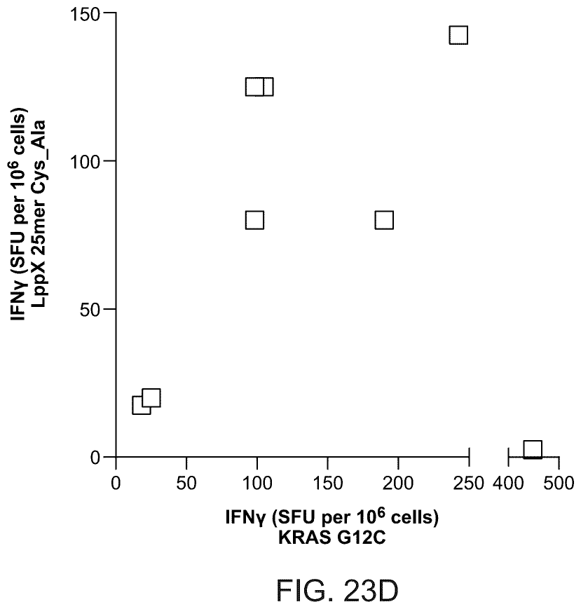

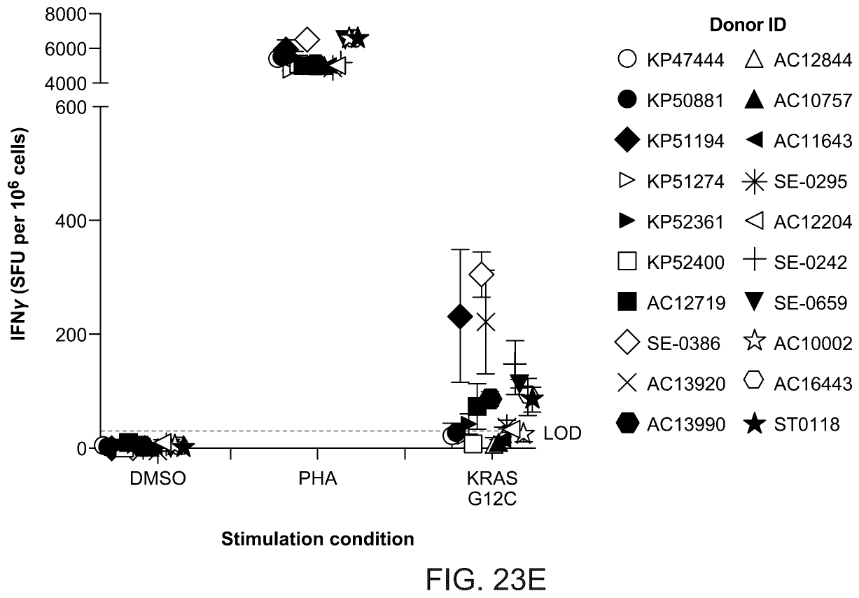

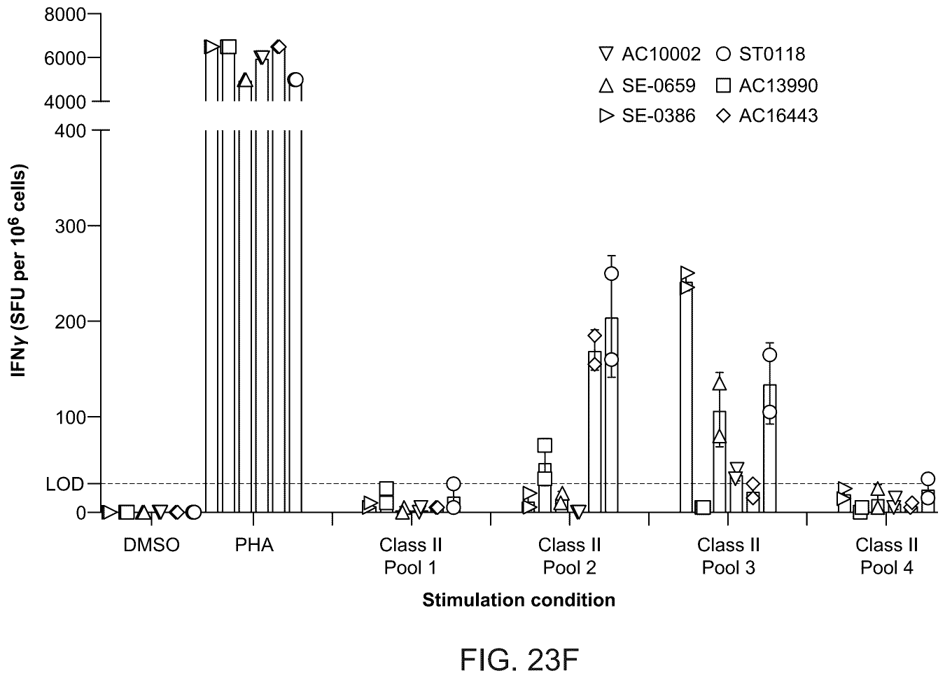

KRAS G12C epitopes predicted by EDGE-II(FIG. 13B, FIG. 23A, FIG. 23B, FIG. 23C, FIG. 23D, FIG. 23E and FIG. 23F) were tested for immunological responses (Table 5). T cell responses against overlapping peptide (OLP) pools containing KRAS G12C epitopes ranging from 10-20 amino acids in length (10mers to 20mers; Table 6) were assessed via ex vivo IFNγ ELISpot assay. All six donors screened had KRAS G12C-specific T cell responses detectable ex vivo; 2 donors showed a marginal response above LOD (>30 SFU/10⁶ cells; donors AC13990 and AC10002), and 4 donors showed positive responses >100 SFU/10⁶ cells (donors SE-0659, SE-0386, AC16443 and ST-0118; FIG. 15A). KRAS G12C-specific T cells in donors with marginal ex vivo responses were readily expanded via a short antigen-specific in vitro stimulation (IVS) culture (FIG. 23A), and deconvolution to single peptides ex vivo or post-IVS confirmed functional responses to EDGE-II predicted class II epitopes across all 6 donors (FIG. 15B, FIG. 23A). The responsive peptides and EDGE-II predicted peptides both consistently contained the “VGACGVGK” motif, being present in 15/15 EDGE-II peptides and all responsive peptides (Table 7). T cell responses were specific for KRAS G12C compared to KRAS WT (FIG. 23B), suggesting a role for the Cysteine at position 12 for the G12C-specific T cell responses identified in the healthy donors.

The concept of harnessing T cell responses to mimotopes from microbial pathogens in anti-cancer immunity has been considered for some time^{15,16}, as such testing of whether KRAS G12C-specific T cell responses in healthy donors was likely driven by cross-reactive responses derived from a pathogen mimotope was investigated. Amino acid sequence alignment using NCBI protein Blast® revealed sequence homology between KRAS G12C and a bacterial lipoprotein (LppX) present in both Escherichia coli and Mycobacterium tuberculosis, which had previously been shown to elicit class II-dependent T cell responses in Bacille Calmette-Guérin (BCG)-vaccinated healthy donors¹⁷ (FIG. 23C). 11/15 of the top scoring EDGE-II predicted peptides, using an a priori threshold of p=0.5, contained the LVVVGAC motif that shares similarity with the LppX LVVLGAC sequence (FIG. 23C). Mimotope-specific T cell responses were assessed using a 25mer representing the relevant LppX peptide sequence with a second Cysteine at position 20 replaced by an Alanine (LppX_Cys_Ala) to reduce cysteinylation of the peptide causing interference with peptide-MHC (pMHC) and T cell receptor (TCR) binding (FIG. 23C). Mimotope-specific T cell responses corresponded with KRAS G12C-specific T cell responses in 5/6 donors, supporting the hypothesis that cross-reactive T cell responses may be driving the KRAS G12C responses in these healthy donors (FIG. 23D). These donors, therefore, provided a model system to further interrogate KRAS G12C-specific T cell responses. While depletion of CD8+ T cells from PBMCs did not change the KRAS G12C T cell responses observed, depletion of CD4+ T cells reduced G12C-specific T cell responses measured by IFNγ ELISpot 2-20 fold in donors with positive PBMC responses, confirming class II-driven presentation and recognition of KRAS G12C epitopes in these donors (FIGS. 15C and 15D).

### Example 6: KRAS G12C-Specific CD4+ T Cells in Healthy Donors are Polyfunctional, and Kill Class II-Matched Target Cells Presenting a KRAS G12C Class II Epitope

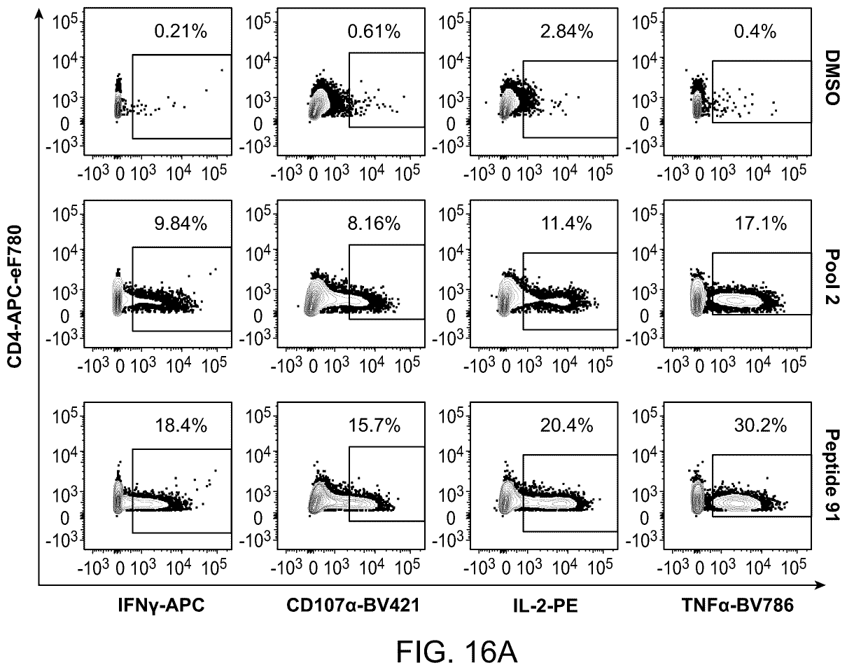

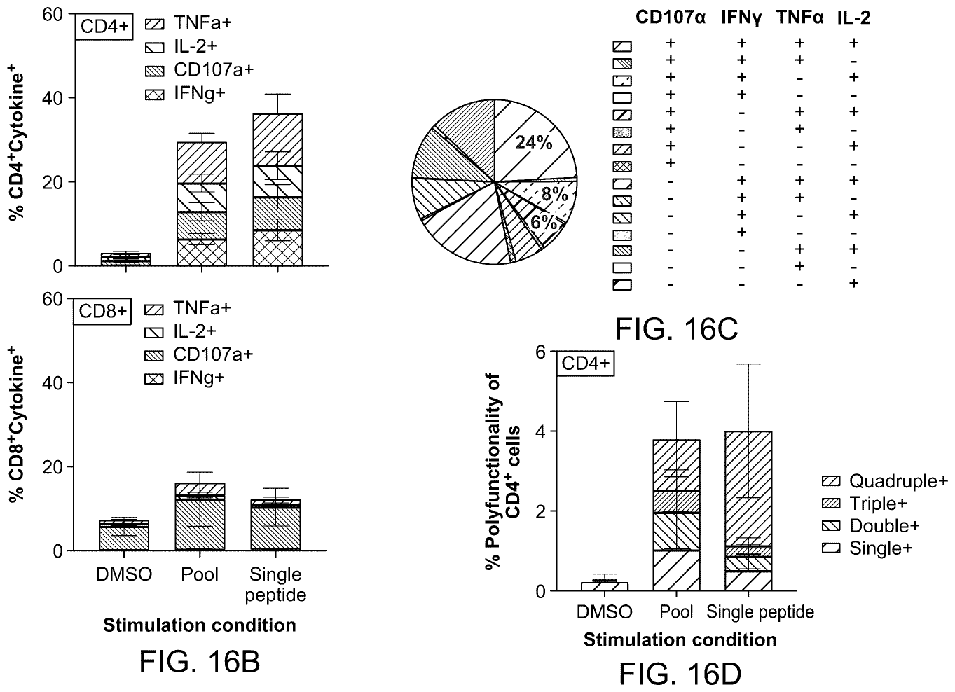

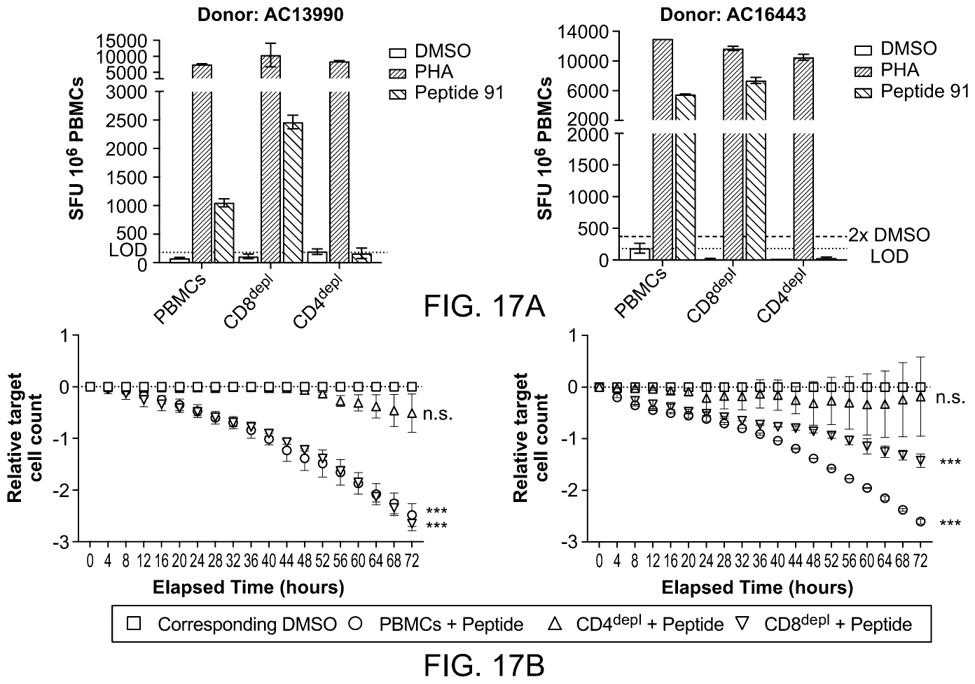

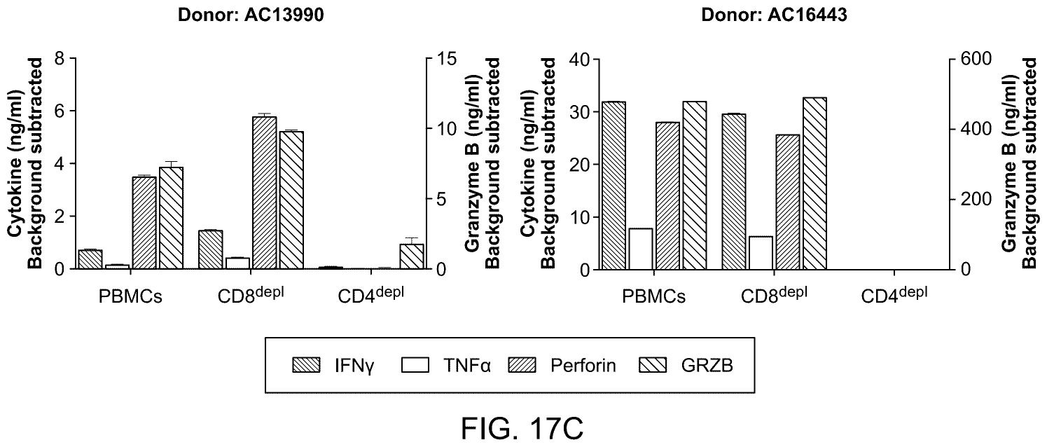

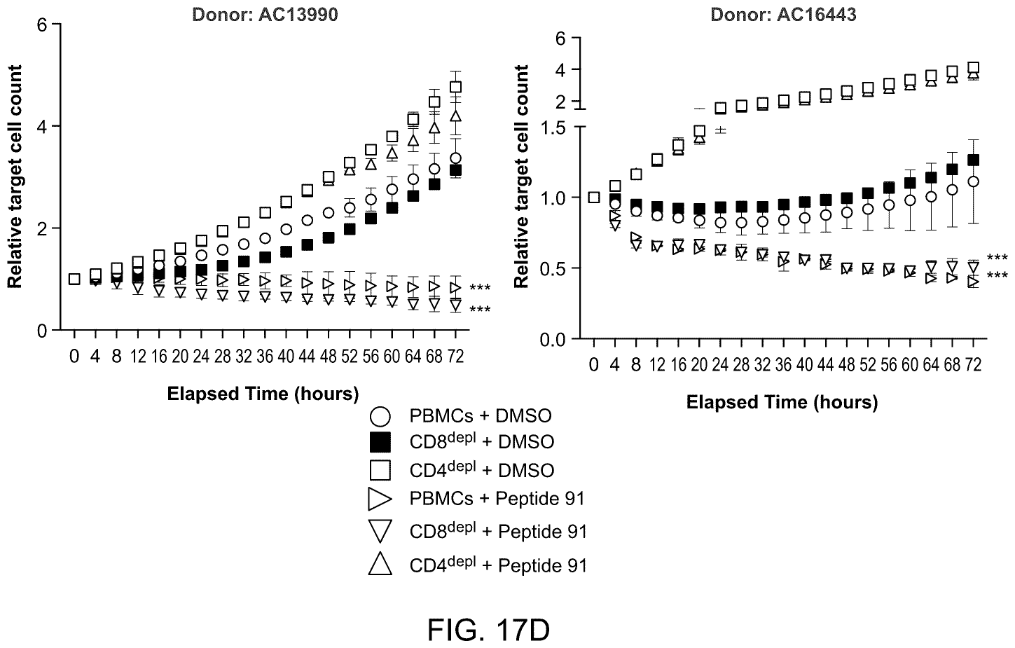

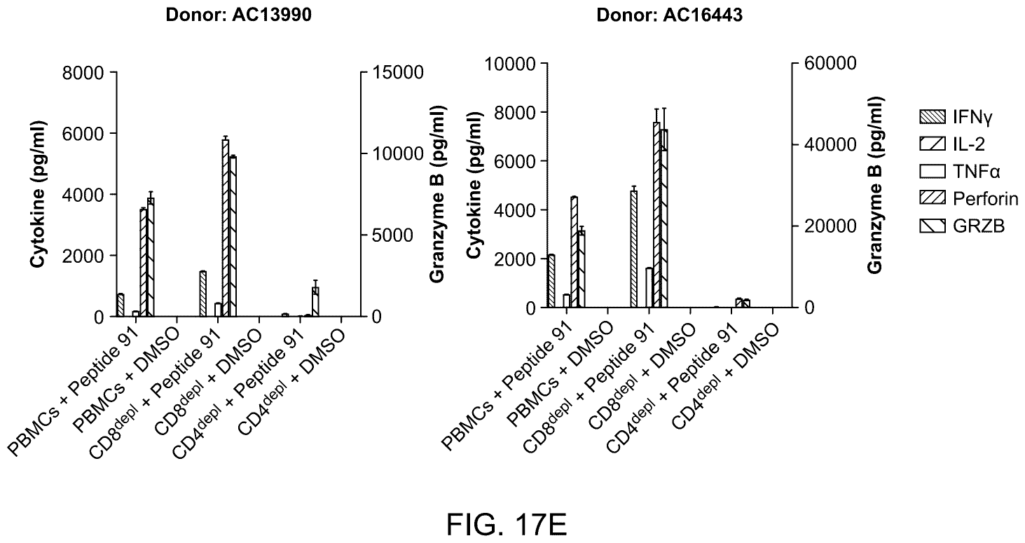

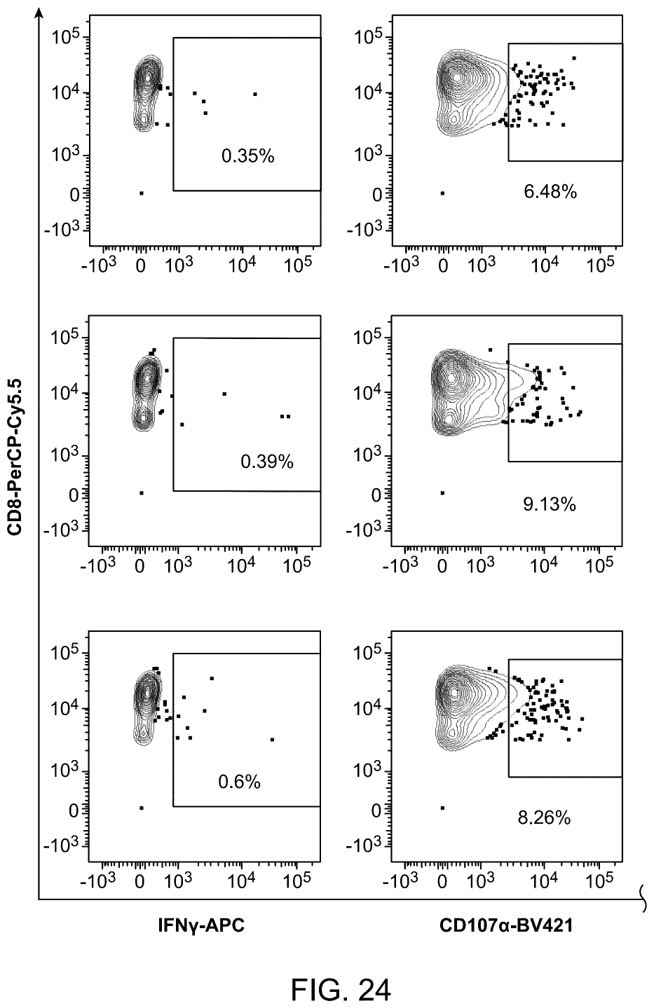

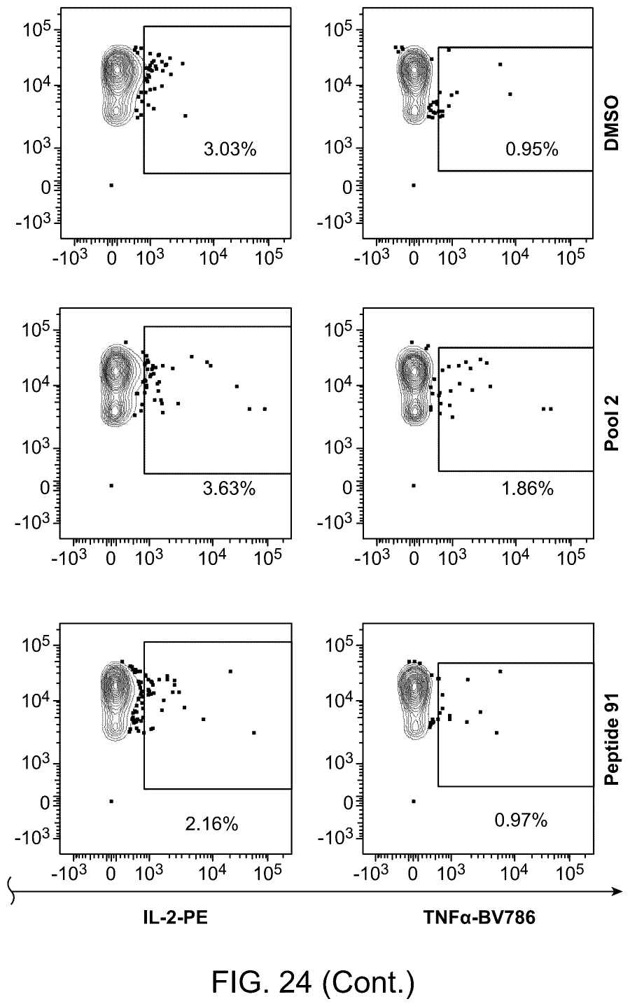

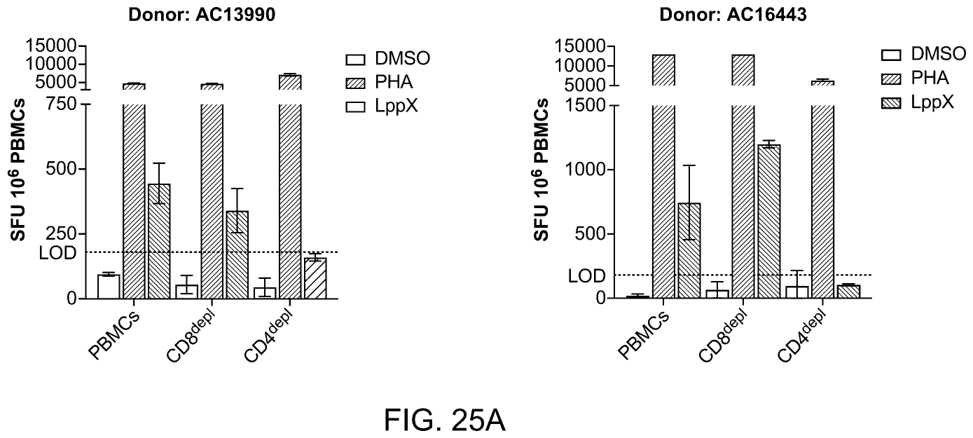

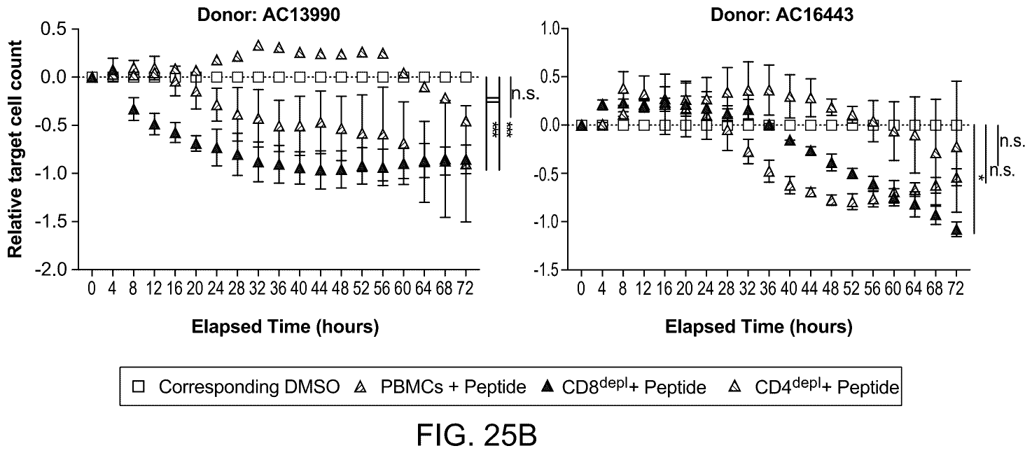

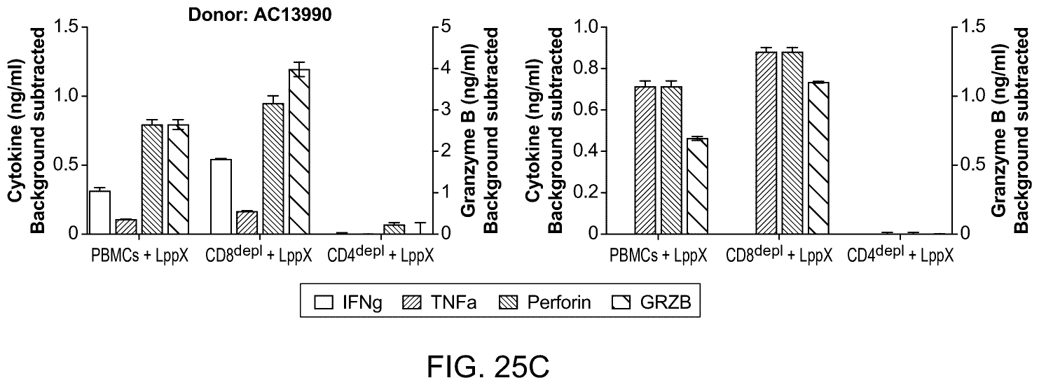

After confirming that KRAS G12C-specific T cell responses in healthy donors were class II-dependent, functionality beyond IFNγ production was assessed via intracellular cytokine staining (ICS). Stimulation with either OLP pools or donor-relevant single peptides resulted in increased production of IFNγ, degranulation marker CD107α, interleukin (IL)-2, and tumor necrosis factor (TNF)-a compared to DMSO control in CD4+ T cells, with CD8+ T cell responses not changing significantly over DMSO background (FIG. 16A, FIG. 16B, FIG. 24). Boolean gating confirmed the presence of quadruple-, triple-, and double-positive polyfunctional CD4⁺ T cells (FIG. 16C and FIG. 16D), suggesting a potentially cytotoxic effector phenotype of G12C-specific CD4⁺ T cells from these healthy donors. Cytotoxic potential of G12C-specific CD4⁺ T cell responses was assessed in two donors with post-IVS responses to a single identified peptide >1,000 SFU/10⁶ cells (AC13990 and AC16443, Peptide_91) and available class II-matched GFP-positive K562 target cells (Table 5) via IncuCyte® assay. Depletion of CD4⁺ or CD8⁺ T cells after IVS expansion confirmed CD4⁺ T cell-driven responses to G12C Peptide_91 in both donors via post-IVS ELISpot assay (FIG. 17A). In both donors, co-culture of whole PBMCs resulted in significant reduction in Peptide_91-pulsed target cells compared to DMSO control (FIGS. 17B and 17D). In line with ex vivo and post-IVS ELISpot data, depletion of CD8⁺ T cells had no effect on Peptide_91-directed killing of target cells, suggesting no direct cytotoxic role for CD8⁺ T cells in these co-cultures. In contrast, depleting CD4⁺ T cells prior to co-culture abrogated Peptide_91-specific killing of target cells for both donors (FIG. 17B). Cell supernatants collected from IncuCyte® co-cultures confirmed that secretion of cytokines Granzyme B (GRZB) and perforin in Peptide_91-stimulated conditions were only detected in the presence of CD4⁺ T cells (FIGS. 17C and 17E), further confirming CD4⁺ T cell-dependent cytotoxicity. Data from HIV infection suggested indirect CD4⁺ T cell-dependent cytotoxicity via augmentation of NK cell function upon CD4⁺ T cell help in HIV¹⁸. Repeat experiments assessing Peptide_91-specific cytotoxicity with or without CD56-dependent depletion of NK cells confirmed direct CD4⁺ T cell-driven cytotoxicity, as depletion of both NK cells and CD8⁺ T cells did not affect killing of Peptide_91-pulsed target cells in both donors (data not shown). Of note, although CD4⁺ T cell-dependent cytotoxicity towards target cells pulsed with the LppX_Cys_Ala mimotope and secretion of cytotoxic cytokines was observed in both donors, responses were less potent compared to G12C-specific responses, in line with the lower levels post-IVS LppX-specific T cell responses observed via ELISpot for PBMCs and depleted populations (FIG. 25A, FIG. 25B, and FIG. 25C).

### Example 7: Functional KRAS G12C-Specific TCRs Identified from a Patient with KRAS G12C-Positive NCSLC Map to Proliferating CD4⁺ T Cell Populations with Cytotoxic Transcriptional Profile

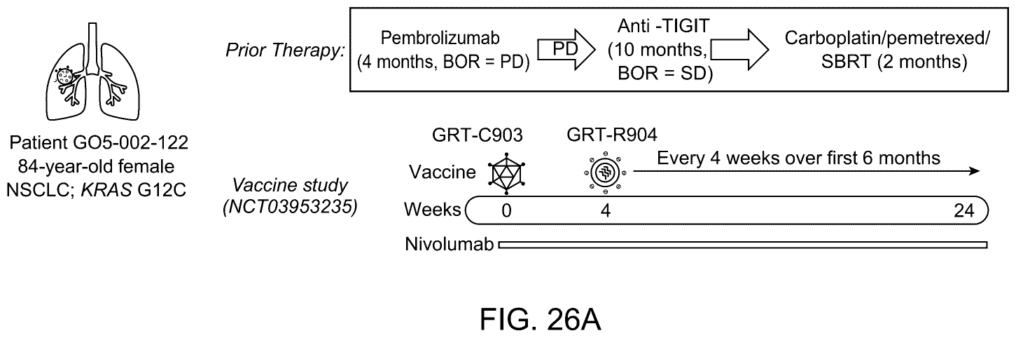

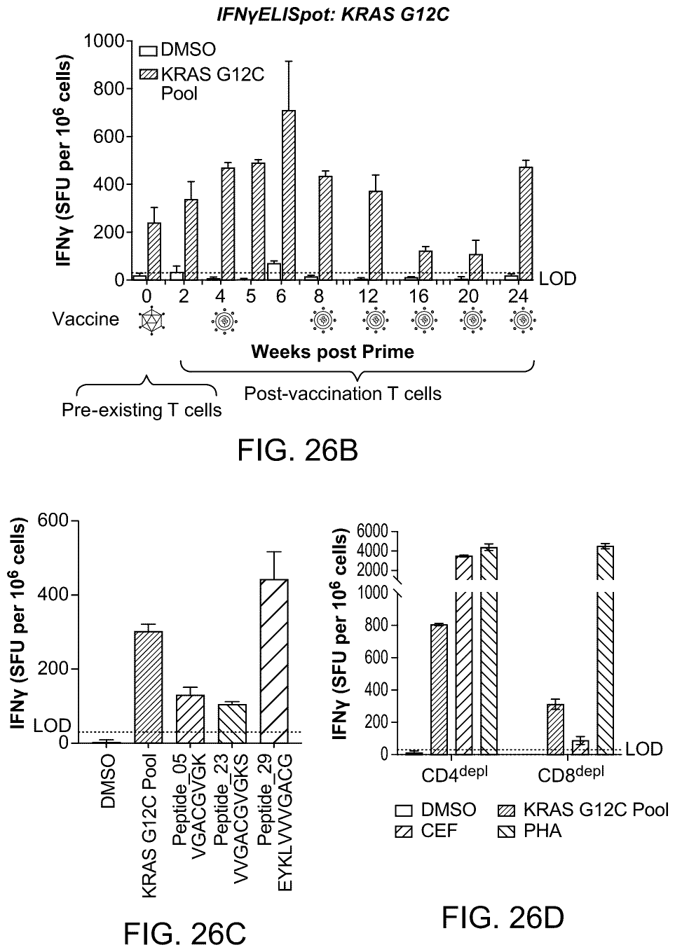

Further confirmation of the relevance of EDGE-II-predicted KRAS G12C epitopes (FIG. 23C, left) in a more clinically relevant context, such as PBMCs from a patient with a KRAS G12C-positive tumor, was desirable to understand the role of CD4⁺ immunogenic peptides in cancer. To this end, PBMC samples from a patient with KRAS-G12C-positive NSCLC (patient ID G05-002-122) were obtained. Upon further characterization, detectable KRAS-G12C-specific T cell responses and a molecular response observed in blood (reduction in KRAS G12C circulating tumor DNA) were found following administration of a KRAS-containing shared neoantigen vaccine regimen as part of a phase 1/2 clinical trial described previously (FIG. 26A; ClinicalTrials.gov ID: NCT03953235). Patient PBMC samples from available timepoints following vaccination were initially assessed for putative KRAS G12C-specific CD8⁺ T cell responses using a pool of short (8-11aa) peptides (Table 6) as part of standardized immune monitoring (FIG. 26B; ClinicalTrials.gov ID: NCT03953235), and deconvolution to single 8-11mer peptides confirmed responses to an 8mer (Peptide_05), a 9mer (Peptide_23), and an 11mer (Peptide_29) with a nested core sequence (VGACGVGK) present in all 3 responsive peptides (FIG. 26C). Of note, the 11mer (Peptide_29) was also represented in the class II long OLP pools and elicited a CD4⁺ T cell-dependent response in healthy donor SE-0386 (FIG. 15B).

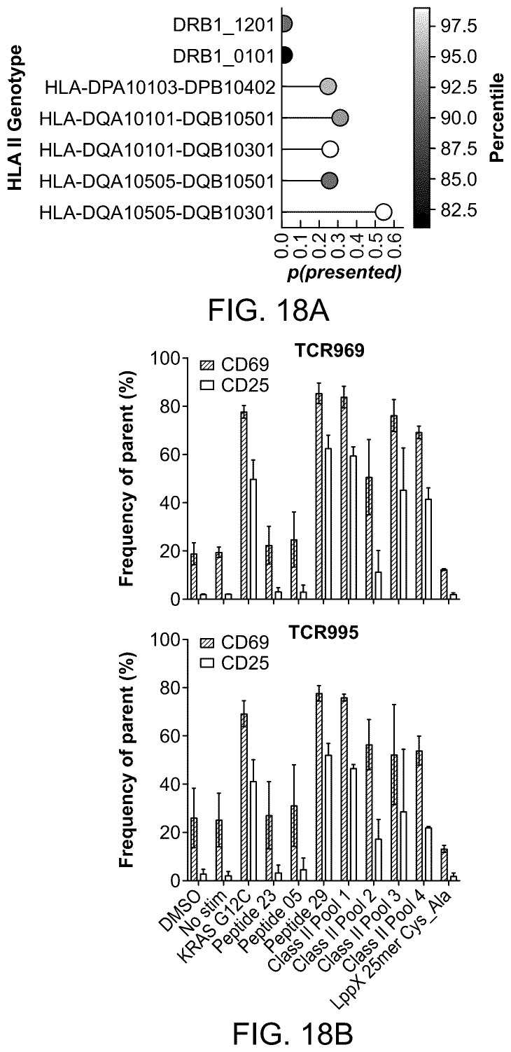

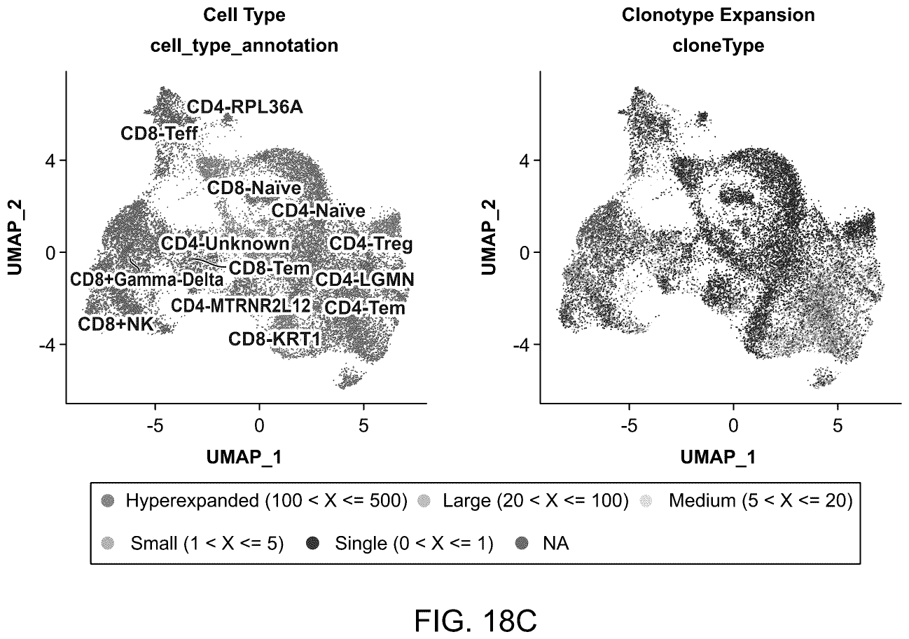

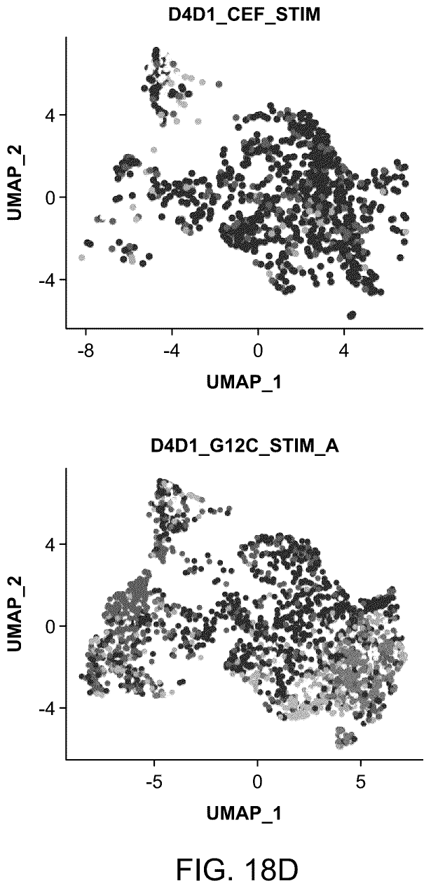

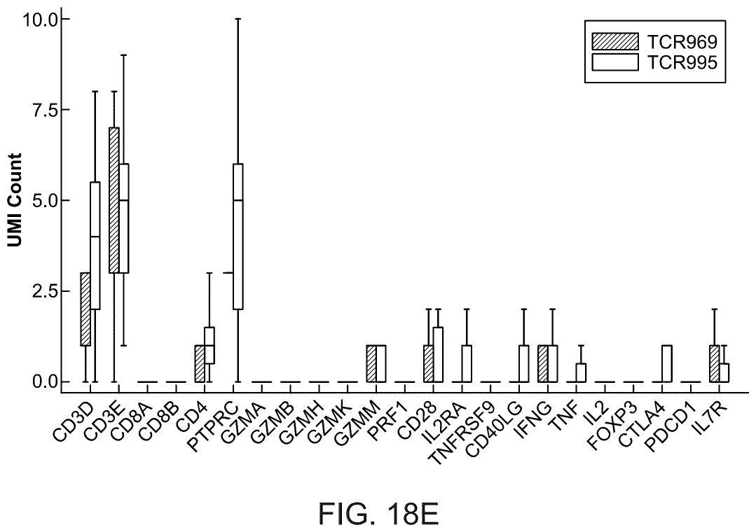

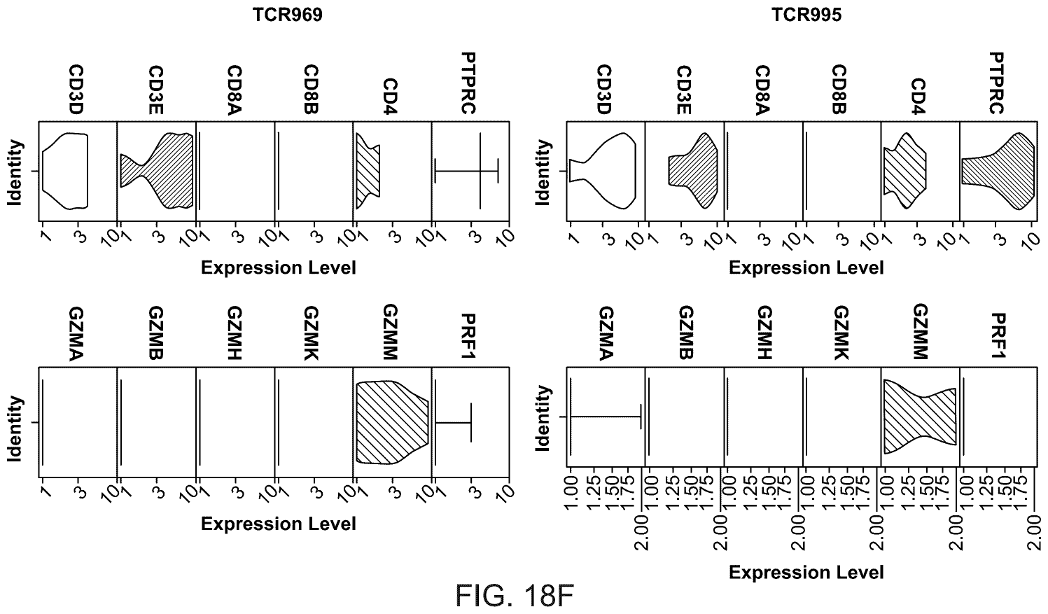

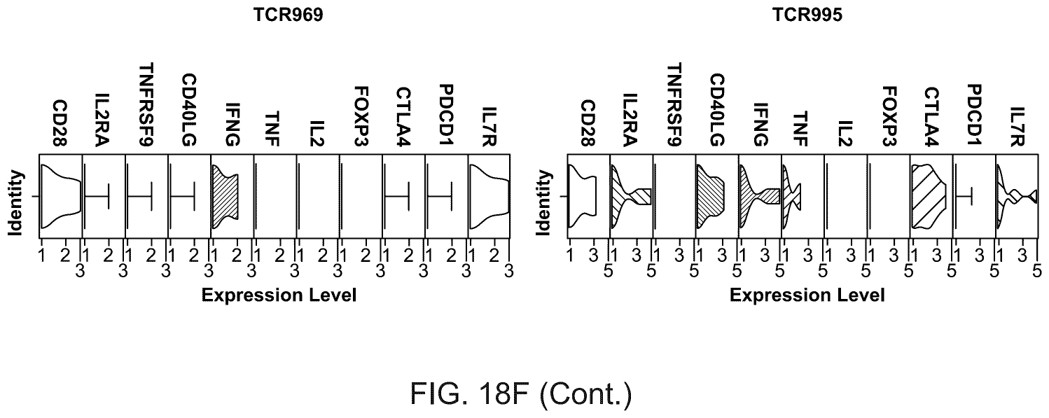

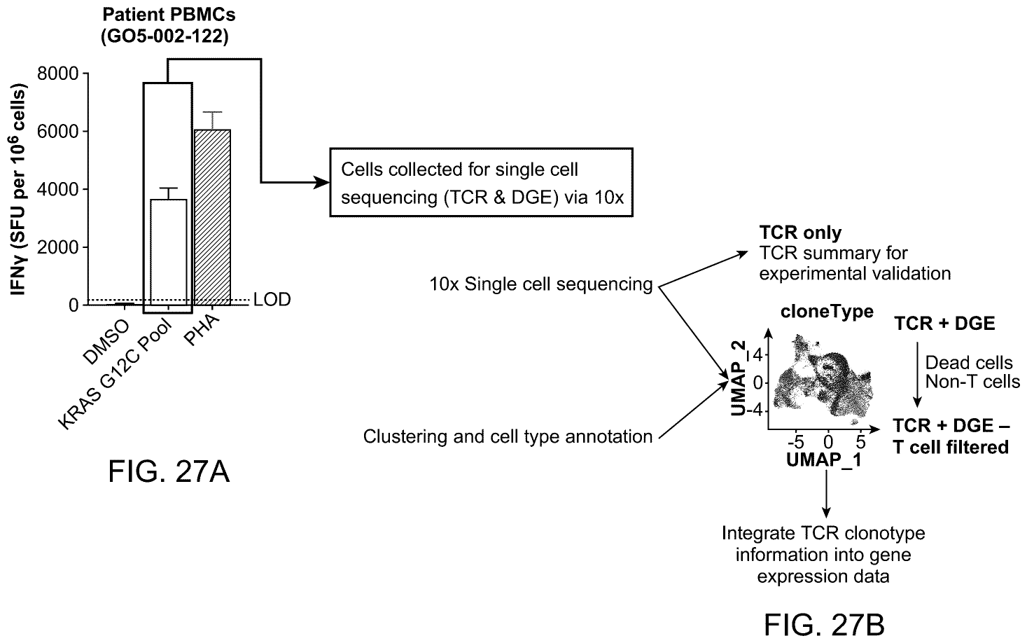

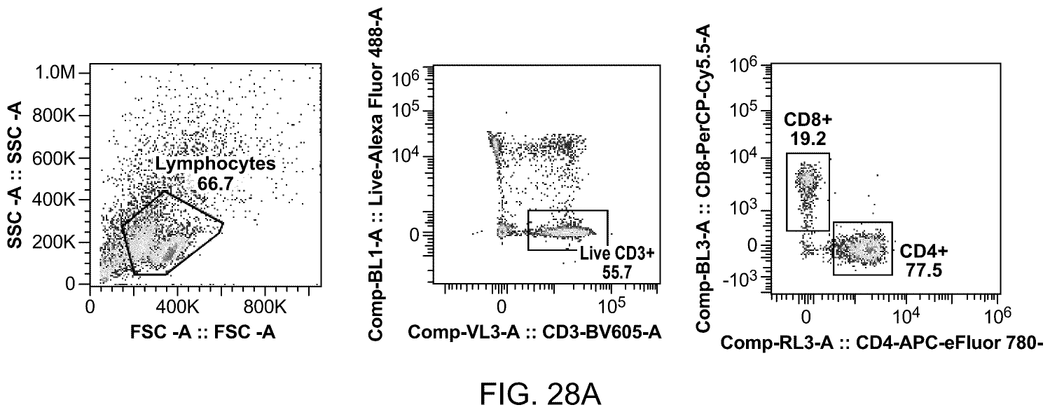

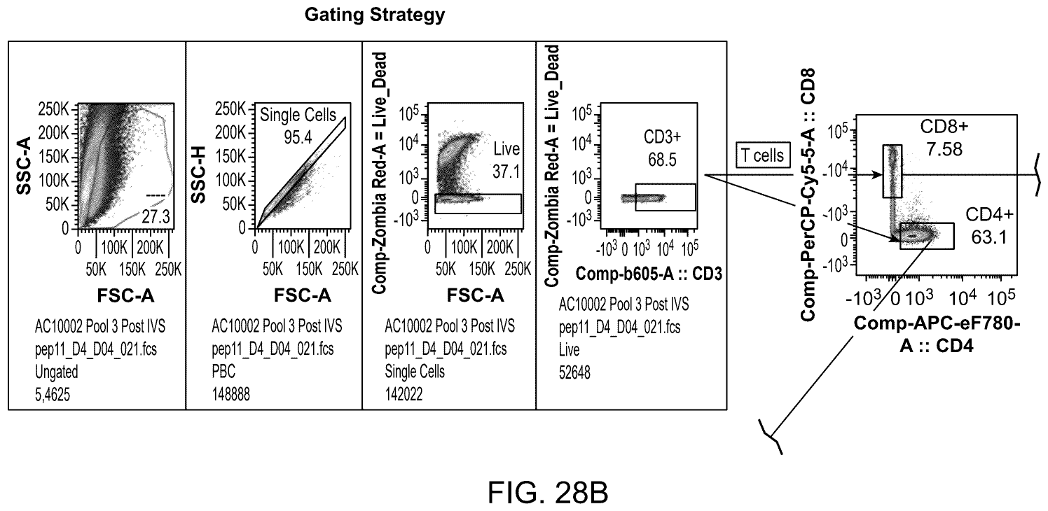

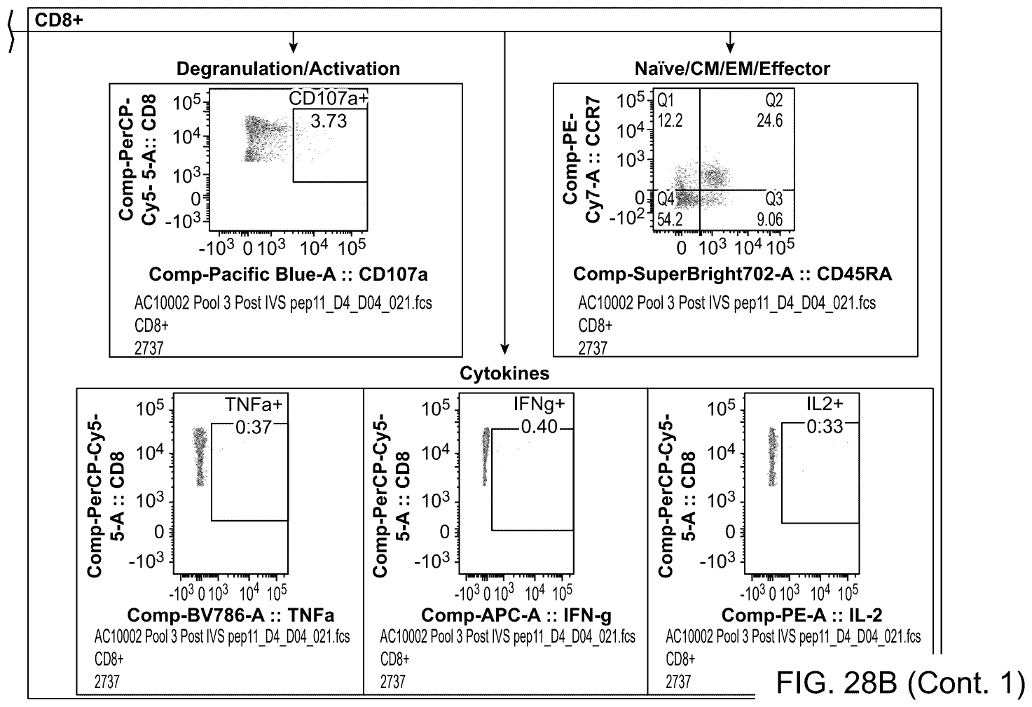

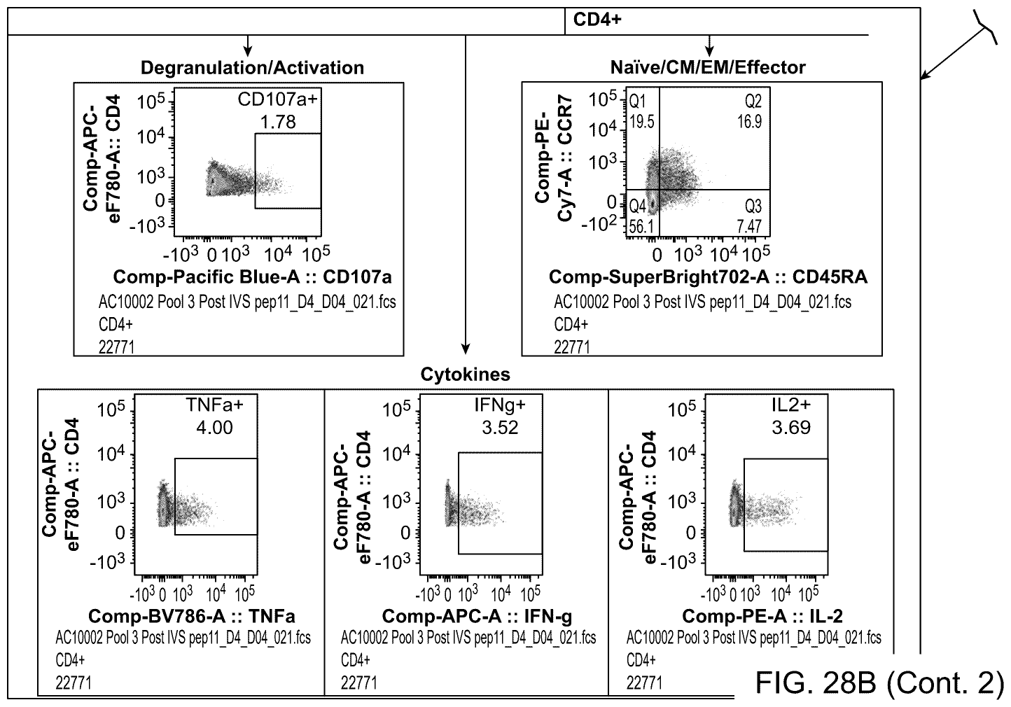

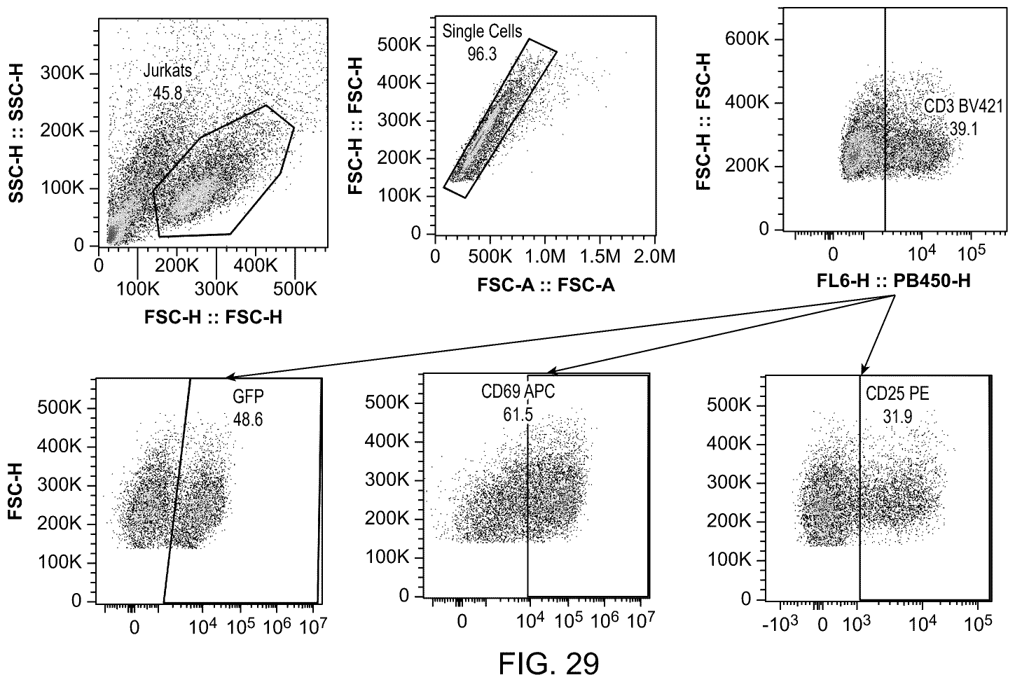

EDGE-II was used to calculate scores for each allele in the patient sample to determine the likelihood of KRAS G12C peptide presentation by HLA class II. Results indicate that EDGE-II yielded a high presentation score for the patient and particularly for HLA-DQA1*05:05-DQB1*03:01, with a score of 0.54 falling into the 99^{th} percentile for that allele (FIG. 18A). Further, the maximally scoring peptide contained the nested core sequence (VGACGVGK) present in all responsive peptides described above. Depletion of either CD4⁺ or CD8⁺ T cells from patient PBMCs revealed a mixed CD4⁺ and CD8⁺ T cell response to KRAS G12C 8mer-11mer peptide pools (FIG. 26D). Limited sample availability for patient G05-002-122 precluded further analyses using PBMC samples, however, collection of PBMC samples following ELISpot functional assays enabled paired TCR sequencing and gene expression profiling via single cell RNA sequencing (scRNASeq; FIG. 27A, and FIG. 27B). Cell line-based functional assays assessing function of the 95 most frequent TCR clonotypes via recombinant TCR (rTCR) expression were performed. Jurkat cells expressing patient G05-002-122 rTCRs co-cultured with K562 cells expressing each of the 6 patient-matched single class I alleles (Table 5) did not result in any positive responses to KRAS G12C 8mer-11mer peptide pools (data not shown). However, co-culture of rTCR-expressing Jurkat cells with class II matched KAS116 cells (Table 5) identified two TCR clonotypes (TCR969 and TCR995) that elicited increased expression of activation markers CD69 and CD25 on rTCR Jurkat cells in response to KRAS G12C peptide pools (both 8-11mer and 10-20mer pools) and KRAS G12C single peptides, but not LppX_Cys_Ala, in 2 independent experiments (FIG. 18B and FIG. 29). Importantly, rTCR functional assays confirmed responses to individual peptides (Peptide_05, Peptide_23, Peptide_29) observed in patient PBMCs via ELISpot (FIG. 26C). In order to understand which cell populations these two functional TCRs were derived from, integrative analyses combining TCR clonotype and transcriptional profiles across multiple samples from patient G05-002-122 either stimulated with KRAS G12C pools or CEF (CD8⁺ T cell-specific responses to CMV, EBV and Flu peptides) controls were performed to identify putative T cell populations (Table 8). Transcriptional clustering analyses revealed distinct CD4⁺ and CD8⁺ T cell phenotype clusters with varying degrees of TCR clonotype expansion across all samples (FIG. 18C). As expected, in control samples stimulated with viral epitopes (CEF), proliferating clonotypes predominantly mapped to a population with CD8⁺ effector memory transcriptional profiles (FIG. 18D). In contrast, in patient PBMC samples stimulated with G12C peptides, hyper-expanding clonotypes predominantly mapped to CD4⁺ populations with effector memory transcriptional profiles, although expanding cells also clustered with CD8⁺ T cell populations, in line with a mixed CD4⁺/CD8⁺ G12C-specific T cell response observed in PBMCs (FIG. 18D, FIG. 26D). The two functional TCRs identified during co-culture with class II matched cell lines (TCR969, TRC995; FIG. 18B) mapped to cell populations with a CD4⁺ cytotoxic transcriptional profiles (FIGS. 18E and 18F), which suggested the presence of G12C-specific cytotoxic CD4⁺ T cell responses in patient PBMCs.

### Example 8: Tumor and Cell Line Specimens for EDGE-II Training

Frozen tissue specimens for mass spectrometry analysis were obtained from commercial sources or hospital centers (Table 9). EBV transformed B cell lines expressing class II alleles were obtained from La Jolla Institute for Allergy and Immunology (La Jolla, CA, Table 9).

### Example 9: IncuCyte Killing Assays

Single HLA-expressing A375 NucLight™ Red cell lines were seeded in 96- or 48-well plates at concentrations of 2.5×10⁴ or 3.5×10⁴ cells per well in DMEM with 10% heat inactivated FBS. The plates were placed in the Incucyte® S3 (Essen Biosciences, Satorius) and 24h after the seeding, the effector cells were plated at a concentration of 2.5×10⁵ or 3.5×10⁵ cells per well in a 96- or 48-well plate, respectively, for an effector to target ratio of 10:1. Individual peptides (Genscript, Table 3) were added to the treated wells for a final concentration of 10 μg/ml and DMSO was used for the control wells. The plates were imaged with the IncuCyte® for up to 72h, after which the data were analyzed using the Incucyte® S3 2018 analysis software. Viability of the A375 was assessed by red cell count, and relative viability was calculated relative to DMSO co-culture control wells.

### Example 10: Identification of MHC Class II KRAS G12C Neoepitopes Peptides

Custom-made, recombinant, lyophilized KRAS G12C MHC Class II peptides were produced by Genscript (Piscataway, NJ, USA) and reconstituted at either 2 mg/ml/peptide (pools) or 5 mg/ml/peptide (single peptides) in sterile DMSO (VWR International, Pittsburgh, PA, USA), aliquoted and stored at −80° C. Peptide pools are shown in Table 10.

### IFNγ ELISpot Assay

Detection of IFNγ-producing T cells was performed by ex vivo ELISpot assay. Briefly, cells were harvested, counted and re-suspended in media at 4×10⁶ cells/ml (ex vivo PBMCs) and cultured in the presence of DMSO (VWR International), Phytohemagglutinin-L (PHA-L; Sigma-Aldrich, Natick, MA, USA), CEF peptide pool, or cognate peptides in ELISpot Multiscreen plates (EMD Millipore) coated with anti-human IFNγ capture antibody (Mabtech, Cincinnati, OH, USA). Following 18-24h incubation in a 5% CO₂, 37° C., humidified incubator, supernatants were collected, cells were removed from the plate, and membrane-bound IFNγ was detected using anti-human IFNγ detection antibody (Mabtech), Vectastain Avidin peroxidase complex (Vector Labs, Burlingame, CA, USA) and AEC Substrate (BD Biosciences, San Jose, CA, USA). ELISpot plates were allowed to dry, stored protected from light and enumerated on an AID iSpot reader (Autoimmun Diagnostika). Data are presented as spot forming units (SFU) per million cells.

### FluoroSpot Assay

A multiplexed FluoroSpot assay was performed according to manufacturer's instructions (Mabtech, Cincinnati, OH, USA) for the detection of IFNγ, IL-2 and Granzyme B producing T cells. Briefly, PBMCs were thawed and rested overnight at 2×10⁶ cells/mL. The following day, cells were harvested, counted and resuspended at 4×10⁶ cells/mL prior to plating. Pre-coated FluoroSpot plates were washed with PBS and blocked with culture media before mutation-specific peptide pools, controls (DMSO [VWR International] and Phytohemagglutinin-L [PHA-L; Sigma-Aldrich, Natick, MA, USA]), and cells were added (200,000 PBMCs/well). After incubating in a 5% CO₂, 37° C., humidified incubator for 18-24 hours, secreted IFNγ, IL-2 and Granzyme B were detected using anti-human IFNγ-BAM/anti-BAM-490, biotinylated anti-human Granzyme B/Streptavidin-550, and anti-human-IL-2-WASP/anti-WASP-650 antibody pairs followed by fluorescence enhancer. Plates were imaged and enumerated on an AID iSpot reader (Autoimmun Diagnostika). Data are presented as spot forming units (SFU) per million cells.

### CD4/CD8 Depletions

CD4 or CD8 T cells were depleted from total PBMCs using Miltenyi MACS microbeads and columns. PBMCs were labeled with anti-CD4 or anti-CD8 microbeads before being run through a MACS Column placed in a MACS Separator magnet. Labeled cells remained in the column and the negative fractions were collected as either the CD8 or CD4 enriched population. Cells were then plated at 200,000 cells/well and stimulated overnight in an IFNγ ELISpot assay or FluoroSpot assay to detect IFNγ or IL-2.

### Results

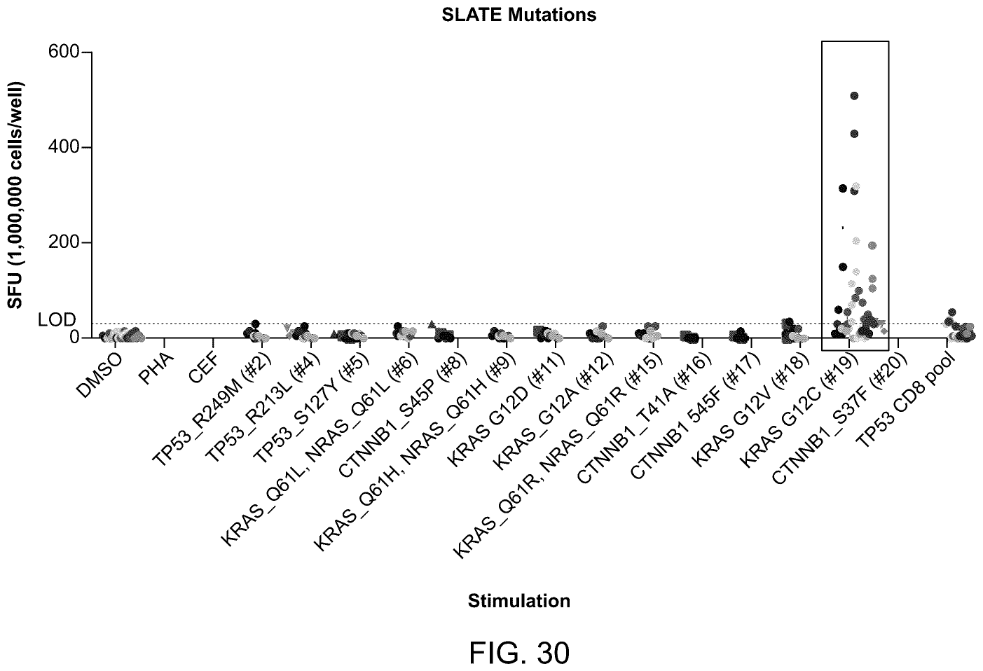

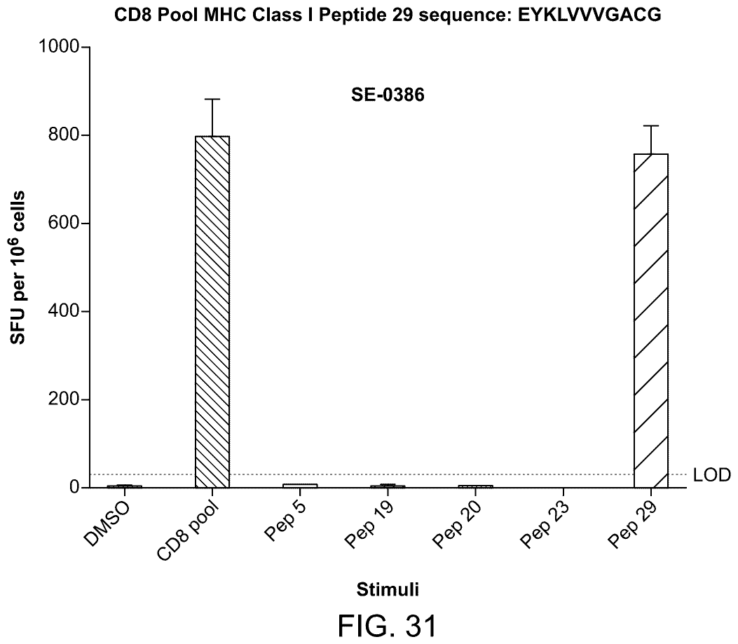

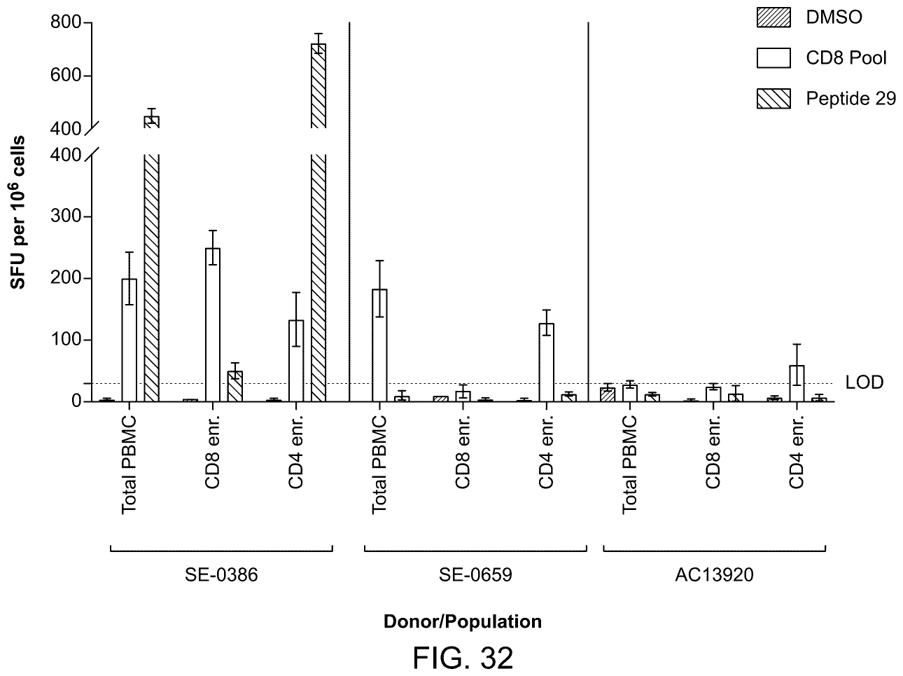

Total PMBCs from healthy donors were analyzed for responses to various KRAS neoepitopes by ex vivo ELISpot. As shown in FIG. 30, KRAS G12C elicited a response in healthy donors. Samples were further assessed against single peptides from CD8 pools. As shown in FIG. 31, PBMCs from a single donor (SE-0386) demonstrated a response to CD8 pool MHC class I peptide #29 (EYKLVVVGACG; also listed as peptide 51 in Table 10). As shown in FIG. 32, PBMCs from single donors demonstrated responses in CD4 enriched (CD8 depleted) samples to CD8 pools and for donor SE-0386 to CD8 pool peptide #29 (EYKLVVVGACG).

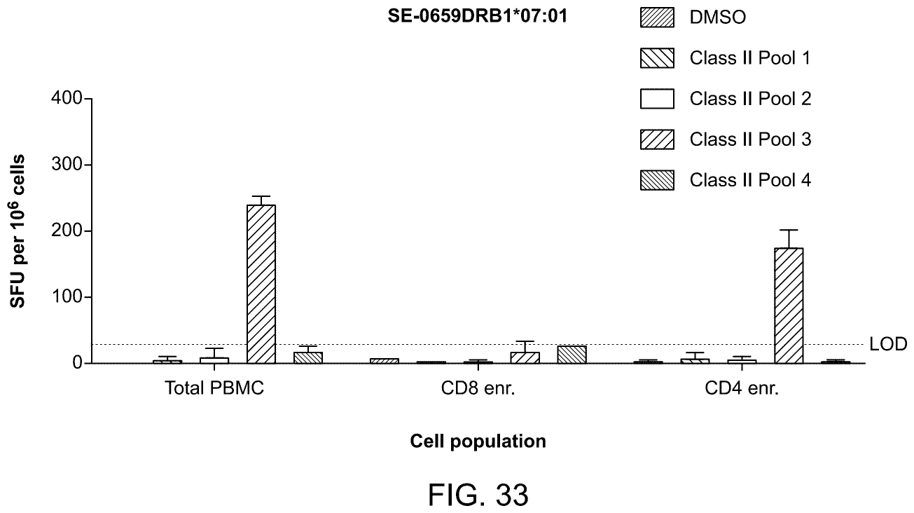

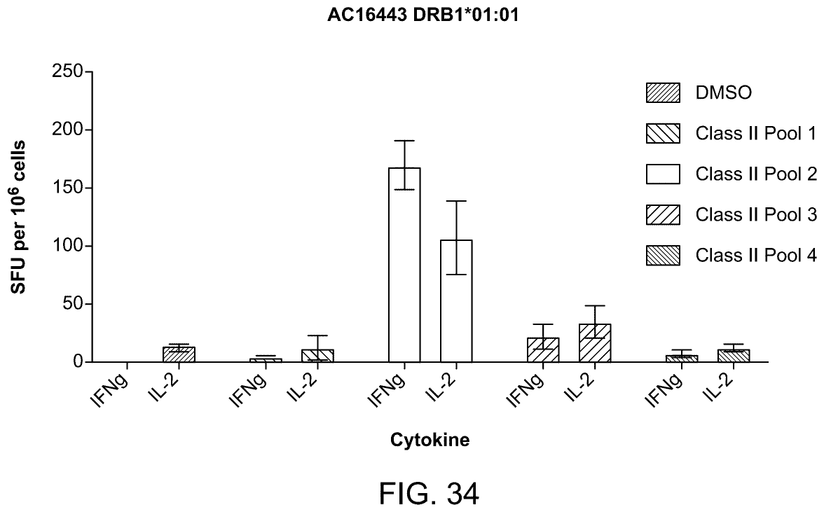

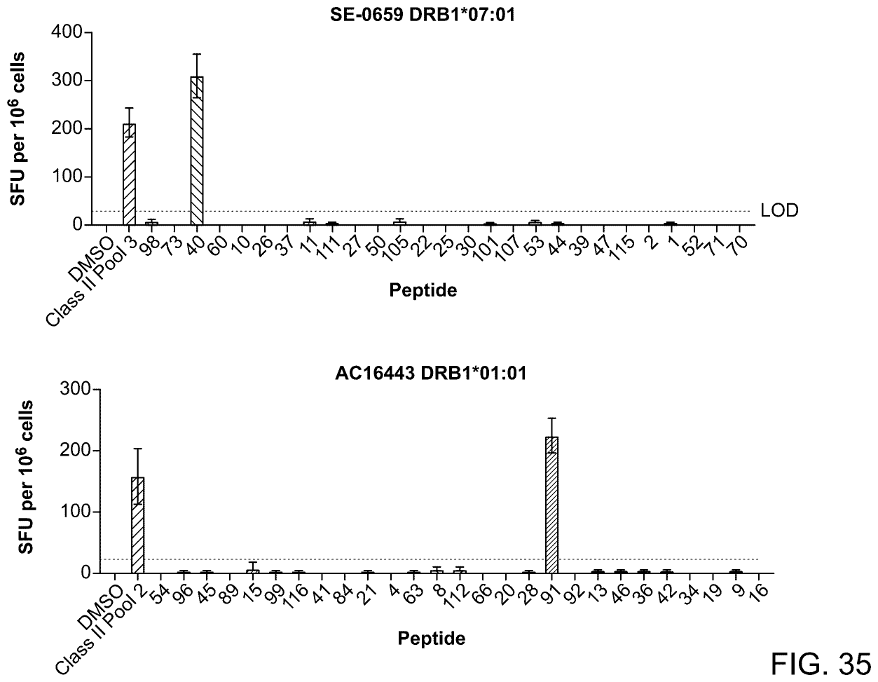

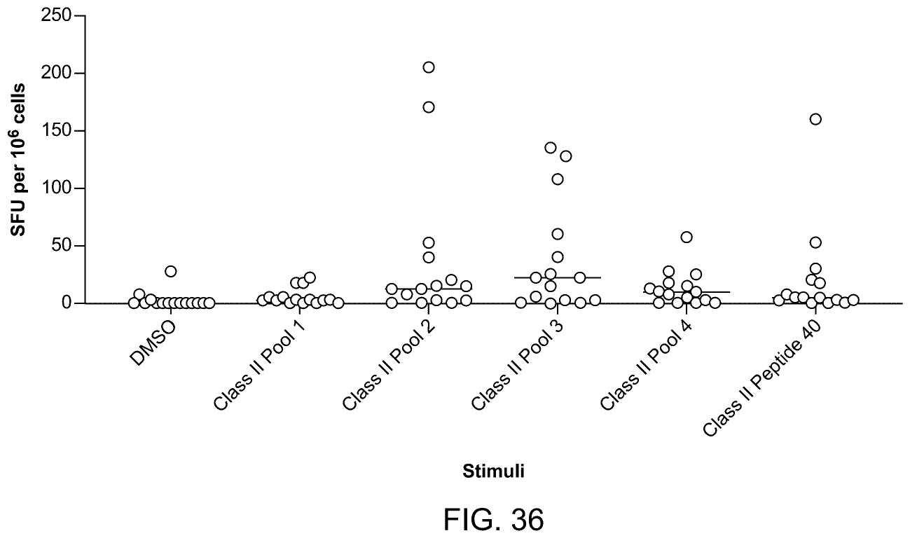

Samples were further assessed against predicted MHC Class II single peptides (108 total peptides split up into 4 pools ranging in length from 10-20 amino acids; Table 10). FIG. 33 shows ex vivo ELISpot responses to both total PBMCs and CD4/CD8 enriched PBMCs for a single donor having HLA DRB1*07:01 identifying pool 3 as stimulating a response. FIG. 34 shows ex vivo fluorospot responses to total PBMCs for a single donor having HLA DRB1*01:01 identifying pool 2 as stimulating a response. Total PBMCs were assessed against deconvoluted pool single peptides for HLA DRB1*07:01 (top panel; ex vivo ELISpot) and HLA DRB1*01:01 (bottom panel; ex vivo fluorospot). As shown in FIG. 35, responses were seen for Class II Peptide 40 (MTEYKLVVVGACGV) for HLA DRB1*07:01 and Class II Peptide 91 (KLVVVGACGVGKSALTIQLI) for HLA DRB1*01:01. FIG. 36 shows a summary of responses from healthy donors with either DRB1*01:01 or DRB1*07:01 when stimulated overnight with KRAS G12C Class II pools or single peptide 40. Table 11 shows a summary of various healthy donors, including their haplotype and responses to pools and single peptides. Class II Pool 2 responses were observed in donors with a DRB1*01:01 allele while Class II Pool 3 responses were generally observed in donors with a DRB1*07:01 allele.

### Example 11: Mass Spectrometry Validation of MHC Class II KRAS G12C Neoepitopes

Mass spectrometry (MS) validation for HLA-presentation for candidate shared neoantigens was performed using targeted mass spectrometry methods and an in vitro system. Briefly, cell lines were engineered to express a candidate shared neoantigen, with the specific lines shown in Table 12. Sequences are shown in Table 13.

Isolation of HLA-peptide molecules was performed using classic immunoprecipitation (IP) methods after lysis and solubilization of the sample (55-58). A clarified lysate was used for HLA specific IP. For isolation of HLA peptides, each cell pellet was lysed with lysis buffer and centrifuged at 20,000×g for 1 hr to clarify the lysate and the HLA peptide complexes were enriched as previously described.

Immunoprecipitation was performed using antibodies coupled to beads where the antibody is specific for HLA molecules. For a pan-Class I HLA immunoprecipitation, a pan-Class I CR antibody W632 was used. For Class II HLA-DR, the HLA-DR antibody L243 was used. For Class II HLA-DP, the HLA-DP antibody B7/21.1 was used. For Class II HLA-DQ, the HLA-DQ antibody SVPL3.1 was used. Antibody was covalently attached to NHS-sepharose beads during overnight incubation. After covalent attachment, the beads were washed and aliquoted for IP. (59, 60) Some antibodies that can be used to selectively enrich MHC/peptide complex are listed below in Table 14.

The clarified tissue lysate was added to the antibody beads for the immunoprecipitation. After immunoprecipitation, the beads were removed from the lysate and the lysate stored for additional experiments, including additional IPs. The IP beads were washed to remove non-specific binding and the HLA/peptide complex was eluted from the beads using 2N acetic acid. The protein components were removed from the peptides using C18 fractionation. The cysteine containing peptides were reduced with dithiothreitol and alkylated with iodoacetamide before desalting with C18 fractionation again. The resultant peptides were taken to dryness by SpeedVac evaporation and stored at −20C prior to MS analysis.

Dried peptides were reconstituted in an HPLC buffer suitable for reverse phase chromatography and loaded onto a C-18 microcapillary HPLC column for gradient elution in a Fusion Lumos mass spectrometer (Thermo).

Heavy peptides—peptides synthesized with amino acids containing isotopically heavy amino acids—were added to the peptides prior to analysis by MS to aid in confirmation of the identity of the peptides detected.

MS1 spectra of peptide mass/charge (m/z) were collected in the Orbitrap detector at high resolution followed by MS2 scans using HCD fragmentation of the selected ion. MS2 spectra of peptide mass/charge (m/z) was collected with high resolution mass accuracy in the Orbitrap detector with targeted method known as parallel reaction monitoring. In targeted PRM, specific peptide precursor ions were isolated in the Orbitrap detector and all resulting HCD fragmentation ions were scanned across the elution of the peptide peak. This enables both peptide identification and quantitation of endogenous peptide in the presence of a co-injected stabile isotopically labeled peptide standard.

Targeted MS1 and MS2 spectra were processed through Skyline (104).

The in vitro system described above was used to validate HLA-specific presentation of predicted neoantigens. Table 15 shows the peptide candidates that were identified by mass spectrometry as being presented by the indicated cell lines. Peptides are further validated as being presented by the indicated cell lines by mass spectrometry using heavy metal standards.

### Example 12: Individualized Neoantigen Cancer Vaccines Administered to Patients with Advanced Metastatic Solid Tumors

A first-in-human Phase 1/2 study was conducted to explore the safety, immunogenicity, and clinical activity of an individualized neoantigen cancer vaccine. The patient-specific manufacturing process begins with neoantigen prediction from a tumor biopsy using Gritstone's EDGE™ platform, which ranks the neoantigens based on their probability of being presented on the tumor cell surface in the context of HLA class I. ChAd and samRNA vaccines are then generated expressing the top 20 EDGE predicted neoantigens. The vaccine regimen consists of intramuscularly (IM) administration of ChAd and samRNA in combination with low-dose subcutaneous (SC) ipilimumab and full-dose intravenous (IV) nivolumab. The novel SC route of ipilimumab administration was chosen to increase anti-CTLA-4 concentration in vaccination site draining lymph nodes to enhance the magnitude and breadth of the vaccine-induced T cells. All patients received concurrent nivolumab to maintain T cell function in the tumor microenvironment (TME). The primary objectives of the study were to assess the safety and tolerability (Phase 1 and 2), anti-tumor activity per objective response rate (ORR) and clinical benefit rate (CBR) per RECIST v1.1 (Phase 2), and to identify and confirm the recommended Phase 2 dose (RP2D) of an individualized ChAd/samRNA-based neoantigen vaccine (Phase 1 and 2). Secondary objectives included assessment of vaccine-induced immune response to the neoantigens, ORR (Phase 1), progression free survival (PFS), overall survival (OS), and changes in the TME following vaccination. Exploratory objectives included ORR per immune-based RECIST (iRECIST), changes in neoantigen detection, and association of biomarkers (such as circulating tumor DNA [ctDNA]) with clinical activity. The study results demonstrate this personalized cancer vaccine regimen is well-tolerated, induces T cells, results in early signals of clinical activity, and associates with changes in the TME providing mechanistic insight into the observed clinical benefit.

Vaccine-Encoded Neoantigens were Patient-Specific and Maintained Over Time

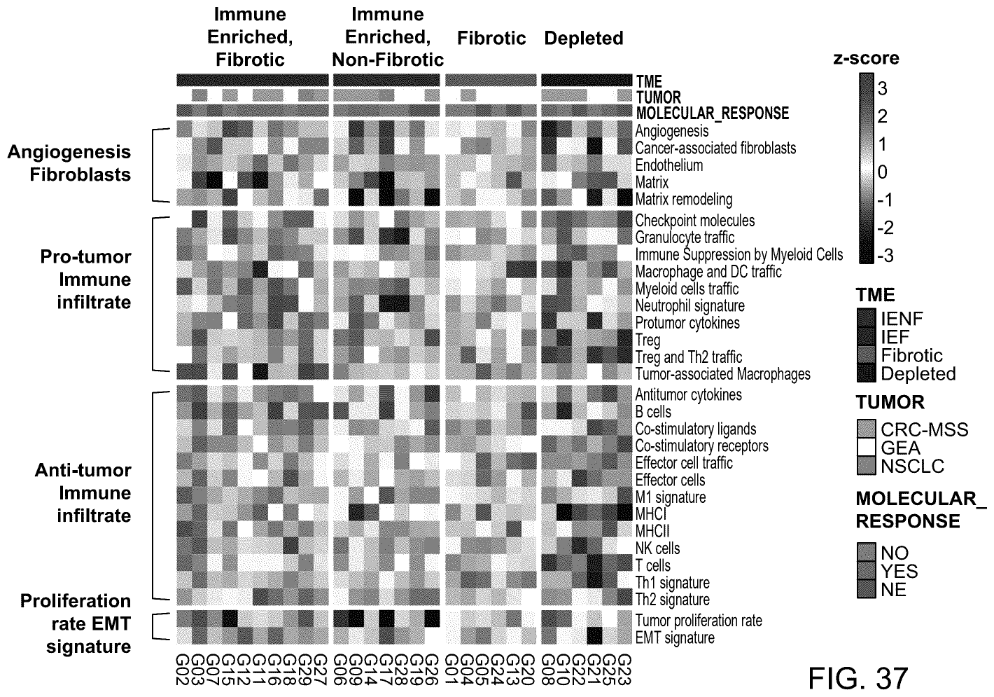

During the first step of the neoantigen prediction process, a median of 115 translated non-synonymous (NS) variants were detected per patient (range 61-382) and assessed for the probability of presentation by the EDGE™ model. This led to the selection of 580 predicted neoantigens for vaccine manufacturing in the 29 treated patients (i.e., 20 per patient). The presence of neoantigens associates with tumor mutation burden (TMB) and TMB was lowest in MSS-CRC tumors (median 2.16 mut/Mb) compared with GEA and NSCLC tumors (median of 3.3 mut/Mb and 6.0 mut/Mb respectively) (FIGS. 37A and 37B). Selected neoantigens for vaccine manufacture were predominantly patient specific with the only shared neoantigens, KRAS G12V (4 patients) and TP53 R248Q (2 patients), being rare (<1%). These results highlight the advantage of broad and deep variant detection to identify tractable neoantigens, especially in tumor types with a low median TMB.

To understand tumor burden, mutation dynamics and the potential loss of vaccine-targeted neoantigens, changes in neoantigens were longitudinally monitored in ctDNA and tumor biopsies over the course of the study. The majority of vaccine-encoded neoantigens were detectable in the periphery in ctDNA (median 92%; range 45%-100%), which exceeded detection in tumor biopsies at baseline and/or post-treatment (median 85%; range 40%-100%). Further, vaccine-encoded neoantigens were detected at a higher proportion than all variants monitored in ctDNA and tumor biopsies (80%, range 21-98% and 70%, range 21-100% respectively). Variants with lower VAF (i.e., likely subclonal) were more likely to be lost due to tumor evolution. This supports the targeting of truncal neoantigens (which have a higher VAF) as these neoantigens will likely be present and maintained in the majority of tumor cells. In totality, these data support the use of archival tissue for neoantigen prediction as the neoantigens are maintained within the tumor over time.

### Patient Subtyping

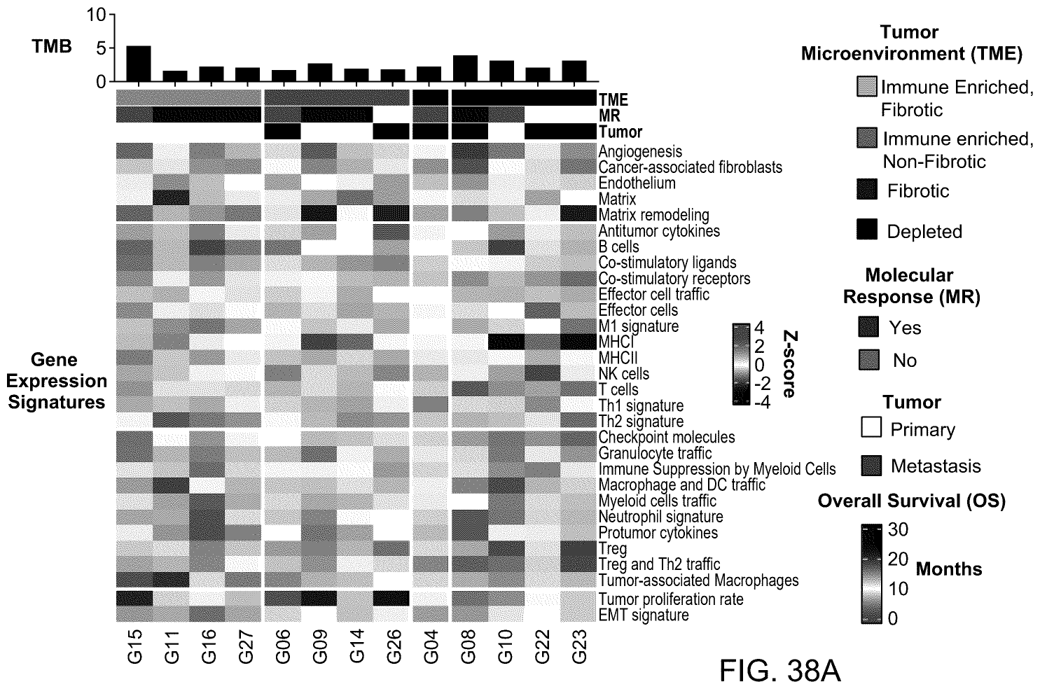

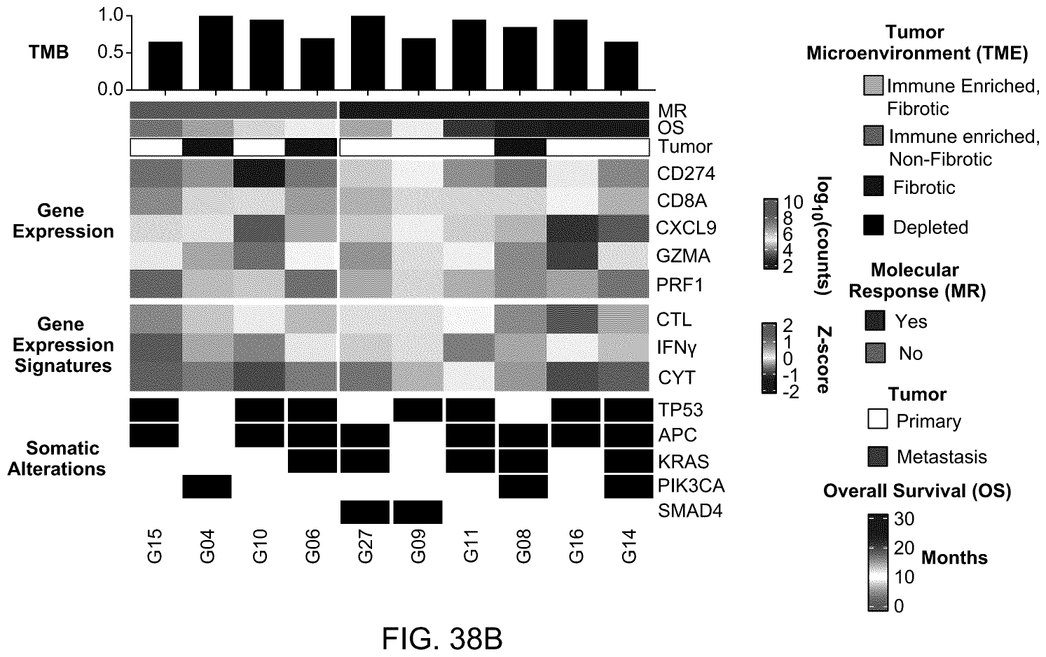

Biomarkers and gene pathways were analyzed as an exploratory endpoint to assess whether certain patient populations described herein were more likely to benefit from the vaccination using personalized neoantigen cancer vaccines. Across all tumor types reported herein, there was no notable enrichment in patients that had molecular responses (MR). MRs were observed in patients with the ICB responsive immune enriched fibrotic TME (FIGS. 38A and 38B). For example, as shown in the heat map of FIG. 37, immune enriched fibrotic subtype was characterized by upregulated activity in angiogenesis fibroblasts, pro-tumor immune infiltrate, anti-tumor infiltrate, and proliferation rate EMT signature were upregulated in the immune enriched, fibrotic patient subtype in comparison to the other subtypes (e.g., immune enriched non-fibrotic, fibrotic, or depleted). The activity of angiogenesis fibroblasts was a measure of the following pathways: angiogenesis, cancer-associated fibroblasts, endothelium, matrix, and matrix modeling. The activity of pro-tumor immune infiltrate was a measure of the following pathways: checkpoint molecules, granulocyte traffic, immune suppression by myeloid cells, macrophage and DC traffic, neutrophil signature, protumor cytokines, Treg, Treg and Th2 traffic, and tumor-associated macrophages. The activity of anti-tumor immune infiltrate was a measure of the following pathways: antitumor cytokines, B cells, co-stimulatory ligands, co-stimulatory receptors, effect cell traffic, effector cells, M1 signature, MHCI, MHCII, NK cells, T cells, Th1 signature, and Th2 signature. The activity of proliferation rate EMT signature was a measure of the following pathways: Tumor proliferation rate and EMT signature.

FIG. 38B shows a heatmap of RNAseq expression of immune-related genes and effector T cell gene signatures relating to TMB, molecular response, and the site of tumor tissue used for neoantigen prediction. RNAseq expression was derived from DEseq2 normalized RSEM expected counts. Specifically, the heatmap of FIG. 38B shows certain genes in patients who were molecular responders (4 left most columns) and patients who were not molecular responders (6 right most columns). The genes included CD274, CD8A, CXCL9, GZMA, PRF1, CTL, IFNγ, and CYT. Additional somatic alterations were determined from the following genes TP53, APC, KRAS, PIK3CA, and SMAD4. Generally, these results indicate that certain biomarkers can be informative for patient subtyping purposes (e.g., for distinguishing between patients who are likely MRs versus non-MRs.

Patients with microsatellite stable colorectal cancer (MSS-CRC) who experienced a MR were not enriched in any particular TME group. In addition, there was no enrichment for effector T cell or IFNγ related gene expression or gene signatures (FIGS. 38A and 38B). Analysis of other clinical and tumor characteristics showed that patients who had liver metastasis were as likely to have a MR and there was no enrichment for other features, such as TMB or PD-L1 expression, among patients experiencing a MR (FIGS. 38A and 38B; and Table 16). Altogether, this suggests that a particular patient subtype (e.g., immune enriched fibrotic tumor microenvironment) can be a promising patient subtype for providing personalized neoantigen cancer vaccines.

### Certain Sequences

Vectors, cassettes, and antibodies referred to herein are described below and referred to by SEQ ID NO.

(SEQ ID NO: 1)
ccatcttcaataatatacctcaaactttttgtgcgcgttaatatgcaaatgaggcgtttgaatttggggaggaagg
gcggtgattggtcgagggatgagcgaccgttaggggcggggcgagtgacgttttgatgacgtggttgcgaggagga
gccagtttgcaagttctcgtgggaaaagtgacgtcaaacgaggtgtggtttgaacacggaaatactcaattttccc
gcgctctctgacaggaaatgaggtgtttctgggcggatgcaagtgaaaacgggccattttcgcgcgaaaactgaat
gaggaagtgaaaatctgagtaatttcgcgtttatggcagggaggagtatttgccgagggccgagtagactttgacc
gattacgtgggggtttcgattaccgtgtttttcacctaaatttccgcgtacggtgtcaaagtccggtgtttttacg
taggtgtcagctgatcgccagggtatttaaacctgcgctctccagtcaagaggccactcttgagtgccagcgagaa
gagttttctcctccgcgccgcgagtcagatctacactttgaaagatgaggcacctgagagacctgcccgatgagaa
aatcatcatcgcttccgggaacgagattctggaactggtggtaaatgccatgatgggcgacgaccctccggagccc
cccaccccatttgagacaccttcgctgcacgatttgtatgatctggaggtggatgtgcccgaggacgatcccaatg
aggaggcggtaaatgatttttttagcgatgccgcgctgctagctgccgaggaggcttcgagctctagctcagacag
cgactcttcactgcatacccctagacccggcagaggtgagaaaaagatccccgagcttaaaggggaagagatggac
ttgcgctgctatgaggaatgcttgcccccgagcgatgatgaggacgagcaggcgatccagaacgcagcgagccagg
gagtgcaagccgccagcgagagctttgcgctggactgcccgcctctgcccggacacggctgtaagtcttgtgaatt
tcatcgcatgaatactggagataaagctgtgttgtgtgcactttgctatatgagagcttacaaccattgtgtttac
agtaagtgtgattaagttgaactttagagggaggcagagagcagggtgactgggcgatgactggtttatttatgta
tatatgttctttatataggtcccgtctctgacgcagatgatgagacccccactacaaagtccacttcgtcaccccc
agaaattggcacatctccacctgagaatattgttagaccagttcctgttagagccactgggaggagagcagctgtg
gaatgtttggatgacttgctacagggtggggttgaacctttggacttgtgtacccggaaacgccccaggcactaag
tgccacacatgtgtgtttacttgaggtgatgtcagtatttatagggtgtggagtgcaataaaaaatgtgttgactt
taagtgcgtggtttatgactcaggggtggggactgtgagtatataagcaggtgcagacctgtgtggttagctcaga
gcggcatggagatttggacggtcttggaagactttcacaagactagacagctgctagagaacgcctcgaacggagt
ctcttacctgtggagattctgcttcggtggcgacctagctaggctagtctacagggccaaacaggattatagtgaa
caatttgaggttattttgagagagtgttctggtctttttgacgctcttaacttgggccatcagtctcactttaacc
agaggatttcgagagcccttgattttactactcctggcagaaccactgcagcagtagccttttttgcttttattct
tgacaaatggagtcaagaaacccatttcagcagggattaccagctggatttcttagcagtagctttgtggagaaca
tggaagtgccagcgcctgaatgcaatctccggctacttgccggtacagccgctagacactctgaggatcctgaatc
tccaggagagtcccagggcacgccaacgtcgccagcagcagcagcaggaggaggatcaagaagagaacccgagagc
cggcctggaccctccggcggaggaggaggagtagctgacctgtttcctgaactgcgccgggtgctgactaggtctt
cgagtggtcgggagagggggattaagcgggagaggcatgatgagactaatcacagaactgaactgactgtgggtct
gatgagtcgcaagcgcccagaaacagtgtggtggcatgaggtgcagtcgactggcacagatgaggtgtcggtgatg
catgagaggttttctctagaacaagtcaagacttgttggttagagcctgaggatgattgggaggtagccatcagga
attatgccaagctggctctgaggccagacaagaagtacaagattactaagctgataaatatcagaaatgcctgcta
catctcagggaatggggctgaagtggagatctgtctccaggaaagggtggctttcagatgctgcatgatgaatatg
tacccgggagtggtgggcatggatggggttacctttatgaacatgaggttcaggggagatgggtataatggcacgg
tctttatggccaataccaagctgacagtccatggctgctccttctttgggtttaataacacctgcatcgaggcctg
gggtcaggtcggtgtgaggggctgcagtttttcagccaactggatgggggtcgtgggcaggaccaagagtatgctg
tccgtgaagaaatgcttgtttgagaggtgccacctgggggtgatgagcgagggcgaagccagaatccgccactgcg
cctctaccgagacgggctgctttgtgctgtgcaagggcaatgctaagatcaagcataatatgatctgtggagcctc
ggacgagcgcggctaccagatgctgacctgcgccggcgggaacagccatatgctggccaccgtacatgtggcttcc
catgctcgcaagccctggcccgagttcgagcacaatgtcatgaccaggtgcaatatgcatctggggtcccgccgag
gcatgttcatgccctaccagtgcaacctgaattatgtgaaggtgctgctggagcccgatgccatgtccagagtgag
cctgacgggggtgtttgacatgaatgtggaggtgtggaagattctgagatatgatgaatccaagaccaggtgccga
gcctgcgagtgcggagggaagcatgccaggttccagcccgtgtgtgtggatgtgacggaggacctgcgacccgatc
atttggtgttgccctgcaccgggacggagttcggttccagcggggaagaatctgactagagtgagtagtgttctgg
ggcgggggaggacctgcatgagggccagaataactgaaatctgtgcttttctgtgtgttgcagcagcatgagcgga
agcggctcctttgagggaggggtattcagcccttatctgacggggcgtctcccctcctgggcgggagtgcgtcaga
atgtgatgggatccacggtggacggccggcccgtgcagcccgcgaactcttcaaccctgacctatgcaaccctgag
ctcttcgtcgttggacgcagctgccgccgcagctgctgcatctgccgccagcgccgtgcgcggaatggccatgggc
gccggctactacggcactctggtggccaactcgagttccaccaataatcccgccagcctgaacgaggagaagctgt
tgctgctgatggcccagctcgaggccttgacccagcgcctgggcgagctgacccagcaggtggctcagctgcagga
gcagacgcgggccgcggttgccacggtgaaatccaaataaaaaatgaatcaataaataaacggagacggttgttga
ttttaacacagagtctgaatctttatttgatttttcgcgcgcggtaggccctggaccaccggtctcgatcattgag
cacccggtggatcttttccaggacccggtagaggtgggcttggatgttgaggtacatgggcatgagcccgtcccgg
gggtggaggtagctccattgcagggcctcgtgctcgggggtggtgttgtaaatcacccagtcatagcaggggcgca
gggcatggtgttgcacaatatctttgaggaggagactgatggccacgggcagccctttggtgtaggtgtttacaaa
tctgttgagctgggagggatgcatgcggggggagatgaggtgcatcttggcctggatcttgagattggcgatgtta
ccgcccagatcccgcctggggttcatgttgtgcaggaccaccagcacggtgtatccggtgcacttggggaatttat
catgcaacttggaagggaaggcgtgaaagaatttggcgacgcctttgtgcccgcccaggttttccatgcactcatc
catgatgatggcgatgggcccgtgcggggcggcctgggcaaagacgtttcgggggtcggacacatcatagttgtgg
tcctgggtgaggtcatcataggccattttaatgaatttggggcggagggtgccggactgggggacaaaggtaccct
cgatcccgggggcgtagttcccctcacagatctgcatctcccaggctttgagctcggagggggggatcatgtccac
ctgcggggcgataaagaacacggtttccggggcgggggagatgagctgggccgaaagcaagttccggagcagctgg
gacttgccgcagccggtggggccgtagatgaccccgatgaccggctgcaggtggtagttgagggagagacagctgc
cgtcctcccggaggaggggggccacctcgttcatcatctcgcgcacgtgcatgttctcgcgcaccagttccgccag
gaggcgctctccccccagggataggagctcctggagcgaggcgaagtttttcagcggcttgagtccgtcggccatg
ggcattttggagagggtttgttgcaagagttccaggcggtcccagagctcggtgatgtgctctacggcatctcgat
ccagcagacctcctcgtttcgcgggttgggacggctgcgggagtagggcaccagacgatgggcgtccagcgcagcc
agggtccggtccttccagggtcgcagcgtccgcgtcagggtggtctccgtcacggtgaaggggtgcgcgccgggct
gggcgcttgcgagggtgcgcttcaggctcatccggctggtcgaaaaccgctcccgatcggcgccctgcgcgtcggc
caggtagcaattgaccatgagttcgtagttgagcgcctcggccgcgtggcctttggcgcggagcttacctttggaa
gtctgcccgcaggcgggacagaggagggacttgagggcgtagagcttgggggcgaggaagacggactcgggggcgt
aggcgtccgcgccgcagtgggcgcagacggtctcgcactccacgagccaggtgaggtcgggctggtcggggtcaaa
aaccagtttcccgccgttctttttgatgcgtttcttacctttggtctccatgagctcgtgtccccgctgggtgaca
aagaggctgtccgtgtccccgtagaccgactttatgggccggtcctcgagcggtgtgccgcggtcctcctcgtaga
ggaaccccgcccactccgagacgaaagcccgggtccaggccagcacgaaggaggccacgtgggacgggtagcggtc
gttgtccaccagcgggtccaccttttccagggtatgcaaacacatgtccccctcgtccacatccaggaaggtgatt
ggcttgtaagtgtaggccacgtgaccgggggtcccggccgggggggtataaaagggtgcgggtccctgctcgtcct
cactgtcttccggatcgctgtccaggagcgccagctgttggggtaggtattccctctcgaaggcgggcatgacctc
ggcactcaggttgtcagtttctagaaacgaggaggatttgatattgacggtgccggcggagatgcctttcaagagc
ccctcgtccatctggtcagaaaagacgatctttttgttgtcgagcttggtggcgaaggagccgtagagggcgttgg
agaggagcttggcgatggagcgcatggtctggtttttttccttgtcggcgcgctccttggcggcgatgttgagctg
cacgtactcgcgcgccacgcacttccattcggggaagacggtggtcagctcgtcgggcacgattctgacctgccag
ccccgattatgcagggtgatgaggtccacactggtggccacctcgccgcgcaggggctcattagtccagcagaggc
gtccgcccttgcgcgagcagaaggggggcagggggtccagcatgacctcgtcgggggggtcggcatcgatggtgaa
gatgccgggcaggaggtcggggtcaaagtagctgatggaagtggccagatcgtccagggcagcttgccattcgcgc
acggccagcgcgcgctcgtagggactgaggggcgtgccccagggcatgggatgggtaagcgcggaggcgtacatgc
cgcagatgtcgtagacgtagaggggctcctcgaggatgccgatgtaggtggggtagcagcgccccccgcggatgct
ggcgcgcacgtagtcatacagctcgtgcgagggggcgaggagccccgggcccaggttggtgcgactgggcttttcg
gcgcggtagacgatctggcggaaaatggcatgcgagttggaggagatggtgggcctttggaagatgttgaagtggg
cgtggggcagtccgaccgagtcgcggatgaagtgggcgtaggagtcttgcagcttggcgacgagctcggcggtgac
taggacgtccagagcgcagtagtcgagggtctcctggatgatgtcatacttgagctgtcccttttgtttccacagc
tcgcggttgagaaggaactcttcgcggtccttccagtactcttcgagggggaacccgtcctgatctgcacggtaag
agcctagcatgtagaactggttgacggccttgtaggcgcagcagcccttctccacggggagggcgtaggcctgggc
ggccttgcgcagggaggtgtgcgtgagggcgaaagtgtccctgaccatgaccttgaggaactggtgcttgaagtcg
atatcgtcgcagcccccctgctcccagagctggaagtccgtgcgcttcttgtaggcggggttgggcaaagcgaaag
taacatcgttgaagaggatcttgcccgcgcggggcataaagttgcgagtgatgcggaaaggttggggcacctcggc
ccggttgttgatgacctgggcggcgagcacgatctcgtcgaagccgttgatgttgtggcccacgatgtagagttcc
acgaatcgcggacggcccttgacgtggggcagtttcttgagctcctcgtaggtgagctcgtcggggtcgctgagcc
cgtgctgctcgagcgcccagtcggcgagatgggggttggcgcggaggaaggaagtccagagatccacggccagggc
ggtttgcagacggtcccggtactgacggaactgctgcccgacggccattttttcgggggtgacgcagtagaaggtg
cgggggtccccgtgccagcgatcccatttgagctggagggcgagatcgagggcgagctcgacgagccggtcgtccc
cggagagtttcatgaccagcatgaaggggacgagctgcttgccgaaggaccccatccaggtgtaggtttccacatc
gtaggtgaggaagagcctttcggtgcgaggatgcgagccgatggggaagaactggatctcctgccaccaattggag
gaatggctgttgatgtgatggaagtagaaatgccgacggcgcgccgaacactcgtgcttgtgtttatacaagcggc
cacagtgctcgcaacgctgcacgggatgcacgtgctgcacgagctgtacctgagttcctttgacgaggaatttcag
tgggaagtggagtcgtggcgcctgcatctcgtgctgtactacgtcgtggtggtcggcctggccctcttctgcctcg
atggtggtcatgctgacgagcccgcgcgggaggcaggtccagacctcggcgcgagcgggtcggagagcgaggacga
gggcgcgcaggccggagctgtccagggtcctgagacgctgcggagtcaggtcagtgggcagcggcggcgcgcggtt
gacttgcaggagtttttccagggcgcgcgggaggtccagatggtacttgatctccaccgcgccattggtggcgacg
tcgatggcttgcagggtcccgtgcccctggggtgtgaccaccgtcccccgtttcttcttgggcggctggggcgacg
ggggcggtgcctcttccatggttagaagcggcggcgaggacgcgcgccgggcggcaggggcggctcggggcccgga
ggcaggggcggcaggggcacgtcggcgccgcgcgcgggtaggttctggtactgcgcccggagaagactggcgtgag
cgacgacgcgacggttgacgtcctggatctgacgcctctgggtgaaggccacgggacccgtgagtttgaacctgaa
agagagttcgacagaatcaatctcggtatcgttgacggcggcctgccgcaggatctcttgcacgtcgcccgagttg
tcctggtaggcgatctcggtcatgaactgctcgatctcctcctcttgaaggtctccgcggccggcgcgctccacgg
tggccgcgaggtcgttggagatgcggcccatgagctgcgagaaggcgttcatgcccgcctcgttccagacgcggct
gtagaccacgacgccctcgggatcgcgggcgcgcatgaccacctgggcgaggttgagctccacgtggcgcgtgaag
accgcgtagttgcagaggcgctggtagaggtagttgagcgtggtggcgatgtgctcggtgacgaagaaatacatga
tccagcggcggagcggcatctcgctgacgtcgcccagcgcctccaaacgttccatggcctcgtaaaagtccacggc
gaagttgaaaaactgggagttgcgcgccgagacggtcaactcctcctccagaagacggatgagctcggcgatggtg
gcgcgcacctcgcgctcgaaggcccccgggagttcctccacttcctcttcttcctcctccactaacatctcttcta
cttcctcctcaggcggcagtggtggcgggggagggggcctgcgtcgccggcggcgcacgggcagacggtcgatgaa
gcgctcgatggtctcgccgcgccggcgtcgcatggtctcggtgacggcgcgcccgtcctcgcggggccgcagcgtg
aagacgccgccgcgcatctccaggtggccgggggggtccccgttgggcagggagagggcgctgacgatgcatctta
tcaattgccccgtagggactccgcgcaaggacctgagcgtctcgagatccacgggatctgaaaaccgctgaacgaa
ggcttcgagccagtcgcagtcgcaaggtaggctgagcacggtttcttctggcgggtcatgttggttgggagcgggg
cgggcgatgctgctggtgatgaagttgaaataggcggttctgagacggcggatggtggcgaggagcaccaggtctt
tgggcccggcttgctggatgcgcagacggtcggccatgccccaggcgtggtcctgacacctggccaggtccttgta
gtagtcctgcatgagccgctccacgggcacctcctcctcgcccgcgcggccgtgcatgcgcgtgagcccgaagccg
cgctggggctggacgagcgccaggtcggcgacgacgcgctcggcgaggatggcttgctggatctgggtgagggtgg
tctggaagtcatcaaagtcgacgaagcggtggtaggctccggtgttgatggtgtaggagcagttggccatgacgga
ccagttgacggtctggtggcccggacgcacgagctcgtggtacttgaggcgcgagtaggcgcgcgtgtcgaagatg
tagtcgttgcaggtgcgcaccaggtactggtagccgatgaggaagtgcggcggcggctggcggtagagcggccatc
gctcggtggcgggggcgccgggcgcgaggtcctcgagcatggtgcggtggtagccgtagatgtacctggacatcca
ggtgatgccggcggcggtggtggaggcgcgcgggaactcgcggacgcggttccagatgttgcgcagcggcaggaag
tagttcatggtgggcacggtctggcccgtgaggcgcgcgcagtcgtggatgctctatacgggcaaaaacgaaagcg
gtcagcggctcgactccgtggcctggaggctaagcgaacgggttgggctgcgcgtgtaccccggttcgaatctcga
atcaggctggagccgcagctaacgtggtattggcactcccgtctcgacccaagcctgcaccaaccctccaggatac
ggaggcgggtcgttttgcaacttttttttggaggccggatgagactagtaagcgcggaaagcggccgaccgcgatg
gctcgctgccgtagtctggagaagaatcgccagggttgcgttgcggtgtgccccggttcgaggccggccggattcc
gcggctaacgagggcgtggctgccccgtcgtttccaagaccccatagccagccgacttctccagttacggagcgag
cccctcttttgttttgtttgtttttgccagatgcatcccgtactgcggcagatgcgcccccaccaccctccaccgc
aacaacagccccctccacagccggcgcttctgcccccgccccagcagcaacttccagccacgaccgccgcggccgc
cgtgagcggggctggacagagttatgatcaccagctggccttggaagagggcgaggggctggcgcgcctgggggcg
tcgtcgccggagcggcacccgcgcgtgcagatgaaaagggacgctcgcgaggcctacgtgcccaagcagaacctgt
tcagagacaggagcggcgaggagcccgaggagatgcgcgcggcccggttccacgcggggcgggagctgcggcgcgg
cctggaccgaaagagggtgctgagggacgaggatttcgaggcggacgagctgacggggatcagccccgcgcgcgcg
cacgtggccgcggccaacctggtcacggcgtacgagcagaccgtgaaggaggagagcaacttccaaaaatccttca
acaaccacgtgcgcaccctgatcgcgcgcgaggaggtgaccctgggcctgatgcacctgtgggacctgctggaggc
catcgtgcagaaccccaccagcaagccgctgacggcgcagctgttcctggtggtgcagcatagtcgggacaacgaa
gcgttcagggaggcgctgctgaatatcaccgagcccgagggccgctggctcctggacctggtgaacattctgcaga
gcatcgtggtgcaggagcgcgggctgccgctgtccgagaagctggcggccatcaacttctcggtgctgagtttggg
caagtactacgctaggaagatctacaagaccccgtacgtgcccatagacaaggaggtgaagatcgacgggttttac
atgcgcatgaccctgaaagtgctgaccctgagcgacgatctgggggtgtaccgcaacgacaggatgcaccgtgcgg
tgagcgccagcaggcggcgcgagctgagcgaccaggagctgatgcatagtctgcagcgggccctgaccggggccgg
gaccgagggggagagctactttgacatgggcgcggacctgcactggcagcccagccgccgggccttggaggcggcg
gcaggaccctacgtagaagaggtggacgatgaggtggacgaggagggcgagtacctggaagactgatggcgcgacc
gtatttttgctagatgcaacaacaacagccacctcctgatcccgcgatgcgggcggcgctgcagagccagccgtcc
ggcattaactcctcggacgattggacccaggccatgcaacgcatcatggcgctgacgacccgcaaccccgaagcct
ttagacagcagccccaggccaaccggctctcggccatcctggaggccgtggtgccctcgcgctccaaccccacgca
cgagaaggtcctggccatcgtgaacgcgctggtggagaacaaggccatccgcggcgacgaggccggcctggtgtac
aacgcgctgctggagcgcgtggcccgctacaacagcaccaacgtgcagaccaacctggaccgcatggtgaccgacg
tgcgcgaggccgtggcccagcgcgagcggttccaccgcgagtccaacctgggatccatggtggcgctgaacgcctt
cctcagcacccagcccgccaacgtgccccggggccaggaggactacaccaacttcatcagcgccctgcgcctgatg
gtgaccgaggtgccccagagcgaggtgtaccagtccgggccggactacttcttccagaccagtcgccagggcttgc
agaccgtgaacctgagccaggctttcaagaacttgcagggcctgtggggcgtgcaggccccggtcggggaccgcgc
gacggtgtcgagcctgctgacgccgaactcgcgcctgctgctgctgctggtggcccccttcacggacagcggcagc
atcaaccgcaactcgtacctgggctacctgattaacctgtaccgcgaggccatcggccaggcgcacgtggacgagc
agacctaccaggagatcacccacgtgagccgcgccctgggccaggacgacccgggcaacctggaagccaccctgaa
ctttttgctgaccaaccggtcgcagaagatcccgccccagtacgcgctcagcaccgaggaggagcgcatcctgcgt
tacgtgcagcagagcgtgggcctgttcctgatgcaggagggggccacccccagcgccgcgctcgacatgaccgcgc
gcaacatggagcccagcatgtacgccagcaaccgcccgttcatcaataaactgatggactacttgcatcgggcggc
cgccatgaactctgactatttcaccaacgccatcctgaatccccactggctcccgccgccggggttctacacgggc
gagtacgacatgcccgaccccaatgacgggttcctgtgggacgatgtggacagcagcgtgttctccccccgaccgg
gtgctaacgagcgccccttgtggaagaaggaaggcagcgaccgacgcccgtcctcggcgctgtccggccgcgaggg
tgctgccgcggcggtgcccgaggccgccagtcctttcccgagcttgcccttctcgctgaacagtatccgcagcagc
gagctgggcaggatcacgcgcccgcgcttgctgggcgaagaggagtacttgaatgactcgctgttgagacccgagc
gggagaagaacttccccaataacgggatagaaagcctggtggacaagatgagccgctggaagacgtatgcgcagga
gcacagggacgatccccgggcgtcgcagggggccacgagccggggcagcgccgcccgtaaacgccggtggcacgac
aggcagcggggacagatgtgggacgatgaggactccgccgacgacagcagcgtgttggacttgggtgggagtggta
acccgttcgctcacctgcgcccccgtatcgggcgcatgatgtaagagaaaccgaaaataaatgatactcaccaagg
ccatggcgaccagcgtgcgttcgtttcttctctgttgttgttgtatctagtatgatgaggcgtgcgtacccggagg
gtcctcctccctcgtacgagagcgtgatgcagcaggcgatggcggcggcggcgatgcagcccccgctggaggctcc
ttacgtgcccccgcggtacctggcgcctacggaggggcggaacagcattcgttactcggagctggcacccttgtac
gataccacccggttgtacctggtggacaacaagtcggcggacatcgcctcgctgaactaccagaacgaccacagca
acttcctgaccaccgtggtgcagaacaatgacttcacccccacggaggccagcacccagaccatcaactttgacga
gcgctcgcggtggggcggccagctgaaaaccatcatgcacaccaacatgcccaacgtgaacgagttcatgtacagc
aacaagttcaaggcgcgggtgatggtctcccgcaagacccccaatggggtgacagtgacagaggattatgatggta
gtcaggatgagctgaagtatgaatgggtggaatttgagctgcccgaaggcaacttctcggtgaccatgaccatcga
cctgatgaacaacgccatcatcgacaattacttggcggtggggcggcagaacggggtgctggagagcgacatcggc
gtgaagttcgacactaggaacttcaggctgggctgggaccccgtgaccgagctggtcatgcccggggtgtacacca
acgaggctttccatcccgatattgtcttgctgcccggctgcggggtggacttcaccgagagccgcctcagcaacct
gctgggcattcgcaagaggcagcccttccaggaaggcttccagatcatgtacgaggatctggaggggggcaacatc
cccgcgctcctggatgtcgacgcctatgagaaaagcaaggaggatgcagcagctgaagcaactgcagccgtagcta
ccgcctctaccgaggtcaggggcgataattttgcaagcgccgcagcagtggcagcggccgaggcggctgaaaccga
aagtaagatagtcattcagccggtggagaaggatagcaagaacaggagctacaacgtactaccggacaagataaac
accgcctaccgcagctggtacctagcctacaactatggcgaccccgagaagggcgtgcgctcctggacgctgctca
ccacctcggacgtcacctgcggcgtggagcaagtctactggtcgctgcccgacatgatgcaagacccggtcacctt
ccgctccacgcgtcaagttagcaactacccggtggtgggcgccgagctcctgcccgtctactccaagagcttcttc
aacgagcaggccgtctactcgcagcagctgcgcgccttcacctcgcttacgcacgtcttcaaccgcttccccgaga
accagatcctcgtccgcccgcccgcgcccaccattaccaccgtcagtgaaaacgttcctgctctcacagatcacgg
gaccctgccgctgcgcagcagtatccggggagtccagcgcgtgaccgttactgacgccagacgccgcacctgcccc
tacgtctacaaggccctgggcatagtcgcgccgcgcgtcctctcgagccgcaccttctaaatgtccattctcatct
cgcccagtaataacaccggttggggcctgcgcgcgcccagcaagatgtacggaggcgctcgccaacgctccacgca
acaccccgtgcgcgtgcgcgggcacttccgcgctccctggggcgccctcaagggccgcgtgcggtcgcgcaccacc
gtcgacgacgtgatcgaccaggtggtggccgacgcgcgcaactacacccccgccgccgcgcccgtctccaccgtgg
acgccgtcatcgacagcgtggtggccgacgcgcgccggtacgcccgcgccaagagccggcggcggcgcatcgcccg
gcggcaccggagcacccccgccatgcgcgcggcgcgagccttgctgcgcagggccaggcgcacgggacgcagggcc
atgctcagggcggccagacgcgcggcttcaggcgccagcgccggcaggacccggagacgcgcggccacggcggcgg
cagcggccatcgccagcatgtcccgcccgcggcgagggaacgtgtactgggtgcgcgacgccgccaccggtgtgcg
cgtgcccgtgcgcacccgcccccctcgcacttgaagatgttcacttcgcgatgttgatgtgtcccagcggcgagga
ggatgtccaagcgcaaattcaaggaagagatgctccaggtcatcgcgcctgagatctacggccctgcggtggtgaa
ggaggaaagaaagccccgcaaaatcaagcgggtcaaaaaggacaaaaaggaagaagaaagtgatgtggacggattg
gtggagtttgtgcgcgagttcgccccccggcggcgcgtgcagtggcgcgggcggaaggtgcaaccggtgctgagac
ccggcaccaccgtggtcttcacgcccggcgagcgctccggcaccgcttccaagcgctcctacgacgaggtgtacgg
ggatgatgatattctggagcaggcggccgagcgcctgggcgagtttgcttacggcaagcgcagccgttccgcaccg
aaggaagaggcggtgtccatcccgctggaccacggcaaccccacgccgagcctcaagcccgtgaccttgcagcagg
tgctgccgaccgcggcgccgcgccgggggttcaagcgcgagggcgaggatctgtaccccaccatgcagctgatggt
gcccaagcgccagaagctggaagacgtgctggagaccatgaaggtggacccggacgtgcagcccgaggtcaaggtg
cggcccatcaagcaggtggccccgggcctgggcgtgcagaccgtggacatcaagattcccacggagcccatggaaa
cgcagaccgagcccatgatcaagcccagcaccagcaccatggaggtgcagacggatccctggatgccatcggctcc
tagtcgaagaccccggcgcaagtacggcgcggccagcctgctgatgcccaactacgcgctgcatccttccatcatc
cccacgccgggctaccgcggcacgcgcttctaccgcggtcataccagcagccgccgccgcaagaccaccactcgcc
gccgccgtcgccgcaccgccgctgcaaccacccctgccgccctggtgcggagagtgtaccgccgcggccgcgcacc
tctgaccctgccgcgcgcgcgctaccacccgagcatcgccatttaaactttcgcctgctttgcagatcaatggccc
tcacatgccgccttcgcgttcccattacgggctaccgaggaagaaaaccgcgccgtagaaggctggcggggaacgg
gatgcgtcgccaccaccaccggcggcggcgcgccatcagcaagcggttggggggaggcttcctgcccgcgctgatc
cccatcatcgccgcggcgatcggggcgatccccggcattgcttccgtggcggtgcaggcctctcagcgccactgag
acacacttggaaacatcttgtaataaaccaatggactctgacgctcctggtcctgtgatgtgttttcgtagacaga
tggaagacatcaatttttcgtccctggctccgcgacacggcacgcggccgttcatgggcacctggagcgacatcgg
caccagccaactgaacgggggcgccttcaattggagcagtctctggagcgggcttaagaatttcgggtccacgctt
aaaacctatggcagcaaggcgtggaacagcaccacagggcaggcgctgagggataagctgaaagagcagaacttcc
agcagaaggtggtcgatgggctcgcctcgggcatcaacggggtggtggacctggccaaccaggccgtgcagcggca
gatcaacagccgcctggacccggtgccgcccgccggctccgtggagatgccgcaggtggaggaggagctgcctccc
ctggacaagcggggcgagaagcgaccccgccccgatgcggaggagacgctgctgacgcacacggacgagccgcccc
cgtacgaggaggcggtgaaactgggtctgcccaccacgcggcccatcgcgcccctggccaccggggtgctgaaacc
cgaaaagcccgcgaccctggacttgcctcctccccagccttcccgcccctctacagtggctaagcccctgccgccg
gtggccgtggcccgcgcgcgacccgggggcaccgcccgccctcatgcgaactggcagagcactctgaacagcatcg
tgggtctgggagtgcagagtgtgaagcgccgccgctgctattaaacctaccgtagcgcttaacttgcttgtctgtg
tgtgtatgtattatgtcgccgccgccgctgtccaccagaaggaggagtgaagaggcgcgtcgccgagttgcaagat
ggccaccccatcgatgctgccccagtgggcgtacatgcacatcgccggacaggacgcttcggagtacctgagtccg
ggtctggtgcagtttgcccgcgccacagacacctacttcagtctggggaacaagtttaggaaccccacggtggcgc
ccacgcacgatgtgaccaccgaccgcagccagcggctgacgctgcgcttcgtgcccgtggaccgcgaggacaacac
ctactcgtacaaagtgcgctacacgctggccgtgggcgacaaccgcgtgctggacatggccagcacctactttgac
atccgcggcgtgctggatcggggccctagcttcaaaccctactccggcaccgcctacaacagtctggcccccaagg
gagcacccaacacttgtcagtggacatataaagccgatggtgaaactgccacagaaaaaacctatacatatggaaa
tgcacccgtgcagggcattaacatcacaaaagatggtattcaacttggaactgacaccgatgatcagccaatctac
gcagataaaacctatcagcctgaacctcaagtgggtgatgctgaatggcatgacatcactggtactgatgaaaagt
atggaggcagagctcttaagcctgataccaaaatgaagccttgttatggttcttttgccaagcctactaataaaga
aggaggtcaggcaaatgtgaaaacaggaacaggcactactaaagaatatgacatagacatggctttctttgacaac
agaagtgcggctgctgctggcctagctccagaaattgttttgtatactgaaaatgtggatttggaaactccagata
cccatattgtatacaaagcaggcacagatgacagcagctcttctattaatttgggtcagcaagccatgcccaacag
acctaactacattggtttcagagacaactttatcgggctcatgtactacaacagcactggcaatatgggggtgctg
gccggtcaggcttctcagctgaatgctgtggttgacttgcaagacagaaacaccgagctgtcctaccagctcttgc
ttgactctctgggtgacagaacccggtatttcagtatgtggaatcaggcggtggacagctatgatcctgatgtgcg
cattattgaaaatcatggtgtggaggatgaacttcccaactattgtttccctctggatgctgttggcagaacagat
acttatcagggaattaaggctaatggaactgatcaaaccacatggaccaaagatgacagtgtcaatgatgctaatg
agataggcaagggtaatccattcgccatggaaatcaacatccaagccaacctgtggaggaacttcctctacgccaa
cgtggccctgtacctgcccgactcttacaagtacacgccggccaatgttaccctgcccaccaacaccaacacctac
gattacatgaacggccgggtggtggcgccctcgctggtggactcctacatcaacatcggggcgcgctggtcgctgg
atcccatggacaacgtgaaccccttcaaccaccaccgcaatgcggggctgcgctaccgctccatgctcctgggcaa
cgggcgctacgtgcccttccacatccaggtgccccagaaatttttcgccatcaagagcctcctgctcctgcccggg
tcctacacctacgagtggaacttccgcaaggacgtcaacatgatcctgcagagctccctcggcaacgacctgcgca
cggacggggcctccatctccttcaccagcatcaacctctacgccaccttcttccccatggcgcacaacacggcctc
cacgctcgaggccatgctgcgcaacgacaccaacgaccagtccttcaacgactacctctcggcggccaacatgctc
taccccatcccggccaacgccaccaacgtgcccatctccatcccctcgcgcaactgggccgccttccgcggctggt
ccttcacgcgtctcaagaccaaggagacgccctcgctgggctccgggttcgacccctacttcgtctactcgggctc
catcccctacctcgacggcaccttctacctcaaccacaccttcaagaaggtctccatcaccttcgactcctccgtc
agctggcccggcaacgaccggctcctgacgcccaacgagttcgaaatcaagcgcaccgtcgacggcgagggctaca
acgtggcccagtgcaacatgaccaaggactggttcctggtccagatgctggcccactacaacatcggctaccaggg
cttctacgtgcccgagggctacaaggaccgcatgtactccttcttccgcaacttccagcccatgagccgccaggtg
gtggacgaggtcaactacaaggactaccaggccgtcaccctggcctaccagcacaacaactcgggcttcgtcggct
acctcgcgcccaccatgcgccagggccagccctaccccgccaactacccctacccgctcatcggcaagagcgccgt
caccagcgtcacccagaaaaagttcctctgcgacagggtcatgtggcgcatccccttctccagcaacttcatgtcc
atgggcgcgctcaccgacctcggccagaacatgctctatgccaactccgcccacgcgctagacatgaatttcgaag
tcgaccccatggatgagtccacccttctctatgttgtcttcgaagtcttcgacgtcgtccgagtgcaccagcccca
ccgcggcgtcatcgaggccgtctacctgcgcacccccttctcggccggtaacgccaccacctaagctcttgcttct
tgcaagccatggccgcgggctccggcgagcaggagctcagggccatcatccgcgacctgggctgcgggccctactt
cctgggcaccttcgataagcgcttcccgggattcatggccccgcacaagctggcctgcgccatcgtcaacacggcc
ggccgcgagaccgggggcgagcactggctggccttcgcctggaacccgcgctcgaacacctgctacctcttcgacc
ccttcgggttctcggacgagcgcctcaagcagatctaccagttcgagtacgagggcctgctgcgccgcagcgccct
ggccaccgaggaccgctgcgtcaccctggaaaagtccacccagaccgtgcagggtccgcgctcggccgcctgcggg
ctcttctgctgcatgttcctgcacgccttcgtgcactggcccgaccgccccatggacaagaaccccaccatgaact
tgctgacgggggtgcccaacggcatgctccagtcgccccaggtggaacccaccctgcgccgcaaccaggaggcgct
ctaccgcttcctcaactcccactccgcctactttcgctcccaccgcgcgcgcatcgagaaggccaccgccttcgac
cgcatgaatcaagacatgtaaaccgtgtgtgtatgttaaatgtctttaataaacagcactttcatgttacacatgc
atctgagatgatttatttagaaatcgaaagggttctgccgggtctcggcatggcccgcgggcagggacacgttgcg
gaactggtacttggccagccacttgaactcggggatcagcagtttgggcagcggggtgtcggggaaggagtcggtc
cacagcttccgcgtcagttgcagggcgcccagcaggtcgggcgcggagatcttgaaatcgcagttgggacccgcgt
tctgcgcgcgggagttgcggtacacggggttgcagcactggaacaccatcagggccgggtgcttcacgctcgccag
caccgtcgcgtcggtgatgctctccacgtcgaggtcctcggcgttggccatcccgaagggggtcatcttgcaggtc
tgccttcccatggtgggcacgcacccgggcttgtggttgcaatcgcagtgcagggggatcagcatcatctgggcct
ggtcggcgttcatccccgggtacatggccttcatgaaagcctccaattgcctgaacgcctgctgggccttggctcc
ctcggtgaagaagaccccgcaggacttgctagagaactggttggtggcgcacccggcgtcgtgcacgcagcagcgc
gcgtcgttgttggccagctgcaccacgctgcgcccccagcggttctgggtgatcttggcccggtcggggttctcct
tcagcgcgcgctgcccgttctcgctcgccacatccatctcgatcatgtgctccttctggatcatggtggtcccgtg
caggcaccgcagcttgccctcggcctcggtgcacccgtgcagccacagcgcgcacccggtgcactcccagttcttg
tgggcgatctgggaatgcgcgtgcacgaagccctgcaggaagcggcccatcatggtggtcagggtcttgttgctag
tgaaggtcagcggaatgccgcggtgctcctcgttgatgtacaggtggcagatgcggcggtacacctcgccctgctc
gggcatcagctggaagttggctttcaggtcggtctccacgcggtagcggtccatcagcatagtcatgatttccata
cccttctcccaggccgagacgatgggcaggctcatagggttcttcaccatcatcttagcgctagcagccgcggcca
gggggtcgctctcgtccagggtctcaaagctccgcttgccgtccttctcggtgatccgcaccggggggtagctgaa
gcccacggccgccagctcctcctcggcctgtctttcgtcctcgctgtcctggctgacgtcctgcaggaccacatgc
ttggtcttgcggggtttcttcttgggcggcagcggcggcggagatgttggagatggcgagggggagcgcgagttct
cgctcaccactactatctcttcctcttcttggtccgaggccacgcggcggtaggtatgtctcttcgggggcagagg
cggaggcgacgggctctcgccgccgcgacttggcggatggctggcagagccccttccgcgttcgggggtgcgctcc
cggcggcgctctgactgacttcctccgcggccggccattgtgttctcctagggaggaacaacaagcatggagactc
agccatcgccaacctcgccatctgcccccaccgccgacgagaagcagcagcagcagaatgaaagcttaaccgcccc
gccgcccagccccgccacctccgacgcggccgtcccagacatgcaagagatggaggaatccatcgagattgacctg
ggctatgtgacgcccgcggagcacgaggaggagctggcagtgcgcttttcacaagaagagatacaccaagaacagc
cagagcaggaagcagagaatgagcagagtcaggctgggctcgagcatgacggcgactacctccacctgagcggggg
ggaggacgcgctcatcaagcatctggcccggcaggccaccatcgtcaaggatgcgctgctcgaccgcaccgaggtg
cccctcagcgtggaggagctcagccgcgcctacgagttgaacctcttctcgccgcgcgtgccccccaagcgccagc
ccaatggcacctgcgagcccaacccgcgcctcaacttctacccggtcttcgcggtgcccgaggccctggccaccta
ccacatctttttcaagaaccaaaagatccccgtctcctgccgcgccaaccgcacccgcgccgacgcccttttcaac
ctgggtcccggcgcccgcctacctgatatcgcctccttggaagaggttcccaagatcttcgagggtctgggcagcg
acgagactcgggccgcgaacgctctgcaaggagaaggaggagagcatgagcaccacagcgccctggtcgagttgga
aggcgacaacgcgcggctggcggtgctcaaacgcacggtcgagctgacccatttcgcctacccggctctgaacctg
ccccccaaagtcatgagcgcggtcatggaccaggtgctcatcaagcgcgcgtcgcccatctccgaggacgagggca
tgcaagactccgaggagggcaagcccgtggtcagcgacgagcagctggcccggtggctgggtcctaatgctagtcc
ccagagtttggaagagcggcgcaaactcatgatggccgtggtcctggtgaccgtggagctggagtgcctgcgccgc
ttcttcgccgacgcggagaccctgcgcaaggtcgaggagaacctgcactacctcttcaggcacgggttcgtgcgcc
aggcctgcaagatctccaacgtggagctgaccaacctggtctcctacatgggcatcttgcacgagaaccgcctggg
gcagaacgtgctgcacaccaccctgcgcggggaggcccggcgcgactacatccgcgactgcgtctacctctacctc
tgccacacctggcagacgggcatgggcgtgtggcagcagtgtctggaggagcagaacctgaaagagctctgcaagc
tcctgcagaagaacctcaagggtctgtggaccgggttcgacgagcgcaccaccgcctcggacctggccgacctcat
tttccccgagcgcctcaggctgacgctgcgcaacggcctgcccgactttatgagccaaagcatgttgcaaaacttt
cgctctttcatcctcgaacgctccggaatcctgcccgccacctgctccgcgctgccctcggacttcgtgccgctga
ccttccgcgagtgccccccgccgctgtggagccactgctacctgctgcgcctggccaactacctggcctaccactc
ggacgtgatcgaggacgtcagcggcgagggcctgctcgagtgccactgccgctgcaacctctgcacgccgcaccgc
tccctggcctgcaacccccagctgctgagcgagacccagatcatcggcaccttcgagttgcaagggcccagcgaag
gcgagggttcagccgccaaggggggtctgaaactcaccccggggctgtggacctcggcctacttgcgcaagttcgt
gcccgaggactaccatcccttcgagatcaggttctacgaggaccaatcccatccgcccaaggccgagctgtcggcc
tgcgtcatcacccagggggcgatcctggcccaattgcaagccatccagaaatcccgccaagaattcttgctgaaaa
agggccgcggggtctacctcgacccccagaccggtgaggagctcaaccccggcttcccccaggatgccccgaggaa
acaagaagctgaaagtggagctgccgcccgtggaggatttggaggaagactgggagaacagcagtcaggcagagga
ggaggagatggaggaagactgggacagcactcaggcagaggaggacagcctgcaagacagtctggaggaagacgag
gaggaggcagaggaggaggtggaagaagcagccgccgccagaccgtcgtcctcggcgggggagaaagcaagcagca
cggataccatctccgctccgggtcggggtcccgctcgaccacacagtagatgggacgagaccggacgattcccgaa
ccccaccacccagaccggtaagaaggagcggcagggatacaagtcctggcgggggcacaaaaacgccatcgtctcc
tgcttgcaggcctgcgggggcaacatctccttcacccggcgctacctgctcttccaccgcggggtgaactttcccc
gcaacatcttgcattactaccgtcacctccacagcccctactacttccaagaagaggcagcagcagcagaaaaaga
ccagcagaaaaccagcagctagaaaatccacagcggcggcagcaggtggactgaggatcgcggcgaacgagccggc
gcaaacccgggagctgaggaaccggatctttcccaccctctatgccatcttccagcagagtcgggggcaggagcag
gaactgaaagtcaagaaccgttctctgcgctcgctcacccgcagttgtctgtatcacaagagcgaagaccaacttc
agcgcactctcgaggacgccgaggctctcttcaacaagtactgcgcgctcactcttaaagagtagcccgcgcccgc
ccagtcgcagaaaaaggcgggaattacgtcacctgtgcccttcgccctagccgcctccacccatcatcatgagcaa
agagattcccacgccttacatgtggagctaccagccccagatgggcctggccgccggtgccgcccaggactactcc
acccgcatgaattggctcagcgccgggcccgcgatgatctcacgggtgaatgacatccgcgcccaccgaaaccaga
tactcctagaacagtcagcgctcaccgccacgccccgcaatcacctcaatccgcgtaattggcccgccgccctggt
gtaccaggaaattccccagcccacgaccgtactacttccgcgagacgcccaggccgaagtccagctgactaactca
ggtgtccagctggcgggcggcgccaccctgtgtcgtcaccgccccgctcagggtataaagcggctggtgatccggg
gcagaggcacacagctcaacgacgaggtggtgagctcttcgctgggtctgcgacctgacggagtcttccaactcgc
cggatcggggagatcttccttcacgcctcgtcaggccgtcctgactttggagagttcgtcctcgcagccccgctcg
ggtggcatcggcactctccagttcgtggaggagttcactccctcggtctacttcaaccccttctccggctcccccg
gccactacccggacgagttcatcccgaacttcgacgccatcagcgagtcggtggacggctacgattgaatgtccca
tggtggcgcagctgacctagctcggcttcgacacctggaccactgccgccgcttccgctgcttcgctcgggatctc
gccgagtttgcctactttgagctgcccgaggagcaccctcagggcccggcccacggagtgcggatcgtcgtcgaag
ggggcctcgactcccacctgcttcggatcttcagccagcgtccgatcctggtcgagcgcgagcaaggacagaccct
tctgactctgtactgcatctgcaaccaccccggcctgcatgaaagtctttgttgtctgctgtgtactgagtataat
aaaagctgagatcagcgactactccggacttccgtgtgttcctgaatccatcaaccagtctttgttcttcaccggg
aacgagaccgagctccagctccagtgtaagccccacaagaagtacctcacctggctgttccagggctccccgatcg
ccgttgtcaaccactgcgacaacgacggagtcctgctgagcggccctgccaaccttactttttccacccgcagaag
caagctccagctcttccaacccttcctccccgggacctatcagtgcgtctcgggaccctgccatcacaccttccac
ctgatcccgaataccacagcgtcgctccccgctactaacaaccaaactaacctccaccaacgccaccgtcgcgacc
tttctgaatctaatactaccacccacaccggaggtgagctccgaggtcaaccaacctctgggatttactacggccc
ctgggaggtggttgggttaatagcgctaggcctagttgcgggtgggcttttggttctctgctacctatacctccct
tgctgttcgtacttagtggtgctgtgttgctggtttaagaaatggggaagatcaccctagtgagctgcggtgcgct
ggtggcggtgttgctttcgattgtgggactgggcggtgcggctgtagtgaaggagaaggccgatccctgcttgcat
ttcaatcccaacaaatgccagctgagttttcagcccgatggcaatcggtgcgcggtactgatcaagtgcggatggg
aatgcgagaacgtgagaatcgagtacaataacaagactcggaacaatactctcgcgtccgtgtggcagcccgggga
ccccgagtggtacaccgtctctgtccccggtgctgacggctccccgcgcaccgtgaataatactttcatttttgcg
cacatgtgcgacacggtcatgtggatgagcaagcagtacgatatgtggccccccacgaaggagaacatcgtggtct
tctccatcgcttacagcctgtgcacggcgctaatcaccgctatcgtgtgcctgagcattcacatgctcatcgctat
tcgccccagaaataatgccgaaaaagaaaaacagccataacgttttttttcacacctttttcagaccatggcctct
gttaaatttttgcttttatttgccagtctcattgccgtcattcatggaatgagtaatgagaaaattactatttaca
ctggcactaatcacacattgaaaggtccagaaaaagccacagaagtttcatggtattgttattttaatgaatcaga
tgtatctactgaactctgtggaaacaataacaaaaaaaatgagagcattactctcatcaagtttcaatgtggatct
gacttaaccctaattaacatcactagagactatgtaggtatgtattatggaactacagcaggcatttcggacatgg
aattttatcaagtttctgtgtctgaacccaccacgcctagaatgaccacaaccacaaaaactacacctgttaccac
tatgcagctcactaccaataacatttttgccatgcgtcaaatggtcaacaatagcactcaacccaccccacccagt
gaggaaattcccaaatccatgattggcattattgttgctgtagtggtgtgcatgttgatcatcgccttgtgcatgg
tgtactatgccttctgctacagaaagcacagactgaacgacaagctggaacacttactaagtgttgaattttaatt
ttttagaaccatgaagatcctaggccttttaattttttctatcattacctctgctctatgcaattctgacaatgag
gacgttactgtcgttgtcggatcaaattatacactgaaaggtccagcgaagggtatgctttcgtggtattgctatt
ttggatctgacactacagaaactgaattatgcaatcttaagaatggcaaaattcaaaattctaaaattaacaatta
tatatgcaatggtactgatctgatactcctcaatatcacgaaatcatatgctggcagttacacctgccctggagat
gatgctgacagtatgattttttacaaagtaactgttgttgatcccactactccacctccacccaccacaactactc
acaccacacacacagatcaaaccgcagcagaggaggcagcaaagttagccttgcaggtccaagacagttcatttgt
tggcattacccctacacctgatcagcggtgtccggggctgctagtcagcggcattgtcggtgtgctttcgggatta
gcagtcataatcatctgcatgttcatttttgcttgctgctatagaaggctttaccgacaaaaatcagacccactgc
tgaacctctatgtttaattttttccagagtcatgaaggcagttagcgctctagttttttgttctttgattggcatt
gttttttgcaatcctattcctaaagttagctttattaaagatgtgaatgttactgaggggggcaatgtgacactgg
taggtgtagagggtgctgaaaacaccacctggacaaaataccacctcaatgggtggaaagatatttgcaattggag
tgtattagtttatacatgtgagggagttaatcttaccattgtcaatgccacctcagctcaaaatggtagaattcaa
ggacaaagtgtcagtgtatctaatgggtattttacccaacatacttttatctatgacgttaaagtcataccactgc
ctacgcctagcccacctagcactaccacacagacaacccacactacacagacaaccacatacagtacattaaatca
gcctaccaccactacagcagcagaggttgccagctcgtctggggtccgagtggcatttttgatgtgggccccatct
agcagtcccactgctagtaccaatgagcagactactgaatttttgtccactgtcgagagccacaccacagctacct
ccagtgccttctctagcaccgccaatctctcctcgctttcctctacaccaatcagtcccgctactactcctagccc
cgctcctcttcccactcccctgaagcaaacagacggcggcatgcaatggcagatcaccctgctcattgtgatcggg
ttggtcatcctggccgtgttgctctactacatcttctgccgccgcattcccaacgcgcaccgcaagccggtctaca
agcccatcattgtcgggcagccggagccgcttcaggtggaagggggtctaaggaatcttctcttctcttttacagt
atggtgattgaactatgattcctagacaattcttgatcactattcttatctgcctcctccaagtctgtgccaccct
cgctctggtggccaacgccagtccagactgtattgggcccttcgcctcctacgtgctctttgccttcaccacctgc
atctgctgctgtagcatagtctgcctgcttatcaccttcttccagttcattgactggatctttgtgcgcatcgcct
acctgcgccaccacccccagtaccgcgaccagcgagtggcgcggctgctcaggctcctctgataagcatgcgggct
ctgctacttctcgcgcttctgctgttagtgctcccccgtcccgtcgacccccggtcccccacccagtcccccgagg
aggtccgcaaatgcaaattccaagaaccctggaaattcctcaaatgctaccgccaaaaatcagacatgcatcccag
ctggatcatgatcattgggatcgtgaacattctggcctgcaccctcatctcctttgtgatttacccctgctttgac
tttggttggaactcgccagaggcgctctatctcccgcctgaacctgacacaccaccacagcaacctcaggcacacg
cactaccaccactacagcctaggccacaatacatgcccatattagactatgaggccgagccacagcgacccatgct
ccccgctattagttacttcaatctaaccggcggagatgactgacccactggccaacaacaacgtcaacgaccttct
cctggacatggacggccgcgcctcggagcagcgactcgcccaacttcgcattcgccagcagcaggagagagccgtc
aaggagctgcaggatgcggtggccatccaccagtgcaagagaggcatcttctgcctggtgaaacaggccaagatct
cctacgaggtcactccaaacgaccatcgcctctcctacgagctcctgcagcagcgccagaagttcacctgcctggt
cggagtcaaccccatcgtcatcacccagcagtctggcgataccaaggggtgcatccactgctcctgcgactccccc
gactgcgtccacactctgatcaagaccctctgcggcctccgcgacctcctccccatgaactaatcacccccttatc
cagtgaaataaagatcatattgatgatgattttacagaaataaaaaataatcatttgatttgaaataaagatacaa
tcatattgatgatttgagtttaacaaaaaaataaagaatcacttacttgaaatctgataccaggtctctgtccatg
ttttctgccaacaccacttcactcccctcttcccagctctggtactgcaggccccggcgggctgcaaacttcctcc
acacgctgaaggggatgtcaaattcctcctgtccctcaatcttcattttatcttctatcagatgtccaaaaagcgc
gtccgggtggatgatgacttcgaccccgtctacccctacgatgcagacaacgcaccgaccgtgcccttcatcaacc
cccccttcgtctcttcagatggattccaagagaagcccctgggggtgttgtccctgcgactggccgaccccgtcac
caccaagaacggggaaatcaccctcaagctgggagagggggtggacctcgattcctcgggaaaactcatctccaac
acggccaccaaggccgccgcccctctcagtttttccaacaacaccatttcccttaacatggatcaccccttttaca
ctaaagatggaaaattatccttacaagtttctccaccattaaatatactgagaacaagcattctaaacacactagc
tttaggttttggatcaggtttaggactccgtggctctgccttggcagtacagttagtctctccacttacatttgat
actgatggaaacataaagcttaccttagacagaggtttgcatgttacaacaggagatgcaattgaaagcaacataa
gctgggctaaaggtttaaaatttgaagatggagccatagcaaccaacattggaaatgggttagagtttggaagcag
tagtacagaaacaggtgttgatgatgcttacccaatccaagttaaacttggatctggccttagctttgacagtaca
ggagccataatggctggtaacaaagaagacgataaactcactttgtggacaacacctgatccatcaccaaactgtc
aaatactcgcagaaaatgatgcaaaactaacactttgcttgactaaatgtggtagtcaaatactggccactgtgtc
agtcttagttgtaggaagtggaaacctaaaccccattactggcaccgtaagcagtgctcaggtgtttctacgtttt
gatgcaaacggtgttcttttaacagaacattctacactaaaaaaatactgggggtataggcagggagatagcatag
atggcactccatataccaatgctgtaggattcatgcccaatttaaaagcttatccaaagtcacaaagttctactac
taaaaataatatagtagggcaagtatacatgaatggagatgtttcaaaacctatgcttctcactataaccctcaat
ggtactgatgacagcaacagtacatattcaatgtcattttcatacacctggactaatggaagctatgttggagcaa
catttggggctaactcttataccttctcatacatcgcccaagaatgaacactgtatcccaccctgcatgccaaccc
ttcccaccccactctgtggaacaaactctgaaacacaaaataaaataaagttcaagtgttttattgattcaacagt
tttacaggattcgagcagttatttttcctccaccctcccaggacatggaatacaccaccctctccccccgcacagc
cttgaacatctgaatgccattggtgatggacatgcttttggtctccacgttccacacagtttcagagcgagccagt
ctcgggtcggtcagggagatgaaaccctccgggcactcccgcatctgcacctcacagctcaacagctgaggattgt
cctcggtggtcgggatcacggttatctggaagaagcagaagagcggcggtgggaatcatagtccgcgaacgggatc
ggccggtggtgtcgcatcaggccccgcagcagtcgctgccgccgccgctccgtcaagctgctgctcagggggtccg
ggtccagggactccctcagcatgatgcccacggccctcagcatcagtcgtctggtgcggcgggcgcagcagcgcat
gcggatctcgctcaggtcgctgcagtacgtgcaacacagaaccaccaggttgttcaacagtccatagttcaacacg
ctccagccgaaactcatcgcgggaaggatgctacccacgtggccgtcgtaccagatcctcaggtaaatcaagtggt
gccccctccagaacacgctgcccacgtacatgatctccttgggcatgtggcggttcaccacctcccggtaccacat
caccctctggttgaacatgcagccccggatgatcctgcggaaccacagggccagcaccgccccgcccgccatgcag
cgaagagaccccgggtcccggcaatggcaatggaggacccaccgctcgtacccgtggatcatctgggagctgaaca
agtctatgttggcacagcacaggcatatgctcatgcatctcttcagcactctcaactcctcgggggtcaaaaccat
atcccagggcacggggaactcttgcaggacagcgaaccccgcagaacagggcaatcctcgcacagaacttacattg
tgcatggacagggtatcgcaatcaggcagcaccgggtgatcctccaccagagaagcgcgggtctcggtctcctcac
agcgtggtaagggggccggccgatacgggtgatggcgggacgcggctgatcgtgttcgcgaccgtgtcatgatgca
gttgctttcggacattttcgtacttgctgtagcagaacctggtccgggcgctgcacaccgatcgccggcggcggtc
tcggcgcttggaacgctcggtgttgaaattgtaaaacagccactctctcagaccgtgcagcagatctagggcctca
ggagtgatgaagatcccatcatgcctgatggctctgatcacatcgaccaccgtggaatgggccagacccagccaga
tgatgcaattttgttgggtttcggtgacggcgggggagggaagaacaggaagaaccatgattaacttttaatccaa
acggtctcggagtacttcaaaatgaagatcgcggagatggcacctctcgcccccgctgtgttggtggaaaataaca
gccaggtcaaaggtgatacggttctcgagatgttccacggtggcttccagcaaagcctccacgcgcacatccagaa
acaagacaatagcgaaagcgggagggttctctaattcctcaatcatcatgttacactcctgcaccatccccagata
attttcatttttccagccttgaatgattcgaactagttcctgaggtaaatccaagccagccatgataaagagctcg
cgcagagcgccctccaccggcattcttaagcacaccctcataattccaagatattctgctcctggttcacctgcag
cagattgacaagcggaatatcaaaatctctgccgcgatccctgagctcctccctcagcaataactgtaagtactct
ttcatatcctctccgaaatttttagccataggaccaccaggaataagattagggcaagccacagtacagataaacc
gaagtcctccccagtgagcattgccaaatgcaagactgctataagcatgctggctagacccggtgatatcttccag
ataactggacagaaaatcgcccaggcaatttttaagaaaatcaacaaaagaaaaatcctccaggtggacgtttaga
gcctcgggaacaacgatgaagtaaatgcaagcggtgcgttccagcatggttagttagctgatctgtagaaaaaaca
aaaatgaacattaaaccatgctagcctggcgaacaggtgggtaaatcgttctctccagcaccaggcaggccacggg
gtctccggcgcgaccctcgtaaaaattgtcgctatgattgaaaaccatcacagagagacgttcccggtggccggcg
tgaatgattcgacaagatgaatacacccccggaacattggcgtccgcgagtgaaaaaaagcgcccgaggaagcaat
aaggcactacaatgctcagtctcaagtccagcaaagcgatgccatgcggatgaagcacaaaattctcaggtgcgta
caaaatgtaattactcccctcctgcacaggcagcaaagcccccgatccctccaggtacacatacaaagcctcagcg
tccatagcttaccgagcagcagcacacaacaggcgcaagagtcagagaaaggctgagctctaacctgtccacccgc
tctctgctcaatatatagcccagatctacactgacgtaaaggccaaagtctaaaaatacccgccaaataatcacac
acgcccagcacacgcccagaaaccggtgacacactcaaaaaaatacgcgcacttcctcaaacgcccaaaactgccg
tcatttccgggttcccacgctacgtcatcaaaacacgactttcaaattccgtcgaccgttaaaaacgtcacccgcc
ccgcccctaacggtcgcccgtctctcagccaatcagcgccccgcatccccaaattcaaacacctcatttgcatatt
aacgcgcacaaaaagtttgaggtatattattgatgatgg
ChAdV68.5WTnt.MAG25mer; AC_000011.1 with El (nt 577 to 3403) and E3
(nt 27,125-31,825) sequences deleted; corresponding ATCC VR-594
nucleotides substituted at five positions; model neoantigen cassette under
the control of the CMV promoter/enhancer inserted in place of deleted E1;
SV40 polyA 3′ of cassette
(SEQ ID NO: 2)
ccatcttcaataatatacctcaaactttttgtgcgcgttaatatgcaaatgaggcgtttgaatttggggaggaagg
gcggtgattggtcgagggatgagcgaccgttaggggcggggcgagtgacgttttgatgacgtggttgcgaggagga
gccagtttgcaagttctcgtgggaaaagtgacgtcaaacgaggtgtggtttgaacacggaaatactcaattttccc
gcgctctctgacaggaaatgaggtgtttctgggcggatgcaagtgaaaacgggccattttcgcgcgaaaactgaat
gaggaagtgaaaatctgagtaatttcgcgtttatggcagggaggagtatttgccgagggccgagtagactttgacc
gattacgtgggggtttcgattaccgtgtttttcacctaaatttccgcgtacggtgtcaaagtccggtgtttttacg
taggtgtcagctgatcgccagggtatttaaacctgcgctctccagtcaagaggccactcttgagtgccagcgagaa
gagttttctcctccgcgccgcgagtcagatctacactttgaaagtagggataacagggtaatgacattgattattg
actagttgttaatagtaatcaattacggggtcattagttcatagcccatatatggagttccgcgttacataactta
cggtaaatggcccgcctggctgaccgcccaacgacccccgcccattgacgtcaataatgacgtatgttcccatagt
aacgccaatagggactttccattgacgtcaatgggtggagtatttacggtaaactgcccacttggcagtacatcaa
gtgtatcatatgccaagtccgccccctattgacgtcaatgacggtaaatggcccgcctggcattatgcccagtaca
tgaccttacgggactttcctacttggcagtacatctacgtattagtcatcgctattaccatggtgatgcggttttg
gcagtacaccaatgggcgtggatagcggtttgactcacggggatttccaagtctccaccccattgacgtcaatggg
agtttgttttggcaccaaaatcaacgggactttccaaaatgtcgtaataaccccgccccgttgacgcaaatgggcg
gtaggcgtgtacggtgggaggtctatataagcagagctcgtttagtgaaccgtcagatcgcctggaacgccatcca
cgctgttttgacctccatagaagacagcgatcgcgccaccatggccgggatgttccaggcactgtccgaaggctgc
acaccctatgatattaaccagatgctgaatgtcctgggagaccaccaggtctctggcctggagcagctggagagca
tcatcaacttcgagaagctgaccgagtggacaagctccaatgtgatgcctatcctgtccccactgaccaagggcat
cctgggcttcgtgtttaccctgacagtgccttctgagcggggcctgtcttgcatcagcgaggcagacgcaaccaca
ccagagtccgccaatctgggcgaggagatcctgtctcagctgtacctgtggccccgggtgacatatcactcccctt
cttacgcctatcaccagttcgagcggagagccaagtacaagagacacttcccaggctttggccagtctctgctgtt
cggctaccccgtgtacgtgttcggcgattgcgtgcagggcgactgggatgccatccggtttagatactgcgcacca
cctggatatgcactgctgaggtgtaacgacaccaattattccgccctgctggcagtgggcgccctggagggccctc
gcaatcaggattggctgggcgtgccaaggcagctggtgacacgcatgcaggccatccagaacgcaggcctgtgcac
cctggtggcaatgctggaggagacaatcttctggctgcaggcctttctgatggccctgaccgacagcggccccaag
acaaacatcatcgtggattcccagtacgtgatgggcatctccaagccttctttccaggagtttgtggactgggaga
acgtgagcccagagctgaattccaccgatcagccattctggcaggcaggaatcctggcaaggaacctggtgcctat
ggtggccacagtgcagggccagaatctgaagtaccagggccagagcctggtcatcagcgcctccatcatcgtgttt
aacctgctggagctggagggcgactatcgggacgatggcaacgtgtgggtgcacaccccactgagccccagaacac
tgaacgcctgggtgaaggccgtggaggagaagaagggcatcccagtgcacctggagctggcctccatgaccaatat
ggagctgatgtctagcatcgtgcaccagcaggtgaggacatacggacccgtgttcatgtgcctgggaggcctgctg
accatggtggcaggagccgtgtggctgacagtgcgggtgctggagctgttcagagccgcccagctggccaacgatg
tggtgctgcagatcatggagctgtgcggagcagcctttcgccaggtgtgccacaccacagtgccatggcccaatgc
ctccctgacccccaagtggaacaatgagacaacacagcctcagatcgccaactgtagcgtgtacgacttcttcgtg
tggctgcactactatagcgtgagggataccctgtggccccgcgtgacataccacatgaataagtacgcctatcaca
tgctggagaggcgcgccaagtataagagaggccctggcccaggcgcaaagtttgtggcagcatggaccctgaaggc
cgccgccggccccggccccggccagtatatcaaggctaacagtaagttcattggaatcacagagctgggacccgga
cctggataatgagtttaaactcccatttaaatgtgagggttaatgcttcgagcagacatgataagatacattgatg
agtttggacaaaccacaactagaatgcagtgaaaaaaatgctttatttgtgaaatttgtgatgctattgctttatt
tgtaaccattataagctgcaataaacaagttaacaacaacaattgcattcattttatgtttcaggttcagggggag
atgtgggaggttttttaaagcaagtaaaacctctacaaatgtggtaaaataactataacggtcctaaggtagcgag
tgagtagtgttctgggggggggaggacctgcatgagggccagaataactgaaatctgtgcttttctgtgtgttgca
gcagcatgagcggaagcggctcctttgagggaggggtattcagcccttatctgacggggcgtctcccctcctgggc
gggagtgcgtcagaatgtgatgggatccacggtggacggccggcccgtgcagcccgcgaactcttcaaccctgacc
tatgcaaccctgagctcttcgtcgttggacgcagctgccgccgcagctgctgcatctgccgccagcgccgtgcgcg
gaatggccatgggcgccggctactacggcactctggtggccaactcgagttccaccaataatcccgccagcctgaa
cgaggagaagctgttgctgctgatggcccagctcgaggccttgacccagcgcctgggcgagctgacccagcaggtg
gctcagctgcaggagcagacgcgggccgcggttgccacggtgaaatccaaataaaaaatgaatcaataaataaacg
gagacggttgttgattttaacacagagtctgaatctttatttgatttttcgcgcgcggtaggccctggaccaccgg
tctcgatcattgagcacccggtggatcttttccaggacccggtagaggtgggcttggatgttgaggtacatgggca
tgagcccgtcccgggggtggaggtagctccattgcagggcctcgtgctcgggggtggtgttgtaaatcacccagtc
atagcaggggcgcagggcatggtgttgcacaatatctttgaggaggagactgatggccacgggcagccctttggtg
taggtgtttacaaatctgttgagctgggagggatgcatgcggggggagatgaggtgcatcttggcctggatcttga
gattggcgatgttaccgcccagatcccgcctggggttcatgttgtgcaggaccaccagcacggtgtatccggtgca
cttggggaatttatcatgcaacttggaagggaaggcgtgaaagaatttggcgacgcctttgtgcccgcccaggttt
tccatgcactcatccatgatgatggcgatgggcccgtgggcggcggcctgggcaaagacgtttcgggggtcggaca
catcatagttgtggtcctgggtgaggtcatcataggccattttaatgaatttggggcggagggtgccggactgggg
gacaaaggtaccctcgatcccgggggcgtagttcccctcacagatctgcatctcccaggctttgagctcggagggg
gggatcatgtccacctgcggggcgataaagaacacggtttccggggcgggggagatgagctgggccgaaagcaagt
tccggagcagctgggacttgccgcagccggtggggccgtagatgaccccgatgaccggctgcaggtggtagttgag
ggagagacagctgccgtcctcccggaggaggggggccacctcgttcatcatctcgcgcacgtgcatgttctcgcgc
accagttccgccaggaggcgctctccccccagggataggagctcctggagcgaggcgaagtttttcagcggcttga
gtccgtcggccatgggcattttggagagggtttgttgcaagagttccaggcggtcccagagctcggtgatgtgctc
tacggcatctcgatccagcagacctcctcgtttcgcgggttgggacggctgcgggagtagggcaccagacgatggg
cgtccagcgcagccagggtccggtccttccagggtcgcagcgtccgcgtcagggtggtctccgtcacggtgaaggg
gtgcgcgccgggctgggcgcttgcgagggtgcgcttcaggctcatccggctggtcgaaaaccgctcccgatcggcg
ccctgcgcgtcggccaggtagcaattgaccatgagttcgtagttgagcgcctcggccgcgtggcctttggcgcgga
gcttacctttggaagtctgcccgcaggcgggacagaggagggacttgagggcgtagagcttgggggcgaggaagac
ggactcgggggcgtaggcgtccgcgccgcagtgggcgcagacggtctcgcactccacgagccaggtgaggtcgggc
tggtcggggtcaaaaaccagtttcccgccgttctttttgatgcgtttcttacctttggtctccatgagctcgtgtc
cccgctgggtgacaaagaggctgtccgtgtccccgtagaccgactttatgggccggtcctcgagcggtgtgccgcg
gtcctcctcgtagaggaaccccgcccactccgagacgaaagcccgggtccaggccagcacgaaggaggccacgtgg
gacgggtagcggtcgttgtccaccagcgggtccaccttttccagggtatgcaaacacatgtccccctcgtccacat
ccaggaaggtgattggcttgtaagtgtaggccacgtgaccgggggtcccggccgggggggtataaaagggtgcggg
tccctgctcgtcctcactgtcttccggatcgctgtccaggagcgccagctgttggggtaggtattccctctcgaag
gcgggcatgacctcggcactcaggttgtcagtttctagaaacgaggaggatttgatattgacggtgccggcggaga
tgcctttcaagagcccctcgtccatctggtcagaaaagacgatctttttgttgtcgagcttggtggcgaaggagcc
gtagagggcgttggagaggagcttggcgatggagcgcatggtctggtttttttccttgtcggcgcgctccttggcg
gcgatgttgagctgcacgtactcgcgcgccacgcacttccattcggggaagacggtggtcagctcgtcgggcacga
ttctgacctgccagccccgattatgcagggtgatgaggtccacactggtggccacctcgccgcgcaggggctcatt
agtccagcagaggcgtccgcccttgcgcgagcagaaggggggcagggggtccagcatgacctcgtcgggggggtcg
gcatcgatggtgaagatgccgggcaggaggtcggggtcaaagtagctgatggaagtggccagatcgtccagggcag
cttgccattcgcgcacggccagcgcgcgctcgtagggactgaggggcgtgccccagggcatgggatgggtaagcgc
ggaggcgtacatgccgcagatgtcgtagacgtagaggggctcctcgaggatgccgatgtaggtggggtagcagcgc
cccccgcggatgctggcgcgcacgtagtcatacagctcgtgcgagggggcgaggagccccgggcccaggttggtgc
gactgggcttttcggcgcggtagacgatctggcggaaaatggcatgcgagttggaggagatggtgggcctttggaa
gatgttgaagtgggcgtggggcagtccgaccgagtcgcggatgaagtgggcgtaggagtcttgcagcttggcgacg
agctcggcggtgactaggacgtccagagcgcagtagtcgagggtctcctggatgatgtcatacttgagctgtccct
tttgtttccacagctcgcggttgagaaggaactcttcgcggtccttccagtactcttcgagggggaacccgtcctg
atctgcacggtaagagcctagcatgtagaactggttgacggccttgtaggcgcagcagcccttctccacggggagg
gcgtaggcctgggcggccttgcgcagggaggtgtgcgtgagggcgaaagtgtccctgaccatgaccttgaggaact
ggtgcttgaagtcgatatcgtcgcagcccccctgctcccagagctggaagtccgtgcgcttcttgtaggcggggtt
gggcaaagcgaaagtaacatcgttgaagaggatcttgcccgcgcggggcataaagttgcgagtgatgcggaaaggt
tggggcacctcggcccggttgttgatgacctgggcggcgagcacgatctcgtcgaagccgttgatgttgtggccca
cgatgtagagttccacgaatcgcggacggcccttgacgtggggcagtttcttgagctcctcgtaggtgagctcgtc
ggggtcgctgagcccgtgctgctcgagcgcccagtcggcgagatgggggttggcgcggaggaaggaagtccagaga
tccacggccagggcggtttgcagacggtcccggtactgacggaactgctgcccgacggccattttttcgggggtga
cgcagtagaaggtgcgggggtccccgtgccagcgatcccatttgagctggagggcgagatcgagggcgagctcgac
gagccggtcgtccccggagagtttcatgaccagcatgaaggggacgagctgcttgccgaaggaccccatccaggtg
taggtttccacatcgtaggtgaggaagagcctttcggtgcgaggatgcgagccgatggggaagaactggatctcct
gccaccaattggaggaatggctgttgatgtgatggaagtagaaatgccgacggcgcgccgaacactcgtgcttgtg
tttatacaagcggccacagtgctcgcaacgctgcacgggatgcacgtgctgcacgagctgtacctgagttcctttg
acgaggaatttcagtgggaagtggagtcgtggcgcctgcatctcgtgctgtactacgtcgtggtggtcggcctggc
cctcttctgcctcgatggtggtcatgctgacgagcccgcgcgggaggcaggtccagacctcggcgcgagcgggtcg
gagagcgaggacgagggcgcgcaggccggagctgtccagggtcctgagacgctgcggagtcaggtcagtgggcagc
ggcggcgcgcggttgacttgcaggagtttttccagggcgcgcgggaggtccagatggtacttgatctccaccgcgc
cattggtggcgacgtcgatggcttgcagggtcccgtgcccctggggtgtgaccaccgtcccccgtttcttcttggg
cggctggggcgacgggggcggtgcctcttccatggttagaagcggcggcgaggacgcgcgccgggggcaggggggc
tcggggcccggaggcaggggcggcaggggcacgtcggcgccgcgcgcgggtaggttctggtactgcgcccggagaa
gactggcgtgagcgacgacgcgacggttgacgtcctggatctgacgcctctgggtgaaggccacgggacccgtgag
tttgaacctgaaagagagttcgacagaatcaatctcggtatcgttgacggcggcctgccgcaggatctcttgcacg
tcgcccgagttgtcctggtaggcgatctcggtcatgaactgctcgatctcctcctcttgaaggtctccgcggccgg
cgcgctccacggtggccgcgaggtcgttggagatgcggcccatgagctgcgagaaggcgttcatgcccgcctcgtt
ccagacgcggctgtagaccacgacgccctcgggatcgcgggcgcgcatgaccacctgggcgaggttgagctccacg
tggcgcgtgaagaccgcgtagttgcagaggcgctggtagaggtagttgagcgtggtggcgatgtgctcggtgacga
agaaatacatgatccagcggcggagcggcatctcgctgacgtcgcccagcgcctccaaacgttccatggcctcgta
aaagtccacggcgaagttgaaaaactgggagttgcgcgccgagacggtcaactcctcctccagaagacggatgagc
tcggcgatggtggcgcgcacctcgcgctcgaaggcccccgggagttcctccacttcctcttcttcctcctccacta
acatctcttctacttcctcctcaggcggcagtggtggggggggggggcctgcgtcgccggcggcgcacgggcagac
ggtcgatgaagcgctcgatggtctcgccgcgccggcgtcgcatggtctcggtgacggcgcgcccgtcctcgcgggg
ccgcagcgtgaagacgccgccgcgcatctccaggtggccgggggggtccccgttgggcagggagagggcgctgacg
atgcatcttatcaattgccccgtagggactccgcgcaaggacctgagcgtctcgagatccacgggatctgaaaacc
gctgaacgaaggcttcgagccagtcgcagtcgcaaggtaggctgagcacggtttcttctggcgggtcatgttggtt
gggagcggggcgggcgatgctgctggtgatgaagttgaaataggcggttctgagacggcggatggtggcgaggagc
accaggtctttgggcccggcttgctggatgcgcagacggtcggccatgccccaggcgtggtcctgacacctggcca
ggtccttgtagtagtcctgcatgagccgctccacgggcacctcctcctcgcccgcgcggccgtgcatgcgcgtgag
cccgaagccgcgctggggctggacgagcgccaggtcggcgacgacgcgctcggcgaggatggcttgctggatctgg
gtgagggtggtctggaagtcatcaaagtcgacgaagcggtggtaggctccggtgttgatggtgtaggagcagttgg
ccatgacggaccagttgacggtctggtggcccggacgcacgagctcgtggtacttgaggcgcgagtaggcgcgcgt
gtcgaagatgtagtcgttgcaggtgcgcaccaggtactggtagccgatgaggaagtgcggggggctggcggtagag
cggccatcgctcggtggcgggggcgccgggcgcgaggtcctcgagcatggtgcggtggtagccgtagatgtacctg
gacatccaggtgatgccggcggcggtggtggaggcgcgcgggaactcgcggacgcggttccagatgttgcgcagcg
gcaggaagtagttcatggtgggcacggtctggcccgtgaggcgcgcgcagtcgtggatgctctatacgggcaaaaa
cgaaagcggtcagcggctcgactccgtggcctggaggctaagcgaacgggttgggctgcgcgtgtaccccggttcg
aatctcgaatcaggctggagccgcagctaacgtggtattggcactcccgtctcgacccaagcctgcaccaaccctc
caggatacggaggcgggtcgttttgcaacttttttttggaggccggatgagactagtaagcgcggaaagcggccga
ccgcgatggctcgctgccgtagtctggagaagaatcgccagggttgcgttgcggtgtgccccggttcgaggccggc
cggattccgcggctaacgagggcgtggctgccccgtcgtttccaagaccccatagccagccgacttctccagttac
ggagcgagcccctcttttgttttgtttgtttttgccagatgcatcccgtactgcggcagatgcgcccccaccaccc
tccaccgcaacaacagccccctccacagccggcgcttctgcccccgccccagcagcaacttccagccacgaccgcc
gcggccgccgtgagcggggctggacagagttatgatcaccagctggccttggaagagggcgaggggctggcgcgcc
tgggggcgtcgtcgccggagcggcacccgcgcgtgcagatgaaaagggacgctcgcgaggcctacgtgcccaagca
gaacctgttcagagacaggagcggcgaggagcccgaggagatgcgcgcggcccggttccacgcggggcgggagctg
cggcgcggcctggaccgaaagagggtgctgagggacgaggatttcgaggcggacgagctgacggggatcagccccg
cgcgcgcgcacgtggccgcggccaacctggtcacggcgtacgagcagaccgtgaaggaggagagcaacttccaaaa
atccttcaacaaccacgtgcgcaccctgatcgcgcgcgaggaggtgaccctgggcctgatgcacctgtgggacctg
ctggaggccatcgtgcagaaccccaccagcaagccgctgacggcgcagctgttcctggtggtgcagcatagtcggg
acaacgaagcgttcagggaggcgctgctgaatatcaccgagcccgagggccgctggctcctggacctggtgaacat
tctgcagagcatcgtggtgcaggagcgcgggctgccgctgtccgagaagctggcggccatcaacttctcggtgctg
agtttgggcaagtactacgctaggaagatctacaagaccccgtacgtgcccatagacaaggaggtgaagatcgacg
ggttttacatgcgcatgaccctgaaagtgctgaccctgagcgacgatctgggggtgtaccgcaacgacaggatgca
ccgtgcggtgagcgccagcaggcggcgcgagctgagcgaccaggagctgatgcatagtctgcagcgggccctgacc
ggggccgggaccgagggggagagctactttgacatgggcgcggacctgcactggcagcccagccgccgggccttgg
aggcggcggcaggaccctacgtagaagaggtggacgatgaggtggacgaggagggcgagtacctggaagactgatg
gcgcgaccgtatttttgctagatgcaacaacaacagccacctcctgatcccgcgatgcgggcggcgctgcagagcc
agccgtccggcattaactcctcggacgattggacccaggccatgcaacgcatcatggcgctgacgacccgcaaccc
cgaagcctttagacagcagccccaggccaaccggctctcggccatcctggaggccgtggtgccctcgcgctccaac
cccacgcacgagaaggtcctggccatcgtgaacgcgctggtggagaacaaggccatccgcggcgacgaggccggcc
tggtgtacaacgcgctgctggagcgcgtggcccgctacaacagcaccaacgtgcagaccaacctggaccgcatggt
gaccgacgtgcgcgaggccgtggcccagcgcgagcggttccaccgcgagtccaacctgggatccatggtggcgctg
aacgccttcctcagcacccagcccgccaacgtgccccggggccaggaggactacaccaacttcatcagcgccctgc
gcctgatggtgaccgaggtgccccagagcgaggtgtaccagtccgggccggactacttcttccagaccagtcgcca
gggcttgcagaccgtgaacctgagccaggctttcaagaacttgcagggcctgtggggcgtgcaggccccggtcggg
gaccgcgcgacggtgtcgagcctgctgacgccgaactcgcgcctgctgctgctgctggtggcccccttcacggaca
gcggcagcatcaaccgcaactcgtacctgggctacctgattaacctgtaccgcgaggccatcggccaggcgcacgt
ggacgagcagacctaccaggagatcacccacgtgagccgcgccctgggccaggacgacccgggcaacctggaagcc
accctgaactttttgctgaccaaccggtcgcagaagatcccgccccagtacgcgctcagcaccgaggaggagcgca
tcctgcgttacgtgcagcagagcgtgggcctgttcctgatgcaggagggggccacccccagcgccgcgctcgacat
gaccgcgcgcaacatggagcccagcatgtacgccagcaaccgcccgttcatcaataaactgatggactacttgcat
cgggggccgccatgaactctgactatttcaccaacgccatcctgaatccccactggctcccgccgccggggttcta
cacgggcgagtacgacatgcccgaccccaatgacgggttcctgtgggacgatgtggacagcagcgtgttctccccc
cgaccgggtgctaacgagcgccccttgtggaagaaggaaggcagcgaccgacgcccgtcctcggcgctgtccggcc
gcgagggtgctgccgcggcggtgcccgaggccgccagtcctttcccgagcttgcccttctcgctgaacagtatccg
cagcagcgagctgggcaggatcacgcgcccgcgcttgctgggcgaagaggagtacttgaatgactcgctgttgaga
cccgagcgggagaagaacttccccaataacgggatagaaagcctggtggacaagatgagccgctggaagacgtatg
cgcaggagcacagggacgatccccgggcgtcgcagggggccacgagccggggcagcgccgcccgtaaacgccggtg
gcacgacaggcagcggggacagatgtgggacgatgaggactccgccgacgacagcagcgtgttggacttgggtggg
agtggtaacccgttcgctcacctgcgcccccgtatcgggcgcatgatgtaagagaaaccgaaaataaatgatactc
accaaggccatggcgaccagcgtgcgttcgtttcttctctgttgttgttgtatctagtatgatgaggcgtgcgtac
ccggagggtcctcctccctcgtacgagagcgtgatgcagcaggcgatggcggcggcggcgatgcagcccccgctgg
aggctccttacgtgcccccgcggtacctggcgcctacggaggggcggaacagcattcgttactcggagctggcacc
cttgtacgataccacccggttgtacctggtggacaacaagtcggcggacatcgcctcgctgaactaccagaacgac
cacagcaacttcctgaccaccgtggtgcagaacaatgacttcacccccacggaggccagcacccagaccatcaact
ttgacgagcgctcgcggtggggcggccagctgaaaaccatcatgcacaccaacatgcccaacgtgaacgagttcat
gtacagcaacaagttcaaggcgcgggtgatggtctcccgcaagacccccaatggggtgacagtgacagaggattat
gatggtagtcaggatgagctgaagtatgaatgggtggaatttgagctgcccgaaggcaacttctcggtgaccatga
ccatcgacctgatgaacaacgccatcatcgacaattacttggcggtggggcggcagaacggggtgctggagagcga
catcggcgtgaagttcgacactaggaacttcaggctgggctgggaccccgtgaccgagctggtcatgcccggggtg
tacaccaacgaggctttccatcccgatattgtcttgctgcccggctgcggggtggacttcaccgagagccgcctca
gcaacctgctgggcattcgcaagaggcagcccttccaggaaggcttccagatcatgtacgaggatctggagggggg
caacatccccgcgctcctggatgtcgacgcctatgagaaaagcaaggaggatgcagcagctgaagcaactgcagcc
gtagctaccgcctctaccgaggtcaggggcgataattttgcaagcgccgcagcagtggcagcggccgaggcggctg
aaaccgaaagtaagatagtcattcagccggtggagaaggatagcaagaacaggagctacaacgtactaccggacaa
gataaacaccgcctaccgcagctggtacctagcctacaactatggcgaccccgagaagggcgtgcgctcctggacg
ctgctcaccacctcggacgtcacctgcggcgtggagcaagtctactggtcgctgcccgacatgatgcaagacccgg
tcaccttccgctccacgcgtcaagttagcaactacccggtggtgggcgccgagctcctgcccgtctactccaagag
cttcttcaacgagcaggccgtctactcgcagcagctgcgcgccttcacctcgcttacgcacgtcttcaaccgcttc
cccgagaaccagatcctcgtccgcccgcccgcgcccaccattaccaccgtcagtgaaaacgttcctgctctcacag
atcacgggaccctgccgctgcgcagcagtatccggggagtccagcgcgtgaccgttactgacgccagacgccgcac
ctgcccctacgtctacaaggccctgggcatagtcgcgccgcgcgtcctctcgagccgcaccttctaaatgtccatt
ctcatctcgcccagtaataacaccggttggggcctgcgcgcgcccagcaagatgtacggaggcgctcgccaacgct
ccacgcaacaccccgtgcgcgtgcgcgggcacttccgcgctccctggggcgccctcaagggccgcgtgcggtcgcg
caccaccgtcgacgacgtgatcgaccaggtggtggccgacgcgcgcaactacacccccgccgccgcgcccgtctcc
accgtggacgccgtcatcgacagcgtggtggccgacgcgcgccggtacgcccgcgccaagagccggcggcggcgca
tcgcccggcggcaccggagcacccccgccatgcgcgcggcgcgagccttgctgcgcagggccaggcgcacgggacg
cagggccatgctcagggcggccagacgcgcggcttcaggcgccagcgccggcaggacccggagacgcgcggccacg
gcggcggcagcggccatcgccagcatgtcccgcccgcggcgagggaacgtgtactgggtgcgcgacgccgccaccg
gtgtgcgcgtgcccgtgcgcacccgcccccctcgcacttgaagatgttcacttcgcgatgttgatgtgtcccagcg
gcgaggaggatgtccaagcgcaaattcaaggaagagatgctccaggtcatcgcgcctgagatctacggccctgcgg
tggtgaaggaggaaagaaagccccgcaaaatcaagcgggtcaaaaaggacaaaaaggaagaagaaagtgatgtgga
cggattggtggagtttgtgcgcgagttcgccccccggcggcgcgtgcagtggcgcgggcggaaggtgcaaccggtg
ctgagacccggcaccaccgtggtcttcacgcccggcgagcgctccggcaccgcttccaagcgctcctacgacgagg
tgtacggggatgatgatattctggagcaggcggccgagcgcctgggcgagtttgcttacggcaagcgcagccgttc
cgcaccgaaggaagaggcggtgtccatcccgctggaccacggcaaccccacgccgagcctcaagcccgtgaccttg
cagcaggtgctgccgaccgcggcgccgcgccgggggttcaagcgcgagggcgaggatctgtaccccaccatgcagc
tgatggtgcccaagcgccagaagctggaagacgtgctggagaccatgaaggtggacccggacgtgcagcccgaggt
caaggtgcggcccatcaagcaggtggccccgggcctgggcgtgcagaccgtggacatcaagattcccacggagccc
atggaaacgcagaccgagcccatgatcaagcccagcaccagcaccatggaggtgcagacggatccctggatgccat
cggctcctagtcgaagaccccggcgcaagtacggcgcggccagcctgctgatgcccaactacgcgctgcatccttc
catcatccccacgccgggctaccgcggcacgcgcttctaccgcggtcataccagcagccgccgccgcaagaccacc
actcgccgccgccgtcgccgcaccgccgctgcaaccacccctgccgccctggtgggagagtgtaccgccgcggccg
cgcacctctgaccctgccgcgcgcgcgctaccacccgagcatcgccatttaaactttcgcctgctttgcagatcaa
tggccctcacatgccgccttcgcgttcccattacgggctaccgaggaagaaaaccgcgccgtagaaggctgggggg
aacgggatgcgtcgccaccaccaccggggggcgcgccatcagcaagggttggggggaggcttcctgcccgcgctga
tccccatcatcgccgcggcgatcggggcgatccccggcattgcttccgtggcggtgcaggcctctcagcgccactg
agacacacttggaaacatcttgtaataaaccaatggactctgacgctcctggtcctgtgatgtgttttcgtagaca
gatggaagacatcaatttttcgtccctggctccgcgacacggcacgcggccgttcatgggcacctggagcgacatc
ggcaccagccaactgaacgggggcgccttcaattggagcagtctctggagcgggcttaagaatttcgggtccacgc
ttaaaacctatggcagcaaggcgtggaacagcaccacagggcaggcgctgagggataagctgaaagagcagaactt
ccagcagaaggtggtcgatgggctcgcctcgggcatcaacggggtggtggacctggccaaccaggccgtgcagcgg
cagatcaacagccgcctggacccggtgccgcccgccggctccgtggagatgccgcaggtggaggaggagctgcctc
ccctggacaagcggggcgagaagcgaccccgccccgatgcggaggagacgctgctgacgcacacggacgagccgcc
cccgtacgaggaggcggtgaaactgggtctgcccaccacgcggcccatcgcgcccctggccaccggggtgctgaaa
cccgaaaagcccgcgaccctggacttgcctcctccccagccttcccgcccctctacagtggctaagcccctgccgc
cggtggccgtggcccgcgcgcgacccgggggcaccgcccgccctcatgcgaactggcagagcactctgaacagcat
cgtgggtctgggagtgcagagtgtgaagcgccgccgctgctattaaacctaccgtagcgcttaacttgcttgtctg
tgtgtgtatgtattatgtcgccgccgccgctgtccaccagaaggaggagtgaagaggcgcgtcgccgagttgcaag
atggccaccccatcgatgctgccccagtgggcgtacatgcacatcgccggacaggacgcttcggagtacctgagtc
cgggtctggtgcagtttgcccgcgccacagacacctacttcagtctggggaacaagtttaggaaccccacggtggc
gcccacgcacgatgtgaccaccgaccgcagccagcggctgacgctgcgcttcgtgcccgtggaccgcgaggacaac
acctactcgtacaaagtgcgctacacgctggccgtgggcgacaaccgcgtgctggacatggccagcacctactttg
acatccgcggcgtgctggatcggggccctagcttcaaaccctactccggcaccgcctacaacagtctggcccccaa
gggagcacccaacacttgtcagtggacatataaagccgatggtgaaactgccacagaaaaaacctatacatatgga
aatgcacccgtgcagggcattaacatcacaaaagatggtattcaacttggaactgacaccgatgatcagccaatct
acgcagataaaacctatcagcctgaacctcaagtgggtgatgctgaatggcatgacatcactggtactgatgaaaa
gtatggaggcagagctcttaagcctgataccaaaatgaagccttgttatggttcttttgccaagcctactaataaa
gaaggaggtcaggcaaatgtgaaaacaggaacaggcactactaaagaatatgacatagacatggctttctttgaca
acagaagtgcggctgctgctggcctagctccagaaattgttttgtatactgaaaatgtggatttggaaactccaga
tacccatattgtatacaaagcaggcacagatgacagcagctcttctattaatttgggtcagcaagccatgcccaac
agacctaactacattggtttcagagacaactttatcgggctcatgtactacaacagcactggcaatatgggggtgc
tggccggtcaggcttctcagctgaatgctgtggttgacttgcaagacagaaacaccgagctgtcctaccagctctt
gcttgactctctgggtgacagaacccggtatttcagtatgtggaatcaggcggtggacagctatgatcctgatgtg
cgcattattgaaaatcatggtgtggaggatgaacttcccaactattgtttccctctggatgctgttggcagaacag
atacttatcagggaattaaggctaatggaactgatcaaaccacatggaccaaagatgacagtgtcaatgatgctaa
tgagataggcaagggtaatccattcgccatggaaatcaacatccaagccaacctgtggaggaacttcctctacgcc
aacgtggccctgtacctgcccgactcttacaagtacacgccggccaatgttaccctgcccaccaacaccaacacct
acgattacatgaacggccgggtggtggcgccctcgctggtggactcctacatcaacatcggggcgcgctggtcgct
ggatcccatggacaacgtgaaccccttcaaccaccaccgcaatgggggctgcgctaccgctccatgctcctgggca
acgggcgctacgtgcccttccacatccaggtgccccagaaatttttcgccatcaagagcctcctgctcctgcccgg
gtcctacacctacgagtggaacttccgcaaggacgtcaacatgatcctgcagagctccctcggcaacgacctgcgc
acggacggggcctccatctccttcaccagcatcaacctctacgccaccttcttccccatggcgcacaacacggcct
ccacgctcgaggccatgctgcgcaacgacaccaacgaccagtccttcaacgactacctctcggcggccaacatgct
ctaccccatcccggccaacgccaccaacgtgcccatctccatcccctcgcgcaactgggccgccttccgcggctgg
tccttcacgcgtctcaagaccaaggagacgccctcgctgggctccgggttcgacccctacttcgtctactcgggct
ccatcccctacctcgacggcaccttctacctcaaccacaccttcaagaaggtctccatcaccttcgactcctccgt
cagctggcccggcaacgaccggctcctgacgcccaacgagttcgaaatcaagcgcaccgtcgacggcgagggctac
aacgtggcccagtgcaacatgaccaaggactggttcctggtccagatgctggcccactacaacatcggctaccagg
gcttctacgtgcccgagggctacaaggaccgcatgtactccttcttccgcaacttccagcccatgagccgccaggt
ggtggacgaggtcaactacaaggactaccaggccgtcaccctggcctaccagcacaacaactcgggcttcgtcggc
tacctcgcgcccaccatgcgccagggccagccctaccccgccaactacccctacccgctcatcggcaagagcgccg
tcaccagcgtcacccagaaaaagttcctctgcgacagggtcatgtggcgcatccccttctccagcaacttcatgtc
catgggcgcgctcaccgacctcggccagaacatgctctatgccaactccgcccacgcgctagacatgaatttcgaa
gtcgaccccatggatgagtccacccttctctatgttgtcttcgaagtcttcgacgtcgtccgagtgcaccagcccc
accgcggcgtcatcgaggccgtctacctgcgcacccccttctcggccggtaacgccaccacctaagctcttgcttc
ttgcaagccatggccggggctccggcgagcaggagctcagggccatcatccgcgacctgggctgcgggccctactt
cctgggcaccttcgataagcgcttcccgggattcatggccccgcacaagctggcctgcgccatcgtcaacacggcc
ggccgcgagaccgggggcgagcactggctggccttcgcctggaacccgcgctcgaacacctgctacctcttcgacc
ccttcgggttctcggacgagcgcctcaagcagatctaccagttcgagtacgagggcctgctgcgccgcagcgccct
ggccaccgaggaccgctgcgtcaccctggaaaagtccacccagaccgtgcagggtccgcgctcggccgcctgcggg
ctcttctgctgcatgttcctgcacgccttcgtgcactggcccgaccgccccatggacaagaaccccaccatgaact
tgctgacgggggtgcccaacggcatgctccagtcgccccaggtggaacccaccctgcgccgcaaccaggaggcgct
ctaccgcttcctcaactcccactccgcctactttcgctcccaccgcgcgcgcatcgagaaggccaccgccttcgac
cgcatgaatcaagacatgtaaaccgtgtgtgtatgttaaatgtctttaataaacagcactttcatgttacacatgc
atctgagatgatttatttagaaatcgaaagggttctgccgggtctcggcatggcccgcgggcagggacacgttgcg
gaactggtacttggccagccacttgaactcggggatcagcagtttgggcagcggggtgtcggggaaggagtcggtc
cacagcttccgcgtcagttgcagggcgcccagcaggtcgggcgcggagatcttgaaatcgcagttgggacccgcgt
tctgcgcgcgggagttgcggtacacggggttgcagcactggaacaccatcagggccgggtgcttcacgctcgccag
caccgtcgcgtcggtgatgctctccacgtcgaggtcctcggcgttggccatcccgaagggggtcatcttgcaggtc
tgccttcccatggtgggcacgcacccgggcttgtggttgcaatcgcagtgcagggggatcagcatcatctgggcct
ggtcggcgttcatccccgggtacatggccttcatgaaagcctccaattgcctgaacgcctgctgggccttggctcc
ctcggtgaagaagaccccgcaggacttgctagagaactggttggtggcgcacccggcgtcgtgcacgcagcagcgc
gcgtcgttgttggccagctgcaccacgctgcgcccccagcggttctgggtgatcttggcccggtcggggttctcct
tcagcgcgcgctgcccgttctcgctcgccacatccatctcgatcatgtgctccttctggatcatggtggtcccgtg
caggcaccgcagcttgccctcggcctcggtgcacccgtgcagccacagcgcgcacccggtgcactcccagttcttg
tgggcgatctgggaatgcgcgtgcacgaagccctgcaggaagcggcccatcatggtggtcagggtcttgttgctag
tgaaggtcagcggaatgccgcggtgctcctcgttgatgtacaggtggcagatgcggcggtacacctcgccctgctc
gggcatcagctggaagttggctttcaggtcggtctccacgcggtagcggtccatcagcatagtcatgatttccata
cccttctcccaggccgagacgatgggcaggctcatagggttcttcaccatcatcttagcgctagcagccgcggcca
gggggtcgctctcgtccagggtctcaaagctccgcttgccgtccttctcggtgatccgcaccggggggtagctgaa
gcccacggccgccagctcctcctcggcctgtctttcgtcctcgctgtcctggctgacgtcctgcaggaccacatgc
ttggtcttgcggggtttcttcttgggcggcagcggcggcggagatgttggagatggcgagggggagcgcgagttct
cgctcaccactactatctcttcctcttcttggtccgaggccacgcggcggtaggtatgtctcttcgggggcagagg
cggaggcgacgggctctcgccgccgcgacttggcggatggctggcagagccccttccgcgttcgggggtgcgctcc
cggcggcgctctgactgacttcctccgcggccggccattgtgttctcctagggaggaacaacaagcatggagactc
agccatcgccaacctcgccatctgcccccaccgccgacgagaagcagcagcagcagaatgaaagcttaaccgcccc
gccgcccagccccgccacctccgacgggccgtcccagacatgcaagagatggaggaatccatcgagattgacctgg
gctatgtgacgcccgcggagcacgaggaggagctggcagtgcgcttttcacaagaagagatacaccaagaacagcc
agagcaggaagcagagaatgagcagagtcaggctgggctcgagcatgacggcgactacctccacctgagcgggggg
gaggacgcgctcatcaagcatctggcccggcaggccaccatcgtcaaggatgcgctgctcgaccgcaccgaggtgc
ccctcagcgtggaggagctcagccgcgcctacgagttgaacctcttctcgccgcgcgtgccccccaagcgccagcc
caatggcacctgcgagcccaacccgcgcctcaacttctacccggtcttcgcggtgcccgaggccctggccacctac
cacatctttttcaagaaccaaaagatccccgtctcctgccgcgccaaccgcacccgcgccgacgcccttttcaacc
tgggtcccggcgccgcctacctgatatcgcctccttggaagaggttcccaagatcttcgagggtctgggcagcgac
gagactcgggccgcgaacgctctgcaaggagaaggaggagagcatgagcaccacagcgccctggtcgagttggaag
gcgacaacgcgcggctggcggtgctcaaacgcacggtcgagctgacccatttcgcctacccggctctgaacctgcc
ccccaaagtcatgagcgcggtcatggaccaggtgctcatcaagcgcgcgtcgcccatctccgaggacgagggcatg
caagactccgaggagggcaagcccgtggtcagcgacgagcagctggcccggtggctgggtcctaatgctagtcccc
agagtttggaagagcggcgcaaactcatgatggccgtggtcctggtgaccgtggagctggagtgcctgcgccgctt
cttcgccgacgcggagaccctgcgcaaggtcgaggagaacctgcactacctcttcaggcacgggttcgtgcgccag
gcctgcaagatctccaacgtggagctgaccaacctggtctcctacatgggcatcttgcacgagaaccgcctggggc
agaacgtgctgcacaccaccctgcgcggggaggcccggcgcgactacatccgcgactgcgtctacctctacctctg
ccacacctggcagacgggcatgggcgtgtggcagcagtgtctggaggagcagaacctgaaagagctctgcaagctc
ctgcagaagaacctcaagggtctgtggaccgggttcgacgagcgcaccaccgcctcggacctggccgacctcattt
tccccgagcgcctcaggctgacgctgcgcaacggcctgcccgactttatgagccaaagcatgttgcaaaactttcg
ctctttcatcctcgaacgctccggaatcctgcccgccacctgctccgcgctgccctcggacttcgtgccgctgacc
ttccgcgagtgccccccgccgctgtggagccactgctacctgctgcgcctggccaactacctggcctaccactcgg
acgtgatcgaggacgtcagcggcgagggcctgctcgagtgccactgccgctgcaacctctgcacgccgcaccgctc
cctggcctgcaacccccagctgctgagcgagacccagatcatcggcaccttcgagttgcaagggcccagcgaaggc
gagggttcagccgccaaggggggtctgaaactcaccccggggctgtggacctcggcctacttgcgcaagttcgtgc
ccgaggactaccatcccttcgagatcaggttctacgaggaccaatcccatccgcccaaggccgagctgtcggcctg
cgtcatcacccagggggcgatcctggcccaattgcaagccatccagaaatcccgccaagaattcttgctgaaaaag
ggccgcggggtctacctcgacccccagaccggtgaggagctcaaccccggcttcccccaggatgccccgaggaaac
aagaagctgaaagtggagctgccgcccgtggaggatttggaggaagactgggagaacagcagtcaggcagaggagg
aggagatggaggaagactgggacagcactcaggcagaggaggacagcctgcaagacagtctggaggaagacgagga
ggaggcagaggaggaggtggaagaagcagccgccgccagaccgtcgtcctcggcgggggagaaagcaagcagcacg
gataccatctccgctccgggtcggggtcccgctcgaccacacagtagatgggacgagaccggacgattcccgaacc
ccaccacccagaccggtaagaaggagcggcagggatacaagtcctggcgggggcacaaaaacgccatcgtctcctg
cttgcaggcctgcgggggcaacatctccttcacccggcgctacctgctcttccaccgcggggtgaactttccccgc
aacatcttgcattactaccgtcacctccacagcccctactacttccaagaagaggcagcagcagcagaaaaagacc
agcagaaaaccagcagctagaaaatccacagcggggcagcaggtggactgaggatcgcggcgaacgagccggcgca
aacccgggagctgaggaaccggatctttcccaccctctatgccatcttccagcagagtcgggggcaggagcaggaa
ctgaaagtcaagaaccgttctctgcgctcgctcacccgcagttgtctgtatcacaagagcgaagaccaacttcagc
gcactctcgaggacgccgaggctctcttcaacaagtactgcgcgctcactcttaaagagtagcccgcgcccgccca
gtcgcagaaaaagggggaattacgtcacctgtgcccttcgccctagccgcctccacccatcatcatgagcaaagag
attcccacgccttacatgtggagctaccagccccagatgggcctggccgccggtgccgcccaggactactccaccc
gcatgaattggctcagcgccgggcccgcgatgatctcacgggtgaatgacatccgcgcccaccgaaaccagatact
cctagaacagtcagcgctcaccgccacgccccgcaatcacctcaatccgcgtaattggcccgccgccctggtgtac
caggaaattccccagcccacgaccgtactacttccgcgagacgcccaggccgaagtccagctgactaactcaggtg
tccagctggcgggcggcgccaccctgtgtcgtcaccgccccgctcagggtataaagcggctggtgatccggggcag
aggcacacagctcaacgacgaggtggtgagctcttcgctgggtctgcgacctgacggagtcttccaactcgccgga
tcggggagatcttccttcacgcctcgtcaggccgtcctgactttggagagttcgtcctcgcagccccgctcgggtg
gcatcggcactctccagttcgtggaggagttcactccctcggtctacttcaaccccttctccggctcccccggcca
ctacccggacgagttcatcccgaacttcgacgccatcagcgagtcggtggacggctacgattgaaactaatcaccc
ccttatccagtgaaataaagatcatattgatgatgattttacagaaataaaaaataatcatttgatttgaaataaa
gatacaatcatattgatgatttgagtttaacaaaaaaataaagaatcacttacttgaaatctgataccaggtctct
gtccatgttttctgccaacaccacttcactcccctcttcccagctctggtactgcaggccccgggggctgcaaact
tcctccacacgctgaaggggatgtcaaattcctcctgtccctcaatcttcattttatcttctatcagatgtccaaa
aagcgcgtccgggtggatgatgacttcgaccccgtctacccctacgatgcagacaacgcaccgaccgtgcccttca
tcaacccccccttcgtctcttcagatggattccaagagaagcccctgggggtgttgtccctgcgactggccgaccc
cgtcaccaccaagaacggggaaatcaccctcaagctgggagagggggtggacctcgattcctcgggaaaactcatc
tccaacacggccaccaaggccgccgcccctctcagtttttccaacaacaccatttcccttaacatggatcacccct
tttacactaaagatggaaaattatccttacaagtttctccaccattaaatatactgagaacaagcattctaaacac
actagctttaggttttggatcaggtttaggactccgtggctctgccttggcagtacagttagtctctccacttaca
tttgatactgatggaaacataaagcttaccttagacagaggtttgcatgttacaacaggagatgcaattgaaagca
acataagctgggctaaaggtttaaaatttgaagatggagccatagcaaccaacattggaaatgggttagagtttgg
aagcagtagtacagaaacaggtgttgatgatgcttacccaatccaagttaaacttggatctggccttagctttgac
agtacaggagccataatggctggtaacaaagaagacgataaactcactttgtggacaacacctgatccatcaccaa
actgtcaaatactcgcagaaaatgatgcaaaactaacactttgcttgactaaatgtggtagtcaaatactggccac
tgtgtcagtcttagttgtaggaagtggaaacctaaaccccattactggcaccgtaagcagtgctcaggtgtttcta
cgttttgatgcaaacggtgttcttttaacagaacattctacactaaaaaaatactgggggtataggcagggagata
gcatagatggcactccatataccaatgctgtaggattcatgcccaatttaaaagcttatccaaagtcacaaagttc
tactactaaaaataatatagtagggcaagtatacatgaatggagatgtttcaaaacctatgcttctcactataacc
ctcaatggtactgatgacagcaacagtacatattcaatgtcattttcatacacctggactaatggaagctatgttg
gagcaacatttggggctaactcttataccttctcatacatcgcccaagaatgaacactgtatcccaccctgcatgc
caacccttcccaccccactctgtggaacaaactctgaaacacaaaataaaataaagttcaagtgttttattgattc
aacagttttacaggattcgagcagttatttttcctccaccctcccaggacatggaatacaccaccctctccccccg
cacagccttgaacatctgaatgccattggtgatggacatgcttttggtctccacgttccacacagtttcagagcga
gccagtctcgggtcggtcagggagatgaaaccctccgggcactcccgcatctgcacctcacagctcaacagctgag
gattgtcctcggtggtcgggatcacggttatctggaagaagcagaagagcggcggtgggaatcatagtccgcgaac
gggatcggccggtggtgtcgcatcaggccccgcagcagtcgctgccgccgccgctccgtcaagctgctgctcaggg
ggtccgggtccagggactccctcagcatgatgcccacggccctcagcatcagtcgtctggtgcgggggcgcagcag
cgcatgcggatctcgctcaggtcgctgcagtacgtgcaacacagaaccaccaggttgttcaacagtccatagttca
acacgctccagccgaaactcatcgcgggaaggatgctacccacgtggccgtcgtaccagatcctcaggtaaatcaa
gtggtgccccctccagaacacgctgcccacgtacatgatctccttgggcatgtggcggttcaccacctcccggtac
cacatcaccctctggttgaacatgcagccccggatgatcctgcggaaccacagggccagcaccgccccgcccgcca
tgcagcgaagagaccccgggtcccggcaatggcaatggaggacccaccgctcgtacccgtggatcatctgggagct
gaacaagtctatgttggcacagcacaggcatatgctcatgcatctcttcagcactctcaactcctcgggggtcaaa
accatatcccagggcacggggaactcttgcaggacagcgaaccccgcagaacagggcaatcctcgcacagaactta
cattgtgcatggacagggtatcgcaatcaggcagcaccgggtgatcctccaccagagaagcgcgggtctcggtctc
ctcacagcgtggtaagggggccggccgatacgggtgatggcgggacgcggctgatcgtgttcgcgaccgtgtcatg
atgcagttgctttcggacattttcgtacttgctgtagcagaacctggtccgggcgctgcacaccgatcgccggcgg
cggtctcggcgcttggaacgctcggtgttgaaattgtaaaacagccactctctcagaccgtgcagcagatctaggg
cctcaggagtgatgaagatcccatcatgcctgatggctctgatcacatcgaccaccgtggaatgggccagacccag
ccagatgatgcaattttgttgggtttcggtgacggcgggggagggaagaacaggaagaaccatgattaacttttaa
tccaaacggtctcggagtacttcaaaatgaagatcgcggagatggcacctctcgcccccgctgtgttggtggaaaa
taacagccaggtcaaaggtgatacggttctcgagatgttccacggtggcttccagcaaagcctccacgcgcacatc
cagaaacaagacaatagcgaaagcgggagggttctctaattcctcaatcatcatgttacactcctgcaccatcccc
agataattttcatttttccagccttgaatgattcgaactagttcctgaggtaaatccaagccagccatgataaaga
gctcgcgcagagcgccctccaccggcattcttaagcacaccctcataattccaagatattctgctcctggttcacc
tgcagcagattgacaagcggaatatcaaaatctctgccgcgatccctgagctcctccctcagcaataactgtaagt
actctttcatatcctctccgaaatttttagccataggaccaccaggaataagattagggcaagccacagtacagat
aaaccgaagtcctccccagtgagcattgccaaatgcaagactgctataagcatgctggctagacccggtgatatct
tccagataactggacagaaaatcgcccaggcaatttttaagaaaatcaacaaaagaaaaatcctccaggtggacgt
ttagagcctcgggaacaacgatgaagtaaatgcaagcggtgcgttccagcatggttagttagctgatctgtagaaa
aaacaaaaatgaacattaaaccatgctagcctggcgaacaggtgggtaaatcgttctctccagcaccaggcaggcc
acggggtctccggcgcgaccctcgtaaaaattgtcgctatgattgaaaaccatcacagagagacgttcccggtggc
cggcgtgaatgattcgacaagatgaatacacccccggaacattggcgtccgcgagtgaaaaaaagcgcccgaggaa
gcaataaggcactacaatgctcagtctcaagtccagcaaagcgatgccatgcggatgaagcacaaaattctcaggt
gcgtacaaaatgtaattactcccctcctgcacaggcagcaaagcccccgatccctccaggtacacatacaaagcct
cagcgtccatagcttaccgagcagcagcacacaacaggcgcaagagtcagagaaaggctgagctctaacctgtcca
cccgctctctgctcaatatatagcccagatctacactgacgtaaaggccaaagtctaaaaatacccgccaaataat
cacacacgcccagcacacgcccagaaaccggtgacacactcaaaaaaatacgcgcacttcctcaaacgcccaaaac
tgccgtcatttccgggttcccacgctacgtcatcaaaacacgactttcaaattccgtcgaccgttaaaaacgtcac
ccgccccgcccctaacggtcgcccgtctctcagccaatcagcgccccgcatccccaaattcaaacacctcatttgc
atattaacgcgcacaaaaagtttgagg
Venezuelan equine encephalitis virus [VEE] GenBank: L01442.2
(SEQ ID NO: 3)
atgggcggcgcatgagagaagcccagaccaattacctacccaaaatggagaaagttcacgttgacatcgaggaaga
cagcccattcctcagagctttgcagcggagcttcccgcagtttgaggtagaagccaagcaggtcactgataatgac
catgctaatgccagagcgttttcgcatctggcttcaaaactgatcgaaacggaggtggacccatccgacacgatcc
ttgacattggaagtgcgcccgcccgcagaatgtattctaagcacaagtatcattgtatctgtccgatgagatgtgc
ggaagatccggacagattgtataagtatgcaactaagctgaagaaaaactgtaaggaaataactgataaggaattg
gacaagaaaatgaaggagctcgccgccgtcatgagcgaccctgacctggaaactgagactatgtgcctccacgacg
acgagtcgtgtcgctacgaagggcaagtcgctgtttaccaggatgtatacgcggttgacggaccgacaagtctcta
tcaccaagccaataagggagttagagtcgcctactggataggctttgacaccaccccttttatgtttaagaacttg
gctggagcatatccatcatactctaccaactgggccgacgaaaccgtgttaacggctcgtaacataggcctatgca
gctctgacgttatggagcggtcacgtagagggatgtccattcttagaaagaagtatttgaaaccatccaacaatgt
tctattctctgttggctcgaccatctaccacgagaagagggacttactgaggagctggcacctgccgtctgtattt
cacttacgtggcaagcaaaattacacatgtcggtgtgagactatagttagttgcgacgggtacgtcgttaaaagaa
tagctatcagtccaggcctgtatgggaagccttcaggctatgctgctacgatgcaccgcgagggattcttgtgctg
caaagtgacagacacattgaacggggagagggtctcttttcccgtgtgcacgtatgtgccagctacattgtgtgac
caaatgactggcatactggcaacagatgtcagtgcggacgacgcgcaaaaactgctggttgggctcaaccagcgta
tagtcgtcaacggtcgcacccagagaaacaccaataccatgaaaaattaccttttgcccgtagtggcccaggcatt
tgctaggtgggcaaaggaatataaggaagatcaagaagatgaaaggccactaggactacgagatagacagttagtc
atggggtgttgttgggcttttagaaggcacaagataacatctatttataagcgcccggatacccaaaccatcatca
aagtgaacagcgatttccactcattcgtgctgcccaggataggcagtaacacattggagatcgggctgagaacaag
aatcaggaaaatgttagaggagcacaaggagccgtcacctctcattaccgccgaggacgtacaagaagctaagtgc
gcagccgatgaggctaaggaggtgcgtgaagccgaggagttgcgcgcagctctaccacctttggcagctgatgttg
aggagcccactctggaagccgatgtcgacttgatgttacaagaggctggggccggctcagtggagacacctcgtgg
cttgataaaggttaccagctacgctggcgaggacaagatcggctcttacgctgtgctttctccgcaggctgtactc
aagagtgaaaaattatcttgcatccaccctctcgctgaacaagtcatagtgataacacactctggccgaaaagggc
gttatgccgtggaaccataccatggtaaagtagtggtgccagagggacatgcaatacccgtccaggactttcaagc
tctgagtgaaagtgccaccattgtgtacaacgaacgtgagttcgtaaacaggtacctgcaccatattgccacacat
ggaggagcgctgaacactgatgaagaatattacaaaactgtcaagcccagcgagcacgacggcgaatacctgtacg
acatcgacaggaaacagtgcgtcaagaaagaactagtcactgggctagggctcacaggcgagctggtggatcctcc
cttccatgaattcgcctacgagagtctgagaacacgaccagccgctccttaccaagtaccaaccataggggtgtat
ggcgtgccaggatcaggcaagtctggcatcattaaaagcgcagtcaccaaaaaagatctagtggtgagcgccaaga
aagaaaactgtgcagaaattataagggacgtcaagaaaatgaaagggctggacgtcaatgccagaactgtggactc
agtgctcttgaatggatgcaaacaccccgtagagaccctgtatattgacgaagcttttgcttgtcatgcaggtact
ctcagagcgctcatagccattataagacctaaaaaggcagtgctctgcggggatcccaaacagtgcggttttttta
acatgatgtgcctgaaagtgcattttaaccacgagatttgcacacaagtcttccacaaaagcatctctcgccgttg
cactaaatctgtgacttcggtcgtctcaaccttgttttacgacaaaaaaatgagaacgacgaatccgaaagagact
aagattgtgattgacactaccggcagtaccaaacctaagcaggacgatctcattctcacttgtttcagaggggggt
gaagcagttgcaaatagattacaaaggcaacgaaataatgacggcagctgcctctcaagggctgacccgtaaaggt
gtgtatgccgttcggtacaaggtgaatgaaaatcctctgtacgcacccacctcagaacatgtgaacgtcctactga
cccgcacggaggaccgcatcgtgtggaaaacactagccggcgacccatggataaaaacactgactgccaagtaccc
tgggaatttcactgccacgatagaggagtggcaagcagagcatgatgccatcatgaggcacatcttggagagaccg
gaccctaccgacgtcttccagaataaggcaaacgtgtgttgggccaaggctttagtgccggtgctgaagaccgctg
gcatagacatgaccactgaacaatggaacactgtggattattttgaaacggacaaagctcactcagcagagatagt
attgaaccaactatgcgtgaggttctttggactcgatctggactccggtctattttctgcacccactgttccgtta
tccattaggaataatcactgggataactccccgtcgcctaacatgtacgggctgaataaagaagtggtccgtcagc
tctctcgcaggtacccacaactgcctcgggcagttgccactggaagagtctatgacatgaacactggtacactgcg
caattatgatccgcgcataaacctagtacctgtaaacagaagactgcctcatgctttagtcctccaccataatgaa
cacccacagagtgacttttcttcattcgtcagcaaattgaagggcagaactgtcctggtggtcggggaaaagttgt
ccgtcccaggcaaaatggttgactggttgtcagaccggcctgaggctaccttcagagctcggctggatttaggcat
cccaggtgatgtgcccaaatatgacataatatttgttaatgtgaggaccccatataaataccatcactatcagcag
tgtgaagaccatgccattaagcttagcatgttgaccaagaaagcttgtctgcatctgaatcccggcggaacctgtg
tcagcataggttatggttacgctgacagggccagcgaaagcatcattggtgctatagcgcggcagttcaagttttc
ccgggtatgcaaaccgaaatcctcacttgaagagacggaagttctgtttgtattcattgggtacgatcgcaaggcc
cgtacgcacaatccttacaagctttcatcaaccttgaccaacatttatacaggttccagactccacgaagccggat
gtgcaccctcatatcatgtggtgcgaggggatattgccacggccaccgaaggagtgattataaatgctgctaacag
caaaggacaacctggcggaggggtgtgcggagcgctgtataagaaattcccggaaagcttcgatttacagccgatc
gaagtaggaaaagcgcgactggtcaaaggtgcagctaaacatatcattcatgccgtaggaccaaacttcaacaaag
tttcggaggttgaaggtgacaaacagttggcagaggcttatgagtccatcgctaagattgtcaacgataacaatta
caagtcagtagcgattccactgttgtccaccggcatcttttccgggaacaaagatcgactaacccaatcattgaac
catttgctgacagctttagacaccactgatgcagatgtagccatatactgcagggacaagaaatgggaaatgactc
tcaaggaagcagtggctaggagagaagcagtggaggagatatgcatatccgacgactcttcagtgacagaacctga
tgcagagctggtgagggtgcatccgaagagttctttggctggaaggaagggctacagcacaagcgatggcaaaact
ttctcatatttggaagggaccaagtttcaccaggcggccaaggatatagcagaaattaatgccatgtggcccgttg
caacggaggccaatgagcaggtatgcatgtatatcctcggagaaagcatgagcagtattaggtcgaaatgccccgt
cgaagagtcggaagcctccacaccacctagcacgctgccttgcttgtgcatccatgccatgactccagaaagagta
cagcgcctaaaagcctcacgtccagaacaaattactgtgtgctcatcctttccattgccgaagtatagaatcactg
gtgtgcagaagatccaatgctcccagcctatattgttctcaccgaaagtgcctgcgtatattcatccaaggaagta
tctcgtggaaacaccaccggtagacgagactccggagccatcggcagagaaccaatccacagaggggacacctgaa
caaccaccacttataaccgaggatgagaccaggactagaacgcctgagccgatcatcatcgaagaggaagaagagg
atagcataagtttgctgtcagatggcccgacccaccaggtgctgcaagtcgaggcagacattcacgggccgccctc
tgtatctagctcatcctggtccattcctcatgcatccgactttgatgtggacagtttatccatacttgacaccctg
gagggagctagcgtgaccagcggggcaacgtcagccgagactaactcttacttcgcaaagagtatggagtttctgg
cgcgaccggtgcctgcgcctcgaacagtattcaggaaccctccacatcccgctccgcgcacaagaacaccgtcact
tgcacccagcagggcctgctcgagaaccagcctagtttccaccccgccaggcgtgaatagggtgatcactagagag
gagctcgaggcgcttaccccgtcacgcactcctagcaggtcggtctcgagaaccagcctggtctccaacccgccag
gcgtaaatagggtgattacaagagaggagtttgaggcgttcgtagcacaacaacaatgacggtttgatgcgggtgc
atacatcttttcctccgacaccggtcaagggcatttacaacaaaaatcagtaaggcaaacggtgctatccgaagtg
gtgttggagaggaccgaattggagatttcgtatgccccgcgcctcgaccaagaaaaagaagaattactacgcaaga
aattacagttaaatcccacacctgctaacagaagcagataccagtccaggaaggtggagaacatgaaagccataac
agctagacgtattctgcaaggcctagggcattatttgaaggcagaaggaaaagtggagtgctaccgaaccctgcat
cctgttcctttgtattcatctagtgtgaaccgtgccttttcaagccccaaggtcgcagtggaagcctgtaacgcca
tgttgaaagagaactttccgactgtggcttcttactgtattattccagagtacgatgcctatttggacatggttga
cggagcttcatgctgcttagacactgccagtttttgccctgcaaagctgcgcagctttccaaagaaacactcctat
ttggaacccacaatacgatcggcagtgccttcagcgatccagaacacgctccagaacgtcctggcagctgccacaa
aaagaaattgcaatgtcacgcaaatgagagaattgcccgtattggattcggcggcctttaatgtggaatgcttcaa
gaaatatgcgtgtaataatgaatattgggaaacgtttaaagaaaaccccatcaggcttactgaagaaaacgtggta
aattacattaccaaattaaaaggaccaaaagctgctgctctttttgcgaagacacataatttgaatatgttgcagg
acataccaatggacaggtttgtaatggacttaaagagagacgtgaaagtgactccaggaacaaaacatactgaaga
acggcccaaggtacaggtgatccaggctgccgatccgctagcaacagcgtatctgtgcggaatccaccgagagctg
gttaggagattaaatgcggtcctgcttccgaacattcatacactgtttgatatgtcggctgaagactttgacgcta
ttatagccgagcacttccagcctggggattgtgttctggaaactgacatcgcgtcgtttgataaaagtgaggacga
cgccatggctctgaccgcgttaatgattctggaagacttaggtgtggacgcagagctgttgacgctgattgaggcg
gctttcggcgaaatttcatcaatacatttgcccactaaaactaaatttaaattcggagccatgatgaaatctggaa
tgttcctcacactgtttgtgaacacagtcattaacattgtaatcgcaagcagagtgttgagagaacggctaaccgg
atcaccatgtgcagcattcattggagatgacaatatcgtgaaaggagtcaaatcggacaaattaatggcagacagg
tgcgccacctggttgaatatggaagtcaagattatagatgctgtggtgggcgagaaagcgccttatttctgtggag
ggtttattttgtgtgactccgtgaccggcacagcgtgccgtgtggcagaccccctaaaaaggctgtttaagcttgg
caaacctctggcagcagacgatgaacatgatgatgacaggagaagggcattgcatgaagagtcaacacgctggaac
cgagtgggtattctttcagagctgtgcaaggcagtagaatcaaggtatgaaaccgtaggaacttccatcatagtta
tggccatgactactctagctagcagtgttaaatcattcagctacctgagaggggcccctataactctctacggcta
acctgaatggactacgacatagtctagtccgccaagatgttcccgttccagccaatgtatccgatgcagccaatgc
cctatcgcaacccgttcgcggccccgcgcaggccctggttccccagaaccgacccttttctggcgatgcaggtgca
ggaattaacccgctcgatggctaacctgacgttcaagcaacgccgggacgcgccacctgaggggccatccgctaag
aaaccgaagaaggaggcctcgcaaaaacagaaagggggaggccaagggaagaagaagaagaaccaagggaagaaga
aggctaagacagggccgcctaatccgaaggcacagaatggaaacaagaagaagaccaacaagaaaccaggcaagag
acagcgcatggtcatgaaattggaatctgacaagacgttcccaatcatgttggaagggaagataaacggctacgct
tgtgtggtcggagggaagttattcaggccgatgcatgtggaaggcaagatcgacaacgacgttctggccgcgctta
agacgaagaaagcatccaaatacgatcttgagtatgcagatgtgccacagaacatgcgggccgatacattcaaata
cacccatgagaaaccccaaggctattacagctggcatcatggagcagtccaatatgaaaatgggcgtttcacggtg
ccgaaaggagttggggccaagggagacagcggacgacccattctggataaccagggacgggtggtcgctattgtgc
tgggaggtgtgaatgaaggatctaggacagccctttcagtcgtcatgtggaacgagaagggagttaccgtgaagta
tactccggagaactgcgagcaatggtcactagtgaccaccatgtgtctgctcgccaatgtgacgttcccatgtgct
caaccaccaatttgctacgacagaaaaccagcagagactttggccatgctcagcgttaacgttgacaacccgggct
acgatgagctgctggaagcagctgttaagtgccccggaaggaaaaggagatccaccgaggagctgtttaaggagta
taagctaacgcgcccttacatggccagatgcatcagatgtgcagttgggagctgccatagtccaatagcaatcgag
gcagtaaagagcgacgggcacgacggttatgttagacttcagacttcctcgcagtatggcctggattcctccggca
acttaaagggcaggaccatgcggtatgacatgcacgggaccattaaagagataccactacatcaagtgtcactcca
tacatctcgcccgtgtcacattgtggatgggcacggttatttcctgcttgccaggtgcccggcaggggactccatc
accatggaatttaagaaagattccgtcacacactcctgctcggtgccgtatgaagtgaaatttaatcctgtaggca
gagaactctatactcatcccccagaacacggagtagagcaagcgtgccaagtctacgcacatgatgcacagaacag
aggagcttatgtcgagatgcacctcccgggctcagaagtggacagcagtttggtttccttgagcggcagttcagtc
accgtgacacctcctgttgggactagcgccctggtggaatgcgagtgtggcggcacaaagatctccgagaccatca
acaagacaaaacagttcagccagtgcacaaagaaggagcagtgcagagcatatcggctgcagaacgataagtgggt
gtataattctgacaaactgcccaaagcaggggagccaccttaaaaggaaaactgcatgtcccattcttgctggcag
acggcaaatgcaccgtgcctctagcaccagaacctatgataacctttggtttcagatcagtgtcactgaaactgca
ccctaagaatcccacatatctaaccacccgccaacttgctgatgagcctcactacacgcacgagctcatatctgaa
ccagctgttaggaattttaccgtcaccgaaaaaggggggagtttgtatggggaaaccacccgccgaaaaggttttg
ggcacaggaaacagcacccggaaatccacatgggctaccgcacgaggtgataactcattattaccacagataccct
atgtccaccatcctgggtttgtcaatttgtgccgccattgcaaccgtttccgttgcagcgtctacctggctgtttt
gcagatctagagttgcgtgcctaactccttaccggctaacacctaacgctaggataccattttgtctggctgtgct
ttgctgcgcccgcactgcccgggccgagaccacctgggagtccttggatcacctatggaacaataaccaacagatg
ttctggattcaattgctgatccctctggccgccttgatcgtagtgactcgcctgctcaggtgcgtgtgctgtgtcg
tgccttttttagtcatggccggcgccgcaggcgccggcgcctacgagcacgcgaccacgatgccgagccaagcggg
aatctcgtataacactatagtcaacagagcaggctacgcaccactccctatcagcataacaccaacaaagatcaag
ctgatacctacagtgaacttggagtacgtcacctgccactacaaaacaggaatggattcaccagccatcaaatgct
gcggatctcaggaatgcactccaacttacaggcctgatgaacagtgcaaagtcttcacaggggtttacccgttcat
gtggggtggtgcatattgcttttgcgacactgagaacacccaagtcagcaaggcctacgtaatgaaatctgacgac
tgccttgcggatcatgctgaagcatataaagcgcacacagcctcagtgcaggcgttcctcaacatcacagtgggag
aacactctattgtgactaccgtgtatgtgaatggagaaactcctgtgaatttcaatggggtcaaattaactgcagg
tccgctttccacagcttggacaccctttgatcgcaaaatcgtgcagtatgccggggagatctataattatgatttt
cctgagtatggggcaggacaaccaggagcatttggagatatacaatccagaacagtctcaagctcagatctgtatg
ccaataccaacctagtgctgcagagacccaaagcaggagcgatccacgtgccatacactcaggcaccttcgggttt
tgagcaatggaagaaagataaagctccatcattgaaatttaccgcccctttcggatgcgaaatatatacaaacccc
attcgcgccgaaaactgtgctgtagggtcaattccattagcctttgacattcccgacgccttgttcaccagggtgt
cagaaacaccgacactttcagcggccgaatgcactcttaacgagtgcgtgtattcttccgactttggtgggatcgc
cacggtcaagtactcggccagcaagtcaggcaagtgcgcagtccatgtgccatcagggactgctaccctaaaagaa
gcagcagtcgagctaaccgagcaagggtcggcgactatccatttctcgaccgcaaatatccacccggagttcaggc
tccaaatatgcacatcatatgttacgtgcaaaggtgattgtcaccccccgaaagaccatattgtgacacaccctca
gtatcacgcccaaacatttacagccgcggtgtcaaaaaccgcgtggacgtggttaacatccctgctgggaggatca
gccgtaattattataattggcttggtgctggctactattgtggccatgtacgtgctgaccaaccagaaacataatt
gaatacagcagcaattggcaagctgcttacatagaactcgcggcgattggcatgccgccttaaaatttttatttta
ttttttcttttcttttccgaatcggattttgtttttaatatttc
VEE-MAG25mer; contains MAG-25merPDTT nucleotide (bases 30-1755)
(SEQ ID NO: 4)
atgggcggcgcatgagagaagcccagaccaattacctacccaaaatggagaaagttcacgttgacatcgaggaaga
cagcccattcctcagagctttgcagcggagcttcccgcagtttgaggtagaagccaagcaggtcactgataatgac
catgctaatgccagagcgttttcgcatctggcttcaaaactgatcgaaacggaggtggacccatccgacacgatcc
ttgacattggaagtgcgcccgcccgcagaatgtattctaagcacaagtatcattgtatctgtccgatgagatgtgc
ggaagatccggacagattgtataagtatgcaactaagctgaagaaaaactgtaaggaaataactgataaggaattg
gacaagaaaatgaaggagctcgccgccgtcatgagcgaccctgacctggaaactgagactatgtgcctccacgacg
acgagtcgtgtcgctacgaagggcaagtcgctgtttaccaggatgtatacgcggttgacggaccgacaagtctcta
tcaccaagccaataagggagttagagtcgcctactggataggctttgacaccaccccttttatgtttaagaacttg
gctggagcatatccatcatactctaccaactgggccgacgaaaccgtgttaacggctcgtaacataggcctatgca
gctctgacgttatggagcggtcacgtagagggatgtccattcttagaaagaagtatttgaaaccatccaacaatgt
tctattctctgttggctcgaccatctaccacgagaagagggacttactgaggagctggcacctgccgtctgtattt
cacttacgtggcaagcaaaattacacatgtcggtgtgagactatagttagttgcgacgggtacgtcgttaaaagaa
tagctatcagtccaggcctgtatgggaagccttcaggctatgctgctacgatgcaccgcgagggattcttgtgctg
caaagtgacagacacattgaacggggagagggtctcttttcccgtgtgcacgtatgtgccagctacattgtgtgac
caaatgactggcatactggcaacagatgtcagtgcggacgacgcgcaaaaactgctggttgggctcaaccagcgta
tagtcgtcaacggtcgcacccagagaaacaccaataccatgaaaaattaccttttgcccgtagtggcccaggcatt
tgctaggtgggcaaaggaatataaggaagatcaagaagatgaaaggccactaggactacgagatagacagttagtc
atggggtgttgttgggcttttagaaggcacaagataacatctatttataagcgcccggatacccaaaccatcatca
aagtgaacagcgatttccactcattcgtgctgcccaggataggcagtaacacattggagatcgggctgagaacaag
aatcaggaaaatgttagaggagcacaaggagccgtcacctctcattaccgccgaggacgtacaagaagctaagtgc
gcagccgatgaggctaaggaggtgcgtgaagccgaggagttgcgcgcagctctaccacctttggcagctgatgttg
aggagcccactctggaagccgatgtcgacttgatgttacaagaggctggggccggctcagtggagacacctcgtgg
cttgataaaggttaccagctacgctggcgaggacaagatcggctcttacgctgtgctttctccgcaggctgtactc
aagagtgaaaaattatcttgcatccaccctctcgctgaacaagtcatagtgataacacactctggccgaaaagggc
gttatgccgtggaaccataccatggtaaagtagtggtgccagagggacatgcaatacccgtccaggactttcaagc
tctgagtgaaagtgccaccattgtgtacaacgaacgtgagttcgtaaacaggtacctgcaccatattgccacacat
ggaggagcgctgaacactgatgaagaatattacaaaactgtcaagcccagcgagcacgacggcgaatacctgtacg
acatcgacaggaaacagtgcgtcaagaaagaactagtcactgggctagggctcacaggcgagctggtggatcctcc
cttccatgaattcgcctacgagagtctgagaacacgaccagccgctccttaccaagtaccaaccataggggtgtat
ggcgtgccaggatcaggcaagtctggcatcattaaaagcgcagtcaccaaaaaagatctagtggtgagcgccaaga
aagaaaactgtgcagaaattataagggacgtcaagaaaatgaaagggctggacgtcaatgccagaactgtggactc
agtgctcttgaatggatgcaaacaccccgtagagaccctgtatattgacgaagcttttgcttgtcatgcaggtact
ctcagagcgctcatagccattataagacctaaaaaggcagtgctctgcggggatcccaaacagtgcggttttttta
acatgatgtgcctgaaagtgcattttaaccacgagatttgcacacaagtcttccacaaaagcatctctcgccgttg
cactaaatctgtgacttcggtcgtctcaaccttgttttacgacaaaaaaatgagaacgacgaatccgaaagagact
aagattgtgattgacactaccggcagtaccaaacctaagcaggacgatctcattctcacttgtttcagagggtggg
tgaagcagttgcaaatagattacaaaggcaacgaaataatgacggcagctgcctctcaagggctgacccgtaaagg
tgtgtatgccgttcggtacaaggtgaatgaaaatcctctgtacgcacccacctcagaacatgtgaacgtcctactg
acccgcacggaggaccgcatcgtgtggaaaacactagccggcgacccatggataaaaacactgactgccaagtacc
ctgggaatttcactgccacgatagaggagtggcaagcagagcatgatgccatcatgaggcacatcttggagagacc
ggaccctaccgacgtcttccagaataaggcaaacgtgtgttgggccaaggctttagtgccggtgctgaagaccgct
ggcatagacatgaccactgaacaatggaacactgtggattattttgaaacggacaaagctcactcagcagagatag
tattgaaccaactatgcgtgaggttctttggactcgatctggactccggtctattttctgcacccactgttccgtt
atccattaggaataatcactgggataactccccgtcgcctaacatgtacgggctgaataaagaagtggtccgtcag
ctctctcgcaggtacccacaactgcctcgggcagttgccactggaagagtctatgacatgaacactggtacactgc
gcaattatgatccgcgcataaacctagtacctgtaaacagaagactgcctcatgctttagtcctccaccataatga
acacccacagagtgacttttcttcattcgtcagcaaattgaagggcagaactgtcctggtggtcggggaaaagttg
tccgtcccaggcaaaatggttgactggttgtcagaccggcctgaggctaccttcagagctcggctggatttaggca
tcccaggtgatgtgcccaaatatgacataatatttgttaatgtgaggaccccatataaataccatcactatcagca
gtgtgaagaccatgccattaagcttagcatgttgaccaagaaagcttgtctgcatctgaatcccggcggaacctgt
gtcagcataggttatggttacgctgacagggccagcgaaagcatcattggtgctatagcgcggcagttcaagtttt
cccgggtatgcaaaccgaaatcctcacttgaagagacggaagttctgtttgtattcattgggtacgatcgcaaggc
ccgtacgcacaatccttacaagctttcatcaaccttgaccaacatttatacaggttccagactccacgaagccgga
tgtgcaccctcatatcatgtggtgcgaggggatattgccacggccaccgaaggagtgattataaatgctgctaaca
gcaaaggacaacctggcggaggggtgtgcggagcgctgtataagaaattcccggaaagcttcgatttacagccgat
cgaagtaggaaaagcgcgactggtcaaaggtgcagctaaacatatcattcatgccgtaggaccaaacttcaacaaa
gtttcggaggttgaaggtgacaaacagttggcagaggcttatgagtccatcgctaagattgtcaacgataacaatt
acaagtcagtagcgattccactgttgtccaccggcatcttttccgggaacaaagatcgactaacccaatcattgaa
ccatttgctgacagctttagacaccactgatgcagatgtagccatatactgcagggacaagaaatgggaaatgact
ctcaaggaagcagtggctaggagagaagcagtggaggagatatgcatatccgacgactcttcagtgacagaacctg
atgcagagctggtgagggtgcatccgaagagttctttggctggaaggaagggctacagcacaagcgatggcaaaac
tttctcatatttggaagggaccaagtttcaccaggcggccaaggatatagcagaaattaatgccatgtggcccgtt
gcaacggaggccaatgagcaggtatgcatgtatatcctcggagaaagcatgagcagtattaggtcgaaatgccccg
tcgaagagtcggaagcctccacaccacctagcacgctgccttgcttgtgcatccatgccatgactccagaaagagt
acagcgcctaaaagcctcacgtccagaacaaattactgtgtgctcatcctttccattgccgaagtatagaatcact
ggtgtgcagaagatccaatgctcccagcctatattgttctcaccgaaagtgcctgcgtatattcatccaaggaagt
atctcgtggaaacaccaccggtagacgagactccggagccatcggcagagaaccaatccacagaggggacacctga
acaaccaccacttataaccgaggatgagaccaggactagaacgcctgagccgatcatcatcgaagaggaagaagag
gatagcataagtttgctgtcagatggcccgacccaccaggtgctgcaagtcgaggcagacattcacgggccgccct
ctgtatctagctcatcctggtccattcctcatgcatccgactttgatgtggacagtttatccatacttgacaccct
ggagggagctagcgtgaccagcggggcaacgtcagccgagactaactcttacttcgcaaagagtatggagtttctg
gcgcgaccggtgcctgcgcctcgaacagtattcaggaaccctccacatcccgctccgcgcacaagaacaccgtcac
ttgcacccagcagggcctgctcgagaaccagcctagtttccaccccgccaggcgtgaatagggtgatcactagaga
ggagctcgaggcgcttaccccgtcacgcactcctagcaggtcggtctcgagaaccagcctggtctccaacccgcca
ggcgtaaatagggtgattacaagagaggagtttgaggcgttcgtagcacaacaacaatgacggtttgatgcgggtg
catacatcttttcctccgacaccggtcaagggcatttacaacaaaaatcagtaaggcaaacggtgctatccgaagt
ggtgttggagaggaccgaattggagatttcgtatgccccgcgcctcgaccaagaaaaagaagaattactacgcaag
aaattacagttaaatcccacacctgctaacagaagcagataccagtccaggaaggtggagaacatgaaagccataa
cagctagacgtattctgcaaggcctagggcattatttgaaggcagaaggaaaagtggagtgctaccgaaccctgca
tcctgttcctttgtattcatctagtgtgaaccgtgccttttcaagccccaaggtcgcagtggaagcctgtaacgcc
atgttgaaagagaactttccgactgtggcttcttactgtattattccagagtacgatgcctatttggacatggttg
acggagcttcatgctgcttagacactgccagtttttgccctgcaaagctgcgcagctttccaaagaaacactccta
tttggaacccacaatacgatcggcagtgccttcagcgatccagaacacgctccagaacgtcctggcagctgccaca
aaaagaaattgcaatgtcacgcaaatgagagaattgcccgtattggattcggcggcctttaatgtggaatgcttca
agaaatatgcgtgtaataatgaatattgggaaacgtttaaagaaaaccccatcaggcttactgaagaaaacgtggt
aaattacattaccaaattaaaaggaccaaaagctgctgctctttttgcgaagacacataatttgaatatgttgcag
gacataccaatggacaggtttgtaatggacttaaagagagacgtgaaagtgactccaggaacaaaacatactgaag
aacggcccaaggtacaggtgatccaggctgccgatccgctagcaacagcgtatctgtgcggaatccaccgagagct
ggttaggagattaaatgcggtcctgcttccgaacattcatacactgtttgatatgtcggctgaagactttgacgct
attatagccgagcacttccagcctggggattgtgttctggaaactgacatcgcgtcgtttgataaaagtgaggacg
acgccatggctctgaccgcgttaatgattctggaagacttaggtgtggacgcagagctgttgacgctgattgaggc
ggctttcggcgaaatttcatcaatacatttgcccactaaaactaaatttaaattcggagccatgatgaaatctgga
atgttcctcacactgtttgtgaacacagtcattaacattgtaatcgcaagcagagtgttgagagaacggctaaccg
gatcaccatgtgcagcattcattggagatgacaatatcgtgaaaggagtcaaatcggacaaattaatggcagacag
gtgcgccacctggttgaatatggaagtcaagattatagatgctgtggtgggcgagaaagcgccttatttctgtgga
gggtttattttgtgtgactccgtgaccggcacagcgtgccgtgtggcagaccccctaaaaaggctgtttaagcttg
gcaaacctctggcagcagacgatgaacatgatgatgacaggagaagggcattgcatgaagagtcaacacgctggaa
ccgagtgggtattctttcagagctgtgcaaggcagtagaatcaaggtatgaaaccgtaggaacttccatcatagtt
atggccatgactactctagctagcagtgttaaatcattcagctacctgagaggggcccctataactctctacggct
aacctgaatggactacgactctagaatagtctttaattaagccaccatggcaggcatgtttcaggcgctgagcgaa
ggctgcaccccgtatgatattaaccagatgctgaacgtgctgggcgatcatcaggtctcaggccttgagcagcttg
agagtataatcaactttgaaaaactgactgaatggaccagttctaatgttatgcctatcctgtctcctctgacaaa
gggcatcctgggcttcgtgtttaccctgaccgtgccttctgagagaggacttagctgcattagcgaagcggatgcg
accaccccggaaagcgcgaacctgggcgaagaaattctgagccagctgtatctttggccaagggtgacctaccatt
cccctagttatgcttaccaccaatttgaaagacgagccaaatataaaagacacttccccggctttggccagagcct
gctgtttggctaccctgtgtacgtgttcggcgattgcgtgcagggcgattgggatgcgattcgctttcgctattgc
gcgccgccgggctatgcgctgctgcgctgcaacgataccaactatagcgctctgctggctgtgggggccctagaag
gacccaggaatcaggactggcttggtgtcccaagacaacttgtaactcggatgcaggctattcagaatgccggcct
gtgtaccctggtggccatgctggaagagacaatcttctggctgcaagcgtttctgatggcgctgaccgatagcggc
ccgaaaaccaacattattgtggatagccagtatgtgatgggcattagcaaaccgagctttcaggaatttgtggatt
gggaaaacgtgagcccggaactgaacagcaccgatcagccgttttggcaagccggaatcctggccagaaatctggt
gcctatggtggccacagtgcagggccagaacctgaagtaccagggtcagtcactagtcatctctgcttctatcatt
gtcttcaacctgctggaactggaaggtgattatcgagatgatggcaacgtgtgggtgcataccccgctgagcccgc
gcaccctgaacgcgtgggtgaaagcggtggaagaaaaaaaaggtattccagttcacctagagctggccagtatgac
caacatggagctcatgagcagtattgtgcatcagcaggtcagaacatacggccccgtgttcatgtgtctcggcgga
ctgcttacaatggtggctggtgctgtgtggctgacagtgcgagtgctcgagctgttccgggccgcgcagctggcca
acgacgtggtcctccagatcatggagctttgtggtgcagcgtttcgccaggtgtgccataccaccgtgccgtggcc
gaacgcgagcctgaccccgaaatggaacaacgaaaccacccagccccagatcgccaactgcagcgtgtatgacttt
tttgtgtggctccattattattctgttcgagacacactttggccaagggtgacctaccatatgaacaaatatgcgt
atcatatgctggaaagacgagccaaatataaaagaggaccaggacctggcgctaaatttgtggccgcctggacact
gaaagccgctgctggtcctggacctggccagtacatcaaggccaacagcaagttcatcggcatcaccgaactcgga
cccggaccaggctgatgattcgaacggccgtatcacgcccaaacatttacagccgcggtgtcaaaaaccgcgtgga
cgtggttaacatccctgctgggaggatcagccgtaattattataattggcttggtgctggctactattgtggccat
gtacgtgctgaccaaccagaaacataattgaatacagcagcaattggcaagctgcttacatagaactcgcggcgat
tggcatgccgccttaaaatttttattttattttttcttttcttttccgaatcggattttgtttttaatatttcaaa
aaaaaaaaaaaaaaaaaaaaaaaaaaaaaaaaaaaaaaaaaaaaaaaaaaaaaaaaaaaaaaaaaaaaaaaaaaaa
a
Venezuelan equine encephalitis virus strain TC-83 [TC-83]
(SEQ ID NO: 5) GenBank: L01443.1
VEE Delivery Vector
(SEQ ID NO: 6); VEE genome with nucleotides 7544-11175
deleted [alphavirus structural proteins removed]
TC-83 Delivery Vector(SEQ ID NO: 7); TC-83 genome with nucleotides
7544-11175 deleted [alphavirus structural proteins removed]
VEE Production Vector(SEQ ID NO: 8); VEE genome with nucleotides
7544-11175 deleted, plus 5′ T7-promoter, plus 3′ restriction sites
TC-83 Production Vector(SEQ ID NO: 9); TC-83 genome with nucleotides
7544-11175 deleted, plus 5′ T7-promoter, plus 3′ restriction sites
VEE-UbAAY(SEQ ID NO: 14); VEE delivery vector with MHC class I mouse
tumor epitopes SIINFEKL and AH1-A5 inserted
VEE-Luciferase
(SEQ ID NO: 15); VEE delivery vector with luciferase
gene inserted at 7545
Tremelimumab VL
(SEQ ID NO: 16)
PSSLSASVGDRVTITCRASQSINSYLDWYQQKPGKAPKLLIYAASSLQSGVPSRFSGSGSGTDFTLTISSLQPEDF
ATYYCQQYYSTPFTFGPGTKVEIKRTVAAPSVFIFPPSDEQLKSGTASVVCLLNNFYPREAKV
Tremelimumab VH
(SEQ ID NO: 17)
GVVQPGRSLRLSCAASGFTFSSYGMHWVRQAPGKGLEWVAVIWYDGSNKYYADSVKGRFTISRDNSKNTLYLQMNS
LRAEDTAVYYCARDPRGATLYYYYYGMDVWGQGTTVTVSSASTKGPSVFPLAPCSRSTSESTAALGCLVKDYFPEP
VTVSWNSGALTSGVH
Tremelimumab VH CDR1
(SEQ ID NO: 18)
GFTFSSYGMH
Tremelimumab VH CDR2
(SEQ ID NO: 19)
VIWYDGSNKYYADSV
Tremelimumab VH CDR3
(SEQ ID NO: 20)
DPRGATLYYYYYGMDV
Tremelimumab VL CDR1
(SEQ ID NO: 21)
RASQSINSYLD
Tremelimumab VL CDR2
(SEQ ID NO: 22)
AASSLQS
Tremelimumab VL CDR3
(SEQ ID NO: 23)
QQYYSTPFT
Durvalumab (MEDI4736) VL
(SEQ ID NO: 24)
EIVLTQSPGTLSLSPGERATLSCRASQRVSSSYLAWYQQKPGQAPRLLIYDASSRATGIPDRFSGSGSGTDFTLTI
SRLEPEDFAVYYCQQYGSLPWTFGQGTKVEIK
MEDI4736 VH
(SEQ ID NO: 25)
EVQLVESGGGLVQPGGSLRLSCAASGFTFSRYWMSWVRQAPGKGLEWVANIKQDGSEKYYVDSVKGRFTISRDNAK
NSLYLQMNSLRAEDTAVYYCAREGGWFGELAFDYWGQGTLVTVSS
MEDI4736 VH CDR1
(SEQ ID NO: 26)
RYWMS
MEDI4736 VH CDR2
(SEQ ID NO: 27)
NIKQDGSEKYYVDSVKG
MEDI4736 VH CDR3
(SEQ ID NO: 28)
EGGWFGELAFDY
MEDI4736 VL CDR1
(SEQ ID NO: 29)
RASQRVSSSYLA
MEDI4736 VL CDR2
(SEQ ID NO: 30)
DASSRAT
MEDI4736 VL CDR3
(SEQ ID NO: 31)
QQYGSLPWT
UbA76-25merPDTT nucleotide
(SEQ ID NO: 32)
gcccgggcatttaaatgcgatcgcatcgattacgactctagaatagtctagtccgcaggccaccatgcagatcttc
gtgaagaccctgaccggcaagaccatcaccctagaggtggagcccagtgacaccatcgagaacgtgaaggccaaga
tccaggataaagagggcatcccccctgaccagcagaggctgatctttgccggcaagcagctggaagatggccgcac
cctctctgattacaacatccagaaggagtcaaccctgcacctggtccttcgcctgagaggtgccatgtttcaggcg
ctgagcgaaggctgcaccccgtatgatattaaccagatgctgaacgtgctgggcgatcatcaggtctcaggccttg
agcagcttgagagtataatcaactttgaaaaactgactgaatggaccagttctaatgttatgcctatcctgtctcc
tctgacaaagggcatcctgggcttcgtgtttaccctgaccgtgccttctgagagaggacttagctgcattagcgaa
gcggatgcgaccaccccggaaagcgcgaacctgggcgaagaaattctgagccagctgtatctttggccaagggtga
cctaccattcccctagttatgcttaccaccaatttgaaagacgagccaaatataaaagacacttccccggctttgg
ccagagcctgctgtttggctaccctgtgtacgtgttcggcgattgcgtgcagggcgattgggatgcgattcgcttt
cgctattgcgcgccgccgggctatgcgctgctgcgctgcaacgataccaactatagcgctctgctggctgtggggg
ccctagaaggacccaggaatcaggactggcttggtgtcccaagacaacttgtaactcggatgcaggctattcagaa
tgccggcctgtgtaccctggtggccatgctggaagagacaatcttctggctgcaagcgtttctgatggcgctgacc
gatagcggcccgaaaaccaacattattgtggatagccagtatgtgatgggcattagcaaaccgagctttcaggaat
ttgtggattgggaaaacgtgagcccggaactgaacagcaccgatcagccgttttggcaagccggaatcctggccag
aaatctggtgcctatggtggccacagtgcagggccagaacctgaagtaccagggtcagtcactagtcatctctgct
tctatcattgtcttcaacctgctggaactggaaggtgattatcgagatgatggcaacgtgtgggtgcataccccgc
tgagcccgcgcaccctgaacgcgtgggtgaaagcggtggaagaaaaaaaaggtattccagttcacctagagctggc
cagtatgaccaacatggagctcatgagcagtattgtgcatcagcaggtcagaacatacggccccgtgttcatgtgt
ctcggcggactgcttacaatggtggctggtgctgtgtggctgacagtgcgagtgctcgagctgttccgggccgcgc
agctggccaacgacgtggtcctccagatcatggagctttgtggtgcagcgtttcgccaggtgtgccataccaccgt
gccgtggccgaacgcgagcctgaccccgaaatggaacaacgaaaccacccagccccagatcgccaactgcagcgtg
tatgacttttttgtgtggctccattattattctgttcgagacacactttggccaagggtgacctaccatatgaaca
aatatgcgtatcatatgctggaaagacgagccaaatataaaagaggaccaggacctggcgctaaatttgtggccgc
ctggacactgaaagccgctgctggtcctggacctggccagtacatcaaggccaacagcaagttcatcggcatcacc
gaactcggacccggaccaggctgatgatttcgaaatttaaataagcttgcggccgctagggataacagggtaatta
tcacgcccaaacatttacagccgcggtgtcaaaaaccgcgtgg
UbA76-25merPDTT polypeptide
(SEQ ID NO: 33)
MQIFVKTLTGKTITLEVEPSDTIENVKAKIQDKEGIPPDQQRLIFAGKQLEDGRTLSDYNIQKESTLHLVLRLRGA
MFQALSEGCTPYDINQMLNVLGDHQVSGLEQLESIINFEKLTEWTSSNVMPILSPLIKGILGFVFTLTVPSERGLS
CISEADATTPESANLGEEILSQLYLWPRVTYHSPSYAYHQFERRAKYKRHFPGFGQSLLFGYPVYVFGDCVQGDWD
AIRFRYCAPPGYALLRCNDTNYSALLAVGALEGPRNQDWLGVPRQLVTRMQAIQNAGLCTLVAMLEETIFWLQAFL
MALTDSGPKTNIIVDSQYVMGISKPSFQEFVDWENVSPELNSTDQPFWQAGILARNLVPMVATVQGQNLKYQGQSL
VISASIIVFNLLELEGDYRDDGNVWVHTPLSPRTLNAWVKAVEEKKGIPVHLELASMTNMELMSSIVHQQVRTYGP
VFMCLGGLLTMVAGAVWLTVRVLELFRAAQLANDVVLQIMELCGAAFRQVCHTTVPWPNASLTPKWNNETTQPQIA
NCSVYDFFVWLHYYSVRDTLWPRVTYHMNKYAYHMLERRAKYKRGPGPGAKFVAAWTLKAAAGPGPGQYIKANSKF
IGITELGPGPG
MAG-25merPDTT nucleotide
(SEQ ID NO: 34)
atggccgggatgttccaggcactgtccgaaggctgcacaccctatgatattaaccagatgctgaatgtcctgggag
accaccaggtctctggcctggagcagctggagagcatcatcaacttcgagaagctgaccgagtggacaagctccaa
tgtgatgcctatcctgtccccactgaccaagggcatcctgggcttcgtgtttaccctgacagtgccttctgagcgg
ggcctgtcttgcatcagcgaggcagacgcaaccacaccagagtccgccaatctgggcgaggagatcctgtctcagc
tgtacctgtggccccgggtgacatatcactccccttcttacgcctatcaccagttcgagcggagagccaagtacaa
gagacacttcccaggctttggccagtctctgctgttcggctaccccgtgtacgtgttcggcgattgcgtgcagggc
gactgggatgccatccggtttagatactgcgcaccacctggatatgcactgctgaggtgtaacgacaccaattatt
ccgccctgctggcagtgggcgccctggagggccctcgcaatcaggattggctgggcgtgccaaggcagctggtgac
acgcatgcaggccatccagaacgcaggcctgtgcaccctggtggcaatgctggaggagacaatcttctggctgcag
gcctttctgatggccctgaccgacagcggccccaagacaaacatcatcgtggattcccagtacgtgatgggcatct
ccaagccttctttccaggagtttgtggactgggagaacgtgagcccagagctgaattccaccgatcagccattctg
gcaggcaggaatcctggcaaggaacctggtgcctatggtggccacagtgcagggccagaatctgaagtaccagggc
cagagcctggtcatcagcgcctccatcatcgtgtttaacctgctggagctggagggcgactatcgggacgatggca
acgtgtgggtgcacaccccactgagccccagaacactgaacgcctgggtgaaggccgtggaggagaagaagggcat
cccagtgcacctggagctggcctccatgaccaatatggagctgatgtctagcatcgtgcaccagcaggtgaggaca
tacggacccgtgttcatgtgcctgggaggcctgctgaccatggtggcaggagccgtgtggctgacagtgcgggtgc
tggagctgttcagagccgcccagctggccaacgatgtggtgctgcagatcatggagctgtgcggagcagcctttcg
ccaggtgtgccacaccacagtgccatggcccaatgcctccctgacccccaagtggaacaatgagacaacacagcct
cagatcgccaactgtagcgtgtacgacttcttcgtgtggctgcactactatagcgtgagggataccctgtggcccc
gcgtgacataccacatgaataagtacgcctatcacatgctggagaggcgcgccaagtataagagaggccctggccc
aggcgcaaagtttgtggcagcatggaccctgaaggccgccgccggccccggccccggccagtatatcaaggctaac
agtaagttcattggaatcacagagctgggacccggacctgga
MAG-25merPDTT polypeptide
(SEQ ID NO: 35)
MAGMFQALSEGCTPYDINQMLNVLGDHQVSGLEQLESIINFEKLTEWTSSNVMPILSPLTKGILGFVFTLTVPSER
GLSCISEADATTPESANLGEEILSQLYLWPRVTYHSPSYAYHQFERRAKYKRHFPGFGQSLLFGYPVYVFGDCVQG
DWDAIRFRYCAPPGYALLRCNDTNYSALLAVGALEGPRNQDWLGVPRQLVTRMQAIQNAGLCTLVAMLEETIFWLQ
AFLMALTDSGPKTNIIVDSQYVMGISKPSFQEFVDWENVSPELNSTDQPFWQAGILARNLVPMVATVQGQNLKYQG
QSLVISASIIVFNLLELEGDYRDDGNVWVHTPLSPRTLNAWVKAVEEKKGIPVHLELASMTNMELMSSIVHQQVRT
YGPVFMCLGGLLTMVAGAVWLTVRVLELFRAAQLANDVVLQIMELCGAAFRQVCHTTVPWPNASLTPKWNNETTQP
QIANCSVYDFFVWLHYYSVRDTLWPRVTYHMNKYAYHMLERRAKYKRGPGPGAKFVAAWTLKAAAGPGPGQYIKAN
SKFIGITELGPGPG
Ub7625merPDTT_NoSFL nucleotide
(SEQ ID NO: 36)
gcccgggcatttaaatgcgatcgcatcgattacgactctagaatagtctagtccgcaggccaccatgcagatcttc
gtgaagaccctgaccggcaagaccatcaccctagaggtggagcccagtgacaccatcgagaacgtgaaggccaaga
tccaggataaagagggcatcccccctgaccagcagaggctgatctttgccggcaagcagctggaagatggccgcac
cctctctgattacaacatccagaaggagtcaaccctgcacctggtccttcgcctgagaggtgccatgtttcaggcg
ctgagcgaaggctgcaccccgtatgatattaaccagatgctgaacgtgctgggcgatcatcagtttaagcacatca
aagcctttgaccggacatttgctaacaacccaggtcccatggttgtgtttgccacacctgggcctatcctgtctcc
tctgacaaagggcatcctgggcttcgtgtttaccctgaccgtgccttctgagagaggacttagctgcattagcgaa
gcggatgcgaccaccccggaaagcgcgaacctgggcgaagaaattctgagccagctgtatctttggccaagggtga
cctaccattcccctagttatgcttaccaccaatttgaaagacgagccaaatataaaagacacttccccggctttgg
ccagagcctgctgtttggctaccctgtgtacgtgttcggcgattgcgtgcagggcgattgggatgcgattcgcttt
cgctattgcgcgccgccgggctatgcgctgctgcgctgcaacgataccaactatagcgctctgctggctgtggggg
ccctagaaggacccaggaatcaggactggcttggtgtcccaagacaacttgtaactcggatgcaggctattcagaa
tgccggcctgtgtaccctggtggccatgctggaagagacaatcttctggctgcaagcgtttctgatggcgctgacc
gatagcggcccgaaaaccaacattattgtggatagccagtatgtgatgggcattagcaaaccgagctttcaggaat
ttgtggattgggaaaacgtgagcccggaactgaacagcaccgatcagccgttttggcaagccggaatcctggccag
aaatctggtgcctatggtggccacagtgcagggccagaacctgaagtaccagggtcagtcactagtcatctctgct
tctatcattgtcttcaacctgctggaactggaaggtgattatcgagatgatggcaacgtgtgggtgcataccccgc
tgagcccgcgcaccctgaacgcgtgggtgaaagcggtggaagaaaaaaaaggtattccagttcacctagagctggc
cagtatgaccaacatggagctcatgagcagtattgtgcatcagcaggtcagaacatacggccccgtgttcatgtgt
ctcggcggactgcttacaatggtggctggtgctgtgtggctgacagtgcgagtgctcgagctgttccgggccgcgc
agctggccaacgacgtggtcctccagatcatggagctttgtggtgcagcgtttcgccaggtgtgccataccaccgt
gccgtggccgaacgcgagcctgaccccgaaatggaacaacgaaaccacccagccccagatcgccaactgcagcgtg
tatgacttttttgtgtggctccattattattctgttcgagacacactttggccaagggtgacctaccatatgaaca
aatatgcgtatcatatgctggaaagacgagccaaatataaaagaggaccaggacctggcgctaaatttgtggccgc
ctggacactgaaagccgctgctggtcctggacctggccagtacatcaaggccaacagcaagttcatcggcatcacc
gaactcggacccggaccaggctgatgatttcgaaatttaaataagcttgcggccgctagggataacagggtaatta
tcacgcccaaacatttacagccgcggtgtcaaaaaccgcgtgg
Ub7625merPDTT_NoSFL polypeptide
(SEQ ID NO: 37)
MQIFVKTLTGKTITLEVEPSDTIENVKAKIQDKEGIPPDQQRLIFAGKQLEDGRTLSDYNIQKESTLHLVLRLRGA
MFQALSEGCTPYDINQMLNVLGDHQFKHIKAFDRTFANNPGPMVVFATPGPILSPLIKGILGFVFTLTVPSERGLS
CISEADATTPESANLGEEILSQLYLWPRVTYHSPSYAYHQFERRAKYKRHFPGFGQSLLFGYPVYVFGDCVQGDWD
AIRFRYCAPPGYALLRCNDTNYSALLAVGALEGPRNQDWLGVPRQLVTRMQAIQNAGLCTLVAMLEETIFWLQAFL
MALTDSGPKTNIIVDSQYVMGISKPSFQEFVDWENVSPELNSTDQPFWQAGILARNLVPMVATVQGQNLKYQGQSL
VISASIIVENLLELEGDYRDDGNVWVHTPLSPRTLNAWVKAVEEKKGIPVHLELASMTNMELMSSIVHQQVRTYGP
VFMCLGGLLTMVAGAVWLTVRVLELFRAAQLANDVVLQIMELCGAAFRQVCHTTVPWPNASLTPKWNNETTQPQIA
NCSVYDFFVWLHYYSVRDTLWPRVTYHMNKYAYHMLERRAKYKRGPGPGAKFVAAWTLKAAAGPGPGQYIKANSKF
IGITELGPGPG
ubiquitin
>UbG76 0-228
(SEQ ID NO: 38)
atgcagatcttcgtgaagaccctgaccggcaagaccatcaccctagaggtggagcccagtgacaccatcgagaacg
tgaaggccaagatccaggataaagagggcatcccccctgaccagcagaggctgatctttgccggcaagcagctgga
agatggccgcaccctctctgattacaacatccagaaggagtcaaccctgcacctggtccttcgcctgagaggtggc
Ubiquitin A76
>UbA76 0-228
(SEQ ID NO: 39)
atgcagatcttcgtgaagaccctgaccggcaagaccatcaccctagaggtggagcccagtgacaccatcgagaacg
tgaaggccaagatccaggataaagagggcatcccccctgaccagcagaggctgatctttgccggcaagcagctgga
agatggccgcaccctctctgattacaacatccagaaggagtcaaccctgcacctggtccttcgcctgagaggtgcc
HLA-A2 (MHC class I) signal peptide
>MHC SignalPep 0-78
(SEQ ID NO: 40)
atggccgtcatggcgccccgaaccctcgtcctgctactctcgggggctctggccctgacccagacctggggggct
ct
HLA-A2 (MHC class I) Trans Membrane domain
>HLA A2 TM Domain 0-201
(SEQ ID NO: 41)
ccgtcttcccagcccaccatccccatcgtgggcatcattgctggcctggttctctttggagctgtgatcactggag
ctgtggtcgctgctgtgatgtggaggaggaagagctcagatagaaaaggagggagctactctcaggctgcaagcag
tgacagtgcccagggctctgatgtgtctctcacagcttgtaaagtgtga
IgK Leader Seq
>IgK Leader Seq 0-60
(SEQ ID NO: 42)
atggagaccgatacactgctgctgtgggtgctgctcctgtgggtgccaggaagcacaggc
Human DC-Lamp
>HumanDCLAMP 0-3178
(SEQ ID NO: 43)
ggcaccgattcggggcctgcccggacttcgccgcacgctgcagaacctcgcccagcgcccaccatgccccggcagc
tcagcgcggcggccgcgctcttcgcgtccctggccgtaattttgcacgatggcagtcaaatgagagcaaaagcatt
tccagaaaccagagattattctcaacctactgcagcagcaacagtacaggacataaaaaaacctgtccagcaacca
gctaagcaagcacctcaccaaactttagcagcaagattcatggatggtcatatcacctttcaaacagcggccacag
taaaaattccaacaactaccccagcaactacaaaaaacactgcaaccaccagcccaattacctacaccctggtcac
aacccaggccacacccaacaactcacacacagctcctccagttactgaagttacagtcggccctagcttagcccct
tattcactgccacccaccatcaccccaccagctcatacagctggaaccagttcatcaaccgtcagccacacaactg
ggaacaccactcaacccagtaaccagaccacccttccagcaactttatcgatagcactgcacaaaagcacaaccgg
tcagaagcctgatcaacccacccatgccccaggaacaacggcagctgcccacaataccacccgcacagctgcacct
gcctccacggttcctgggcccacccttgcacctcagccatcgtcagtcaagactggaatttatcaggttctaaacg
gaagcagactctgtataaaagcagagatggggatacagctgattgttcaagacaaggagtcggttttttcacctcg
gagatacttcaacatcgaccccaacgcaacgcaagcctctgggaactgtggcacccgaaaatccaaccttctgttg
aattttcagggcggatttgtgaatctcacatttaccaaggatgaagaatcatattatatcagtgaagtgggagcct
atttgaccgtctcagatccagagacagtttaccaaggaatcaaacatgcggtggtgatgttccagacagcagtcgg
gcattccttcaagtgcgtgagtgaacagagcctccagttgtcagcccacctgcaggtgaaaacaaccgatgtccaa
cttcaagcctttgattttgaagatgaccactttggaaatgtggatgagtgctcgtctgactacacaattgtgcttc
ctgtgattggggccatcgtggttggtctctgccttatgggtatgggtgtctataaaatccgcctaaggtgtcaatc
atctggataccagagaatctaattgttgcccggggggaatgaaaataatggaatttagagaactctttcatccctt
ccaggatggatgttgggaaattccctcagagtgtgggtccttcaaacaatgtaaaccaccatcttctattcaaatg
aagtgagtcatgtgtgatttaagttcaggcagcacatcaatttctaaatactttttgtttattttatgaaagatat
agtgagctgtttattttctagtttcctttagaatattttagccactcaaagtcaacatttgagatatgttgaatta
acataatatatgtaaagtagaataagccttcaaattataaaccaagggtcaattgtaactaatactactgtgtgtg
cattgaagattttattttacccttgatcttaacaaagcctttgctttgttatcaaatggactttcagtgcttttac
tatctgtgttttatggtttcatgtaacatacatattcctggtgtagcacttaactccttttccactttaaatttgt
ttttgttttttgagacggagtttcactcttgtcacccaggctggagtacagtggcacgatctcggcttatggcaac
ctccgcctcccgggttcaagtgattctcctgcttcagcttcccgagtagctgggattacaggcacacactaccacg
cctggctaatttttgtatttttattatagacgggtttcaccatgttggccagactggtcttgaactcttgacctca
ggtgatccacccacctcagcctcccaaagtgctgggattacaggcatgagccattgcgcccggccttaaatgtttt
ttttaatcatcaaaaagaacaacatatctcaggttgtctaagtgtttttatgtaaaaccaacaaaaagaacaaatc
agcttatattttttatcttgatgactcctgctccagaattgctagactaagaattaggtggctacagatggtagaa
ctaaacaataagcaagagacaataataatggcccttaattattaacaaagtgccagagtctaggctaagcacttta
tctatatctcatttcattctcacaacttataagtgaatgagtaaactgagacttaagggaactgaatcacttaaat
gtcacctggctaactgatggcagagccagagcttgaattcatgttggtctgacatcaaggtctttggtcttctccc
tacaccaagttacctacaagaacaatgacaccacactctgcctgaaggctcacacctcataccagcatacgctcac
cttacagggaaatgggtttatccaggatcatgagacattagggtagatgaaaggagagctttgcagataacaaaat
agcctatccttaataaatcctccactctctggaaggagactgaggggctttgtaaaacattagtcagttgctcatt
tttatgggattgcttagctgggctgtaaagatgaaggcatcaaataaactcaaagtatttttaaatttttttgata
atagagaaacttcgctaaccaactgttctttcttgagtgtatagccccatcttgtggtaacttgctgcttctgcac
ttcatatccatatttcctattgttcactttattctgtagagcagcctgccaagaattttatttctgctgttttttt
tgctgctaaagaaaggaactaagtcaggatgttaacagaaaagtccacataaccctagaattcttagtcaaggaat
aattcaagtcagcctagagaccatgttgactttcctcatgtgtttccttatgactcagtaagttggcaaggtcctg
actttagtcttaataaaacattgaattgtagtaaaggtttttgcaataaaaacttactttgg
Mouse LAMP1
> MouseLamp1 0-1858
(SEQ ID NO: 44)
attccggaggtgaaaaacaatggcacaacgtgtataatggccagcttctctgcctcctttctgaccacctacgaga
ctgcgaatggttctcagatcgtgaacatttccctgccagcctctgcagaagtactgaaaaatggcagttcttgtgg
taaagaaaatgtttctgaccccagcctcacaattacttttggaagaggatatttactgacactcaacttcacaaaa
aatacaacacgttacagtgtccagcatatgtattttacatataacttgtcagatacagaacattttcccaatgcca
tcagcaaagagatctacaccatggattccacaactgacatcaaggcagacatcaacaaagcataccggtgtgtcag
tgatatccgggtctacatgaagaatgtgaccgttgtgctccgggatgccactatccaggcctacctgtcgagtggc
aacttcagcaaggaagagacacactgcacacaggatggaccttccccaaccactgggccacccagcccctcaccac
cacttgtgcccacaaaccccactgtatccaagtacaatgttactggtaacaacggaacctgcctgctggcctctat
ggcactgcaactgaatatcacctacctgaaaaaggacaacaagacggtgaccagagcgttcaacatcagcccaaat
gacacatctagtgggagttgcggtatcaacttggtgaccctgaaagtggagaacaagaacagagccctggaattgc
agtttgggatgaatgccagctctagcctgtttttcttgcaaggagtgcgcttgaatatgactcttcctgatgccct
agtgcccacattcagcatctccaaccattcactgaaagctcttcaggccactgtgggaaactcatacaagtgcaac
actgaggaacacatctttgtcagcaagatgctctccctcaatgtcttcagtgtgcaggtccaggctttcaaggtgg
acagtgacaggtttgggtctgtggaagagtgtgttcaggatggtaacaacatgttgatccccattgctgtgggcgg
tgccctggcagggctgatcctcatcgtcctcattgcctacctcattggcaggaagaggagtcacgccggctatcag
accatctagcctggtgggcaggtgcaccagagatgcacaggggcctgttctcacatccccaagcttagataggtgt
ggaagggaggcacactttctggcaaactgttttaaaatctgctttatcaaatgtgaagttcatcttgcaacattta
ctatgcacaaaggaataactattgaaatgacggtgttaattttgctaactgggttaaatattgatgagaaggctcc
actgatttgacttttaagacttggtgtttggttcttcattcttttactcagatttaagcctatcaaagggatactc
tggtccagaccttggcctggcaagggtggctgatggttaggctgcacacacttaagaagcaacgggagcagggaag
gcttgcacacaggcacgcacagggtcaacctctggacacttggcttgggctacctggccttgggggggctgaactc
tggcatctggctgggtacacacccccccaatttctgtgctctgccacccgtgagctgccactttcctaaatagaaa
atggcattatttttatttacttttttgtaaagtgatttccagtcttgtgttggcgttcagggtggccctgtctctg
cactgtgtacaataatagattcacactgctgacgtgtcttgcagcgtaggtgggttgtacactgggcatcagctca
cgtaatgcattgcctgtaacgatgctaataaaaa
Human Lamp1 cDNA
>Human Lamp1 0-2339
(SEQ ID NO: 45)
ggcccaaccgccgcccgcgcccccgctctccgcaccgtacccggccgcctcgcgccatggcggcccccggcagcgc
ccggcgacccctgctgctgctactgctgttgctgctgctcggcctcatgcattgtgcgtcagcagcaatgtttatg
gtgaaaaatggcaacgggaccgcgtgcataatggccaacttctctgctgccttctcagtgaactacgacaccaaga
gtggccctaagaacatgacctttgacctgccatcagatgccacagtggtgctcaaccgcagctcctgtggaaaaga
gaacacttctgaccccagtctcgtgattgcttttggaagaggacatacactcactctcaatttcacgagaaatgca
acacgttacagcgtccagctcatgagttttgtttataacttgtcagacacacaccttttccccaatgcgagctcca
aagaaatcaagactgtggaatctataactgacatcagggcagatatagataaaaaatacagatgtgttagtggcac
ccaggtccacatgaacaacgtgaccgtaacgctccatgatgccaccatccaggcgtacctttccaacagcagcttc
agcaggggagagacacgctgtgaacaagacaggccttccccaaccacagcgccccctgcgccacccagcccctcgc
cctcacccgtgcccaagagcccctctgtggacaagtacaacgtgagcggcaccaacgggacctgcctgctggccag
catggggctgcagctgaacctcacctatgagaggaaggacaacacgacggtgacaaggcttctcaacatcaacccc
aacaagacctcggccagcgggagctgcggcgcccacctggtgactctggagctgcacagcgagggcaccaccgtcc
tgctcttccagttcgggatgaatgcaagttctagccggtttttcctacaaggaatccagttgaatacaattcttcc
tgacgccagagaccctgcctttaaagctgccaacggctccctgcgagcgctgcaggccacagtcggcaattcctac
aagtgcaacgcggaggagcacgtccgtgtcacgaaggcgttttcagtcaatatattcaaagtgtgggtccaggctt
tcaaggtggaaggtggccagtttggctctgtggaggagtgtctgctggacgagaacagcatgctgatccccatcgc
tgtgggtggtgccctggcggggctggtcctcatcgtcctcatcgcctacctcgtcggcaggaagaggagtcacgca
ggctaccagactatctagcctggtgcacgcaggcacagcagctgcaggggcctctgttcctttctctgggcttagg
gtcctgtcgaaggggaggcacactttctggcaaacgtttctcaaatctgcttcatccaatgtgaagttcatcttgc
agcatttactatgcacaacagagtaactatcgaaatgacggtgttaattttgctaactgggttaaatattttgcta
actggttaaacattaatatttaccaaagtaggattttgagggtgggggtgctctctctgagggggtgggggtgccg
ctgtctctgaggggtgggggtgccgctgtctctgaggggtgggggtgccgctctctctgagggggtgggggtgccg
ctttctctgagggggtgggggtgccgctctctctgagggggtgggggtgctgctctctccgaggggtggaatgccg
ctgtctctgaggggtgggggtgccgctctaaattggctccatatcatttgagtttagggttctggtgtttggtttc
ttcattctttactgcactcagatttaagccttacaaagggaaagcctctggccgtcacacgtaggacgcatgaagg
tcactcgtggtgaggctgacatgctcacacattacaacagtagagagggaaaatcctaagacagaggaactccaga
gatgagtgtctggagcgcttcagttcagctttaaaggccaggacgggccacacgtggctggcggcctcgttccagt
ggcggcacgtccttgggcgtctctaatgtctgcagctcaagggctggcacttttttaaatataaaaatgggtgtta
tttttatttttttttgtaaagtgatttttggtcttctgttgacattcggggtgatcctgttctgcgctgtgtacaa
tgtgagatcggtgcgttctcctgatgttttgccgtggcttggggattgtacacgggaccagctcacgtaatgcatt
gcctgtaacaatgtaataaaaagcctctttcttttaaaaaaaaaaaaaaaaaaaaaaaa
Tetanus toxoid nulceic acid sequence
(SEQ ID NO: 46)
cagtacatcaaggccaacagcaagttcatcggcatcaccgaactc
Tetanus toxoid amino acid sequence
(SEQ ID NO: 47)
QYIKANSKFIGITEL
PADRE nulceotide sequence
(SEQ ID NO: 48)
gctaaatttgtggctgcctggacactgaaagccgccgct
PADRE amino acid sequence
(SEQ ID NO: 49)
AKFVAAWTLKAAA
WPRE
> WPRE 0-593
(SEQ ID NO: 50)
aatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatc
ctggttgctgtctctttatgaggagttgtggcccgttgtcaggcaacgtggcgtggtgtgcactgtgtttgctgac
gcaacccccactggttggggcattgccaccacctgtcagctcctttccgggactttcgctttccccctccctattg
ccacggcggaactcatcgccgcctgccttgcccgctgctggacaggggctcggctgttgggcactgacaattccgt
ggtgttgtcggggaagctgacgtcctttccatggctgctcgcctgtgttgccacctggattctgcgcgggacgtcc
ttctgctacgtcccttcggccctcaatccagcggaccttccttcccgcggcctgctgccggctctgcggcctcttc
cgcgtcttcgccttcgccctcagacgagtcggatctccctttgggccgcctccccgcctgt
IRES
>eGFP_IRES_SEAP_Insert 1746-2335
(SEQ ID NO: 51)
tctcccccccccccctctccctcccccccccctaacgttactggccgaagccgcttggaataaggccggtgtgcgt
ttgtctatatgttattttccaccatattgccgtcttttggcaatgtgagggcccggaaacctggccctgtcttctt
gacgagcattcctaggggtctttcccctctcgccaaaggaatgcaaggtctgttgaatgtcgtgaaggaagcagtt
cctctggaagcttcttgaagacaaacaacgtctgtagcgaccctttgcaggcagcggaaccccccacctggcgaca
ggtgcctctgcggccaaaagccacgtgtataagatacacctgcaaaggcggcacaaccccagtgccacgttgtgag
ttggatagttgtggaaagagtcaaatggctctcctcaagcgtattcaacaaggggctgaaggatgcccagaaggta
ccccattgtatgggatctgatctggggcctcggtgcacatgctttacatgtgtttagtcgaggttaaaaaaacgtc
taggccccccgaaccacggggacgtggttttcctttgaaaaacacgatgataatatg
GFP
(SEQ ID NO: 52)
atggtgagcaagggcgaggagctgttcaccggggtggtgcccatcctggtcgagctggacggcgacgtaaacggcc
acaagttcagcgtgtccggcgagggcgagggcgatgccacctacggcaagctgaccctgaagttcatctgcaccac
cggcaagctgcccgtgccctggcccaccctcgtgaccaccctgacctacggcgtgcagtgcttcagccgctacccc
gaccacatgaagcagcacgacttcttcaagtccgccatgcccgaaggctacgtccaggagcgcaccatcttcttca
aggacgacggcaactacaagacccgcgccgaggtgaagttcgagggcgacaccctggtgaaccgcatcgagctgaa
gggcatcgacttcaaggaggacggcaacatcctggggcacaagctggagtacaactacaacagccacaacgtctat
atcatggccgacaagcagaagaacggcatcaaggtgaacttcaagatccgccacaacatcgaggacggcagcgtgc
agctcgccgaccactaccagcagaacacccccatcggcgacggccccgtgctgctgcccgacaaccactacctgag
cacccagtccgccctgagcaaagaccccaacgagaagcgcgatcacatggtcctgctggagttcgtgaccgccgcc
gggatcactctcggcatggacgagctgtacaagtag
SEAP
(SEQ ID NO: 53)
atgctgctgctgctgctgctgctgggcctgaggctacagctctccctgggcatcatcccagttgaggaggagaacc
cggacttctggaaccgcgaggcagccgaggccctgggtgccgccaagaagctgcagcctgcacagacagccgccaa
gaacctcatcatcttcctgggcgatgggatgggggtgtctacggtgacagctgccaggatcctaaaagggcagaag
aaggacaaactggggcctgagatacccctggccatggaccgcttcccatatgtggctctgtccaagacatacaatg
tagacaaacatgtgccagacagtggagccacagccacggcctacctgtgcggggtcaagggcaacttccagaccat
tggcttgagtgcagccgcccgctttaaccagtgcaacacgacacgcggcaacgaggtcatctccgtgatgaatcgg
gccaagaaagcagggaagtcagtgggagtggtaaccaccacacgagtgcagcacgcctcgccagccggcacctacg
cccacacggtgaaccgcaactggtactcggacgccgacgtgcctgcctcggcccgccaggaggggtgccaggacat
cgctacgcagctcatctccaacatggacattgacgtgatcctaggtggaggccgaaagtacatgtttcgcatggga
accccagaccctgagtacccagatgactacagccaaggtgggaccaggctggacgggaagaatctggtgcaggaat
ggctggcgaagcgccagggtgcccggtatgtgtggaaccgcactgagctcatgcaggcttccctggacccgtctgt
gacccatctcatgggtctctttgagcctggagacatgaaatacgagatccaccgagactccacactggacccctcc
ctgatggagatgacagaggctgccctgcgcctgctgagcaggaacccccgcggcttcttcctcttcgtggagggtg
gtcgcatcgaccatggtcatcatgaaagcagggcttaccgggcactgactgagacgatcatgttcgacgacgccat
tgagagggcgggccagctcaccagcgaggaggacacgctgagcctcgtcactgccgaccactcccacgtcttctcc
ttcggaggctaccccctgcgagggagctccatcttcgggctggcccctggcaaggcccgggacaggaaggcctaca
cggtcctcctatacggaaacggtccaggctatgtgctcaaggacggcgcccggccggatgttaccgagagcgagag
cgggagccccgagtatcggcagcagtcagcagtgcccctggacgaagagacccacgcaggcgaggacgtggcggtg
ttcgcgcgcggcccgcaggcgcacctggttcacggcgtgcaggagcagaccttcatagcgcacgtcatggccttcg
ccgcctgcctggagccctacaccgcctgcgacctggcgccccccgccggcaccaccgacgccgcgcacccgggtta
ctctagagtcggggcggccggccgcttcgagcagacatgataa
Firefly Luciferase
(SEQ ID NO: 54)
atggaagatgccaaaaacattaagaagggcccagcgccattctacccactcgaagacgggaccgccggcgagcagc
tgcacaaagccatgaagcgctacgccctggtgcccggcaccatcgcctttaccgacgcacatatcgaggtggacat
tacctacgccgagtacttcgagatgagcgttcggctggcagaagctatgaagcgctatgggctgaatacaaaccat
cggatcgtggtgtgcagcgagaatagcttgcagttcttcatgcccgtgttgggtgccctgttcatcggtgtggctg
tggccccagctaacgacatctacaacgagcgcgagctgctgaacagcatgggcatcagccagcccaccgtcgtatt
cgtgagcaagaaagggctgcaaaagatcctcaacgtgcaaaagaagctaccgatcatacaaaagatcatcatcatg
gatagcaagaccgactaccagggcttccaaagcatgtacaccttcgtgacttcccatttgccacccggcttcaacg
agtacgacttcgtgcccgagagcttcgaccgggacaaaaccatcgccctgatcatgaacagtagtggcagtaccgg
attgcccaagggcgtagccctaccgcaccgcaccgcttgtgtccgattcagtcatgcccgcgaccccatcttcggc
aaccagatcatccccgacaccgctatcctcagcgtggtgccatttcaccacggcttcggcatgttcaccacgctgg
gctacttgatctgcggctttcgggtcgtgctcatgtaccgcttcgaggaggagctattcttgcgcagcttgcaaga
ctataagattcaatctgccctgctggtgcccacactatttagcttcttcgctaagagcactctcatcgacaagtac
gacctaagcaacttgcacgagatcgccagcggcggggcgccgctcagcaaggaggtaggtgaggccgtggccaaac
gcttccacctaccaggcatccgccagggctacggcctgacagaaacaaccagcgccattctgatcacccccgaagg
ggacgacaagcctggcgcagtaggcaaggtggtgcccttcttcgaggctaaggtggtggacttggacaccggtaag
acactgggtgtgaaccagcgcggcgagctgtgcgtccgtggccccatgatcatgagcggctacgttaacaaccccg
aggctacaaacgctctcatcgacaaggacggctggctgcacagcggcgacatcgcctactgggacgaggacgagca
cttcttcatcgtggaccggctgaagagcctgatcaaatacaagggctaccaggtagccccagccgaactggagagc
atcctgctgcaacaccccaacatcttcgacgccggggtcgccggcctgcccgacgacgatgccggcgagctgcccg
ccgcagtcgtcgtgctggaacacggtaaaaccatgaccgagaaggagatcgtggactatgtggccagccaggttac
aaccgccaagaagctgcgcggtggtgttgtgttcgtggacgaggtgcctaaaggactgaccggcaagttggacgcc
cgcaagatccgcgagattctcattaaggccaagaagggcggcaagatcgccgtgtaa
FMDV 2A
(SEQ ID NO: 55)
gtaaagcaaacactgaactttgaccttctcaagttggctggagacgttgagtccaatcctgggccc
GPGPG linker
(SEQ ID NO: 56)
GPGPG
KRAS 4X (4x4) Cassette Nucleotide Sequence
ATGGCTGGCATGACCGAGTATAAACTAGTAGTTGTGGGAGCGTGTGGTGTAGGCAAGTCGGCACTTA
CAATTCAGTTGATACAAATGACGGAATATAAGCTCGTAGTAGTCGGAGCAGACGGCGTGGGGAAAT
CAGCGTTGACTATCCAGTTAATACAGGAAACTTGCCTATTAGACATCTTGGATACGGCAGGTCATG
AGGAATATTCCGCTATGAGAGATCAGTATATGCGCATGACGGAGTATAAGCTTGTGGTTGTCGGGG
CCGACGGGGTAGGTAAGTCAGCGCTCACGATACAATTAATTCAAATGACCGAATACAAGTTGGTCG
TGGTGGGGGCAGTTGGGGTCGGTAAATCCGCGTTAACGATCCAACTTATCCAAATGACAGAATATA
AACTCGTTGTTGTAGGTGCATGTGGCGTAGGAAAAAGCGCATTGACCATCCAGCTAATTCAGGAGA
CGTGTCTCCTTGATATCCTAGACACGGCGGGGCACGAAGAATACTCGGCTATGCGCGACCAGTACA
TGAGAATGACGGAATACAAACTTGTTGTCGTGGGTGCGGATGGAGTAGGGAAAAGTGCTCTAACAA
TACAACTCATTCAGATGACAGAGTACAAATTGGTAGTCGTCGGTGCGGTAGGAGTTGGGAAGTCTG
CACTAACTATTCAGCTCATACAGATGACCGAGTACAAGCTGGTGGTGGTAGGCGCTTGCGGTGTGG
GTAAGAGTGCATTAACCATACAGCTTATACAAGAGACATGTCTGCTAGATATATTAGATACCGCCG
GGCATGAAGAGTACTCTGCCATGCGAGACCAATACATGCGTATGACAGAGTATAAATTAGTAGTGG
TTGGGGCGGACGGTGTTGGCAAGAGCGCCTTAACTATACAGTTGATCCAGATGACGGAGTACAAAC
TGGTCGTCGTTGGTGCAGTGGGAGTGGGAAAATCTGCGCTGACGATTCAACTAATCCAAGAAACAT
GTTTACTTGACATCCTCGACACTGCGGGTCACGAGGAGTATTCGGCGATGCGTGATCAATATATGA
GGATGACTGAGTATAAGTTAGTCGTAGTTGGAGCGGTCGGTGTCGGAAAGTCCGCGCTAACCATTC
AATTGATTCAAATGACTGAATACAAGCTAGTGGTAGTAGGAGCATGCGGCGTCGGCAAATCGGCTT
TAACAATCCAACTGATACAGGGACCCGGACCAGGCGCCAAATTTGTTGCTGCTTGGACACTGAAAG
CTGCTGCTGGGCCCGGACCAGGCCAGTACATCAAGGCCAACTCTAAGTTTATCGGCATCACCGAAT
TGGGACCTGGACCCGGCTAG
KRAS G12C MHC Class I Neoepitope
VVVGACGVGK
KRAS G12C MHC Class I Neoepitope
KLVVVGACGV
KRAS G12D MHC Class I Neoepitope
VVGADGVGK
KRAS G12D MHC Class I Neoepitope
VVVGADGVGK
KRAS G12V MHC Class I Neoepitope
VVGAVGVGK
KRAS G12V MHC Class I Neoepitope
AVGVGKSAL
KRAS G12V MHC Class I Neoepitope
VVVGAVGVGK
KRAS Q61H MHC Class I Neoepitope
ILDTAGHEEY

### REFERENCES

1. Reynisson, B., Alvarez, B., Paul, S., Peters, B., and Nielsen, M. (2020). NetMHCpan-4.1 and NetMHCIIpan-4.0: improved predictions of MHC antigen presentation by concurrent motif deconvolution and integration of MS MHC eluted ligand data. Nucleic Acids Res 48, W449-W454. 10.1093/nar/gkaa379.
2. Cheng, J., Bendjama, K., Rittner, K., and Malone, B. (2021). BERTMHC: Improved MHC-peptide class II interaction prediction with transformer and multiple instance learning. Bioinformatics 37, 4172-4179. 10.1093/bioinformatics/btab422.
3. Lin, Z., Akin, H., Rao, R., Hie, B., Zhu, Z., Lu, W., Smetanin, N., Verkuil, R., Kabeli, O., Shmueli, Y., et al. (2023). Evolutionary-scale prediction of atomic-level protein structure with a language model. Science 379, 1123-1130. doi:10.1126/science.ade2574.
4. Rives, A., Meier, J., Sercu, T., Goyal, S., Lin, Z., Liu, J., Guo, D., Ott, M., Zitnick, C. L., Ma, J., and Fergus, R. (2021). Biological structure and function emerge from scaling unsupervised learning to 250 million protein sequences. Proc Natl Acad Sci USA 118. 10.1073/pnas.2016239118.
5. Devlin, J., Chang, M.-W., Lee, K. & Toutanova, K. (2018). BERT: Pre-training of Deep Bidirectional Transformers for Language Understanding. arXiv:1810.04805.
6. Rao, R., Bhattacharya, N., Thomas, N., Duan, Y., Chen, X., Canny, J., Abbeel, P., and Song, Y. S. (2019). Evaluating Protein Transfer Learning with TAPE. Adv Neural Inf Process Syst 32, 9689-9701.
7. Lin, Z., Akin, H., Rao, R., Hie, B., Zhu, Z., Lu, W., Santos Costa, A.d., Fazel-Zarandi, M., Sercu, T., Candido, S., and Rives, A. (2022). Language models of protein sequences at the scale of evolution enable accurate structure prediction. bioRxiv, 2022.2007.2020.500902. 10.1101/2022.07.20.500902.
8. Reynisson, B., Barra, C., Kaabinejadian, S., Hildebrand, W. H., Peters, B., and Nielsen, M. (2020). Improved Prediction of MHC II Antigen Presentation through Integration and Motif Deconvolution of Mass Spectrometry MHC Eluted Ligand Data. J Proteome Res 19, 2304-2315. 10.1021/acs.jproteome.9b00874.
9. Chen, B., Khodadoust, M. S., Olsson, N., Wagar, L. E., Fast, E., Liu, C.L., Muftuoglu, Y., Sworder, B. J., Diehn, M., Levy, R., et al. (2019). Predicting HLA class II antigen presentation through integrated deep learning. Nat Biotechnol 37, 1332-1343. 10.1038/s41587-019-0280-2.
10. Ott, P. A., Hu, Z., Keskin, D. B., Shukla, S. A., Sun, J., Bozym, D. J., Zhang, W., Luoma, A., Giobbie-Hurder, A., Peter, L., et al. (2017). An immunogenic personal neoantigen vaccine for patients with melanoma. Nature 547, 217-221. 10.1038/nature22991.
11. Skoulidis, F., Li, B. T., Dy, G. K., Price, T. J., Falchook, G. S., Wolf, J., Italiano, A., Schuler, M., Borghaei, H., Barlesi, F., et al. (2021). Sotorasib for Lung Cancers with KRAS p. G12C Mutation. N Engl J Med 384, 2371-2381. 10.1056/NEJMoa2103695.
12. Jänne, P. A., Riely, G. J., Gadgeel, S. M., Heist, R. S., Ou, S.-H. I., Pacheco, J. M., Johnson, M. L., Sabari, J. K., Leventakos, K., Yau, E., et al. (2022). Adagrasib in Non-Small-Cell Lung Cancer Harboring a KRASG12C Mutation. New England Journal of Medicine 387, 120-131. 10.1056/NEJMoa2204619.
13. Sattler, M., Mohanty, A., Kulkarni, P., and Salgia, R. (2023). Precision oncology provides opportunities for targeting KRAS-inhibitor resistance. Trends in Cancer 9, 42-54. 10.1016/j.trecan.2022.10.001.
14. Huang, L., Guo, Z., Wang, F., and Fu, L. (2021). KRAS mutation: from undruggable to druggable in cancer. Signal Transduction and Targeted Therapy 6, 386. 10.1038/s41392-021-00780-4.
15. Sharav, T., Wiesmuller, K. H., and Walden, P. (2007). Mimotope vaccines for cancer immunotherapy. Vaccine 25, 3032-3037. 10.1016/j.vaccine.2007.01.033.
16. Deocaris, C. C., Taira, K., Kaul, S. C., and Wadhwa, R. (2005). Mimotope-hormesis and mortalin/grp75/mthsp70: a new hypothesis on how infectious disease-associated epitope mimicry may explain low cancer burden in developing nations. FEBS Lett 579, 586-590. 10.1016/j.febslet.2004.11.108.
17. Al-Attiyah, R., and Mustafa, A. S. (2004). Computer-assisted prediction of HLA-DR binding and experimental analysis for human promiscuous Th1-cell peptides in the 24 kDa secreted lipoprotein (LppX) of Mycobacterium tuberculosis. Scand J Immunol 59, 16-24. 10.1111/j.0300-9475.2004.01349.x.
18. Porichis, F., Hart, M. G., Massa, A., Everett, H. L., Morou, A., Richard, J., Brassard, N., Veillette, M., Hassan, M., Ly, N. L., et al. (2018). Immune Checkpoint Blockade Restores HIV-Specific CD4 T Cell Help for NK Cells. J Immunol 201, 971-981. 10.4049/jimmunol.1701551.
19. Bear A S, Blanchard T, Cesare J, Ford M J, Richman L P, Xu C, Baroja M L, McCuaig S, Costeas C, Gabunia K, Scholler J, Posey A D Jr, O'Hara M H, Smole A, Powell D J Jr, Garcia B A, Vonderheide R H, Linette G P, Carreno B M. (2021) Biochemical and functional characterization of mutant KRAS epitopes validates this oncoprotein for immunological targeting. Nat Commun. 2021 Jul. 16; 12(1):4365. doi: 10.1038/s41467-021-24562-2.
20. Tumeh, P., Harview, C., Yearley, J. et al. PD-1 blockade induces responses by inhibiting adaptive immune resistance. Nature 515, 568-571 (2014). https://doi.org/10 1038/nature13954.
21. Palmer, C. D., Rappaport, A. R., Davis, M. J. et al. Individualized, heterologous chimpanzee adenovirus and self-amplifying mRNA neoantigen vaccine for advanced metastatic solid tumors: phase 1 trial interim results. Nat Med 28, 1619-1629 (2022). https://doi.org/10.1038/s41591-022-01937-6

TABLE 1
Identification of patient biomarkers by name,
abbreviation and UniProt identifier.

| | | UniProt |
|---|---|---|
| Name | Abbreviation | identifier |
| Programmed cell death 1 | CD274 | Q9NZQ7 |
| ligand 1 | | |
| T-cell surface glycoprotein | CD8A | P01732 |
| CD8 alpha chain | | |
| C-X-C motif chemokine 9 | CXCL9 | Q07325 |
| Granzyme A | GZMA | Q7YRZ7 |
| Profilin-1 | PRF1 | Q42449 |
| TP53-binding protein 1 | TP53 | Q12888 |
| Adenomatous polyposis coli | APC | P25054 |
| protein | | |
| GTPase KRas | KRAS | P01116 |
| Phosphatidylinositol 4,5- | PIK3CA | P42336 |
| bisphosphate 3-kinase | | |
| catalytic subunit alpha | | |
| isoform | | |
| Mothers against | SMAD4 | Q13485 |
| decapentaplegic homolog 4 | | |

TABLE 2
KRAS G12C Neoepitopes

ACGVGKSALT
ACGVGKSALTI
ACGVGKSALTIQ
ACGVGKSALTIQL
ACGVGKSALTIQLI
ACGVGKSALTIQLIQ
CGVGKSALTI
CGVGKSALTIQ
CGVGKSALTIQL
CGVGKSALTIQLI
CGVGKSALTIQLIQ
EYKLVVVGAC
EYKLVVVGACG
EYKLVVVGACGV
EYKLVVVGACGVG
EYKLVVVGACGVGK
EYKLVVVGACGVGKS
EYKLVVVGACGVGKSA
EYKLVVVGACGVGKSAL
EYKLVVVGACGVGKSALT
EYKLVVVGACGVGKSALTI
EYKLVVVGACGVGKSALTIQ
GACGVGKSAL
GACGVGKSALT
GACGVGKSALTI
GACGVGKSALTIQ
GACGVGKSALTIQL
GACGVGKSALTIQLI
GACGVGKSALTIQLIQ
KLVVVGACGV
KLVVVGACGVG
KLVVVGACGVGK
KLVVVGACGVGKS
KLVVVGACGVGKSA
KLVVVGACGVGKSAL
KLVVVGACGVGKSALT
KLVVVGACGVGKSALTI
KLVVVGACGVGKSALTIQ
KLVVVGACGVGKSALTIQL
KLVVVGACGVGKSALTIQLI
LVVVGACGVG
LVVVGACGVGK
LVVVGACGVGKS
LVVVGACGVGKSA
LVVVGACGVGKSAL
LVVVGACGVGKSALT
LVVVGACGVGKSALTI
LVVVGACGVGKSALTIQ
LVVVGACGVGKSALTIQL
LVVVGACGVGKSALTIQLI
LVVVGACGVGKSALTIQLIQ
MTEYKLVVVGAC
MTEYKLVVVGACG
MTEYKLVVVGACGV
MTEYKLVVVGACGVG
MTEYKLVVVGACGVGK
MTEYKLVVVGACGVGKS
MTEYKLVVVGACGVGKSA
MTEYKLVVVGACGVGKSAL
MTEYKLVVVGACGVGKSALT
TEYKLVVVGAC
TEYKLVVVGACG
TEYKLVVVGACGV
TEYKLVVVGACGVG
TEYKLVVVGACGVGK
TEYKLVVVGACGVGKS
TEYKLVVVGACGVGKSA
TEYKLVVVGACGVGKSAL
TEYKLVVVGACGVGKSALT
TEYKLVVVGACGVGKSALTI
VGACGVGKSA
VGACGVGKSAL
VGACGVGKSALT
VGACGVGKSALTI
VGACGVGKSALTIQ
VGACGVGKSALTIQL
VGACGVGKSALTIQLI
VGACGVGKSALTIQLIQ
VVGACGVGKS
VVGACGVGKSA
VVGACGVGKSAL
VVGACGVGKSALT
VVGACGVGKSALTI
VVGACGVGKSALTIQ
VVGACGVGKSALTIQL
VVGACGVGKSALTIQLI
VVGACGVGKSALTIQLIQ
VVVGACGVGK
VVVGACGVGKS
VVVGACGVGKSA
VVVGACGVGKSAL
VVVGACGVGKSALT
VVVGACGVGKSALTI
VVVGACGVGKSALTIQ
VVVGACGVGKSALTIQL
VVVGACGVGKSALTIQLI
VVVGACGVGKSALTIQLIQ
YKLVVVGACG
YKLVVVGACGV
YKLVVVGACGVG
YKLVVVGACGVGK
YKLVVVGACGVGKS
YKLVVVGACGVGKSA
YKLVVVGACGVGKSAL
YKLVVVGACGVGKSALT
YKLVVVGACGVGKSALTI
YKLVVVGACGVGKSALTIQ
YKLVVVGACGVGKSALTIQL

TABLE 3
Sequence identity and associated MARIA and EDGE-II scores of selected
peptides used in IncuCyte killing assays

| | MARIA | | |
|---|---|---|---|
| Sequence | percentile | EDGE-II logit | y |
| SPIKLVQKVASKIPFPDRITEESV | 97.918 | −2.585154259 | 0 |
| NNSKKKWFLFQDSKKIQVEQPQ | 99.559 | 4.517350402 | 0 |
| DRSVLAKKLKFVTLVFRHGDRSPID | 94.553 | −3.076193523 | 0 |
| SHTQTTLFHTFYELLIQKNKHK | 93.732 | −2.227591283 | 0 |
| TKRQVILLHTELERFLEYLPLRF | 93.742 | 4.912554155 | 0 |
| EDSDKLFESKAELADHQKF | 98.358 | 4.605271191 | 0 |
| SHNELADSGIPENSFNVSSLVE | 93.953 | 3.607636532 | 0 |
| RLVLGKFGDLTNNFSSPHAR | 97.717 | 3.611534309 | 0 |
| RRGGALFASRPRFTPL | 96.526 | −5.875331934 | 0 |
| LCPREEFLRLCKKIMMRSIQ | 88.076 | −5.988961417 | 0 |
| SGSPPLRVSVGDFSQEFSPIQEAQQD | 95.905 | 4.747355882 | 1 |
| LSPREEFLRLCKKIMMRSIQ | 96.906 | −5.988961417 | 1 |
| RPAGRTQLLWTPAAPTAMAEVGPGHTP | 90.759 | 3.220541104 | 1 |
| WTPAAPTAMAEVGPGHTPAHPSQGAVPP | 84.121 | 0.533933199 | 1 |
| AAVRPEQRPAARGSRV | 73.378 | −2.605270332 | 0 |
| PGGDSGELITDAHELGVAHPPGY | 98.458 | 6.072543507 | 0 |
| VTSPKASPVTFPAAAFPTASPANKD | 96.436 | 0.605878238 | 1 |
| LENNANHDETSFLLPRKESNIVD | 98.158 | −1.926846824 | 0 |
| RGQIKLADFRLARLYSSEESR | 98.418 | 0.881340845 | 0 |
| KHLPGVNFPGNQWNPVEGILPS | 95.314 | −2.758649743 | 0 |
| PAPPPAVPKEHPAPPAPPPASAPTP | 85.002 | −0.964891023 | 0 |
| LGETMGQVTEKLQPTYMEET | 96.205 | 0.259445587 | 0 |
| PETGEIQVKTFLDREQRESYELKV | 99.6 | 0.825487779 | 0 |
| TFPKKIQMLARDFLDEY | 99.569 | −2.677735281 | 0 |
| EVVGGYTWPSGNIYQGYWAQGKR | 99.349 | −1.802886963 | 0 |
| PAHPSQGAVPPSRAAAEPHLKPSPSELQTA | 83.26 | −1.747997103 | 1 |
| DEQGREAELARSGPSAAGPVRLKPGLVPGL | 94.584 | 3.412729378 | 0 |
| TIKNSDKNVVLEHFG | 81.049 | −4.453841607 | 0 |
| DGGRQHSGPRRHSGAGPKPSSSEWAVCWAP | 91.179 | −2.436924617 | 0 |
| FCGTPDYIAPKIIAYQPYGKSVD | 99.65 | 4.361378412 | 0 |
| RGRLPAGAVRTLLSQVNKVWDQSS | 98.829 | −3.627270999 | 0 |
| DRASFLLTDYALSPDGSIRKATG | 98.688 | 4.845800966 | 1 |
| SPGPRTAPRPGSQKQAGKDWQ | 87.245 | −0.642418673 | 0 |
| HASHLQEHQRIYTGEKPFKCDT | 91.97 | −1.949572318 | 0 |
| SLPSNVLSSLVLVPLHTTPK | 94.183 | −0.632693703 | 0 |
| GHEHQPDMQKSLLRAAFFGKCFLDR | 76.352 | −0.948433335 | 1 |
| SSHYKFSKPALQSQSISLVQQS | 99.61 | 2.398331716 | 1 |
| TRNSFALVPSLQRLMLRKVALKNVDSSPS | 99.299 | −1.725999524 | 0 |
| NLKAPRLLFAPEYGPKLKLRALEDRHS | 95.935 | 2.2230455 | 0 |
| TETVNHHYLLFQNTDLGSFHDLLR | 97.998 | 2.843851742 | 1 |
| EDLDANLRKLNFRLFVIRGQPAD | 97.317 | −5.161572797 | 0 |
| ERFWRNILLLSLHKGSLYPRIPGLGKE | 98.098 | −1.802886963 | 0 |
| ELQYRGRELRFNLIANQHLLAPGFVSETR | 99.049 | 5.253883086 | 1 |
| STLPVISDSTTKRRWSALVIGL | 94.553 | −3.780616196 | 0 |
| KMQRRNDDKSILMHGLVSLRESSRG | 93.943 | 1.010408062 | 0 |
| TTVTHERKQAKVVNPPIQEVGKGARK | 97.607 | 1.603449871 | 0 |
| KGEKNGMTFSSTKDYVNNV | 97.597 | −3.223252656 | 0 |
| SLTEESGGAVAFFPGNLSTSSSA | 89.808 | 4.353371951 | 0 |
| DSYHLYAYHEELSATVPSQWKKIG | 97.797 | 3.881665865 | 0 |
| GHQKLPGKIHLFEAEFTQVAKKEPDG | 99.299 | 4.093937976 | 1 |
| GDQYKATDFVADWAGTFKMVFTPKDGSG | 99.68 | 6.318166992 | 1 |
| TTPSGSAEYMASEVVEVFTDQAT | 92.171 | 1.151583331 | 0 |
| PENDDLFMMPRIVDVTSLATEGG | 99.239 | 2.869146642 | 0 |
| MSQDIKKADEQIESMTYSTERKT | 96.726 | 1.270918854 | 0 |
| DGVSEEFWLVDLLPSTHYT | 97.707 | 4.261868929 | 1 |
| RYNSTAATNEVSEVTVFSKSPVT | 80.757 | 1.185861054 | 0 |
| GRMSPSQFARVPGYVGSPLAAMNPK | 99.549 | 4.724164609 | 1 |
| SHHTHSYQRYSHPLFLPGHRLDPPI | 97.707 | 1.335203184 | 0 |
| SHQIHSYQLYTHPLLHPWDHRD | 96.666 | −0.828321959 | 0 |
| DKGHQFHVHPLLHSGDDLDP | 98.008 | −0.07964207 | 0 |
| KLRTIPLSDNTIFRRICTIAKHLE | 99.009 | −2.309977414 | 1 |
| ASATEPANDSLFSPGAANLFSTYLAR | 93.462 | −1.343713732 | 0 |
| FPVVQSTEDVFPQGLPNEYAFVT | 82.409 | 2.580561123 | 0 |
| AASAAAFPSQRTSWEFLQSLVSIKQEK | 96.686 | 1.619310374 | 1 |
| GSVLQFMPFTTVSELMKVSAMSSPKV | 98.368 | −2.681064269 | 0 |
| NQVLASRYGIRGFSTIKIFQKGESPV | 95.725 | 4.100155865 | 0 |
| ARLQSKEYPVIFKSIMRQRLISPQL | 96.165 | 2.347056777 | 0 |
| DVTGPHLYSIYLHGSTDKLPYVTMGS | 99.539 | 2.490880059 | 0 |
| SHLASLKNNVSPVLRSHSFSDPSPKFA | 97.897 | −0.24928294 | 1 |
| TAQFAPSPGQPPALSPSYPGHRLPLQQG | 87.245 | −0.597569284 | 0 |
| PASAKSRREFDKIELAYRR | 69.904 | −2.787562264 | 0 |
| MAGPKGFQYRALYPFRRER | 99.029 | −2.649821696 | 0 |
| SDAFSGLTALPQSILLFGP | 89.327 | 3.083333279 | 0 |
| STQHADLTIIDNIKEMNFLRRYK | 98.098 | 1.318307615 | 0 |
| LHTHYDYVSALHPVSTPSKEYTSA | 97.287 | 3.340731129 | 0 |
| SSPLGRANGRRFANPRDSFSAMGFQR | 97.527 | −1.762320114 | 0 |
| EIHGKCENMTITSRGTTVTPTKETVSLG | 93.542 | 3.785212861 | 0 |
| LNTGLFRIKFKEPLENLI | 96.686 | −5.355481691 | 0 |
| SPQSGGAATLAAQARLQPVHLDVWGEHERG | 94.083 | −2.871116245 | 0 |
| GSGSQMPAWRTRGAISASSTQKTPTTRL | 98.738 | −3.928161506 | 0 |
| GLTRISIQRAQPLPPCLPSFRPPTALQGLS | 97.097 | −2.65469866 | 0 |
| SRLQTRKNKKLALSSTPSNIAPSD | 98.819 | −1.856598648 | 1 |
| WCTEMKRVFGFPVHYTDVSNMS | 94.744 | −2.701243836 | 0 |
| GPLQLPVTRKNMPLPGVVKLPPLPGS | 88.666 | −3.267588135 | 0 |
| ALLQNVELRRNVLVSPTPLAN | 91.94 | −5.253883086 | 0 |
| VNGISSQPQVPFYPNLQKSQYYSTV | 92.791 | 2.545935753 | 0 |
| YLSHTLGAASSFMRPTVPPPQF | 91.78 | −5.444830985 | 0 |
| SLRNNMFEISDRFIGIYKTYNITK | 98.708 | 3.170269267 | 0 |
| VTLNDMKARQKALVRERERQLA | 95.795 | −4.075505542 | 0 |
| VKQLERGEASVVDFKKNLEYAAT | 98.338 | −0.055214023 | 0 |
| TKLKSKAPHWTNCILHEYKNLSTS | 97.687 | −5.42174103 | 0 |
| FAKGFRESDLNSWPVAPRPLLSV | 94.473 | −3.172857934 | 0 |
| HLLQKQTSIQSPSLYGNSSPPLNK | 89.487 | 1.985818008 | 0 |
| STEVEPKESPHLARHRHLMKTLVKSLST | 89.618 | −4.845800966 | 0 |
| DGAWPVLLDKFVEWYKDKQMS | 97.918 | 1.41338946 | 0 |
| SHKLESIKEITNFKDAKQLL | 97.948 | −3.196437667 | 0 |
| TGKPEMDFVRLAQLFARARPMGLF | 96.436 | −0.556317269 | 0 |

TABLE 4
NetMHCIIpan4.0 presentation scores for binders for epitopes containing G12C mutation

| | | | | | Core_ | | Score_ | %Rank_ | Bind- |
|---|---|---|---|---|---|---|---|---|---|
| Pos | MHC | Peptide | Of | Core | Rel | Identity | EL | EL | Level |
| 18 | HLA- | ALTIQLIQNHFVDE | 3 | IQLIQNHFV | 0.907 | Sequence | 0.088209 | 0.48 | <=SB |
| | DQA10102- | | | | | | | | |
| | DQB10501 | | | | | | | | |
| 17 | HLA- | SALTIQLIQNHFVDE | 4 | IQLIQNHFV | 0.88 | Sequence | 0.072668 | 0.84 | <=SB |
| | DQA10102- | | | | | | | | |
| | DQB10501 | | | | | | | | |
| 18 | HLA- | ALTIQLIQNHFVDEY | 3 | IQLIQNHFV | 0.873 | Sequence | 0.078823 | 0.67 | <=SB |
| | DQA10102- | | | | | | | | |
| | DQB10501 | | | | | | | | |
| 18 | HLA- | ALTIQLIQNHFVDE | 3 | IQLIQNHFV | 0.9 | Sequence | 0.086239 | 0.78 | <=SB |
| | DQA10103- | | | | | | | | |
| | DQB10501 | | | | | | | | |
| 18 | HLA- | ALTIQLIQNHFVDE | 3 | IQLIQNHFV | 0.907 | Sequence | 0.088209 | 0.48 | <=SB |
| | DQA10102- | | | | | | | | |
| | DQB10501 | | | | | | | | |
| 17 | HLA- | SALTIQLIQNHFVDE | 4 | IQLIQNHFV | 0.88 | Sequence | 0.072668 | 0.84 | <=SB |
| | DQA10102- | | | | | | | | |
| | DQB10501 | | | | | | | | |
| 18 | HLA- | ALTIQLIQNHFVDEY | 3 | IQLIQNHFV | 0.873 | Sequence | 0.078823 | 0.67 | <=SB |
| | DQA10102- | | | | | | | | |
| | DQB10501 | | | | | | | | |
| 17 | HLA- | SALTIQLIQNHFVDEY | 4 | IQLIQNHFV | 0.867 | Sequence | 0.072015 | 0.86 | <=SB |
| | DQA10102- | | | | | | | | |
| | DQB10501 | | | | | | | | |
| 18 | HLA- | ALTIQLIQNHFVDE | 3 | IQLIQNHFV | 0.9 | Sequence | 0.086239 | 0.78 | <=SB |
| | DQA10103- | | | | | | | | |
| | DQB10501 | | | | | | | | |
| 18 | DRB1_1201 | ALTIQLIQNHFVD | 3 | IQLIQNHFV | 0.933 | Sequence | 0.596092 | 0.79 | <=SB |
| 19 | DRB1_1201 | LTIQLIQNHFVDE | 2 | IQLIQNHFV | 0.96 | Sequence | 0.571775 | 0.92 | <=SB |
| 17 | DRB1_1201 | SALTIQLIQNHFVD | 4 | IQLIQNHFV | 0.867 | Sequence | 0.582932 | 0.86 | <=SB |
| 18 | DRB1_1201 | ALTIQLIQNHFVDE | 3 | IQLIQNHFV | 0.953 | Sequence | 0.6845 | 0.44 | <=SB |
| 16 | DRB1_1201 | KSALTIQLIQNHFVD | 5 | IQLIQNHFV | 0.833 | Sequence | 0.568198 | 0.94 | <=SB |
| 17 | DRB1_1201 | SALTIQLIQNHFVDE | 4 | IQLIQNHFV | 0.873 | Sequence | 0.694216 | 0.41 | <=SB |
| 18 | DRB1_1201 | ALTIQLIQNHFVDEY | 3 | IQLIQNHFV | 0.967 | Sequence | 0.6916 | 0.42 | <=SB |
| 16 | DRB1_1201 | KSALTIQLIQNHFVDE | 5 | IQLIQNHFV | 0.873 | Sequence | 0.605735 | 0.75 | <=SB |
| 17 | DRB1_1201 | SALTIQLIQNHFVDEY | 4 | IQLIQNHFV | 0.92 | Sequence | 0.621696 | 0.67 | <=SB |
| 16 | DRB1_1201 | KSALTIQLIQNHFVDE | 5 | IQLIQNHFV | 0.9 | Sequence | 0.578072 | 0.88 | <=SB |
| | | Y | | | | | | | |
| 18 | DRB1_1501 | ALTIQLIQNHFVD | 3 | IQLIQNHFV | 1 | Sequence | 0.917192 | 0.2 | <=SB |
| 19 | DRB1_1501 | LTIQLIQNHFVDE | 2 | IQLIQNHFV | 1 | Sequence | 0.906477 | 0.23 | <=SB |
| 20 | DRB1_1501 | TIQLIQNHFVDEY | 1 | IQLIQNHFV | 1 | Sequence | 0.738752 | 0.69 | <=SB |
| 17 | DRB1_1501 | SALTIQLIQNHFVD | 4 | IQLIQNHFV | 1 | Sequence | 0.885914 | 0.29 | <=SB |
| 18 | DRB1_1501 | ALTIQLIQNHFVDE | 3 | IQLIQNHFV | 1 | Sequence | 0.944924 | 0.12 | <=SB |
| 19 | DRB1_1501 | LTIQLIQNHFVDEY | 2 | IQLIQNHFV | 1 | Sequence | 0.885485 | 0.29 | <=SB |
| 16 | DRB1_1501 | KSALTIQLIQNHFVD | 5 | IQLIQNHFV | 1 | Sequence | 0.82509 | 0.45 | <=SB |
| 17 | DRB1_1501 | SALTIQLIQNHFVDE | 4 | IQLIQNHFV | 1 | Sequence | 0.924267 | 0.17 | <=SB |
| 18 | DRB1_1501 | ALTIQLIQNHFVDEY | 3 | IQLIQNHFV | 1 | Sequence | 0.930517 | 0.16 | <=SB |
| 15 | DRB1_1501 | GKSALTIQLIQNHFVD | 6 | IQLIQNHFV | 1 | Sequence | 0.732559 | 0.71 | <=SB |
| 16 | DRB1_1501 | KSALTIQLIQNHFVDE | 5 | IQLIQNHFV | 1 | Sequence | 0.900479 | 0.24 | <=SB |
| 17 | DRB1_1501 | SALTIQLIQNHFVDEY | 4 | IQLIQNHFV | 1 | Sequence | 0.92063 | 0.19 | <=SB |
| 15 | DRB1_1501 | GKSALTIQLIQNHFVD | 6 | IQLIQNHFV | 1 | Sequence | 0.841036 | 0.41 | <=SB |
| | | E | | | | | | | |
| 16 | DRB1_1501 | KSALTIQLIQNHFVDE | 5 | IQLIQNHFV | 1 | Sequence | 0.884594 | 0.3 | <=SB |
| | | Y | | | | | | | |
| 14 | DRB1_1501 | VGKSALTIQLIQNHFV | 7 | IQLIQNHFV | 1 | Sequence | 0.744596 | 0.68 | <=SB |
| | | DE | | | | | | | |
| 15 | DRB1_1501 | GKSALTIQLIQNHFVD | 6 | IQLIQNHFV | 1 | Sequence | 0.816869 | 0.47 | <=SB |
| | | EY | | | | | | | |
| 14 | DRB1_1501 | VGKSALTIQLIQNHFV | 7 | IQLIQNHFV | 1 | Sequence | 0.710297 | 0.77 | <=SB |
| | | DEY | | | | | | | |
| 18 | DRB1_1503 | ALTIQLIQNHFVD | 3 | IQLIQNHFV | 1 | Sequence | 0.62164 | 0.52 | <=SB |
| 19 | DRB1_1503 | LTIQLIQNHFVDE | 2 | IQLIQNHFV | 1 | Sequence | 0.616986 | 0.53 | <=SB |
| 17 | DRB1_1503 | SALTIQLIQNHFVD | 4 | IQLIQNHFV | 0.993 | Sequence | 0.560524 | 0.71 | <=SB |
| 18 | DRB1_1503 | ALTIQLIQNHFVDE | 3 | IQLIQNHFV | 1 | Sequence | 0.707464 | 0.32 | <=SB |
| 19 | DRB1_1503 | LTIQLIQNHFVDEY | 2 | IQLIQNHFV | 1 | Sequence | 0.575767 | 0.66 | <=SB |
| 16 | DRB1_1503 | KSALTIQLIQNHFVD | 5 | IQLIQNHFV | 0.987 | Sequence | 0.517596 | 0.87 | <=SB |
| 17 | DRB1_1503 | SALTIQLIQNHFVDE | 4 | IQLIQNHFV | 1 | Sequence | 0.701898 | 0.33 | <=SB |
| 18 | DRB1_1503 | ALTIQLIQNHFVDEY | 3 | IQLIQNHFV | 1 | Sequence | 0.712932 | 0.3 | <=SB |
| 16 | DRB1_1503 | KSALTIQLIQNHFVDE | 5 | IQLIQNHFV | 0.993 | Sequence | 0.599043 | 0.59 | <=SB |
| 17 | DRB1_1503 | SALTIQLIQNHFVDEY | 4 | IQLIQNHFV | 1 | Sequence | 0.6392 | 0.47 | <=SB |
| 15 | DRB1_1503 | GKSALTIQLIQNHFVD | 6 | IQLIQNHFV | 0.993 | Sequence | 0.508774 | 0.91 | <=SB |
| | | E | | | | | | | |
| 16 | DRB1_1503 | KSALTIQLIQNHFVDE | 5 | IQLIQNHFV | 1 | Sequence | 0.574609 | 0.67 | <=SB |
| | | Y | | | | | | | |
| 18 | DRB1_1601 | ALTIQLIQNHFVDE | 3 | IQLIQNHFV | 1 | Sequence | 0.793123 | 0.48 | <=SB |
| 17 | DRB1_1601 | SALTIQLIQNHFVDE | 4 | IQLIQNHFV | 1 | Sequence | 0.776869 | 0.55 | <=SB |
| 18 | DRB1_1601 | ALTIQLIQNHFVDEY | 3 | IQLIQNHFV | 1 | Sequence | 0.799323 | 0.45 | <=SB |
| 16 | DRB1_1601 | KSALTIQLIQNHFVDE | 5 | IQLIQNHFV | 1 | Sequence | 0.692628 | 0.97 | <=SB |
| 17 | DRB1_1601 | SALTIQLIQNHFVDEY | 4 | IQLIQNHFV | 1 | Sequence | 0.75063 | 0.67 | <=SB |
| 16 | DRB1_1601 | KSALTIQLIQNHFVDE | 5 | IQLIQNHFV | 1 | Sequence | 0.688804 | 0.99 | <=SB |
| | | Y | | | | | | | |
| 16 | DRB4_0101 | KSALTIQLIQNHFVD | 3 | LTIQLIQNH | 0.953 | Sequence | 0.356733 | 0.8 | <=SB |
| 16 | DRB4_0103 | KSALTIQLIQNHFVD | 3 | LTIQLIQNH | 0.953 | Sequence | 0.356733 | 0.8 | <=SB |

TABLE 5
Subject demographics of patient PBMCs used in EDGE-II development and validation

| | | | | | | | KRAS |
|---|---|---|---|---|---|---|---|
| | | | | | | | G12C |
| | | | | | | KRAS | post- |
| | | | | | | G12C | IVS |
| | Commercial | Collection | Sample | | | ELIS | ELIS |
| Donor ID | Source | type* | type | Sex | Age | pot | pot |
| AC10002 | AllCells | Leukopak | Prospective | Male | 53 | negative | positive |
| | | | collection | | | | |
| AC13990 | AllCells | Leukopak | Prospective | Female | 33 | positive | positive |
| | | | collection | | | | |
| AC16443 | AllCells | Leukopak | Prospective | Male | 31 | marginal | positive |
| | | | collection | | | | |
| SE0386 | StemExpress | Leukopak | Prospective | Female | 44 | positive | positive |
| | | | collection | | | | |
| SE0659 | StemExpress | Leukopak | Prospective | Male | 57 | positive | positive |
| | | | collection | | | | |
| ST0118 | STEMCELL | Leukopak | Prospective | Male | 56 | positive | positive |
| | | | collection | | | | |
| K562 | ATCC | n/a | Cell line | n/a | n/a | n/a | n/a |
| G05-002-0122 | n/a | EDTA | Clinical trial | Female | 83 | positive | positive |
| | | | sample | | | | |
| KAS116 | ATCC | n/a | Cell line | n/a | n/a | n/a | n/a |

| Donor ID | HLA-A | HLA-B | HLA-C | HLA-DRB1 | HLA-DRB3/4/5 |
|---|---|---|---|---|---|
| AC10002 | 02:01/ | 44:03/ | 05:01/ | 07:01:01/ | DRB3*02:02:01/ |
| | 29:02 | 44:02 | 16:01 | 13:01:01 | DRB4*01:01:01 |
| AC13990 | 02:01/ | 40:01/ | 03:04/ | 01:01:01/ | n/a/ |
| | 31:01 | 49:01 | 07:01 | 15:01:01 | DRB5*01:01:01 |
| AC16443 | 03:01/ | 07:02/ | 07:02/ | 01:01:01/ | —/ |
| | 03:01 | 44:02 | 07:04 | 15:01:01 | DRB5*01:01:01 |
| SE0386 | 02:01/ | 44:03/ | 02:02/ | 07:01:01/ | DRB3*02:02:01/ |
| | 31:01 | 44:05 | 16:01 | 11:04:01 | DRB4*01:01:01 |
| SE0659 | 02:05/ | 44:03/ | 04:01/ | 07:01:01/ | DRB4*01:01:01/ |
| | 03:01 | 50:01 | 06:02 | 15:03:01 | DRB5*01:01:01 |
| ST0118 | 02:01/ | 14:02/ | 01:02/ | 01:01:01/ | n/a |
| | 68:02 | 27:05 | 08:02 | 01:02:01 | |
| K562 | 11:01/ | 18:01/ | 03:04/ | 03:01:01/ | DRB3*02:02:01/ |
| | 31:01 | 40:01 | 05:01 | 03:31 | DRB4*01:03:01 |
| G05-002-0122 | 02:01/ | 35:01/ | 04:01/ | 01:01:01/ | DRB3*02:02:01/ |
| | 23:01 | 44:02 | 05:01 | 12:01:01 | n/a |
| KAS116 | 24:02/ | 51:01/ | 12:03/ | 01:01:01/ | DRB3-03:01:01/ |
| | 24:02 | 51:01 | 12:03 | 01:01:01 | n/a |

| | | | | | | | KRAS | | | | |
|---|---|---|---|---|---|---|---|---|---|---|---|
| | | | | | | | G12C | | | | |
| | | | | | | KRAS | post- | | | | |
| | | | | | | G12C | IVS | | | | |
| | Commercial | Collection | Sample | | | ELIS | ELIS | | | | |
| Donor ID | Source | type* | type | Sex | Age | pot | pot | HLA-DQA | HLA-DQB | HLA-DPA | HLA-DPB |
| AC10002 | AllCells | Leukopak | Prospective | Male | 53 | negative | positive | 01:03:01/ | 02:02:01/ | 02:01:01/ | 11:01:01/ |
| | | | collection | | | | | 02:01:01 | 06:03:01 | 02:01:01 | 14:01:01 |
| AC13990 | AllCells | Leukopak | Prospective | Female | 33 | positive | positive | 01:02:01/ | 05:04/ | 01:03:01/ | 04:01:01/ |
| | | | collection | | | | | 01:02:01 | 06:02:01 | 01:03:01 | 104:01:01 |
| AC16443 | AllCells | Leukopak | Prospective | Male | 31 | marginal | positive | 01:01:01/ | 05:01:01 | 01:03:01/ | 04:01:01/ |
| | | | collection | | | | | 01:02:01 | 06:02:01 | 01:03:01 | 04:01:01 |
| SE0386 | StemExpress | Leukopak | Prospective | Female | 44 | positive | positive | 02:01:01/ | 02:01:01/ | 02:01:01/ | 10:01:01/ |
| | | | collection | | | | | 05:05:01 | 03:01:01 | 02:01:01 | 11:01:01 |
| SE0659 | StemExpress | Leukopak | Prospective | Male | 57 | positive | positive | 01:02:01/ | 02:01:01/ | 01:03:01/ | 03:01:01/ |
| | | | collection | | | | | 02:01:01 | 06:02:01 | 03:01:01 | 04:02:01 |
| ST0118 | STEMCELL | Leukopak | Prospective | Male | 56 | positive | positive | 01:01:01/ | 05:01:01/ | 01:03:01/ | 04:01:01/ |
| | | | collection | | | | | 01:01:02 | 05:01:01 | 01:03:01 | 04:02:01 |
| K562 | ATCC | n/a | Cell line | n/a | n/a | n/a | n/a | 03:01:01/ | 02:01:01/ | 01:03:01/ | 04:01:01/ |
| | | | | | | | | 05:01:01 | 03:02:01 | 01:03:01 | 04:02:01 |
| G05-002-0122 | n/a | EDTA | Clinical trial | Female | 83 | positive | positive | 01:01:01/ | 03:01:01/ | 01:03:01/ | 04:02:01/ |
| | | | sample | | | | | 05:05:01 | 05:01:01 | 01:03:01 | 04:02:01 |
| KAS116 | ATCC | n/a | Cell line | n/a | n/a | n/a | n/a | 01:01:01/ | 05:01:01/ | 02:01:01/ | 13:01:01/ |
| | | | | | | | | 01:01:01 | 05:01:01 | 02:01:01 | 13:01:01 |

*Leukopak collections included class I and class II HLA typing; RBC Whole blood collections had class I HLA typing performed retrospecitvely (UCLA); Patient sample and cell lines HLA-typed in-house

TABLE 6
List of custom-made, recombinant, lyphilized peptides specific for G12C

| Peptide number | Amino Acid Sequence | Included in Peptide Pool(s) |
|---|---|---|
| Peptide_07 | ACGVGKSA | KRAS G12C |
| Peptide_16 | ACGVGKSAL | KRAS G12C |
| Peptide_102 | ACGVGKSALT | KRAS G12C/Class II Pool 1 |
| Peptide_37 | ACGVGKSALTI | KRAS G12C/Class II Pool 1 |
| Peptide_104class2 | ACGVGKSALTIQ | Class II Pool 1 |
| Peptide_088class2 | ACGVGKSALTIQL | Class II Pool 1 |
| Peptide_065class2 | ACGVGKSALTIQLI | Class II Pool 1 |
| Peptide_048class2 | ACGVGKSALTIQLIQ | Class II Pool 1 |
| Peptide_08 | CGVGKSAL | KRAS G12C |
| Peptide_17 | CGVGKSALT | KRAS G12C |
| Peptide_27 | CGVGKSALTI | KRAS G12C/Class II Pool 1 |
| Peptide_38 | CGVGKSALTIQ | KRAS G12C/Class II Pool 1 |
| Peptide_018class2 | CGVGKSALTIQL | Class II Pool 1 |
| Peptide_024class2 | CGVGKSALTIQLI | Class II Pool 1 |
| Peptide_057class2 | CGVGKSALTIQLIQ | Class II Pool 1 |
| Peptide_18 | EYKLVVVGAC | KRAS G12C/Class II Pool 1 |
| Peptide_29 | EYKLVVVGACG | KRAS G12C/Class II Pool 1 |
| Peptide_081class2 | EYKLVVVGACGV | Class II Pool 1 |
| Peptide_055class2 | EYKLVVVGACGVG | Class II Pool 1 |
| Peptide_109class2 | EYKLVVVGACGVGK | Class II Pool 1 |
| Peptide_085class2 | EYKLVVVGACGVGKS | Class II Pool 1 |
| Peptide_064class2 | EYKLVVVGACGVGKSA | Class II Pool 1 |
| Peptide_106class2 | EYKLVVVGACGVGKSAL | Class II Pool 1 |
| Peptide_100class2 | EYKLVVVGACGVGKSALT | Class II Pool 1 |
| Peptide_035class2 | EYKLVVVGACGVGKSALT | Class II Pool 1 |
| | I | |
| Peptide_058class2 | EYKLVVVGACGVGKSALT | Class II Pool 1 |
| | IQ | |
| Peptide_06 | GACGVGKS | KRAS G12C |
| Peptide_15 | GACGVGKSA | KRAS G12C |
| Peptide_25 | GACGVGKSAL | KRAS G12C/Class II Pool 2 |
| Peptide_36 | GACGVGKSALT | KRAS G12C/Class II Pool 2 |
| Peptide_045class2 | GACGVGKSALTI | Class II Pool 2 |
| Peptide_089class2 | GACGVGKSALTIQ | Class II Pool 2 |
| Peptide_015class2 | GACGVGKSALTIQL | Class II Pool 2 |
| Peptide_099class2 | GACGVGKSALTIQLI | Class II Pool 2 |
| Peptide_116class2 | GACGVGKSALTIQLIQ | Class II Pool 2 |
| Peptide_39 | KLVVVGAC | KRAS G12C |
| Peptide_10 | KLVVVGACG | KRAS G12C |
| Peptide_20 | KLVVVGACGV | KRAS G12C/Class II Pool 2 |
| Peptide_31 | KLVVVGACGVG | KRAS G12C/Class II Pool 2 |
| Peptide_021class2 | KLVVVGACGVGK | Class II Pool 2 |
| Peptide_004class2 | KLVVVGACGVGKS | Class II Pool 2 |
| Peptide_063class2 | KLVVVGACGVGKSA | Class II Pool 2 |
| Peptide_008class2 | KLVVVGACGVGKSAL | Class II Pool 2 |
| Peptide_112class2 | KLVVVGACGVGKSALT | Class II Pool 2 |
| Peptide_066class2 | KLVVVGACGVGKSALTI | Class II Pool 2 |
| Peptide_020class2 | KLVVVGACGVGKSALTIQ | Class II Pool 2 |
| Peptide_028class2 | KLVVVGACGVGKSALTIQ | Class II Pool 2 |
| | L | |
| Peptide_91 | KLVVVGACGVGKSALTIQ | Class II Pool 2 |
| | LI | |
| Peptide_02 | LVVVGACG | KRAS G12C |
| Peptide_41 | LVVVGACGV | KRAS G12C |
| Peptide_21 | LVVVGACGVG | KRAS G12C/Class II Pool 2 |
| Peptide_32 | LVVVGACGVGK | KRAS G12C/Class II Pool 2 |
| Peptide_046class2 | LVVVGACGVGKS | Class II Pool 2 |
| Peptide_036class2 | LVVVGACGVGKSA | Class II Pool 2 |
| Peptide_042class2 | LVVVGACGVGKSAL | Class II Pool 2 |
| Peptide_034class2 | LVVVGACGVGKSALT | Class II Pool 2 |
| Peptide_019class2 | LVVVGACGVGKSALTI | Class II Pool 2 |
| Peptide_009class2 | LVVVGACGVGKSALTIQ | Class II Pool 2 |
| Peptide_113class2 | LVVVGACGVGKSALTIQL | Class II Pool 2 |
| Peptide_016class2 | LVVVGACGVGKSALTIQLI | Class II Pool 2 |
| Peptide_079class2 | LVVVGACGVGKSALTIQLI | Class II Pool 2 |
| | Q | |
| Peptide_098class2 | MTEYKLVVVGAC | Class II Pool 3 |
| Peptide_073class2 | MTEYKLVVVGACG | Class II Pool 3 |
| Peptide_40 | MTEYKLVVVGACGV | Class II Pool 3 |
| Peptide_060class2 | MTEYKLVVVGACGVG | Class II Pool 3 |
| Peptide_010class2 | MTEYKLVVVGACGVGK | Class II Pool 3 |
| Peptide_26 | MTEYKLVVVGACGVGKS | Class II Pool 3 |
| Peptide_037class2 | MTEYKLVVVGACGVGKS | Class II Pool 3 |
| | A | |
| Peptide_11 | MTEYKLVVVGACGVGKS | Class II Pool 3 |
| | AL | |
| Peptide_111class2 | MTEYKLVVVGACGVGKS | Class II Pool 3 |
| | ALT | |
| Peptide_28 | TEYKLVVVGAC | KRAS G12C/Class II Pool 3 |
| Peptide_050class2 | TEYKLVVVGACG | Class II Pool 3 |
| Peptide_105class2 | TEYKLVVVGACGV | Class II Pool 3 |
| Peptide_022class2 | TEYKLVVVGACGVG | Class II Pool 3 |
| Peptide_025class2 | TEYKLVVVGACGVGK | Class II Pool 3 |
| Peptide_030class2 | TEYKLVVVGACGVGKS | Class II Pool 3 |
| Peptide_101class2 | TEYKLVVVGACGVGKSA | Class II Pool 3 |
| Peptide_053class2 | TEYKLVVVGACGVGKSAL | Class II Pool 3 |
| Peptide_107class2 | TEYKLVVVGACGVGKSAL | Class II Pool 3 |
| | T | |
| Peptide_044class2 | TEYKLVVVGACGVGKSAL | Class II Pool 3 |
| | TI | |
| Peptide_05 | VGACGVGK | KRAS G12C |
| Peptide_14 | VGACGVGKS | KRAS G12C |
| Peptide_24 | VGACGVGKSA | KRAS G12C/Class II Pool 3 |
| Peptide_35 | VGACGVGKSAL | KRAS G12C/Class II Pool 3 |
| Peptide_115class2 | VGACGVGKSALT | Class II Pool 3 |
| Peptide_002class2 | VGACGVGKSALTI | Class II Pool 3 |
| Peptide_01 | VGACGVGKSALTIQ | Class II Pool 3 |
| Peptide_052class2 | VGACGVGKSALTIQL | Class II Pool 3 |
| Peptide_071class2 | VGACGVGKSALTIQLI | Class II Pool 3 |
| Peptide_070class2 | VGACGVGKSALTIQLIQ | Class II Pool 3 |
| Peptide_04 | VVGACGVG | KRAS G12C |
| Peptide_13 | VVGACGVGK | KRAS G12C |
| Peptide_23 | VVGACGVGKS | KRAS G12C/Class II Pool 4 |
| Peptide_34 | VVGACGVGKSA | KRAS G12C/Class II Pool 4 |
| Peptide_049class2 | VVGACGVGKSAL | Class II Pool 4 |
| Peptide_077class2 | VVGACGVGKSALT | Class II Pool 4 |
| Peptide_023class2 | VVGACGVGKSALTI | Class II Pool 4 |
| Peptide_000class2 | VVGACGVGKSALTIQ | Class II Pool 4 |
| Peptide_067class2 | VVGACGVGKSALTIQL | Class II Pool 4 |
| Peptide_076class2 | VVGACGVGKSALTIQLI | Class II Pool 4 |
| Peptide_033class2 | VVGACGVGKSALTIQLIQ | Class II Pool 4 |
| Peptide_03 | VVVGACGV | KRAS G12C |
| Peptide_12 | VVVGACGVG | KRAS G12C |
| Peptide_22 | VVVGACGVGK | KRAS G12C/Class II Pool 4 |
| Peptide_33 | VVVGACGVGKS | KRAS G12C/Class II Pool 4 |
| Peptide_017class2 | VVVGACGVGKSA | Class II Pool 4 |
| Peptide_069class2 | VVVGACGVGKSAL | Class II Pool 4 |
| Peptide_006class2 | VVVGACGVGKSALT | Class II Pool 4 |
| Peptide_103class2 | VVVGACGVGKSALTI | Class II Pool 4 |
| Peptide_080class2 | VVVGACGVGKSALTIQ | Class II Pool 4 |
| Peptide_038class2 | VVVGACGVGKSALTIQL | Class II Pool 4 |
| Peptide_110class2 | VVVGACGVGKSALTIQLI | Class II Pool 4 |
| Peptide_032class2 | VVVGACGVGKSALTIQLI | Class II Pool 4 |
| | Q | |
| Peptide_09 | YKLVVVGAC | KRAS G12C |
| Peptide_19 | YKLVVVGACG | KRAS G12C/Class II Pool 4 |
| Peptide_30 | YKLVVVGACGV | KRAS G12C/Class II Pool 4 |
| Peptide_029class2 | YKLVVVGACGVG | Class II Pool 4 |
| Peptide_003class2 | YKLVVVGACGVGK | Class II Pool 4 |
| Peptide_075class2 | YKLVVVGACGVGKS | Class II Pool 4 |
| Peptide_072class2 | YKLVVVGACGVGKSA | Class II Pool 4 |
| Peptide_114class2 | YKLVVVGACGVGKSAL | Class II Pool 4 |
| Peptide_068class2 | YKLVVVGACGVGKSALT | Class II Pool 4 |
| Peptide_007class2 | YKLVVVGACGVGKSALTI | Class II Pool 4 |
| Peptide_108class2 | YKLVVVGACGVGKSALTI | Class II Pool 4 |
| | Q | |
| Peptide_059class2 | YKLVVVGACGVGKSALTI | Class II Pool 4 |
| | QL | |

TABLE 7
Peptides and associated EDGE scores

| Peptide | EDGE Score |
|---|---|
| KLVVVGACGVGKS | 0.515 |
| VVGACGVGKSALTI | 0.569 |
| LVVVGACGVGKSALTI | 0.698 |
| LVVVGACGVGKSALTIQ | 0.717 |
| YKLVVVGACGVGKSALT | 0.735 |
| YKLVVVGACGVGKSA | 0.771 |
| KLVVVGACGVGKSAL | 0.954 |
| KLVVVGACGVGKSA | 0.973 |
| VVGACGVGKSALT | 0.978 |
| KLVVVGACGVGKSALT | 0.983 |
| VVVGACGVGKSAL | 0.988 |
| VVVGACGVGKSALT | 0.989 |
| LVVVGACGVGKSA | 0.993 |
| LVVVGACGVGKSAL | 0.993 |
| LVVVGACGVGKSALT | 0.996 |

TABLE 8
Single cell clusters and their cell type annotation. The first column indicates the single cell cluster id. The second column
represents the expression score of CD8A and CD8B defined using AddModuleScore( ) function in Seurat. The clusters with CD8
score greater than 0 were defined as CD8 clusters. The third column (CD8/4 annotation) indicates the CD8/CD4 specific annotation
of clusters. The fourth column (Cell type) indicates the cell type annotation assigned to each cluster. The fifth column
(Top CIPR annotations) indicates the top cell type annotation(s) predicted by CIPR package for each cluster. The last column
(Top 5 differentially expressed genes) indicates the top 5 differentially expressed genes in each cluster.

| | CD8 | | | | Top 5 |
|---|---|---|---|---|---|
| | expression | CD8/4 | | Top CIPR | differentially |
| Cluster | score | annotation | Cell type | annotation(s) | expressed genes |
| 0 | −0.280 | CD4 | CD4-Tem | Th17 cells | S100A4, LGALS1, S100A6, CD52, S100A11 |
| 1 | −0.197 | CD4 | CD4-Naïve | Naïve | MAL, CCR7, EEF1B2, SARAF, IL4R |
| 2 | −0.217 | CD4 | CD4-Naïve | Naïve | IL7R, PASK, TCF7, MALAT1, LINC00861 |
| 3 | 0.292 | CD8 | CD8 + Gamma-Delta | Non-Vd2 gd T cells | GNLY, NKG7, CCL5, TYROBP, CST7 |
| 4 | 0.015 | CD8 | CD8 + Gamma-Delta | Non-Vd2 gd T cells | NKG7, CCL5, GZMA, CD74, CST7 |
| 5 | −0.279 | CD4 | CD4-LGMN | Treg/FhT | LGMN, STAT1, TNFRSF4, SRGN, GBP5 |
| 6 | −0.195 | CD4 | CD4-Unknown | Th1/Naïve | IL7R, RPS18, TNFSF13B, RPS12, EEF1B2 |
| 7 | 0.429 | CD8 | CD8-Tem | Effector memory (EM) | GZMK, CCL5, CD8B, LYAR, CD8A |
| 8 | 0.379 | CD8 | CD8-KRT1 | Th1/Naïve | KRT1, CD8B, IL7R, FXYD5, SERP1 |
| 9 | 0.558 | CD8 | CD8-Teff | Terminal effector/EM | CCL4, XCL1, CCL4L2, GZMB, XCL2 |
| 10 | −0.217 | CD4 | CD4-MTRNR2L12 | Th17/Th2 | MTRNR2L12, MTRNR2L8, IL7R, HIST1H1E, TCF7 |
| 11 | −0.238 | CD4 | CD4-Treg | T regulatory cells (Treg) | LTB, FOXP3, TNFRSF4, IL32, CORO1B |
| 12 | −0.146 | CD4 | CD4-Unknown | Th1/Naïve | AC090498.1, RPS29, RPL39, RPS21, RPL32 |
| 13 | 1.014 | CD8 | CD8-Naïve | Naïve | CD8B, RP11-291B21.2, CD8A, CCR7, NELL2 |
| 14 | 0.029 | CD8 | CD8 + NK | NK | FCER1G, TYROBP, GNLY, KLRB1, CTSW |
| 15 | 0.157 | CD8 | CD8-Teff | EM | FABP5, MIR155HG, PKM, TNFRSF9, CRTAM |
| 16 | −0.182 | CD4 | CD4-RPL36A | Naïve/Th1 | AC090498.1, RPS29, RPL36A, RPL41, RPL39 |
| 17 | −0.324 | CD4 | CD4-Tem | EM/Treg | HIST1H4C, TUBA1B, STMN1, TUBB, HMGB2 |
| 18 | −0.265 | CD4 | CD4-Tem | Th17 | KLRB1, C1GALT1, AC092580.4, IL32, CXCR6 |
| 19 | −0.211 | CD4 | CD4-Unknown | Central memory/Th1 | PLCG2, RPS10, AC090498.1, RPL36A, RPS29 |

TABLE 9
Frozen tissue and cell line specimens sources and HLA annotations

| | | | | | | Cell Line | HLA | HLA | HLA |
|---|---|---|---|---|---|---|---|---|---|
| LIMS No | Vendor or Investigator | Location | Tissue Location | Cancer Type | Other | Name | 1 | 2 | 3 |
| A0000036 | Bioserve | Beltsville, MD | Colon | COAD | | | | | DR |
| A0000051 | Bioserve | Beltsville, MD | Colon | COAD | | | | | DR |
| A0000056 | Bioserve | Beltsville, MD | Lung | LUAD | | | | | DR |
| A0000062 | Bioserve | Beltsville, MD | Colon | COAD | | | | | DR |
| A0000196 | ICON Plc | Dublin, IRL | Lung | NSCLC | | | | | DR |
| A0000199 | ICON Plc | Dublin, IRL | Lung | NSCLC | | | | | DR |
| A0000204 | iSpecimen | Lexington, MA | Ovarian | OV | | | | | DR |
| A0000213 | iSpecimen | Lexington, MA | Ovarian | OV | | | | | DR |
| A0000238 | Asterand Bioscience | Detroit, MI | Ovarian | OV | | | | | DR |
| A0000240 | Asterand Bioscience | Detroit, MI | Ovarian | OV | | | | | DR |
| A0000251 | ICON Plc | Dublin, IRL | Lung | NSCLC | | | | | DR |
| A0000252 | ICON Plc | Dublin, IRL | Lung | NSCLC | | | | | DR |
| A0000284 | ICON Plc | Dublin, IRL | Lung | NSCLC | | | | | DR |
| A0000285 | ICON Plc | Dublin, IRL | Lung | NSCLC | | | | | DR |
| A0000287 | ICON Plc | Dublin, IRL | Lung | NSCLC | | | | | DR |
| A0000289 | ICON Plc | Dublin, IRL | Lung | NSCLC | | | | | DR |
| A0000290 | ICON Plc | Dublin, IRL | Lung | NSCLC | | | | | DR |
| A0000293 | ICON Plc | Dublin, IRL | Lung | NSCLC | | | | | DR |
| A0000294 | ICON Plc | Dublin, IRL | Lung | NSCLC | | | | | DR |
| A0000295 | ICON Plc | Dublin, IRL | Lung | NSCLC | | | | | DR |
| A0000296 | ICON Plc | Dublin, IRL | Lung | NSCLC | | | | | DR |
| A0000297 | ICON Plc | Dublin, IRL | Lung | NSCLC | | | | | DR |
| A0000317 | ICON Plc | Dublin, IRL | Lung | NSCLC | | | | | DR |
| A0000318 | ICON Plc | Dublin, IRL | Lung | NSCLC | | | | | DR |
| A0000319 | ICON Plc | Dublin, IRL | Lung | NSCLC | | | | | DR |
| A0000345 | Proteogenex | Culver City, CA | Ovarian | OV | | | | | DR |
| A0000346 | Proteogenex | Culver City, CA | Ovarian | Normal | | | | | DR |
| A0000348 | Proteogenex | Culver City, CA | Colon | COAD | | | | | DR |
| A0000349 | Proteogenex | Culver City, CA | Lung | Normal | | | | | DR |
| A0000350 | Proteogenex | Culver City, CA | Lung | LUAD | | | | | DR |
| A0000352 | Proteogenex | Culver City, CA | Lung | Normal | | | | | DR |
| A0000353 | Proteogenex | Culver City, CA | Lung | LUAD | | | | | DR |
| A0000359 | Brigham & | Boston, MA | Lung | LUAD | | | | | DR |
| | Women's Hospital | | | | | | | | |
| A0000361 | Brigham & | Boston, MA | Lung | LUAD | | | | | DR |
| | Women's Hospital | | | | | | | | |
| A0000363 | Brigham & | Boston, MA | Lung | NSCLC | | | | | DR |
| | Women's Hospital | | | | | | | | |
| A0000367 | Brigham & | Boston, MA | Lung | LUSC | | | | | DR |
| | Women's Hospital | | | | | | | | |
| A0000369 | Brigham & | Boston, MA | Lung | LUAD | | | | | DR |
| | Women's Hospital | | | | | | | | |
| A0000371 | Brigham & | Boston, MA | Lung | LUAD | | | | | DR |
| | Women's Hospital | | | | | | | | |
| A0000373 | Brigham & | Boston, MA | Lung | LUAD | | | | | DR |
| | Women's Hospital | | | | | | | | |
| A0000375 | Brigham & | Boston, MA | Lung | LUSC | | | | | DR |
| | Women's Hospital | | | | | | | | |
| A0000379 | Brigham & | Boston, MA | Lung | LUAD | | | | | DR |
| | Women's Hospital | | | | | | | | |
| A0000381 | Brigham & | Boston, MA | Lung | LUSC | | | | | DR |
| | Women's Hospital | | | | | | | | |
| A0000383 | Brigham & | Boston, MA | Lung | LUAD | | | | | DR |
| | Women's Hospital | | | | | | | | |
| A0000385 | Brigham & | Boston, MA | Lung | LUAD | | | | | DR |
| | Women's Hospital | | | | | | | | |
| A0000387 | Brigham & | Boston, MA | Lung | LUAD | | | | | DR |
| | Women's Hospital | | | | | | | | |
| A0000389 | Brigham & | Boston, MA | Lung | LUAD | | | | | DR |
| | Women's Hospital | | | | | | | | |
| A0000391 | Brigham & | Boston, MA | Lung | LUAD | | | | | DR |
| | Women's Hospital | | | | | | | | |
| A0000393 | Brigham & | Boston, MA | Lung | LUAD | | | | | DR |
| | Women's Hospital | | | | | | | | |
| A0000395 | Brigham & | Boston, MA | Lung | LUAD | | | | | DR |
| | Women's Hospital | | | | | | | | |
| A0000460 | ICON Plc | Dublin, IRL | Lung | NSCLC | | | | | DR |
| A0000461 | ICON Plc | Dublin, IRL | Lung | NSCLC | | | | | DR |
| A0000462 | ICON Plc | Dublin, IRL | Lung | NSCLC | | | | | DR |
| A0000463 | ICON Plc | Dublin, IRL | Lung | NSCLC | | | | | DR |
| A0000464 | ICON Plc | Dublin, IRL | Lung | NSCLC | | | | | DR |
| A0000465 | ICON Plc | Dublin, IRL | Lung | NSCLC | | | | | DR |
| A0000468 | ICON Plc | Dublin, IRL | Lung | NSCLC | | | | | DR |
| A0000471 | ICON Plc | Dublin, IRL | Lung | NSCLC | | | | | DR |
| A0000472 | ICON Plc | Dublin, IRL | Lung | NSCLC | | | | | DR |
| A0000474 | ICON Plc | Dublin, IRL | Lung | NSCLC | | | | | DR |
| A0000479 | ICON Plc | Dublin, IRL | Lung | NSCLC | | | | | DR |
| A0000482 | ICON Plc | Dublin, IRL | Lung | NSCLC | | | | | DR |
| A0000496 | ICON Plc | Dublin, IRL | Lung | NSCLC | | | | | DR |
| A0000499 | ICON Plc | Dublin, IRL | Lung | NSCLC | | | | | DR |
| A0000504 | ICON Plc | Dublin, IRL | Lung | NSCLC | | | | | DR |
| A0000508 | Proteogenex | Culver City, CA | Lymphatic | HL | | | | | DR |
| A0000509 | Proteogenex | Culver City, CA | Lymphatic | HL | | | | | DR |
| A0000512 | Proteogenex | Culver City, CA | Lymphatic | HL | | | | | DR |
| A0000515 | Proteogenex | Culver City, CA | Lymphatic | HL | | | | | DR |
| A0000517 | Proteogenex | Culver City, CA | Lymphatic | HL | | | | | DR |
| A0000519 | Proteogenex | Culver City, CA | Lymph Nodes | HL | | | | | DR |
| A0000527 | Proteogenex | Culver City, CA | Lymph Nodes | HL | | | | | DR |
| A0000529 | iSpecimen | Lexington, MA | Lymph Nodes | Lymphoma | | | | | DR |
| A0000531 | iSpecimen | Lexington, MA | Lymph Nodes | Lymphoma | | | | | DR |
| A0000532 | iSpecimen | Lexington, MA | Lymph Nodes | Lymphoma | | | | | DR |
| A0000534 | iSpecimen | Lexington, MA | Lymph Nodes | Lymphoma | | | | | DR |
| A0000536 | iSpecimen | Lexington, MA | Lymph Nodes | Lymphoma | | | | | DR |
| A0000537 | iSpecimen | Lexington, MA | Lymph Nodes | Lymphoma | | | | | DR |
| A0000603 | Proteogenex | Culver City, CA | Gastroesophageal | Normal | | | | | DR |
| | | | Junction | | | | | | |
| A0000604 | Proteogenex | Culver City, CA | Gastroesophageal | Gej - | | | | | DR |
| | | | Junction | Adenocarcinoma | | | | | |
| A0000621 | Proteogenex | Culver City, CA | Esophagus | Esophagus- | | | | | DR |
| | | | | Normal | | | | | |
| A0000622 | Proteogenex | Culver City, CA | Esophagus | Esophagus - | | | | | DR |
| | | | | Squamous | | | | | |
| A0000631 | Proteogenex | Culver City, CA | Head And Neck | Normal | | | | | DR |
| A0000632 | Proteogenex | Culver City, CA | Head And Neck | HNSC | | | | | DR |
| A0000637 | Proteogenex | Culver City, CA | Gastroesophageal | Gej-Normal | | | | | DR |
| | | | Junction | | | | | | |
| A0000638 | Proteogenex | Culver City, CA | Gastroesophageal | Gej - | | | | | DR |
| | | | Junction | Andenocarcinoma | | | | | |
| A0000641 | Proteogenex | Culver City, CA | Gastric | Gastric- | | | | | DR |
| | | | | Normal | | | | | |
| A0000642 | Proteogenex | Culver City, CA | Gastric | Gastric- | | | | | DR |
| | | | | Adenocarcinoma | | | | | |
| A0000643 | Proteogenex | Culver City, CA | Gastric | Gastric- | | | | | DR |
| | | | | Normal | | | | | |
| A0000644 | Proteogenex | Culver City, CA | Gastric | Gastric- | | | | | DR |
| | | | | Adenocarcinoma | | | | | |
| A0000645 | Proteogenex | Culver City, CA | Gastroesophageal | Gej - | | | | | DR |
| | | | Junction | Andenocarcinoma | | | | | |
| A0000646 | Proteogenex | Culver City, CA | Gastroesophageal | Gej-Normal | | | | | DR |
| | | | Junction | | | | | | |
| A0000649 | Proteogenex | Culver City, CA | Gastric | Gastric- | | | | | DR |
| | | | | Normal | | | | | |
| A0000653 | Proteogenex | Culver City, CA | Gastric | Gastric- | | | | | DR |
| | | | | Normal | | | | | |
| A0000654 | Proteogenex | Culver City, CA | Gastric | Gastric- | | | | | DR |
| | | | | Adenocarcinoma | | | | | |
| A0000658 | Proteogenex | Culver City, CA | Skin | Melanoma | | | | | DR |
| A0000674 | ICON Plc | Dublin, IRL | Lung | NSCLC | | | | | DR |
| A0000676 | ICON Plc | Dublin, IRL | Lung | NSCLC | | | | | DR |
| A0000682 | ICON Plc | Dublin, IRL | Lung | NSCLC | | | | | DR |
| A0000686 | ICON Plc | Dublin, IRL | Lung | NSCLC | | | | | DR |
| A0000688 | ICON Plc | Dublin, IRL | Lung | NSCLC | | | | | DR |
| A0000690 | ICON Plc | Dublin, IRL | Lung | NSCLC | | | | | DR |
| A0000692 | ICON Plc | Dublin, IRL | Lung | NSCLC | | | | | DR |
| A0000704 | ICON Plc | Dublin, IRL | Lung | NSCLC | | | | | DR |
| A0000711 | Brigham & | Boston, MA | Lung | LUSC | | | | | DR |
| | Women's Hospital | | | | | | | | |
| A0000713 | Brigham & | Boston, MA | Lung | LUSC | | | | | DR |
| | Women's Hospital | | | | | | | | |
| A0000717 | Brigham & | Boston, MA | Lung | LUSC | | | | | DR |
| | Women's Hospital | | | | | | | | |
| A0000723 | Brigham & | Boston, MA | Lung | LUAD | | | | | DR |
| | Women's Hospital | | | | | | | | |
| A0000725 | Brigham & | Boston, MA | Lung | LUSC | | | | | DR |
| | Women's Hospital | | | | | | | | |
| A0000727 | Brigham & | Boston, MA | Lung | LUSC | | | | | DR |
| | Women's Hospital | | | | | | | | |
| A0000729 | Brigham & | Boston, MA | Lung | LUSC | | | | | DR |
| | Women's Hospital | | | | | | | | |
| A0000731 | Brigham & | Boston, MA | Lung | LUSC | | | | | DR |
| | Women's Hospital | | | | | | | | |
| A0000735 | Brigham & | Boston, MA | Lung | LUAD | | | | | DR |
| | Women's Hospital | | | | | | | | |
| A0000737 | Brigham & | Boston, MA | Lung | LUAD | | | | | DR |
| | Women's Hospital | | | | | | | | |
| A0000739 | Brigham & | Boston, MA | Lung | LUAD | | | | | DR |
| | Women's Hospital | | | | | | | | |
| A0000741 | Brigham & | Boston, MA | Lung | LUAD | | | | | DR |
| | Women's Hospital | | | | | | | | |
| A0000743 | Brigham & | Boston, MA | Lung | LUAD | | | | | DR |
| | Women's Hospital | | | | | | | | |
| A0000745 | Brigham & | Boston, MA | Lung | LUSC | | | | | DR |
| | Women's Hospital | | | | | | | | |
| A0000747 | Brigham & | Boston, MA | Lung | LUSC | | | | | DR |
| | Women's Hospital | | | | | | | | |
| A0000785 | ICON Plc | Dublin, IRL | Lung | NSCLC | | | | | DR |
| A0000787 | ICON Plc | Dublin, IRL | Lung | NSCLC | | | | | DR |
| A0000804 | ICON Plc | Dublin, IRL | Lung | NSCLC | | | | | DR |
| A0000806 | ICON Plc | Dublin, IRL | Lung | NSCLC | | | | | DR |
| A0000827 | ICON Plc | Dublin, IRL | Lung | NSCLC | | | | | DR |
| A0000829 | ICON Plc | Dublin, IRL | Lung | NSCLC | | | | | DR |
| A0000855 | ICON Plc | Dublin, IRL | Lung | NSCLC | | | | | DR |
| A0000856 | ICON Plc | Dublin, IRL | Lung | NSCLC | | | | | DR |
| A0000892 | ICON Plc | Dublin, IRL | Lung | NSCLC | | | | | DR |
| A0001093 | ICON Plc | Dublin, IRL | Lung | NSCLC | | | | | DR |
| A0001106 | ICON Plc | Dublin, IRL | Lung | NSCLC | | | | | DR |
| A0001107 | ICON Plc | Dublin, IRL | Lung | NSCLC | | | | | DR |
| A0001112 | ICON Plc | Dublin, IRL | Lung | NSCLC | | | | | DR |
| A0001116 | ICON Plc | Dublin, IRL | Lung | NSCLC | | | | | DR |
| A0001137 | iSpecimen | Lexington, MA | Kidney | KIRC | | | | | DR |
| A0001140 | iSpecimen | Lexington, MA | Kidney | KIRC | | | | | DR |
| A0001141 | iSpecimen | Lexington, MA | Kidney | KIRC | | | | | DR |
| A0001143 | iSpecimen | Lexington, MA | Kidney | KIRC | | | | | DR |
| A0001144 | iSpecimen | Lexington, MA | Kidney | KIRC | | | | | DR |
| A0001151 | ICON Plc | Dublin, IRL | Lung | NSCLC | | | | | DR |
| A0001153 | ICON Plc | Dublin, IRL | Lung | NSCLC | | | | | DR |
| A0001158 | ICON Plc | Dublin, IRL | Lung | NSCLC | | | | | DR |
| A0001173 | ICON Plc | Dublin, IRL | Lung | NSCLC | | | | | DR |
| A0001175 | ICON Plc | Dublin, IRL | Lung | NSCLC | | | | | DR |
| A0001183 | ICON Plc | Dublin, IRL | Lung | NSCLC | | | | | DR |
| A0001193 | ICON Plc | Dublin, IRL | Lung | NSCLC | | | | | DR |
| A0001195 | ICON Plc | Dublin, IRL | Lung | NSCLC | | | | | DR |
| A0001199 | ICON Plc | Dublin, IRL | Lung | NSCLC | | | | | DR |
| A0001201 | ICON Plc | Dublin, IRL | Lung | NSCLC | | | | | DR |
| A0001203 | ICON Plc | Dublin, IRL | Lung | NSCLC | | | | | DR |
| A0001209 | ICON Plc | Dublin, IRL | Lung | NSCLC | | | | | DR |
| A0001211 | ICON Plc | Dublin, IRL | Lung | NSCLC | | | | | DR |
| A0001215 | ICON Plc | Dublin, IRL | Lung | NSCLC | | | | | DR |
| A0001218 | ICON Plc | Dublin, IRL | Lung | NSCLC | | | | | DR |
| A0001220 | ICON Plc | Dublin, IRL | Lung | NSCLC | | | | | DR |
| A0001282 | iSpecimen | Lexington, MA | Ovary | OV | | | | | DR |
| A0001284 | iSpecimen | Lexington, MA | Ovary | OV | | | | | DR |
| A0001285 | iSpecimen | Lexington, MA | Ovary | OV | | | | | DR |
| A0001311 | ICON Plc | Dublin, IRL | Lung | NSCLC | | | | | DR |
| A0001313 | ICON Plc | Dublin, IRL | Lung | NSCLC | | | | | DR |
| A0001315 | ICON Plc | Dublin, IRL | Lung | NSCLC | | | | | DR |
| A0001371 | Hôpital Marie | Le Plessis- | Lung | NSCLC | | | | | DR |
| | Lannelongue | Robinson, FR | | | | | | | |
| A0001375 | Hôpital Marie | Le Plessis- | Lung | NSCLC | | | | | DR |
| | Lannelongue | Robinson, FR | | | | | | | |
| A0001386 | ICON Plc | Dublin, IRL | Lung | NSCLC | | | | | DR |
| A0001822 | iSpecimen | Lexington, MA | Lung | LUSC | | | | | DR |
| A0001823 | iSpecimen | Lexington, MA | Colon | COAD | | | | | DR |
| A0001824 | iSpecimen | Lexington, MA | Colon | COAD | | | | | DR |
| A0001826 | iSpecimen | Lexington, MA | Lung | LUAD | | | | | DR |
| A0001896 | Hôpital Marie | Le Plessis- | Lung | LUAD | | | | | DR |
| | Lannelongue | Robinson, FR | | | | | | | |
| A0001907 | Hôpital Marie | Le Plessis- | Lung | LUAD | | | | | DR |
| | Lannelongue | Robinson, FR | | | | | | | |
| A0001908 | Hôpital Marie | Le Plessis- | Lung | LUAD | | | | | DR |
| | Lannelongue | Robinson, FR | | | | | | | |
| A0001934 | Proteogenex | Culver City, CA | Colon | COAD | | | | | DR |
| A0001935 | Proteogenex | Culver City, CA | Colon | COAD | | | | | DR |
| A0001936 | Proteogenex | Culver City, CA | Rectum | READ | | | | | DR |
| A0001939 | Proteogenex | Culver City, CA | Rectum | READ | | | | | DR |
| A0001942 | Proteogenex | Culver City, CA | Rectum | READ | | | | | DR |
| A0001946 | Proteogenex | Culver City, CA | Rectum | READ | | | | | DR |
| A0001947 | Proteogenex | Culver City, CA | Rectum | READ | | | | | DR |
| A0001948 | Proteogenex | Culver City, CA | Rectum | READ | | | | | DR |
| A0002264 | La Jolla Institute for | La Jolla, CA | | EBV | Cell | HARA | | DQ | DR |
| | Immunology | | | transformed | line | | | | |
| | | | | B-cell | | | | | |
| A0002272 | La Jolla Institute for | La Jolla, CA | | EBV | Cell | TEM | DP | DQ | DR |
| | Immunology | | | transformed | line | | | | |
| | | | | B-cell | | | | | |
| A0002274 | La Jolla Institute for | La Jolla, CA | | EBV | Cell | AMAI | DP | DQ | DR |
| | Immunology | | | transformed | line | | | | |
| | | | | B-cell | | | | | |
| A0002275 | La Jolla Institute for | La Jolla, CA | | EBV | Cell | BIN40 | | DQ | DR |
| | Immunology | | | transformed | line | | | | |
| | | | | B-cell | | | | | |
| A0002276 | La Jolla Institute for | La Jolla, CA | | EBV | Cell | DBB | DP | DQ | DR |
| | Immunology | | | transformed | line | | | | |
| | | | | B-cell | | | | | |
| A0002277 | La Jolla Institute for | La Jolla, CA | | EBV | Cell | HID | | | DR |
| | Immunology | | | transformed | line | | | | |
| | | | | B-cell | | | | | |
| A0002278 | La Jolla Institute for | La Jolla, CA | | EBV | Cell | HERLUF | DP | DQ | DR |
| | Immunology | | | transformed | line | | | | |
| | | | | B-cell | | | | | |
| A0002279 | La Jolla Institute for | La Jolla, CA | | EBV | Cell | HARA | | DQ | DR |
| | Immunology | | | transformed | line | | | | |
| | | | | B-cell | | | | | |
| A0002280 | La Jolla Institute for | La Jolla, CA | | EBV | Cell | KAS116 | | DQ | DR |
| | Immunology | | | transformed | line | | | | |
| | | | | B-cell | | | | | |
| A0002285 | La Jolla Institute for | La Jolla, CA | | EBV | Cell | LG2 | | | DR |
| | Immunology | | | transformed | line | | | | |
| | | | | B-cell | | | | | |
| A0002286 | La Jolla Institute for | La Jolla, CA | | EBV | Cell | TISI | | | DR |
| | Immunology | | | transformed | line | | | | |
| | | | | B-cell | | | | | |
| A0002287 | La Jolla Institute for | La Jolla, CA | | EBV | Cell | TEM | | | DR |
| | Immunology | | | transformed | line | | | | |
| | | | | B-cell | | | | | |
| A0002625 | iSpecimen | Lexington, MA | Lung | LUAD | | | | | DR |
| A0002660 | La Jolla Institute for | La Jolla, CA | | EBV | Cell | DUCAF | | | DR |
| | Immunology | | | transformed | line | | | | |
| | | | | B-cell | | | | | |
| A0002661 | La Jolla Institute for | La Jolla, CA | | EBV | Cell | KAS116 | DP | DQ | DR |
| | Immunology | | | transformed | line | | | | |
| | | | | B-cell | | | | | |
| A0002666 | La Jolla Institute for | La Jolla, CA | | EBV | Cell | LZL | DP | DQ | DR |
| | Immunology | | | transformed | line | | | | |
| | | | | B-cell | | | | | |
| A0002667 | La Jolla Institute for | La Jolla, CA | | EBV | Cell | RM3 (Raji | | | DR |
| | Immunology | | | transformed | line | derivative) | | | |
| | | | | B-cell | | | | | |
| A0002668 | La Jolla Institute for | La Jolla, CA | | EBV | Cell | RML | DP | DQ | DR |
| | Immunology | | | transformed | line | | | | |
| | | | | B-cell | | | | | |
| A0002669 | La Jolla Institute for | La Jolla, CA | | EBV | Cell | SPACH | DP | | DR |
| | Immunology | | | transformed | line | | | | |
| | | | | B-cell | | | | | |
| A0002670 | La Jolla Institute for | La Jolla, CA | | EBV | Cell | VAVY | DP | DQ | DR |
| | Immunology | | | transformed | line | | | | |
| | | | | B-cell | | | | | |
| A0002671 | La Jolla Institute for | La Jolla, CA | | EBV | Cell | WT47 | DP | DQ | DR |
| | Immunology | | | transformed | line | | | | |
| | | | | B-cell | | | | | |
| A0002672 | La Jolla Institute for | La Jolla, CA | | EBV | Cell | WT51 | | DQ | DR |
| | Immunology | | | transformed | line | | | | |
| | | | | B-cell | | | | | |
| A0002682 | La Jolla Institute for | La Jolla, CA | | EBV | Cell | AMAI | DP | DQ | DR |
| | Immunology | | | transformed | line | | | | |
| | | | | B-cell | | | | | |
| A0002683 | La Jolla Institute for | La Jolla, CA | | EBV | Cell | BM15 | DP | DQ | DR |
| | Immunology | | | transformed | line | | | | |
| | | | | B-cell | | | | | |
| A0002684 | La Jolla Institute for | La Jolla, CA | | EBV | Cell | COX | DP | DQ | DR |
| | Immunology | | | transformed | line | | | | |
| | | | | B-cell | | | | | |
| A0002685 | La Jolla Institute for | La Jolla, CA | | EBV | Cell | HHKB | | | DR |
| | Immunology | | | transformed | line | | | | |
| | | | | B-cell | | | | | |
| A0002686 | La Jolla Institute for | La Jolla, CA | | EBV | Cell | HID | DP | DQ | DR |
| | Immunology | | | transformed | line | | | | |
| | | | | B-cell | | | | | |
| A0002687 | La Jolla Institute for | La Jolla, CA | | EBV | Cell | H0301 | | | DR |
| | Immunology | | | transformed | line | | | | |
| | | | | B-cell | | | | | |
| A0002688 | La Jolla Institute for | La Jolla, CA | | EBV | Cell | KT3 | | | DR |
| | Immunology | | | transformed | line | | | | |
| | | | | B-cell | | | | | |
| A0002689 | La Jolla Institute for | La Jolla, CA | | EBV | Cell | MGAR | DP | DQ | DR |
| | Immunology | | | transformed | line | | | | |
| | | | | B-cell | | | | | |
| A0002690 | La Jolla Institute for | La Jolla, CA | | EBV | Cell | PITOUT | DP | DQ | DR |
| | Immunology | | | transformed | line | | | | |
| | | | | B-cell | | | | | |
| A0002691 | Proteogenex | Culver City, CA | Breast | IDC | | | | | DR |
| A0002692 | Proteogenex | Culver City, CA | Breast | IDC | | | | | DR |
| A0002694 | Proteogenex | Culver City, CA | Breast | IDC | | | | | DR |
| A0002701 | Proteogenex | Culver City, CA | Breast | IDC | | | | | DR |
| A0002844 | Proteogenex | Culver City, CA | Lung | LUSC | | | | | DR |
| A0002914 | La Jolla Institute for | La Jolla, CA | | EBV | Cell | C1R-A*6801 | | DQ | DR |
| | Immunology | | | transformed | line | | | | |
| | | | | B-cell | | | | | |
| A0002916 | La Jolla Institute for | La Jolla, CA | | EBV | Cell | KAS011 | DP | | DR |
| | Immunology | | | transformed | line | | | | |
| | | | | B-cell | | | | | |
| A0002920 | La Jolla Institute for | La Jolla, CA | | EBV | Cell | LUY | DP | | DR |
| | Immunology | | | transformed | line | | | | |
| | | | | B-cell | | | | | |
| A0002921 | La Jolla Institute for | La Jolla, CA | | EBV | Cell | LWAGS | DP | | DR |
| | Immunology | | | transformed | line | | | | |
| | | | | B-cell | | | | | |
| A0002922 | La Jolla Institute for | La Jolla, CA | | EBV | Cell | PF97387 | | DQ | |
| | Immunology | | | transformed | line | | | | |
| | | | | B-cell | | | | | |
| A0002923 | La Jolla Institute for | La Jolla, CA | | EBV | Cell | PREISS | | | DR |
| | Immunology | | | transformed | line | | | | |
| | | | | B-cell | | | | | |
| A0002924 | La Jolla Institute for | La Jolla, CA | | EBV | Cell | SWEIG | DP | DQ | |
| | Immunology | | | transformed | line | | | | |
| | | | | B-cell | | | | | |
| A0002925 | La Jolla Institute for | La Jolla, CA | | EBV | Cell | WTAIL | | DQ | DR |
| | Immunology | | | transformed | line | | | | |
| | | | | B-cell | | | | | |
| A0003020 | Proteogenex | Culver City, CA | Ovary | OV | | | | | DR |
| A0003021 | Proteogenex | Culver City, CA | Ovary | OV | | | | | DR |
| A0003028 | Proteogenex | Culver City, CA | Ovary | OV | | | | | DR |
| A0003031 | Proteogenex | Culver City, CA | Ovary | OV | | | | | DR |
| A0003032 | Proteogenex | Culver City, CA | Ovary | OV | | | | | DR |
| A0003033 | Proteogenex | Culver City, CA | Ovary | OV | | | | | DR |
| A0003034 | Proteogenex | Culver City, CA | Ovary | OV | | | | | DR |
| A0003046 | Proteogenex | Culver City, CA | Ovary | OV | | | | | DR |
| A0003047 | Proteogenex | Culver City, CA | Ovary | OV | | | | | DR |
| A0003067 | Proteogenex | Culver City, CA | Lung | LUSC | | | | | DR |
| A0003068 | Proteogenex | Culver City, CA | Lung | LUSC | | | | | DR |
| A0003069 | Proteogenex | Culver City, CA | Lung | LUSC | | | | | DR |
| A0003072 | Proteogenex | Culver City, CA | Lung | LUSC | | | | | DR |
| A0003150 | iSpecimen | Lexington, MA | Colon | COAD | | | | | DR |
| A0003170 | iSpecimen | Lexington, MA | Colon | COAD | | | | DQ | |
| A0003337 | ProteoGenex | Culver City, CA | Larynx | HNSC | | | DP | DQ | |
| A0003338 | ProteoGenex | Culver City, CA | Larynx | HNSC | | | | DQ | |
| A0003340 | ProteoGenex | Culver City, CA | Larynx | HNSC | | | | | DR |
| A0003341 | ProteoGenex | Culver City, CA | Larynx | HNSC | | | DP | DQ | |
| A0003342 | ProteoGenex | Culver City, CA | Larynx | HNSC | | | | | DR |
| A0003344 | ProteoGenex | Culver City, CA | Larynx | HNSC | | | | DQ | |
| A0003345 | ProteoGenex | Culver City, CA | Larynx | HNSC | | | | | DR |
| A0003353 | ProteoGenex | Culver City, CA | Larynx | HNSC | | | DP | | DR |
| A0003354 | ProteoGenex | Culver City, CA | Larynx | HNSC | | | DP | | DR |
| A0003355 | ProteoGenex | Culver City, CA | Larynx | HNSC | | | | | DR |
| A0003356 | ProteoGenex | Culver City, CA | Larynx | HNSC | | | | | DR |
| A0003357 | ProteoGenex | Culver City, CA | Larynx | HNSC | | | | | DR |

TABLE 10
KRAS MHC II Peptides

| Peptide | Peptide Sequence |
|---|---|
| 000 | VVGACGVGKSALTIQ |
| 001 | VGACGVGKSALTIQ |
| 002 | VGACGVGKSALTI |
| 003 | YKLVVVGACGVGK |
| 004 | KLVVVGACGVGKS |
| 005 | EYKLVVVGAC |
| 006 | VVVGACGVGKSALT |
| 007 | YKLVVVGACGVGKSALTI |
| 008 | KLVVVGACGVGKSAL |
| 009 | LVVVGACGVGKSALTIQ |
| 010 | MTEYKLVVVGACGVGK |
| 011 | MTEYKLVVVGACGVGKSAL |
| 013 | LVVVGACGVGK |
| 015 | GACGVGKSALTIQL |
| 016 | LVVVGACGVGKSALTIQLI |
| 017 | VVVGACGVGKSA |
| 018 | CGVGKSALTIQL |
| 019 | LVVVGACGVGKSALTI |
| 020 | KLVVVGACGVGKSALTIQ |
| 021 | KLVVVGACGVGK |
| 022 | TEYKLVVVGACGVG |
| 023 | VVGACGVGKSALTI |
| 024 | CGVGKSALTIQLI |
| 025 | TEYKLVVVGACGVGK |
| 026 | MTEYKLVVVGACGVGKS |
| 027 | TEYKLVVVGAC |
| 028 | KLVVVGACGVGKSALTIQL |
| 029 | YKLVVVGACGVG |
| 030 | TEYKLVVVGACGVGKS |
| 031 | VVGACGVGKS |
| 032 | VVVGACGVGKSALTIQLIQ |
| 033 | VVGACGVGKSALTIQLIQ |
| 034 | LVVVGACGVGKSALT |
| 035 | EYKLVVVGACGVGKSALTI |
| 036 | LVVVGACGVGKSA |
| 037 | MTEYKLVVVGACGVGKSA |
| 038 | VVVGACGVGKSALTIQL |
| 039 | VGACGVGKSA |
| 040 | MTEYKLVVVGACGV |
| 041 | KLVVVGACGV |
| 042 | LVVVGACGVGKSAL |
| 044 | TEYKLVVVGACGVGKSALTI |
| 045 | GACGVGKSALTI |
| 046 | LVVVGACGVGKS |
| 047 | VGACGVGKSAL |
| 048 | ACGVGKSALTIQLIQ |
| 049 | VVGACGVGKSAL |
| 050 | TEYKLVVVGACG |
| 051 | EYKLVVVGACG |
| 052 | VGACGVGKSALTIQL |
| 053 | TEYKLVVVGACGVGKSAL |
| 054 | GACGVGKSAL |
| 055 | EYKLVVVGACGVG |
| 057 | CGVGKSALTIQLIQ |
| 058 | EYKLVVVGACGVGKSALTIQ |
| 059 | YKLVVVGACGVGKSALTIQL |
| 060 | MTEYKLVVVGACGVG |
| 061 | VVVGACGVGKS |
| 062 | CGVGKSALTI |
| 063 | KLVVVGACGVGKSA |
| 064 | EYKLVVVGACGVGKSA |
| 065 | ACGVGKSALTIQLI |
| 066 | KLVVVGACGVGKSALTI |
| 067 | VVGACGVGKSALTIQL |
| 068 | YKLVVVGACGVGKSALT |
| 069 | VVVGACGVGKSAL |
| 070 | VGACGVGKSALTIQLIQ |
| 071 | VGACGVGKSALTIQLI |
| 072 | YKLVVVGACGVGKSA |
| 073 | MTEYKLVVVGACG |
| 074 | YKLVVVGACG |
| 075 | YKLVVVGACGVGKS |
| 076 | VVGACGVGKSALTIQLI |
| 077 | VVGACGVGKSALT |
| 079 | LVVVGACGVGKSALTIQLIQ |
| 080 | VVVGACGVGKSALTIQ |
| 081 | EYKLVVVGACGV |
| 082 | CGVGKSALTIQ |
| 083 | ACGVGKSALTI |
| 084 | KLVVVGACGVG |
| 085 | EYKLVVVGACGVGKS |
| 086 | VVVGACGVGK |
| 088 | ACGVGKSALTIQL |
| 089 | GACGVGKSALTIQ |
| 090 | YKLVVVGACGV |
| 091 | KLVVVGACGVGKSALTIQLI |
| 092 | LVVVGACGVG |
| 096 | GACGVGKSALT |
| 097 | VVGACGVGKSA |
| 098 | MTEYKLVVVGAC |
| 099 | GACGVGKSALTIQLI |
| 100 | EYKLVVVGACGVGKSALT |
| 101 | TEYKLVVVGACGVGKSA |
| 102 | ACGVGKSALT |
| 103 | VVVGACGVGKSALTI |
| 104 | ACGVGKSALTIQ |
| 105 | TEYKLVVVGACGV |
| 106 | EYKLVVVGACGVGKSAL |
| 107 | TEYKLVVVGACGVGKSALT |
| 108 | YKLVVVGACGVGKSALTIQ |
| 109 | EYKLVVVGACGVGK |
| 110 | VVVGACGVGKSALTIQLI |
| 111 | MTEYKLVVVGACGVGKSALT |
| 112 | KLVVVGACGVGKSALT |
| 113 | LVVVGACGVGKSALTIQL |
| 114 | YKLVVVGACGVGKSAL |
| 115 | VGACGVGKSALT |
| 116 | GACGVGKSALTIQLIQ |

TABLE 11
Summary of MHC Class II Responses for Healthy Donors

| Sample | HLA-A | HLA-B | HLA-C | DRB1 | Response | Single peptide |
|---|---|---|---|---|---|---|
| SE-0386 | A*02:01 | B*44:03 | C*02:02 | *07:01:01 | Pool 3 | N/A |
| | A*31:01 | B*44:05 | C*16:01 | *11:04:01 | | |
| SE-0659 | A*02:05 | B*44:03 | C*04:01 | *07:01:01 | Pool 3 | Peptide 40 |
| | A*03:01 | B*50:01 | C*06:02 | *15:03:01 | | |
| AC16443 | A*03:01 | B*07:02 | C*07:02 | *01:01:01 | Pool 2 | Peptide 91 |
| | A*03:01 | B*44:02 | C*07:04 | *15:01:01 | | |
| AC13990 | A*02:01 | B*40:01 | C*03:04 | *01:01:01 | Pool 2 | N/A |
| | A*31:01 | B*49:01 | C*07:01 | *15:01:01 | | |
| AC10002 | A*02:01 | B*44:02 | C*05:01 | *07:01:01 | Pool 3 | N/A |
| | A*29:02 | B*44:03 | C*16:01 | *13:01:01 | | |
| AC11223 | A*02:01 | B*13:02 | C*06:02 | *07:01:01 | Pool 3 | Peptide 40 |
| | A*29:02 | B*41:02 | C*17:03 | *13:03:01 | | |
| ST0118 | A*02:01 | B*14:02 | C*01:02 | *01:01:01 | Pool 2 + Pool 3 | N/A |
| | A*68:02 | B*27:05 | C*08:02 | *01:02:01 | | |

TABLE 12
Cell lines for MHC Class II KRAS
G12C mass spectrometry validation

| Cell | | KRAS |
|---|---|---|
| Line | DRB1 Haplotype | G12C Construct |
| Daudi | DRB1*13:01 | KRAS G12C 25mer |
| | DRB1*13:02 | |
| Raji | DRB1*03:01 | KRAS G12C 25mer |
| Ramos | DRB1*07:01 | KRAS G12C 25mer |
| MGar | DRB1*15:01 | KRAS G12C 25mer |
| | DRB1*15:01 | |
| KAS116 | DRB1*01:01 | KRAS G12C 25mer |
| | DRB1*01:01 | |
| DBB | DRB1*07:01 | KRAS G12C 25mer |
| | DRB1*07:01 | |
| MGar | DRB1*15:01 | KRAS 4x4 |
| | DRB1*15:01 | |
| Ramos | DRB1*07:01 | KRAS 4x4 |

TABLE 13
KRAS Neoepitope Cassettes

KRAS 4X (4x4) Cassette
MTEYKLVVVGACGVGKSALTIQLIQMTEYKLVVVGADGVGKSALTIQLI
QETCLLDILDTAGHEEYSAMRDQYMRMTEYKLVVVGADGVGKSALTIQL
IQMTEYKLVVVGAVGVGKSALTIQLIQMTEYKLVVVGACGVGKSALTIQ
LIQETCLLDILDTAGHEEYSAMRDQYMRMTEYKLVVVGADGVGKSALTI
QLIQMTEYKLVVVGAVGVGKSALTIQLIQMTEYKLVVVGACGVGKSALT
IQLIQETCLLDILDTAGHEEYSAMRDQYMRMTEYKLVVVGADGVGKSAL
TIQLIQMTEYKLVVVGAVGVGKSALTIQLIQETCLLDILDTAGHEEYSA
MRDQYMRMTEYKLVVVGAVGVGKSALTIQLIQMTEYKLVVVGACGVGKS
ALTIQLIQGPGPGAKFVAAWTLKAAAGPGPGQYIKANSKFIGITELGPG
PG
KRAS G12C Cassette
MTEYKLVVVGACGVGKSALTIQLIQ

TABLE 14
Antibodies used for immunoprecipitation for mass spectrometry

| Antibody | |
|---|---|
| Name | Specificity |
| W6/32 | Class I HLA-A, B, C |
| L243 | Class II - HLA-DR |
| Tu36 | Class II - HLA-DR |
| LN3 | Class II - HLA-DR |
| Tu39 | Class II - HLA-DR, DP, DQ |
| B7/21.1 | Class II - HLA-DP |
| SVPL3.1 | Class II - HLA-DQ |

TABLE 15
MHC Class II Peptide Candidates Identified by Mass Spectrometry

| | Daudi_ | Raji_ | Ramos_ | MGar_ | KAS116_ | DBB_ | MGar_ | Ramos_ |
|---|---|---|---|---|---|---|---|---|
| Peptide/HLA | 25 mer | 25 mer | 25 mer | 25 mer | 25 mer | 25 mer | 4x4 | 4x4 |
| GACGVGKSALTIQLIQ | DR | | | | | | | |
| GACGVGKSALTIQLI | | DP, DQ | | | | | | |
| KLVVVGACGVGKSALT | DQ | | | | | | | |
| KLVVVGACGVGKSALTI | | | | | | DP | | DP |
| KLVVVGACGVGKSALTIQ | DR, DQ | | DR | | | | | |
| KLVVVGACGVGKSALTIQL | DP | | | | DQ | | | |
| LVVVGACGVGKSAL | | | | | | DR | | |
| LVVVGACGVGKSALTIQLI | | | | | DP | | | |
| LVVVGACGVGKSALTIQLIQ | | | DP | | | | | DP |
| MTEYKLVVVGAC | | | | | | DP | | |
| MTEYKLVVVGACGVGK | | | DR | | | | | DR |
| MTEYKLVVVGACGVGKSA | | | DP | | | | | |
| MTEYKLVVVGACGVGKSAL | | | | | DR | | | |
| TEYKLVVVGACGVGKSALTI | | | DR | | | | | |
| VGACGVGKSALTIQL | DQ | | | | DP | | | |
| VVGACGVGKSAL | | | | | | | | DP |
| VVGACGVGKSALTIQ | | DP | | | | | | |
| VVVGACGVGKSAL | | | | | | DC | | |
| VVVGACGVGKSALT | DQ | | | | | | | |
| VVVGACGVGKSALTI | | | | | DR | | | |
| YKLVVVGACGVG | | | | DQ | | | | |
| YKLVVVGACGVGKSALTI | DR | | | | | | | |
| YKLVVVGACGVGKSALTIQ | DP | | | | | | | |
| YKLVVVGACGVGKSALTIQL | | | | | DP | | | |
| DRB1 | DRB1*13: | DRB1* | DRB1* | DRB1* | DRB1*01: | DRB1*07: | DRB1* | DRB1* |
| | 01, | 03:01 | 07:01 | 15:01 | 01 | 01 | 15:01 | 07:01 |
| | DRB1*13: | | | | | | | |
| | 02 | | | | | | | |
| DRB | DRB3*02: | DRB3* | DRB4* | DRB5*01: | | DRB4* | DRB5* | DRB4* |
| | 02, | 02:02 | 01:01 | 01 | | 01:03 | 01:01 | 01:01 |
| | DRB3*03: | | | | | | | |
| | 01 | | | | | | | |
| DQB1 | DQB1*06: | DQB1* | DQB1* | DQB1*03: | DQB1*05: | DQB1* | DQB1* | DQB1* |
| | 02, | 02:01, | 02:02 | 01, | 01, | 03:03, | 03:01, | 02:02 |
| | DQB1*06: | DQB1* | | DQB1*06: | DQB1*05: | DQB1* | DQB1* | |
| | 04 | 05:01 | | 02 | 01 | 03:03 | 06:02 | |
| DQA1 | DQA1*01: | DQA1* | DQA1* | DQA1*01: | DQA1*01: | DQA1* | DQA1* | DQA1* |
| | 02, | 01:05, | 02:01 | 02 | 01 | 02:01 | 01:02 | 02:01 |
| | DQA1*01: | DQA1* | | | | | | |
| | 03 | 05:01 | | | | | | |
| DPB1 | DPB1*02: | DPB1* | DPB1* | DPB1*04: | DPB1*13: | DPB1* | DPB1* | DPB1* |
| | 01, | 01:01 | 04:01, | 01 | 01 | 04:01 | 04:01 | 04:01, |
| | DPB1*106: | | | | | | | DPB1* |
| | 01 | | DPB1* | | | | | 104:01 |
| | | | 104:01 | | | | | |
| DPA1 | DPA1*01: | DPA1* | DPA1* | DPA1*01 | DPA1*02: | DPA1* | DPA1* | DPA1* |
| | 03, | 02:02 | 01:03 | | 01 | 01:03 | 01 | 01:03 |
| | DPA1*02: | | | | | | | |
| | 01 | | | | | | | |

TABLE 16
No clinical or pathologic features associate with
patients with MSS-CRC who have molecular response.

| | | | Median TMB | | | | |
|---|---|---|---|---|---|---|---|
| | | | (mutations | | Baseline | SD | Best % Change |
| | Primary | Presence | per | | ctDNA | per | in Target |
| | Tumor | of Liver | megabase, | | (mean VAF, | RECIS | Lesions |
| | Location | lesions | range) | PD-L1¹ | range) | Tv1.1 | (mean, range) |
| MR | Colon: 3 | 4 | 2.2 | <1%: 3 | 4.6% | 5 | 1.2 |
| (n = 6) | Rectum: 3 | | (1.6-3.9) | NE: 3 | (0.06-13.8%) | | (−21.7%-10.5%) |
| No MR | Colon: 3 | 4 | 2.7 | <1%: 3 | 21.2% | 1 | 36.2 |
| (n = 4) | Rectum: 1 | | (1.7-5.3) | NE: 1 | (2.6-32.8%) | | (15.5%-56%) |

¹PD-L1 staining on tumor tissue prior to the vaccine regimen.

1. A method for predicting whether an epitope sequence is presented or not presented by one or more class II MHC alleles of a genotype, the method comprising:
combining the epitope sequence and sequences of the one or more class II MHC alleles of the genotype to generate one or more epitope-allele encodings;
providing the one or more epitope-allele encodings as input to a first machine learning model to generate one or more learned representations of the one or more epitope-allele encodings;
transforming the one or more learned representations of the one or more epitope-allele encodings using a learned genotype network to generate a single prediction vector accounting for contributions of each of the one or more class II MHC alleles; and
analyzing the prediction vector using a second machine learning model to generate a genotype presentation score representing a likelihood of presentation of the epitope sequence by the one or more of the class II MHC alleles of the genotype.

2. The method of claim 1, wherein transforming the learned representation of the one or more epitope-allele encoding using a learned genotype network comprises combining weighted combinations of the one or more learned representations.

3. The method of claim 2, wherein the learned genotype network comprises a plurality of learned weights, wherein each learned weight is specific for a class II MHC allele.

4. The method of claim 2, wherein combining weighted combinations of the one or more learned representations comprises:
for each of the one or more learned representations, modifying the learned representation using a learned weight of the learned genotype network; and
summating the one or more modified learned representations.

5. The method of claim 3, wherein a larger value of a learned weight indicates that a corresponding class II MHC allele contributes more heavily towards presentation of the epitope sequence in comparison to a class II MHC allele corresponding to a smaller value of a learned weight.

6. The method of claim 3, wherein a learned weight of the learned genotype network is specific for a kth class II MHC allele and is determined based on at least a non-linear transform of a learned representation an epitope-allele encoding of the kth class II MHC allele.

7. The method of claim 6, wherein the non-linear transform influences the learned weight specific for the kth class II MHC allele based on a learned importance of the kth class II MHC allele for presentation of epitopes.

8-9. (canceled)

10. The method of claim 1, wherein the first machine learning model comprises a protein language model.

11. The method of claim 1, wherein the first machine learning model comprises a neural network.

12. (canceled)

13. The method of claim 1, wherein combining the epitope sequence and sequences of the one or more class II MHC alleles comprises concatenating the epitope sequence and sequences of the one or more class II MHC alleles.

14. (canceled)

15. The method of claim 1, wherein the one or more class II MHC alleles are expressed in the genotype of a patient.

16-20. (canceled)

21. The method of claim 1, wherein one or more of the first machine learning model, the learned genotype network, or the second machine learning model are trained using training data generated by performing mass spectrometry.

22-23. (canceled)

24. The method of claim 1, wherein one or more of the first machine learning model, the learned genotype network, or the second machine learning model are trained using intermediate resolution data generated by performing HLA-DR, HLA-DQ, and HLA-DP specific pulldown of class II MHC alleles.

25. (canceled)

26. The method of claim 1, wherein the first machine learning model, the learned genotype network, and the second machine learning model are jointly trained.

27. The method of claim 1, wherein the first machine learning model, the learned genotype network, and the second machine learning model are trained through two or more phases, wherein a training phase of the two or more training phases comprises one or more of:
a training phase using single allelic training data;
a training phase using intermediate resolution data comprising DR-specific, DQ-specific, and DP-specific immunoaffinity purified mass spectrometry presentation data; and
a training phase using multi-allelic training data.

28-30. (canceled)

31. The method of claim 1, wherein the epitope sequence comprises a KRAS epitope sequence, optionally wherein the KRAS epitope sequence comprises a G12 mutation, optionally wherein the G12 mutation is a G12C, G12V, G12D, or G12A mutation, optionally wherein the KRAS epitope sequence comprises a Q61 mutation, optionally wherein the Q61 mutation is a 061H mutation.

32-34. (canceled)

35. The method of claim 1, further comprising selecting the epitope sequence for inclusion in a vaccine.

36-37. (canceled)

38. The method of claim 1, further comprising identifying one or more T-cells that are antigen-specific for the selected epitope sequence.

39-41. (canceled)

42. A composition comprising one or more epitope sequences, wherein at least one of the one or more epitope sequences are predicted to be presented by one or more class II MHC alleles of a genotype using the method of claim 1.

43-44. (canceled)

45. A non-transitory computer readable medium for predicting whether an epitope sequence is presented or not presented by one or more class II MHC alleles of a genotype, the non-transitory computer readable medium comprising instructions that, when executed by a processor, cause the processor to:
combine the epitope sequence and sequences of the one or more class II MHC alleles of the genotype to generate one or more epitope-allele encodings;
provide the one or more epitope-allele encodings as input to a first machine learning model to generate one or more learned representations of the one or more epitope-allele encodings;
transform the one or more learned representations of the one or more epitope-allele encodings using a learned genotype network to generate a single prediction vector accounting for contributions of each of the one or more class II MHC alleles; and
analyze the prediction vector using a second machine learning model to generate a genotype presentation score representing a likelihood of presentation of the epitope sequence by the one or more of the class II MHC alleles of the genotype.

46-90. (canceled)
